(19) United States
(12) Patent Application Publication
(10) Pub. No.: US 2007/0087363 A1
(43) Pub. Date: Apr. 19, 2007

(54) Therapeutic methods, compounds and compositions

(75) Inventors: Paul L. Bartel, Salt Lake City, UT (US); Jean-Marc Roch, Salt Lake City, UT (US); Janice Sugiyama, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/523,767

(22) Filed: Sep. 18, 2006

Related U.S. Application Data
(63) Continuation-in-part of application No. 10/776,013, filed on Feb. 9, 2004, now abandoned.
Application No. 10/776,013 is a continuation-in-part of application No. 09/948,904, filed on Sep. 10, 2001, now abandoned.
Application No. 10/776,013 is a continuation-in-part of application No. 09/975,072, filed on Oct. 12, 2001, now abandoned.
Application No. 10/776,013 is a continuation-in-part of application No. 10/194,967, filed on Jul. 15, 2002, now abandoned.
Application No. 09/948,904 is a division of application No. 09/466,139, filed on Dec. 21, 1999, now abandoned.
(60) Provisional application No. 60/717,799, filed on Sep. 16, 2005.
(60) Provisional application No. 60/748,419, filed on Dec. 7, 2005.
(60) Provisional application No. 60/751,918, filed on Dec. 19, 2005.
(60) Provisional application No. 60/802,018, filed on May 19, 2006.
(60) Provisional application No. 60/113,534, filed on Dec. 22, 1998.
(60) Provisional application No. 60/124,120, filed on Mar. 12, 1999.
(60) Provisional application No. 60/141,243, filed on Jun. 30, 1999.
(60) Provisional application No. 60/240,790, filed on Oct. 17, 2000.
(60) Provisional application No. 60/304,775, filed on Jul. 13, 2001.

(51) Int. Cl. A61K 48/00; A61K 31/506; A61K 31/501; A61K 31/497; A61K 31/496; C12Q 1/68; C12Q 1/26

(52) U.S. Cl. 435/6; 435/25; 514/44; 514/252.02; 514/252.14; 514/253.01; 514/252.13

(57) ABSTRACT

The invention provides methods of treating, preventing, delaying the onset, slowing the progression, or reversing the symptoms of Alzheimer's disease and other neurodegenerative diseases characterized by the accumulation of amyloid plaques comprising the Aβ42 peptide. The invention also provides compounds that reduce the production or secretion of the Aβ42-peptide by cells, and pharmaceutical compositions comprising such compounds, for the treatment of neurodegenerative diseases characterized by the accumulation of amyloid plaques comprising the Aβ42 peptide.

### CROSS-REFERENCE TO RELATED APPLICATIONS

This application is a continuation-in-part of U.S. patent application Ser. No. 10/776,013 filed Feb. 9, 2004, which is a continuation-in-part of U.S. patent application Ser. No. 09/948,904 filed Sep. 10, 2001, U.S. patent application Ser. No. 09/975,072 filed Oct. 12, 2001, and U.S. patent application Ser. No. 10/194,967 filed Jul. 15, 2002, each of which is hereby incorporated by reference in its entirety. This application also claims the benefit of U.S. provisional patent application Ser. No. 60/717,799, filed Sep. 16, 2005, U.S. provisional patent application Ser. No. 60/748,419, filed Dec. 7, 2005, U.S. provisional patent application Ser. No. 60/751,918, filed Dec. 19, 2005, and U.S. provisional patent application Ser. No. 60/802,018, filed May 19, 2006, each of which is hereby incorporated by reference in its entirety.

U.S. patent application Ser. No. 09/948,904 filed Sep. 10, 2001 is a divisional application of U.S. patent application Ser. No. 09/466,139 filed Dec. 21, 1999, which claims the benefit of U.S. provisional patent application Ser. No. 60/113,534 filed Dec. 22, 1998, U.S. provisional patent application Ser. No. 60/124,120 filed Mar. 12, 1999, and U.S. provisional patent application Ser. No. 60/141,243 filed Jun. 30, 1999 each of which is hereby incorporated by reference in its entirety.

U.S. patent application Ser. No. 09/975,072 filed Oct. 12, 2001 claims the benefit of U.S. provisional patent application Ser. No. 60/240,790 filed Oct. 17, 2000, which is hereby incorporated by reference in its entirety.

U.S. patent application Ser. No. 10/194,967 filed Jul. 15, 2002 claims the benefit of U.S. provisional patent application Ser. No. 60/304,775 filed Jul. 13, 2001, which is hereby incorporated by reference in its entirety.

### FIELD OF THE INVENTION

The invention relates to the use of therapeutic compounds in treating diseases. In particular, the present invention is in the field of medicinal chemistry and relates to the use of compounds that lower Aβ42 peptide production in vivo. Such methods are potentially useful for the treatment and prevention of Alzheimer's disease and other neurodegenerative diseases in which excessive quantities of Aβ42 are produced and secreted from cells, and accumulate in amyloid plaques in brain tissue.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure. AD most commonly begins after the age of 60 with the risk increasing with age. Younger people can also get AD, but it is much less common. It is estimated that 3 percent of men and women ages 65 to 74 have AD. Almost half of those ages 85 and older may have the disease. AD is not a normal part of aging. AD is a complex disease that can be caused by genetic and environmental factors.

In 1906, Dr. Alois Alzheimer, noticed changes in the brain tissue of a woman who had died of an unusual mental illness. In her brain tissue, he found abnormal clumps (now known as amyloid plaques) and tangled bundles of fibers (now known as neurofibrillary tangles) which, today, are considered the pathological hallmarks of AD. Other brain changes in people with AD have been discovered. For example, with AD, there is a loss of nerve cells in areas of the brain that are vital to memory and other mental abilities. Scientists have also found that there are lower levels of chemicals in the brain that carry complex messages back and forth between nerve cells. AD may disrupt normal thinking and memory by blocking these messages between nerve cells.

Plaques and tangles are found in the same brain regions that are affected by neuronal and synaptic loss. Neuronal and synaptic loss is universally recognized as the primary cause in decline of cognitive function. The number of tangles is more highly correlated with cognitive decline than amyloid load in patients with AD (Albert PNAS 93:13547-13551 (1996)). The specific cellular, biochemical, and molecular events responsible for neuronal and synaptic loss in AD are not known. A number of studies have demonstrated that amyloid can be directly toxic to neurons (Iversen et al. Biochem. J. 311:1-16 (1995); Weiss et al. J. Neurochem. 62:372-375 (1994); Lorenzo et al. Ann N Y Acad. Sci. 777:89-95 (1996); Storey et al. Neuropathol. Appl. Neurobiol. 2:81-97 (1999)), resulting in behavioral impairment. The toxicity of amyloid or tangles is potentially aggravated by activation of the complement cascade (Rogers et al. PNAS 21:10016-10020 (1992); Rozemuller et al. Res. Immunol. 6:646-9 (1992); Rogers et al. Res Immunol. 6:624-30 (1992); Webster et al. J. Neurochem. 69(1):388-98 (1997)). This suggests involvement of inflammatory processes in AD and neuronal death seen in AD (Fagarasan et al. Brain Res. 723(1-2):231-4. (1996); Kalaria et al. Neurodegeneration. 5(4):497-503 (1996); Kalaria et al. Neurobiol Aging. 17(5):687-93 (1996); Farlow Am J Health Syst Pharm. 55 Suppl. 2:S5-10 (1998)).

Evidence that amyloid β protein (Aβ) deposition into plaques causes some forms of AD was provided by genetic and molecular studies of certain familial forms of AD (FAD). (See, e.g., Ii Drugs Aging 7(2):97-109 (1995); Hardy PNAS 94(6):2095-7 (1997); Selkoe J. Biol. Chem. 271(31):18295-8 (1996)). The amyloid plaque buildup seen in AD patients suggests that abnormal processing of Aβ may be a cause of AD. Recently, the link between APP over-expression, and Aβ amyloidosis, in both Down Syndrome (DS) and AD patients, has been further strengthened by the discovery of several independent duplications of the APP locus on chromosome 21 in French families with a variable, autosomal dominant phenotype between the pure AD phenotype seen in most families with APP mutations, and the cerebral hemorrhage phenotype of Dutch angiopathy associated with the APP E693Q (Dutch) mutation. These findings highlight the importance of APP gene dosage and provide strong support for the amyloid hypothesis, which postulates that accumulation of β-amyloid in amyloid plaques in the brain drives the neuropathogenesis seen in both AD and DS patients. Rovelet-Lecrux, et al. Nat. Genet. 38:24-26 (2006). The Aβ peptide can contain from 39 to 42 amino acids, with the longest form (Aβ142) forming the core of senile plaques observed in all AD cases. If abnormal processing is the primary cause of AD, then familial Alzheimer's disease (FAD) mutations that are linked (genetically) to FAD may induce changes that, in one way or another, foster Aβ deposition. There are 3 FAD genes known so far (Hardy et al. Science 282:1075-9 (1998); Ray et al. (1998)). Mutations in any of these FAD genes can result in increased A# deposition.

The first of the 3 FAD genes codes for the Aβ precursor, amyloid precursor protein (APP) (Selkoe J. Biol. Chem. 271(31):18295-8 (1996)). Mutations in the APP gene are very rare, but all of them cause AD with 100% penetrance and result in elevated production of either total Aβ or Aβ42, both in model transfected cells and transgenic animals. The other two FAD genes code for presenilin 1 and presenilin 2 (PS1, PS2) (Hardy PNAS 94(6):2095-7 (1997)). The presenilins contain 8 transmembrane domains and several lines of evidence suggest that they are involved in intracellular protein trafficking. Other studies suggest that the presenilins function as proteases. Mutations in the presenilin genes are more common than in the APP genes, and all of them also cause FAD with 100% penetrance. Similar to APP mutants, studies have demonstrated that PS1 and PS2 mutations alter APP metabolism, resulting in elevated Aβ42 production (in vitro and in vivo).

In the United States alone, four million adults suffer from AD (AD). Not only is AD significantly impacting the lives of countless families today, it is threatening to become even more of a problem as the baby boom generation matures. The economic burden of AD is estimated to cost over $ 100 billion a year and the average lifetime cost per patient is estimated to be $174,000. Unfortunately, there is no cure available for AD. Of the five drugs currently being used in the US for the treatment of AD, four of them-tacrine (Cognex®), donepezil (Aricept®), rivastigmine (Exelon®), and galantamine (Reminyl®)—are inhibitors of acetylcholinesterase. Another drug, memantine, was recently approved for treating moderate-to-severe AD. More recently it was reported that memantine showed efficacy in treating mild-to-moderate AD. Memantine is a NMDA receptor antagonist.

The drugs currently used for treating AD, including memantine and the acetylcholine esterase inhibitors, are marginally efficacious and have undesirable side-effects. Thus, there is a large unmet need for better and safer drugs for the treatment or prevention, or for the delay of onset of symptoms, or the reversal of symptoms, of AD and other neurodegenerative diseases characterized by the deposition of amyloid plaques comprising the Aβ42 peptide.

Cerebral amyloid angiopathy (CAA)—also known as cerebrovascular amyloidosis, congophilic angiopathy, and dysphoric angiopathy—is characterized by the deposition of β-amyloid in the media and adventitia of small- and medium-sized arteries (and less frequently, veins) of the cerebral cortex and leptomeninges. Widely recognized as a component of other disorders in which β-amyloid is deposited in the brain, such as AD and DS, CAA is not associated with systemic amyloidosis, which is caused by the aggregation of proteins other than β-amyloid. Although CAA is recognized as one of the morphologic hallmarks of AD, it is often found in the brains of elderly patients who are otherwise neurologically healthy, and show no signs of dementia. However, while often asymptomatic, CAA can result in, and present as, intracranial hemorrhage (ICH), dementia, or transient neurologic events, with ICH being the most commonly observed effect of CAA. While the vast majority of CAA cases are sporadic, at least two familiar forms are known (i.e., hereditary cerebral hemorrhage with amyloidosis [HCHWA]-Dutch type and HCHWA-Icelandic type).

CAA is recognized by its characteristic pathophysiology. Specifically, the deposition of β-amyloid damages the media and adventitia of cortical and leptomeningeal vessels, leading to thickening of the basal membrane, stenosis of the vessel lumen, and fragmentation of the internal elastic lamina. This can result in fibrinoid necrosis and micro-aneurysm formation, predisposing a patient to ICH. Impaired elimination and accumulation of soluble and insoluble β-amyloid peptide likely underlies the pathogenesis and explains the link between CAA and AD.

At present, CAA can only be accurately diagnosed postmortem, hence its true incidence and prevalence is hard to quantify. However, estimates can be made based on autopsies and the incidence of ICH events. For example, a series of 400 autopsies found evidence of CAA in the brains of 18.3% of men and 28% of women aged 40-90 years. In a series of 117 autopsies of brains of patients with confirmed AD, 83% had evidence of CAA. The prevalence of CAA increases with advancing age; in some autopsy series it has been found in 5% of the brains of individuals in the seventh decade (aged 60-69), but in 50% of the brains of individuals older than 90 years.

CAA is estimated to account for up to 15% of all ICH in patients older than 60 years of age, and up to 50% of nontraumatic lobar ICH in patients older than 70 years, which, in turn, accounts for approximately 15-20 cases per 100,000 people per year. CAA and CAA-related hemorrhage are particularly common in elderly individuals with AD and middle-aged patients with DS.

The growing appreciation of the incidence of CAA in elderly individuals, both with and without AD, and in middle-aged DS patients indicates that there is a large unmet need for safe and effective drugs for the treatment, prevention, delay of onset, or reversal, of symptoms of CAA in such patients. Drugs that effectively lower Aβ42 peptide production in the brains of such patients, thereby slowing or stopping the deposition of β-amyloid in the media and adventitia of small- and medium-sized arteries (and less frequently, veins) of the cerebral cortex and leptomeninges, should meet this need in these patients.

Individuals with trisomy 21, or Down syndrome (DS), develop a clinical syndrome of dementia that has the same neuropathological characteristics as described in AD patients without DS. The principle difference in AD neuropathology between individuals with DS and those without DS, is the age of onset. It is estimated that 10-25% of patients with DS develop AD-like dementia at age 40-49, 20-50% develop AD-like dementia at age 50-59, and 60-75% develop AD-like dementia when older than 60 years. AD-like dementia decreases survival in people with DS who are older than 45 years, but not ever person with DS will develop symptoms of AD-like dementia, even if, upon autopsy, their brain reveals the neuropathologic changes commonly associated with AD.

The first evidence for a link between DS and AD came when Blenner and Wong reported the isolation and identification of the same β-amyloid peptide in the meningeal vessels of individuals with either DS or AD. Glenner & Wong Biochem. Biophys. Res. Commun. 122:1131-1135 (1984). Subseqent mapping of the gene encoding the amyloid β precursor protein (APP) to chromosome 21 suggested that the extra copy of the APP gene possessed by trisomy-21 (DS) patients resulted in elevated expression of APP, which, in turn, resulted in increased levels of β-amyloid peptide and accelerated accumulation of β-amyloid plaques. Recently, the link between APP over-expression, and Aβ amyloidosis, in both DS and AD patients, has been further strengthened by the discovery of several independent duplications of the APP locus on chromosome 21 in French families with a variable, autosomal dominant phenotype between the pure AD phenotype seen in most families with APP mutations, and the cerebral hemorrhage phenotype of Dutch angiopathy associated with the APP E693Q (Dutch) mutation. These findings highlight the importance of APP gene dosage and provide strong support for the amyloid hypothesis, which postulates that accumulation of β-amyloid in the brain drives the neuropathogenesis seen in both AD and DS patients. Rovelet-Lecrux, et al. Nat. Genet. 38:24-26 (2006).

As improved health care leads to more and more DS patients surviving into middle age and beyond, there is a increasing need for safe, effective drugs to treat, slow or prevent the onset of dementia that almost inevitably occurs in aging DS patients. Drugs that effectively lower Aβ42 peptide production in the brains of such patients, and thereby slow or stop the aggregation of β-amyloid plaques in these patients' brains, should meet this need, and should reduce the incidence of dementia in aging DS patients.

The present invention provides novel methods for identifying compounds useful for the treatment of mild cognitive impairment (MCI), Alzheimer's disease (AD), cerebral amyloid angiopathy (CAA) and dementia associated with Down syndrome (DS), and other diseases or disorders associated with the accumulation of β-amyloid plaques. The present invention further provides novel methods of treating, delaying the onset of symptoms, slowing the progression of symptoms, or reversing the symptoms of MCI, AD, CAA, or dementia associated with DS. The present invention also provides novel methods for altering the amount of Aβ42 in a tissue or organ of a human patient. Finally, the present invention provides specific compounds for use in these methods.

### BRIEF SUMMARY OF THE INVENTION

The present invention is in the field of medicinal chemistry and relates to the use of compounds to decrease the production of the amyloid plaque forming peptide, Aβ42, by human cells. The methods described herein are useful for treating, delaying the onset of symptoms, or slowing the progression of symptoms of MCI, AD, CAA, or dementia associated with DS and other neurodegenerative diseases characterized by the formation or accumulation of amyloid plaques comprising the Aβ42 peptide.

The nexus of the invention is the discovery by the inventors that overexpression of Stearoyl-CoA desaturase (SCD) in human cells in culture leads to a specific increase in the production of the amyloid plaque-forming peptide Aβ42, and, conversely, that reductions in SCD activity in human cells in culture leads leads to a specific decrease in the production of Aβ42. The reductions in SCD activity that lead to a specific decrease in the production of Aβ42 can result from decreased expression of SCD in the cell, or alternatively, from inhibiting the enzymatic activity of SCD in the cell.

The present invention provides methods for identifying compounds useful for the treatment of MCI, AD, CAA, and dementia associated with DS that comprise selecting compounds that reduce SCD activity in cells, wherein the selected compounds that reduce SCD activity are useful for the treatment of MCI, AD, CAA, and dementia associated with DS, and any other disease associated with the accumulation of β-amyloid plaques. The methods provided include those wherein the selecting step comprises contacting test compounds with SCD protein, or with a cell expressing SCD protein. The test compounds to be selected by these methods include compounds that either reduce the expression of SCD, or inhibit the enzymatic activity of SCD.

Compounds that reduce the expression of SCD include compounds that reduce the amount of SCD-encoding transcripts in cells, and compounds that reduce the translation of such transcripts. Examples of the former are small interfering RNAs (siRNAs) or small hairpin RNAS (shRNAs) that act by inducing RNA interference, which leads to cleavage and destruction of SCD-encoding transcripts. Examples of the latter include antisense nucleic acids that interfere with the initiation, or some other aspect of the translation of SCD-encoding transcripts. Both of these classes of compounds, which are nucleic acids based, necessarily possess sufficient sequence complementarity to the SCD-encoding transcripts to be acted upon, such that these compounds are able to specifically hybridize to the SCD-encoding transcripts within cells.

Compounds that inhibit the enzymatic activity of SCD may do so in a competitive, or non-competitive fashion. They can be any manner of chemical compound, so long as they have sufficient affinity for SCD to allow them to bind specifically to the enzyme, and so long as they have sufficient efficacy (e.g., ability to inhibit the enzymatic activity of SCD). Such SCD-inhibiting compounds can include both natural products, or small molecules synthesized by man. Ideally, these SCD-inhibiting compounds exhibit sufficient solubility to allow them to be contacted with SCD protein directly in vitro, or to be contacted with cells expressing SCD, either in vitro, or in vivo. Also, ideally, these SCD-inhibiting compounds, as well as those compound that reduce the expression of SCD in cells, will exhibit chemical and physical characteristics to allow them to be used to make pharmaceutical formulations, which can be administered in therapeutically effective, SCD-inhibiting, amounts to a patient in need of such treatment.

The present invention also provides methods for determining whether the compounds identified as either effective in reduce the expression of SCD, or inhibiting the enzymatic activity of SCD, also reduce the amount of Aβ42 produced by a test cell. These methods include methods that may be conducted on test cells in a cell culture, or test cells isolated from a cell culture. These methods also include methods that can be conducted in a cell-free assay, such as an assay of conditioned media isolated from a cell culture treated with a compound of the invention.

The present invention further provides methods of treating, delaying the onset of symptoms, or slowing the progression of symptoms of MCI, AD, CAA or dementia associated with DS, comprising identifying a patient in need of such treatment, and administering a a therapeutically effective amount of a compound that reduces SCD activity in cells. Additionally, the present invention provides methods of altering the amount of Aβ42 in a tissue or organ of a human patient comprising identifying a patient in need of such treatment, administering a a therapeutically effective amount of a compound that reduces SCD activity in cells. Such methods may further comprise the step of confirming that the amount of Aβ42 in the tissue of the patient has been altered. In such methods, the compound that reduces SCD activity in cells ideally lowers the amount or concentration of Aβ42 in the brain, or in the plasma, or, alternatively, raises the amount or concentration of Aβ42 in the cerebrospinal fluid (CSF). Confirmation of the alteration can be via any appropriate assay, such as by enzyme-linked immunosorbant assays (ELISAs) of plasma or CSF. However, confirmation of the alteration in the brain of a living patient can involve determining whether or not the administered compound leads to a decrease in the density, number or size of amyloid plaques in the patient's brain. Such determinations will require a more sophisticated techniques, involving, e.g., positron emission tomography (PET scanning) with an appropriate tracer compound that specifically binds to, or accumulates in amyloid plaques.

The present invention provides specific examples of compounds that reduce SCD activity in cells by reducing the expression of SCD in cells. Examples of such compounds include siRNAs designed to specifically target the SCD1 transcript and promote its destruction by inducing RNA interference. Examples of such compounds also include antisense nucleic acids that are designed to hybridize to the SCD1 transcript and block the initiation of its translation.

The present invention also provides specific examples of compounds that reduce SCD activity in cells by inhibiting the enzymatic activity of SCD, and can be used in the methods of the present invention. Among these compounds are various piperazine, pyridazine, and pyridyl derivatives, which were described in PCT International Patent Application Publications WO 2005/011653 (published Feb. 10, 2005), WO 2005/011654 (published Feb. 10, 2005), WO 2005/011655 (published Feb. 10, 2005), WO 2005/011656 (published Feb. 10, 2005), and WO 2005/011657 (published Feb. 10, 2005), which correspond to PCT International Patent Application Serial Nos. PCT/US2004/024541, PCT/US2004/024542, PCT/US2004/024548, PCT/US2004/024657, and PCT/US2004/024658, respectively, and which are hereby incorporated by reference in their entirety. A subset of the pyridazine compounds described in these PCT International Patent Application Publications, were also described in U.S. Patent Application Publications 2005/0065143, 2005/0119251, and US2006/0205713, which correspond to U.S. patent application Ser. Nos. 10/901,563 and 10/566,856, respectively. The contents of the U.S. patent Applications are hereby incorporated by reference in their entirety. These various patent applications and publications not only provide a description of SCD-inhibiting compounds that can be used in the methods of the present invention, but they also provide the methods by which these compounds can be synthesized. These methods are hereby specifically incorporated by reference in their entirety.

In certain embodiments, the present invention provides methods of treating, preventing, delaying the onset or progression of symptoms, or reversing the symptoms of neurodegenerative diseases, comprising administration of a therapeutically effective amount of an SCD-inhibiting, Aβ42-lowering, composition, to a patient in need of such treatment; said composition comprising a compound according to any of Formulae I-V(g)(2), wherein the substituent groups and their locations are as described below:

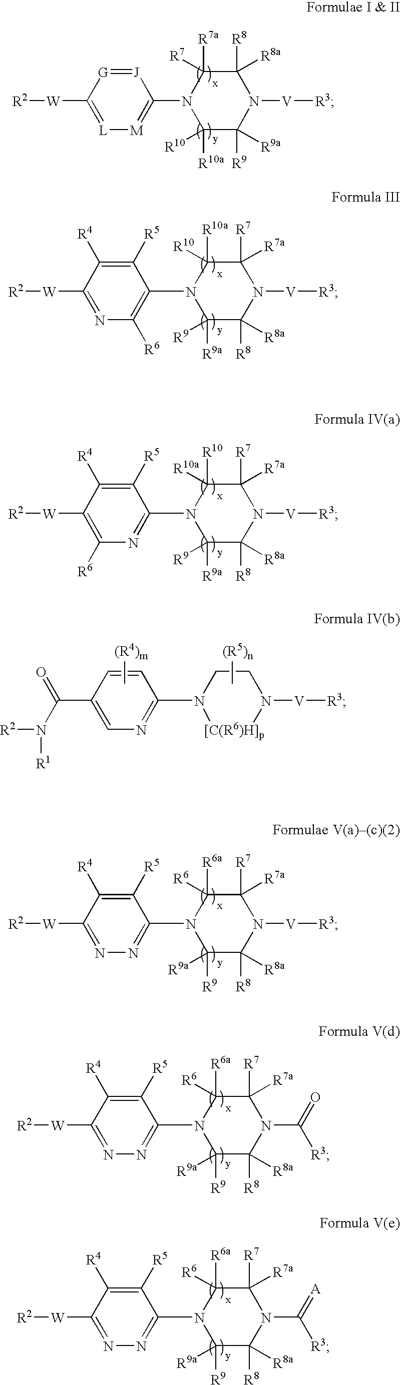

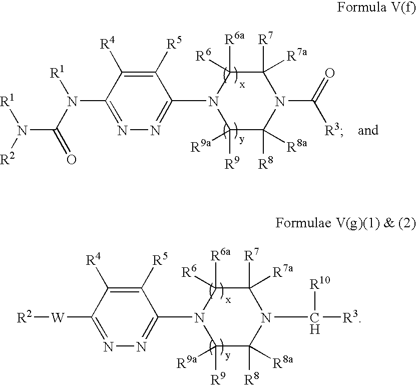

The present invention further encompasses the use of the compounds of the invention for the preparation of pharmaceutical compositions and the use of such compositions for treating, delaying the onset of symptoms, slowing the progression of symptoms, or reversing the symptoms, of neurodegenerative diseases characterized by the accumulation of amyloid plaques comprising the Aβ42 peptide, in patients in need of such treatment. In particular, the pharmaceutical compositions of the present invention can be used for treating, delaying the onset of symptoms, slowing the progression of symptoms, or reversing the symptoms, of such diseases and disorders as MCI, AD, CAA or dementia associated with DS, which are characterized by the formation or accumulation of amyloid plaques, comprising the Aβ42 peptide, in the brains of patients in need of such treatment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 depicts siRNAs specifically designed to target human transcripts encoding SCD1, and promote their degradation through the induction of RNA interference;

FIG. 2 shows the effect of SCD overexpression of SCD on the amount of Aβ40 and Aβ42 peptides secreted by transfected H4 cells;

FIG. 3 shows the effect of active and inactive isoforms of conjugated linoleic acids on the secretion of Aβ40 and Aβ42 peptides when contacted with HEK293 cells;

FIG. 4 shows the effect of siRNAs targeting SCD transcripts on the secretion of Aβ40 and Aβ42 peptides when contacted with HEK293 cells at different concentrations, for different times; and

FIG. 5 shows the effect of siRNAs targeting SCD transcripts on the secretion of Aβ40 and Aβ42 peptides when contacted with HEK293 cells for 24 hours (top panels) or 48 hours (bottom panels), plated and grown at low densities (left panels) or high densities (right panels), relative to cell survival.

### DETAILED DESCRIPTION OF THE INVENTION

### Stearoyl-CoA Desaturases and Aβ42 Production by Cells

Stearoyl-CoA desaturases (SCDs) are acyl desaturases that catalyze a rate-limiting step in the synthesis of unsaturated fatty acids. They are integral membrane proteins of the endoplasmic reticulum (ER) that introduce a double bond in the C9-C10 position of saturated fatty acids, particularly in preferred substrates palmitoyl-CoA (16:0) and stearoyl-CoA (18:0). The products of the SCD reaction, such as palmitoleoyl-CoA (16:1) and oleoyl-CoA (18:1), are incorporated into phospholipids, triglycerides, and cholesteryl esters, some of which end up in the membranes of the cells in which they are produced, and thereby influence membrane fluidity and signal transduction.

Although four different SCD isoforms have been identified in the mouse, in contrast, only two isoforms (SCD1 & SCD5) have been identified in humans (Zhang et al., Biochem. J. 340 (Pt 1):255-264 (1999)). SCD1 shares about 85% amino acid identity with all 4 mouse SCD isoforms, as well as with rat Scd1 and Scd2. In contrast, SCD5 shares limited homology with the rodent SCDs and appears to be unique to primates (Zhang et al., Biochem. J. 340 (Pt 1):255-264 (1999); Wang et al., Biochem. Biophys. Res. Commun. 332:735-742 (2005)). While SCD1 and SCD5 are distinct enzymes encoded by two different genes, when overexpressed in transfected cells, they displayed similar delta 9 desaturase activity (Wang et al., Biochem. Biophys. Res. Commun. 332:735-742 (2005).

As described in U.S. patent application Ser. No. 10/776,013, the inventors discovered that a carboxyl-terminal fragment of SCD1 (comprising amino acid residues 320-359) interacts with the calcium and integrin binding protein 1 (CIB1), which itself interacts with both PS1 and PS2 (Stabler et al., J Cell Biol 145:1277-1292, (1999)). See Example 1, below. Based on the indirect association of SCD with the presenilins, and knowing that Aβ production can be modulated by local membrane lipid composition (Puglielli et al., Nat Neurosci 6:345-351 (2003)), the inventors hypothesized that SCD activity might affect APP processing Aβ42 production. In agreement with this hypothesis, the inventors discovered that overexpression of human SCD in neuronal H4 cells expressing APP resulted in increased secretion of Aβ42 (the more pathogenic, but less abundant Aβ peptide species) without changing the amount of secreted Aβ40 (the more abundant peptide species). See Example 2, below. This observation suggested that decreasing SCD activity in cells might result in reduced levels of Aβ42 being produced—a therapeutically desirable outcome.

Reports from other research groups have shown that conjugated linoleic acid (CLA) isomers can inhibit SCD and diminish its enzymatic activity (Gomez et al., BBRC 300:316-326 (2003); Park et al., Biochim Biophys Acta 1486:285-292 (2000); Choi et al., J Nutr 130:1920-1924 (2000); Choi et al., BBRC 284:689-693 (2001); Choi et al., BBRC 294:785-790 (2002)). These reports and other results reported in U.S. patent application Ser. No. 10/776,013, indicating that CLA inhibition of SCD lowers Aβ42 secretion, strengthen the connection between SCD activity and Aβ42 production. See Example 3, below. The implication of these observations is clear: by reducing SCD activity within cells, Aβ42 production can be decreased, which would be expected to reduce the amount of Aβ42 available to form, or otherwise increase the density, numbers or size of amyloid plaques in the brains of patients suffering from neurodegenerative disorders characterized by the deposition of such plaques.

Thus, while not wishing to be bound by theory, the inventors believe that they have identified a novel therapeutic approach for of treating, delaying the onset of symptoms, slowing the progression of symptoms, or reversing the symptoms of MCI, AD, CAA, dementia associated with DS, or any other neurodegenerative diseases or disorders characterized by the accumulation of amyloid plaques in the brain. Specifically, the inventors believe that by reducing SCD activity in cells, Aβ42 production can be decreased, such that less Aβ42 peptide is available to form, or otherwise increase the density, numbers or size of amyloid plaques in the brains of patients suffering from neurodegenerative disorders characterized by the deposition of such plaques.

Consequently, the present invention provides methods for identifying compounds that reduce SCD activity in cells; thereby identifying compounds that lower the production of Aβ42 peptide by such cells; and thereby identifying compounds that can be used for treating, delaying the onset of symptoms, slowing the progression of symptoms, or reversing the symptoms of MCI, AD, CAA, dementia associated with DS, or any other neurodegenerative diseases or disorders characterized by the accumulation of amyloid plaques in the brain. The present invention also provides specific compounds that can be used in these methods, and that act by either reducing the amount of SCD expressed in cells, or by inhibiting the enzymatic activity of the SCD protein itself. The present invention further provides pharmaceutical compositions comprising the therapeutic compounds of the present invention and a pharmaceutically acceptable excipient or carrier, for treating, delaying the onset of symptoms, slowing the progression of symptoms, or reversing the symptoms of MCI, AD, CAA, dementia associated with DS, or any other neurodegenerative diseases or disorders characterized by the accumulation of amyloid plaques in the brain. Such pharmaceutical compositions are formulated in order to deliver a therapeutically effective, or prophylactically effective, amount of the compound to a patient in need of such treatment. The present invention also provides therapeutic methods that make use of therapeutic compounds and compositions of the invention for the treatment of treatment of patients in need of such treatment.

The present invention and various embodiments thereof are described in more detail following these definitions.

### Definitions

As used herein, the term “preventing,” when used in the context of “preventing a disease or disorder,” refers to both not allowing a symptom to increase or worsen, as well as reducing or slowing the rate of increase or worsening of the symptoms of the disease or disorder. For example, a symptom can be measured as the amount of particular disease marker, i.e., Aβ42 peptide, present in a patient tissue sample. In another example the symptom can be cognitive or behavioral decline in a patient. Preventing an increase, according to the definition provided herein, means that the amount of symptom (e.g., Aβ42 peptide, or cognitive or behavioral decline) does not increase or worsen, or that the rate at which it increases or worsens is reduced.

As used herein, the phrases “treating a neurodegenerative disease,” “treating Alzheimer's disease,” or “treating AD,” or “treating MCI,” or “treating CAA,” or “treating dementia associated with DS” refer to a slowing or stopping of the progression of the disease or disorder, or its symptoms, or refer to a reversal of the disease or disorder, or its symptoms. For example, “treating AD” includes not only treating a disease, but reducing or reversing a symptom or symptoms of that disease.

As used herein, the phrase “preventing a neurodegenerative disease,” or “preventing AD, CAA or dementia associated with DS” refers to a slowing of the disease progression or slowing of the onset of the disease or the symptoms thereof. Preventing Alzheimer's disease can include stopping the onset of the disease or the symptoms thereof, or reversing the symptoms of the disease once they are manifest.

As used herein, the term “Aβ42-lowering” refers the capability of a compound or composition to reduce the amount of Aβ42 present in and/or being produced by cells, either in vitro, in cell culture, or in a patient. Levels of Aβ42 can be determined by a variety of assays, in patient tissue samples and fluids, such as serum, in vitro, in cell culture media, or within living patients. For example, levels of Aβ42 can be determined in patient tissue samples, or cell culture media, by an enzyme-linked immunoabsorbent assay (ELISA) configured to specifically detect Aβ42. Methods for determining Aβ42 levels are described in the examples and in the references cited therein.

In certain embodiments of the present invention, the “Aβ42-lowering” activity of the compound or composition is determined or monitored in tissue samples taken from patients. These tissue samples may include, but are not limited to, serum, plasma, cerebrospinal fluid (CSF), and brain tissue from biopsies. In other embodiments, the “Aβ42-lowering” activity of the compound or composition is determined or monitored within the bodies of living patients using non-invasive imaging techniques, including, but not limited to, positron emission tomography (PET) combined with radioligands that bind amyloid plaques. Such techniques were the subject of a recent review by Mathis et al. (See Mathis et al., Curr Pharm Des. 10:1469-92 (2004)), which is incorporated herein by reference in its entirety. Advances in such techniques, as well as specific methods used, have been described more recently in Klunk et al., Ann. Neurol. 55:306-319 (2004); Price et al., J. Cereb. Blood Flow Metab. 25:1528-1547 (2005); Lopresti et al., J. Nucl. Med. 46:1959-1972 (2005); and Fagan et al., Ann. Neurol. 59:512-519 (2006); which are all incorporated by reference herein in their entirety.

As used herein, the terms “Alzheimer's Disease” or “AD,” include specific gradations thereof referred to herein as “mild,” “moderate,” and “severe.” These terms; “mild,” “moderate,” and “severe;” have specific meaning, in accordance with standard medical practice. Further, the terms “Mild Cognitive Impairment,” “MCI,” “Cerebral Amyloid Angiopathy,” and “CAA” have specific meaning, in accordance with standard medical practice, as does dementia associated with Down Syndrome. Nevertheless, the diagnosis of AD, MCI, CAA, dementia associated with DS, or cognitive decline in general, can be made using any known method in the art. Typically, AD is diagnosed using a combination of clinical and pathological assessments. For example, progression or severity of AD can be determined using: Mini Mental State Examination (MMSE) as described by Mohs et al. Int Psychogeriatr 8:195-203 (1996); Alzheimer's Disease Assessment Scale-cognitive component (ADAS-cog) as described by Galasko et al. Alzheimer Dis Assoc Disord, 11 suppl 2:S33-9 (1997); the Alzheimer's Disease Cooperative Study Activities of Daily Living scale (ADCS-ADL) as described by McKhann et al. Neurology 34:939-944 (1984); and the NINCDS-ADRDA criteria as described by Folstein et al. J. Psychiatr. Res. 12:189-198 (1975). In addition, methods that allow for evaluating different regions of the brain and estimating amyloid plaque and neurofibrillary tangle abundance can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993); and, as mentioned above, non-invasive methods utilizing PET scanning have been reviewed in Mathis et al., Curr Pharm Des. 10:1469-92 (2004), and described further in Klunk et al., Ann. Neurol. 55:306-319 (2004); Price et al., J. Cereb. Blood Flow Metab. 25:1528-1547 (2005); Lopresti et al., J. Nucl. Med. 46:1959-1972 (2005); and Fagan et al., Ann. Neurol. 59:512-519 (2006).

As used herein, the phrase “compounds that reduce SCD activity” means compounds capable of lowering of the total measurable amount of SCD enzymatic activity within a cell, tissue or organ. The phrase “compounds that reduce SCD activity” is meant to include not only those compounds that act by reducing the activity of SCD isoform SCD 1, but also those compounds that reduce the activity of SCD isoform SCD5. (See Zhang et al., Biochem. J. 340 (Pt 1):255-264 (1999); Wang et al., Biochem. Biophys. Res. Commun. 332:735-742 (2005).) Importantly, the phrase “compounds that reduce SCD activity” includes compounds that reduce the total measurable amount of SCD enzymatic activity within a cell, tissue or organ, by reducing the amount of SCD expressed within a cell, tissue or organ, as well as compounds that specifically inhibit the enzymatic activity of SCD. Further, as explained below, compounds that reduce the amount of SCD expressed within a cell, tissue or organ, include compounds that reduce the amount of SCD-encoding transcripts (mRNA) within a cell (e.g., siRNAs and shRNAs), as well as compounds that interfere with the translation of such SCD-encoding transcripts (e.g., antisense nucleic acids).

As used herein, the terms pertaining to the compounds of the invention that specifically inhibit the enzymatic activity of SCD have the meanings as set forth below.

A shorthand notation indicating the total number of carbon atoms that are to be found in the indicated chemical group precedes certain chemical groups named herein. For example; C₇-C₁₂ alkyl describes an alkyl group, as defined below, having a total of seven to twelve carbon atoms, and C₄-C₁₂ cycloalkylalkyl describes a cycloalkylalkyl group, as defined below, having a total of four to twelve carbon atoms. The total number of carbons in the shorthand notation does not include carbons that may exist in substituents of the group described.

Accordingly, as used in the specification and appended claims, unless specified to the contrary, the following terms have the meaning indicated:

“Methoxy” refers to the —OCH₃ radical.

“Cyano” refers to the —CN radical.

“Nitro” refers to the —NO₂ radical.

“Trifluoromethyl” refers to the —CF₃ radical.

“Oxo” refers to the ═O substituent.

“Thioxo” refers to the ═S substituent.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl(iso-propyl), n-butyl, n-pentyl, 1,1-dimethylethyl (t-butyl), and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶) (where t is 1 to 2), —S(O)ₜOR¹⁶ (where t is 1 to 2), —S(O)ₜR¹⁶ (where t is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl (optionally substituted with one or more halo groups), aralkyl, heterocyclyl, heterocylylalkyl, heteroaryl or heteroarylalkyl; and each R¹⁶ is alkyl, haloalkyl, cycloalkyl, cycloalkylalkyl, aryl, aralkyl, heterocyclyl, heterocylylalkyl, heteroaryl or heteroarylalkyl, and where each of the above substituents is unsubstituted unless otherwise indicated.

“C₁-C₃ alkyl” refers to an alkyl radical as defined above containing one to three carbon atoms. The C₁-C₃ alkyl radical may be optionally substituted as defined for an alkyl group.

“C₁-C₆ alkyl” refers to an alkyl radical as defined above containing one to six carbon atoms. The C₁-C₆alkyl radical may be optionally substituted as defined for an alkyl group.

“C₁-C₁₂ alkyl” refers to an alkyl radical as defined above containing one to twelve carbon atoms. The C₁-C₁₂ alkyl radical may be optionally substituted as defined for an alkyl group.

“C₂-C₆ alkyl” refers to an alkyl radical as defined above containing two to six carbon atoms. The C₂-C₆ alkyl radical may be optionally substituted as defined for an alkyl group.

“C₃-C₆ alkyl” refers to an alkyl radical as defined above containing three to six carbon atoms. The C₃-C₆ alkyl radical may be optionally substituted as defined for an alkyl group.

“C₃-C₁₂ alkyl” refers to an alkyl radical as defined above containing three to twelve carbon atoms. The C₃-C₁₂ alkyl radical may be optionally substituted as defined for an alkyl group.

“C₆-C₁₂alkyl” refers to an alkyl radical as defined above containing six to twelve carbon atoms. The C₆-C₁₂alkyl radical may be optionally substituted as defined for an alkyl group.

“C₇-C₁₂ alkyl” refers to an alkyl radical as defined above containing seven to twelve carbon atoms. The C₇-C₁₂ alkyl radical may be optionally substituted as defined for an alkyl group.

“Alkenyl” refers to a straight or branched hydrocarbon chain radical group consisting solely of carbon and hydrogen atoms, containing at least one double bond, having from two to twelve carbon atoms, preferably one to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —OR¹⁴, —OC(O)R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶) (where t is 1 to 2), —S(O)ₜOR¹⁶ (where t is 1 to 2), —S(O)ₜR¹⁶ (where t is 0 to 2), and —S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocylylalkyl, heteroaryl or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“C₃-C₁₂ alkenyl” refers to an alkenyl radical as defined above containing three to twelve carbon atoms. The C₃-C₁₂alkenyl radical may be optionally substituted as defined for an alkenyl group.

“C₂-C₁₂ alkenyl” refers to an alkenyl radical as defined above containing two to twelve carbon atoms. The C₂-C₁₂alkenyl radical may be optionally substituted as defined above for an alkenyl group.

“Alkylene” and “alkylene chain” refer to a straight or branched divalent hydrocarbon chain, linking the rest of the molecule to a radical group, consisting solely of carbon and hydrogen, containing no unsaturation and having from one to twelve carbon atoms, preferably having from one to eight carbons, e.g., methylene, ethylene, propylene, n-butylene, and the like. The alkylene chain may be attached to the rest of the molecule and to the radical group through one carbon within the chain or through any two carbons within the chain.

“Alkenylene” and “alkenylene chain” refer to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain.

“Alkylene bridge” refers to a straight or branched divalent hydrocarbon bridge, linking two different carbons of the same ring structure, consisting solely of carbon and hydrogen, containing no unsaturation and having from one to twelve carbon atoms, preferably having from one to eight carbons, e.g., methylene, ethylene, propylene, n-butylene, and the like. The alkylene bridge may link any two carbons within the ring structure.

“Alkoxy” refers to a radical of the formula —ORₐ where Rₐ is an alkyl radical as defined above. The alkyl part of the alkoxy radical may be optionally substituted as defined above for an alkyl radical.

“C₁-C₆ alkoxy” refers to an alkoxy radical as defined above containing one to six carbon atoms. The alkyl part of the C₁-C₆ alkoxy radical may be optionally substituted as defined above for an alkyl group.

“C₁-C₁₂ alkoxy” refers to an alkoxy radical as defined-above containing one to twelve carbon atoms. The alkyl part of the C₁-C₁₂ alkoxy radical may be optionally substituted as defined above for an alkyl group.

“C₃-C₁₂alkoxy” refers to an alkoxy radical as defined above containing three to twelve carbon atoms. The alkyl-part of the C₃-C₁₂ alkoxy radical may be optionally substituted as defined above for an alkyl group.

“Alkoxyalkyl” refers to a radical of the formula —Rₐ—O—Rₐ where each Rₐ is independently an alkyl radical as defined above. The oxygen atom may be bonded to any carbon in either alkyl radical. Each alkyl part of the alkoxyalkyl radical may be optionally substituted as defined above for an alkyl group.

“C₂-C₁₂ alkoxyalkyl” refers to an alkoxyalkyl radical as defined above containing two to twelve carbon atoms. Each alkyl part of the C₂-C₁₂ alkoxyalkyl radical may be optionally substituted as defined above for an alkyl group.

“C₃ alkoxyalkyl” refers to an alkoxyalkyl radical as defined above containing three carbon atoms. Each alkyl part of the C₃ alkoxyalkyl radical may be optionally substituted as defined above for an alkyl group.

“C₃-C₁₂ alkoxyalkyl” refers to an alkoxyalkyl radical as defined above containing three to twelve carbon atoms. Each alkyl part of the C₃-C₁₂ alkoxyalkyl radical may be optionally substituted as defined above for an alkyl group.

“Alkylsulfonyl” refers to a radical of the formula —S(O)₂Rₐ where Rₐ is an alkyl group as defined above. The alkyl part of the alkylsulfonyl radical may be optionally substituted as defined above for an alkyl group.

“C₁-C₆ alkylsulfonyl” refers to an alkylsulfonyl radical as defined above having one to six carbon atoms. The C₁-C₆ alkylsulfonyl group may be optionally substituted as defined above for an alkylsulfonyl group.

“Aryl” refers to aromatic monocyclic or multicyclic hydrocarbon ring system consisting only of hydrogen and carbon and containing from 6 to 19 carbon atoms, preferably 6 to 10 carbon atoms, where the ring system may be partially or fully saturated. Aryl groups include, but are not limited to, groups such as fluorenyl, phenyl and naphthyl. Unless stated otherwise specifically in the specification, the term “aryl” or the prefix “ar-” (such as in “aralkyl”) is meant to include aryl radicals optionally substituted by one or more substituents selected from the group consisting of alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)R¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR.—sup.¹⁶) (where t is 1 to 2), —R¹⁵—S(O)ₜOR¹⁶ (where t is 1 to 2), —R¹⁵—S(O)ₜR¹⁶ (where t is 0 to 2), and —R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl part of the aralkyl radical may be optionally substituted as described above for an aryl group. The alkyl part of the aralkyl radical may be optionally substituted as defined above for an alkyl group.

“C₇-C₁₂ aralkyl” refers to an aralkyl group as defined above containing seven to twelve carbon atoms. The aryl part of the C₇-C₁₂ aralkyl radical may be optionally substituted as described above for an aryl group. The alkyl part of the C₇-C₁₂aralkyl radical may be optionally substituted as defined above for an alkyl group.

“C₁₃-C₁₉ aralkyl” refers to an aralkyl group as defined above containing thirteen to nineteen carbon atoms. The aryl part of the C₁₃-C₁₉ aralkyl radical may be optionally substituted as described above for an aryl group. The alkyl part of the C₁₃-C₁₉aralkyl radical may be optionally substituted as defined above for an alkyl group.

“Aralkenyl” refers to a radical of the formula —R_{c}R_{b} where R_{c} is an alkenyl radical as defined above and R_{b} is one or more aryl radicals as defined above, which may be optionally substituted as described above. The aryl part of the aralkenyl radical may be optionally substituted as described above for an aryl group. The alkenyl part of the aralkenyl radical may be optionally substituted as defined above for an alkenyl group.

“Aryloxy”-refers to a radical of the formula —OR_{b} where R_{b} is an aryl group as defined above. The aryl part of the aryloxy radical may be optionally substituted as defined above.

Aryl-C₁-C₆ alkyl” refers to a radical of the formula —Rₕ—R¹ where Rₕ is an unbranched alkyl radical having one to six carbons and R¹ is an aryl group attached to the terminal carbon of the alkyl radical.

“Cycloalkyl” refers to a stable non-aromatic monocyclic or bicyclic hydrocarbon radical consisting solely of carbon and hydrogen atoms, having from three to fifteen carbon atoms, preferably having from three to twelve carbon atoms, and which is saturated or unsaturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents selected from the group consisting of alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴—R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶—, —R¹⁵—N(R¹⁴)(S(O)ₜR¹⁶) (where t is 1 to 2), —R¹⁵—S(O)ₜOR¹⁶ (where t is 1 to 2), —R¹⁵—S(O)ₜR¹⁶ (where t is 0 to 2), and —R¹⁵—S(O)ₜN(R¹⁴)₂ (where t is 1 to 2) where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“C₃-C₆ cycloalkyl” refers to a cycloalkyl radical as defined above having three to six carbon atoms. The C₃-C₆ cycloalkyl radical may be optionally substituted as defined above for a cycloalkyl group.

“C₃-C₁₂ cycloalkyl” refers to a cycloalkyl radical as defined above having three to twelve carbon atoms. The C₃-C₁₂ cycloalkyl radical may be optionally substituted as defined above for a cycloalkyl group.

“Cycloalkylalkyl” refers to a radical of the formula —RₐR_{d} where Rₐ is an alkyl radical as defined above and R_{d} is a cycloalkyl radical as defined above. The cycloalkyl part of the cycloalkyl radical may be optionally substituted as defined above for an cycloalkyl radical. The alkyl part of the cycloalkyl radical may be optionally substituted as defined above for an alkyl radical.

“C₄-C₁₂ cycloalkylalkyl” refers to a cycloalkylalkyl radical as defined above having four to twelve carbon atoms. The C₄-C₁₂ cycloalkylalkyl radical may be optionally substituted as defined above for a cycloalkylalkyl group.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkenyl” refers to an alkenyl radical, as defined above, that is substituted by one or more halo radicals, as defined above, e.g., 2-bromoethenyl, 3-bromoprop-1-enyl, and the like. The alkenyl part of the haloalkenyl radical may be optionally substituted as defined above for an alkyl group.

“Heterocyclyl” refers to a stable 3- to 18-membered non-aromatic ring radical which consists of carbon atoms and from one to five heteroatoms selected from the group consisting of nitrogen, oxygen and sulfur. For purposes of this invention, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴—R¹⁵—OC(O)—R¹⁴, —R¹⁵—N(R¹⁴)₂, —R¹⁵—C(O)R¹⁴, —R¹⁵C(O)OR¹⁴, —R¹⁵—C(O)N(R¹⁴)₂, —R¹⁵—N(R¹⁴)C(O)OR¹⁶, —R¹⁵—N(R¹⁴)C(O)R⁶—, —R¹⁵—N(R¹⁴)(S(O)ₜR¹⁶) (where t is 1 to 2), —R¹⁵—S(O)ₜOR¹⁶ (where t is 1 to 2), —R¹⁵—S(O)ₜR¹⁶ (where t is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“C₃-C₁₂ heterocyclyl” refers to a heterocyclyl radical as defined above having three to twelve carbons. The C₃-C₁₂ heterocyclyl may be optionally substituted as defined above for a heterocyclyl group.

“Heterocyclylalkyl” refers to a radical of the formula —RₐRₑ where Rₐ is an alkyl radical as defined above and Rₑ is a heterocyclyl radical as defined above, and if the heterocyclyl is a nitrogen-containing heterocyclyl, the heterocyclyl may be attached to the alkyl radical at the nitrogen atom. The alkyl part of the heterocyclylalkyl radical may be optionally substituted as defined above for an alkyl group. The heterocyclyl part of the heterocyclylalkyl radical may be optionally substituted as defined above for a heterocyclyl group.

“C₃-C₁₂ heterocyclylalkyl” refers to a heterocyclylalkyl radical as defined above having three to twelve carbons. The C₃-C₁₂ heterocyclylalkyl radical may be optionally substituted as defined above for a heterocyclylalkyl group.

“Heteroaryl” refers to a 5- to 18-membered aromatic ring radical which consists of carbon atoms and from one to five heteroatoms selected from the group consisting of nitrogen, oxygen and sulfur. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzothiadiazolyl, benzonaphtho furanyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, furanyl, furanonyl, isothiazolyl, imidazolyl, indolyl, indazolyl, isoindolyl, indolinyl, isoindolinyl, indolizinyl, isoxazolyl, naphthyridinyl, oxadiazolyl, 2-oxoazepinyl, oxazolyl, oxiranyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, triazinyl, and thiophenyl. Unless stated otherwise specifically in the specification, the term “heteroaryl” is meant to include heteroaryl radicals as defined above which are optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR.-sup.¹⁶) (where t is 1 to 2), —R¹⁵—S(O)ₜOR¹⁶ (where t is 1 to 2), —R¹⁵—S(O)ₜR¹⁶ (where t is 0 to 2), and —R¹⁵—S(O)ₜN(R¹⁴)₂ (where t is 1 to 2) where each R¹⁴ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R¹⁵ is independently a direct bond or a straight or branched alkylene or alkenylene chain; and each R¹⁶ is alkyl, alkenyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted.

“C₁-C₁₂ heteroaryl” refers to a heteroaryl radical as defined above having one to twelve carbon atoms. The C₁-C₁₂ heteroaryl group may be optionally substituted as defined above for a heteroaryl group.

“C₅-C₁₂ heteroaryl” refers to a heteroaryl radical as defined above having five to twelve carbon atoms. The C₅-C₁₂ heteroaryl group may be optionally substituted as defined above for a heteroaryl group.

“Heteroarylalkyl” refers to a radical of the formula —RₐR_{f} where Rₐ is an alkyl radical as defined above and R_{f} is a heteroaryl radical as defined above. The heteroaryl part of the heteroarylalkyl radical may be optionally substituted as defined above for a heteroaryl group. The alkyl part of the heteroarylalkyl radical may be optionally substituted as defined above for an alkyl group.

“C₃-C₁₂ heteroarylalkyl” refers to a heteroarylalkyl radical as defined above having three to twelve carbon atoms. The C₃-C₁₂ heteroarylalkyl group may be optionally substituted as defined above for a heteroarylalkyl group.

“Heteroarylcycloalkyl” refers to a radical of the formula —R_{d}R_{f} where R_{d} is a cycloalkyl radical as defined above and R_{f} is a heteroaryl radical as defined above. The cycloalkyl part of the heteroarylcycloalkyl radical may be optionally substituted as defined above for a cycloalkyl group. The heteroaryl part of the heteroarylcycloalkyl radical may be optionally substituted as defined above for a heteroaryl group.

“Heteroarylalkenyl” refers to a radical of the formula —R_{b}R_{f} where R_{b} is an alkenyl radical as defined above and R_{f} is a heteroaryl radical as defined above. The heteroaryl part of the heteroarylalkenyl radical may be optionally substituted as defined above for a heteroaryl group. The alkenyl part of the heteroarylalkenyl radical may be optionally substituted as defined above for an alkenyl group.

“Hydroxyalkyl” refers to a radical of the formula —Rₐ—OH where Rₐ is an alkyl radical as defined above. The hydroxy group may be attached to the alkyl radical on any carbon within the alkyl radical. The alkyl part of the hydroxyalkyl group may be optionally substituted as defined above for an alkyl group.

“C₂-C₁₂ hydroxyalkyl” refers to an hydroxyalkyl radical as defined above containing two to twelve carbon atoms. The alkyl part of the C₂-C₁₂ hydroxyalkyl radical may be optionally substituted as defined above for an alkyl group.

“C₃-C₁₂ hydroxyalkyl” refers to an hydroxyalkyl radical as defined above containing three to twelve carbon atoms. The alkyl part of the C₃-C₁₂ hydroxyalkyl radical may be optionally substituted as defined above for an alkyl group.

“C₇-C₁₂ hydroxyalkyl” refers to an hydroxyalkyl radical as defined above containing seven to twelve carbon atoms. The alkyl part of the C₇-C₁₂ hydroxyalkyl radical may be optionally substituted as defined above for an alkyl group.

“Hydroxyalkenyl” refers to a radical of the formula —R_{c}—OH where R_{c} is an alkenyl radical as defined above. The hydroxy group may be attached to the alkenyl radical on any carbon within the alkenyl radical. The alkenyl part of the hydroxyalkenyl group may be optionally substituted as defined above for an alkenyl group.

“C₂-C₁12hydroxyalkenyl” refers to an hydroxyalkenyl radical as defined above containing two to twelve carbon atoms. The alkenyl part of the C₂-C₁12hydroxyalkenyl radical may be optionally substituted as defined above for an alkenyl group.

“C₃-C₁12hydroxyalkenyl” refers to an hydroxyalkenyl radical as defined above containing three to twelve carbon atoms. The alkenyl part of the C₃-C₁12hydroxyalkenyl radical may be optionally substituted as defined above for an alkenyl group.

“Hydroxyl-C₁-C₆-alkyl” refers to a radical of the formula —Rₕ—OH where Rₕ is an unbranched alkyl radical having one to six carbons and the hydroxy radical is attached to the terminal carbon.

“Trihaloalkyl” refers to an alkyl radical, as defined above, that is substituted by three halo radicals, as defined above, e.g., trifluoromethyl. The alkyl part of the trihaloalkyl radical may be optionally substituted as defined above for an alkyl group.

“C₁-C₆ trihaloalkyl” refers to a trihaloalkyl radical as defined above having one to six carbon atoms. The C₁-C₆ trihaloalkyl may be optionally substituted as defined above for a trihaloalkyl group.

“Trihaloalkoxy” refers to a radical of the formula —OR₉ where R_{g} is a trihaloalkyl group as defined above. The trihaloalkyl part of the trihaloalkoxy group may be optionally substituted as defined above for a trihaloalkyl group.

“C₁-C₆ trihaloalkoxy” refers to a trihaloalkoxy radical as defined above having one to six carbon atoms. The C₁-C₆ trihaloalkoxy group may be optionally substituted as defined above for a trihaloalkoxy group.

“A multi-ring structure” refers to a multicyclic ring system comprised of two to four rings wherein the rings are independently selected from cycloalkyl, aryl, heterocyclyl or heteroaryl as defined above. Each cycloalkyl may be optionally substituted as defined above for a cycloalkyl group. Each aryl may be optionally substituted as defined above for an aryl group. Each heterocyclyl may be optionally substituted as defined above for a heterocyclyl group. Each heteroaryl may be optionally substituted as defined above for a heteroaryl group. The rings may be attached to other through direct bonds or some or all of the rings may be fused to each other. Examples include, but are not limited to a cycloalkyl radical substituted by aryl group; a cycloalkyl group substituted by an aryl group, which, in turn, is substituted by another aryl group; and so forth.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

Prodrugs and active metabolites of compound may be identified using routine techniques known in the art. See, e.g., Bertolini, G et al., J. Med. Chem., 40, 2011-2016 (1997); Shan, D. et al., J. Pharm. Sci., 86 (7), 756-767; Bagshawe K., Drug Dev. Res., 34, 220-230 (1995); Bodor N, Advance in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

The term “prodrug” is also meant to include any covalently bonded carriers which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amine functional groups in the compounds of the invention and the like.

“Stable compound” and “stable structure” are meant to indicate a compound that is sufficiently robust to survive isolation to a useful degree of purity from a reaction mixture, and formulation into an efficacious therapeutic agent.

“Mammal” includes humans and domestic animals, such as cats, dogs, swine, cattle, sheep, goats, horses, rabbits, and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Therapeutically effective amount” refers to that amount of a compound of the invention which, when administered to a mammal, preferably a human, is sufficient to effect treatment, as defined below, of a neurodegenerative disease characterized by the deposition of amyloid plaques in the brain tissue of a mammal, preferably a human. The amount of a compound of the invention which constitutes a “therapeutically effective amount” will vary depending on the compound, the condition and its severity, and the age of the mammal to be treated, but can be determined routinely by one of ordinary skill in the art having regard to his own knowledge and to this disclosure.

The compounds used in the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)- isomers may be prepared using chiral synthons or chiral reagents, or resolved using conventional techniques, such as HPLC using a chiral column. When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

The chemical naming protocol and structure diagrams used herein employ and rely on the chemical naming features as utilized by Chemdraw version 7.0.1. (available from Cambridgesoft Corp., Cambridge, Mass.). For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency. For example, a compound of the following formula:

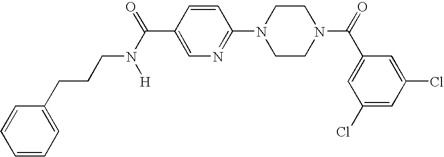

is named herein as 6-[4-(3,5-dichloro-benzoyl)-piperazin-1-yl]-N-(3-phenyl-propyl)-nicotinamide.

### Methods of Identifying Compounds of the Present Invention

The present invention provides methods for identifying compounds of the present invention that reduce the SCD activity within a cell, and thereby decrease the amount of Aβ42 being produced by the cell. Such methods include those methods that directly assess whether SCD1 and or SCD5 expression is reduced in a cell, and methods that determine whether SCD enzymatic activity is reduced through inhibition.

Examples of the former type of assay include assays commonly practiced by molecular biologist assessing level of expression of specific gene products. For example, assays of this type include Northern blot analysis of SCD1-encoding transcripts, and quantitative, or semi-quantitative reverse transcription polymerase chain reactions (RT-PCR). In both of these types of assays, specificity is achieved through the use of nucleic acid probes or primers that specifically hybridize to the SCD1 transcript. General examples of such methods and assays are well known in the art, and can readily be adapted to specifically detect SCD1 and SCD5-encoding transcripts.

Assays that directly assess whether SCD expression is reduced in a cell can also be designed to directly detect levels of the SCD protein expressed. Examples of these types of assays include various immunoassays using antibodies directed to the SCD protein, such as enzyme-linked immunosorbant assays (ELISAs) that use anti-SCD antibodies as the primary binding antibody. In one embodiment, the primary binding antibody is a monoclonal antibody that is specifically immunoreactive with SCD1 or SCD5. General examples of such methods and assays are also well known in the art, and can also be readily adapted to specifically detect SCD1 or SCD5 protein.

The present invention also provides methods that assess whether a test compound inhibits the enzymatic activity of SCD. Such assays are commonly practiced by biochemists skilled in the art of enzymology. Specific examples of such assays are provided in Examples 5 and 6, below.

As a consequence of the inventors discovery that reductions in SCD activity in cells results in reduced production or secretion of Aβ42 peptide by the cell, assays designed to assess Aβ42 peptide production or secretion can also be used to indirectly assess the level of SCD activity in a cell. Such assays are also provided in the Examples 7 and 8 below.

While these assays indirectly assess the level of SCD activity in a cell, they are particularly useful for the methods of the present invention, because they facilitate the selection of compounds of the present invention that can be used to lower Aβ42 peptide production or secretion by lowering the SCD activity in a cell. These assays further allow for the confirmation that SCD-inhibiting compounds can be used in the therapeutic methods of the invention. Specific examples where such assays have been employed are provided in Examples 3 and 4, below.

### Therapeutic Compounds

The therapeutic compound of the present invention can generally be divided into those compounds that reduce SCD activity within a cell by reducing the expression of SCD, and those that reduce SCD activity within a cell by inhibiting the enzymatic activity of SCD. Compounds in the first category include those that reduce the amount of SCD-encoding transcripts (mRNAs) within the cell, and those that interfere with the translation of existing SCD-encoding transcripts. The types of chemical entities that fall within these various groups, and additional differentiation between their respective mechanisms of action will be more apparent after consideration of the following discussion.

A. Compounds that Reduce the Expression of SCD:

The present invention provides short interfering nucleic acids (siNAs), such as siRNAs and shRNAs, that, when contacted with cells, reduce the expression of SCD in those cells, for use in the methods of the invention. Specific examples of such siNAs, siRNAs and shRNAs have been described in U.S. patent application Ser. No. 10/923,451 (filed Aug. 20, 2004), which is hereby incorporated by reference herein in its entirety.

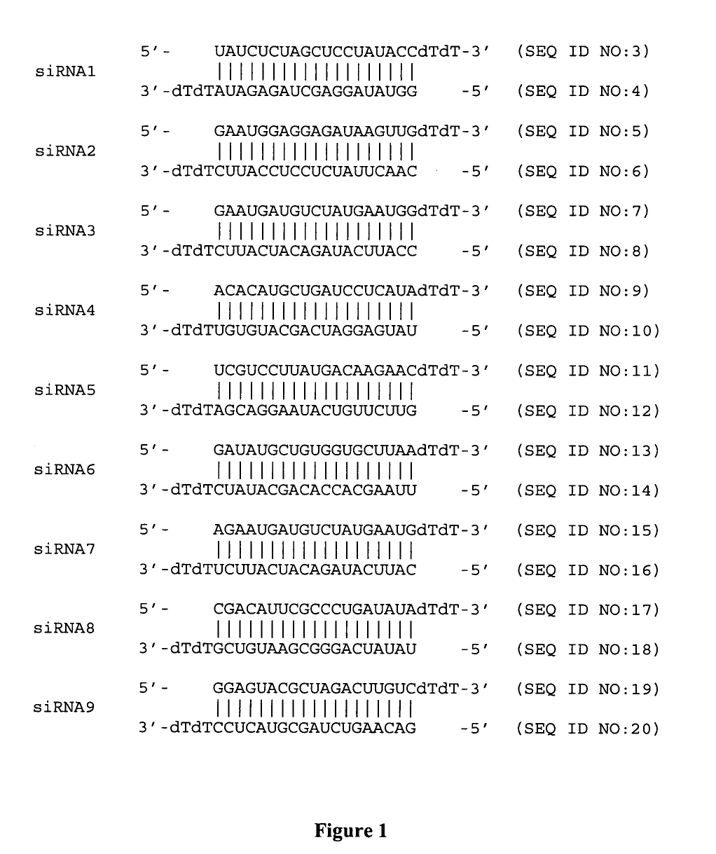

Specific examples of siRNAs targeting SCD1 transcripts are also provided herein, in FIG. 1. Further, the sense and antisense strands that are annealed to form these siRNAs are provided as SEQ ID NOs:3-20 in the Sequence Listing being filed concurrently herewith.

The present invention further provides antisense nucleic acids that reduce the expression of SCD in cells, for use in the methods of the invention. Specific examples of such antisense nucleic acids, and their use in treating diseases other than MCI, AD, CAA, and dementia associated with DS, and other diseases associated with the accumulation of β-amyloid plaques, have been described in U.S. patent application Ser. No. 09/918,187 (filed Jul. 30, 2001), Ser. No. 10/484,442 (filed Jul. 16, 2002), and Ser. No. 10/619,253 (filed Jul. 15, 2003), respectively. The contents, teachings, and specific antisense compounds of these U.S. patent applications are hereby incorporated herein in their entirety.

Specific examples of antisense nucleic acids targeting SCD1 are also provided herein, as SEQ ID NOs:21-32 in the Sequence Listing being filed concurrently herewith.

Methods for the use and formulation of such compounds are known in the art and specifically described in the patent applications listed above.

B. Compounds that Inhibit SCD Enzymatic Activity:

The methods of the present invention also comprise treating MCI, AD, CAA, and dementia associated with DS, and other diseases associated with the accumulation of β-amyloid plaques, by the administration of an effective amount of an SCD-inhibiting, Aβ42-lowering, composition to a patient in need of such treatment. Such therapeutic compositions can comprise compounds that are known to inhibit the enzymatic activity of SCD.

Importantly, while others have suggested the use of pyridyl, pyridazine, piperazine, and nicotinamide derivatives and other inhibitors of SCD for the treatment of diseases such as obesity, diabetes, cancer, atherosclerosis, and inflammation, the inventors are suggesting a novel use of such pyridyl, pyridazine, piperazine, and nicotinamide derivatives and other inhibitors of SCD. Specifically, the inventors are suggesting the use of SCD inhibitors for the treatment of neurodegenerative diseases characterized by the accumulation of amyloid plaques, since the inventors have discovered that reducing SCD activity in cells can reduce the amount of Aβ42 peptide produced or secreted by such cells.

SCD-inhibiting compounds that can be used in the compositions of the present invention can comprise those of Formulae I-V(g)(2). Specifically, compounds in this category can comprise those according to Formulae I-V(g)(2), as defined below.

Specifically, the methods of the present invention comprise administration of an effective amount of an SCD-inhibiting, Aβ42-lowering, composition, to a patient in need of such treatment. Such therapeutic composition can comprises a compound according to Formula I: 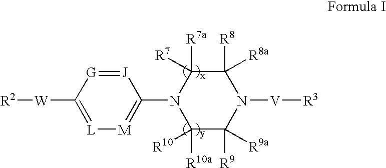
wherein:
x and y are each independently 1, 2 or 3;
W is —N(R¹)C(O)—, —C(O)N(R¹)—, —C(O)N[C(O)R^{1a}]—, —OC(O)N(R¹)—, —N(R¹)C(O)N(R¹)—, —O—, —N(R¹)—, —S(O)ₜ— (where t is 0, 1 or 2), —N(R¹)S(O)ₜ— (where t is 1 or 2), —S(O)₂N(R¹)—, —C(O)—, —OS(O)₂N(R¹)—, —OC(O)—, —C(O)O—, —C(S)N(R¹)—, —OC(S)N(R¹)—, —C(R¹)₂, —N(R¹)C(S)N(R¹)— or —N(R¹)C(O)O—;
V is —C(O)—, —C(O)O—, —C(S)—, —C(S)O—, —C(O)N(R₁)—, —S(O)ₜ— (where t is 1 or 2), —S(O)ₜN(R¹)—(where t is 1 or 2) or —C(R¹¹)H—;
G, J, L and M are each independently selected from —N— or —C(R⁴)—;
each R¹ is independently selected from the group consisting of H, C₁-C₆alkyl optionally substituted with one or more substituents selected from the group consisting of halo, methyl and trifluoromethyl, C₂-C₆alkenyl optionally substituted with one or more substituents selected from the group consisting of methoxy and hydroxyl, C₇-C₁₂alkyl, C₂-C₁₂hydroxyalkyl, C₄-C₁₂cycloalkylalkyl and C₇-C₁₉aralkyl;
R^{1a} is selected from the group consisting of H, C₁-C₆alkyl and cycloalkyl;
R² is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂ heteroarylalkyl; optionally substituted with one or more halo, cyano, oxo, thioxo, nitro, hydroxy, C₁-C₆alkyl, C₁-C₆trihaloalkyl, C₁-C₆trihaloalkoxy, C₁-C₆alkylsulfonyl, —OR¹², —C(O)R¹², N(R¹²)₂, —OC(O)R¹², —C(O)OR¹², —C(O)N(R¹²)₂, or —S(O)₂N(R¹²)₂, cycloalkyl, heterocyclyl, aryl, aralkyl, heteroaryl, and hetreoarylcycloalkyl;
or R² is a multi-ring structure having 2 to 4 rings wherein the rings are independently selected from the group consisting of cycloalkyl, heterocyclyl, aryl and heteroaryl and where some or all of the rings may be fused to each other;
R³ is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl and C₃-C₁₂heteroarylalkyl;
or R³ is an aryl optionally substituted with one or more substituents chosen from halo, cyano, nitro, hydroxy, C₁-C₆alkyl, C₁-C₆trihaloalkyl, C₁-C₆trihaloalkoxy, C₁-C₆alkylsulfonyl, —N(R₁₂)₂, —OC(O)R², —C(O)OR¹², —S(O)₂N(R¹²)₂, cycloalkyl, heterocyclyl, aryl, aralkyl, heteroaryl, and hetreoarylcycloalkyl;
or R³ is a multi-ring structure having 2 to 4 rings wherein the rings are independently selected from the group consisting of cycloalkyl, heterocyclyl, aryl and heteroaryl and where some or all of the rings may be fused to each other;
each R⁴ is independently selected from H, fluoro, chloro, bromo, methyl, methoxy, trifluoromethyl, cyano, nitro or —N(R¹³)₂;
R⁷, R^{7a}, R⁸, R^{8a}, R⁹, R^{9a}, R¹⁰, R^{10a} are each independently selected from H or C₁-C₃alkyl;
or R⁷ and R^{7a} together, or R⁸ and R^{8a} together, or R⁹ and R^{9a} together, or R¹⁰ and R^{10a} together are an oxo group, provided that when V is —C(O)—, R⁸ and R^{8a} together or R⁹ and R^{9a} together do not form an oxo group, while the remaining R⁷, R^{7a} , R⁸, R^{8a}, R⁹, R^{9a}, R¹⁰ and R^{10a} are each independently selected from H or C₁-C₃alkyl;
or one of R⁷, R^{7a}, R⁸, and R^{8a} together with one of R⁹, R^{9a}, R¹⁰ and R^{10a} form an alkylene bridge, while the remaining R⁷, R^{7a}, R⁸, R^{8a}, R⁹, R^{9a}, R¹⁰, and R^{10a} are each independently selected from H or C₁-C₃alkyl;
R¹¹ is H or C₁-C₃alkyl; and
each R¹³ is independently selected from H or C₁-C₆alkyl; or
a stereoisomer, enantiomer or tautomer thereof, a pharmaceutically acceptable salt thereof, a prodrug thereof, and a pharmaceutically acceptable excipient.

The methods of the present invention also comprise administration of an effective amount of an SCD-inhibiting, Aβ42-lowering, composition, to a patient in need of such treatment, wherein the composition comprises a compound according to Formula II:

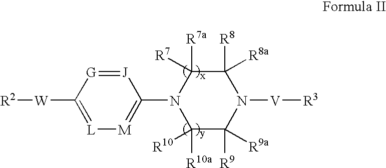

wherein: x and y are each independently 1, 2 or 3;

W is —N(R¹)C(O)—, —C(O)N(R¹)—, —OC(O)N(R¹)—, —N(R¹)C(O)N(R¹)—, —O—, —N(R¹)—, —S(O)ₜ—(where t is 0, 1 or 2), —N(R¹)S(O)₂—, —S(O)₂N(R¹)—, —C(O)—, —OS(O)₂N(R¹)—, —OC(O)—, —C(O)O—, or —N(R¹)C(O)O—;

V is —C(O)—, —C(O)O—, —C(S)—, —C(O)N(R¹)—, —S(O)₂—, —S(O)₂N(R¹)— or —C(R¹⁰)H—;

G, J, L and M are each independently selected from —N═or —C(R^{4)—;}

provided that at least two of G, J, L and M are —N═, and provided that when G and J are both —C(R⁴)—, L and M can not both be —N═, and when L and M are both —C(R⁴)═, G and J can not both be —N═;

each R¹ is independently selected from the group consisting of hydrogen, C₁-C₁₂alkyl, C₂-C₁₂hydroxyalkyl, C₄-C₁₂cycloalkylalkyl and C₇-C₁₉aralkyl;

R² is selected from the group consisting of C₁-C₁₂alkyl, C₂-C₁₂alkenyl, C₂-C₁₂hydroxyalkyl, C₂-C₂hydroxyalkenyl, C₂-C₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂heteroarylalkyl;

or R² is a multi-ring structure having 2 to 4 rings wherein the rings are independently selected from the group consisting of cycloalkyl, heterocyclyl, aryl and heteroaryl and where some or all of the rings may be fused to each other;

R³ is selected from the group consisting of C₁-C₁₂alkyl, C₂-C₁₂alkenyl, C₂-C₁₂hydroxyalkyl, C₂-C₁₂hydroxyalkenyl, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl and C₃-C₁₂heteroarylalkyl;

or R³ is a multi-ring structure having 2 to 4 rings wherein the rings are independently selected from the group consisting of cycloalkyl, heterocyclyl, aryl and heteroaryl and where some or all of the rings may be fused to each other; each R⁴ is independently selected from hydrogen, fluoro, chloro, methyl, methoxy, trifluoromethyl, cyano, nitro or —N(R⁹)₂;

each R⁵, R^{5a} R⁶, R^{6a}, R⁷, R^{7a}, R⁸ and R^{8a} is independently selected from hydrogen or C₁-C₃alkyl; or R⁵ and R^{5a} together, or R⁶ and R^{6a} together, or R⁷ and R^{7a} together, or R⁸ and R^{8a} together are an oxo group, provided that when V is —C(O)—, R⁶ and R^{6a} together or R⁵ and R^{5a} together do not form an oxo group, while the remaining R⁵, R^{5a}, R⁶, R^{6a}, R⁷, R^{7a}, R⁸ and R^{8a} are each independently selected from hydrogen or C₁-C₃alkyl;

or one of R⁵, R^{5a}, R⁶, and R^{6a} together with one of R⁷, R^{7a}, R⁹ and R^{8a} form an alkylen bridge, while the remaining R⁵, R^{5a}, R⁶, R^{6a}, R⁷, R^{7a}, R⁸, and R^{8a} independently selected from hydrogen or C₁-C₃alkyl;

R¹⁰ is hydrogen or C₁-C₃alkyl; and each R⁹ is independently selected from hydrogen or C₁-C₆alkyl;

a stereoisomer, enantiomer or tautomer thereof, a pharmaceutically acceptable salt thereof, a pharmaceutical composition thereof or a prodrug thereof.

The compounds of Formula II include: 4-(2-Trifluoromethyl-benzoyl)-3,4,5,6 -tetrahydro-2H-[1, 2′]bipyrazinyl-5′-carboxylic acid phenethyl-amide; 4-(2-Trifluoromethyl-benzoyl)-3,4,5,6-tetrahydro-2H-[1, 2′]bipyrazinyl-5′-carboxylic acid (3-phenyl-propyl)-amide; 4-(2-Trifluoromethyl-benzoyl)-3,4,5,6-tetrahydro-2H-[1, 2′] bipyrazinyl-5′-carboxylic acid [2-(4-fluoro-phenyl)-ethyl]-amide; 4-(2-Trifluoromethyl-benzoyl)-3,4,5,6-tetrahydro-2H-[1, 2′]bipyrazinyl-5′-carboxylic acid [3-(4-fluoro-phenyl)-propyl]-amide; and 4-(2-Trifluoromethyl-benzoyl)-3,4,5,6-tetrahydro-2H-[1, 2′]bipyrazinyl-5′-carboxylic acid [3-(4-fluoro-phenyl)-propyl]-amide.

The compounds of formula II also include 4-(2-Trifluoromethyl-benzoyl)-3,4,5,6-tetrahydro-2H-[1, 2′]bipyrazinyl-5′-carboxylic acid (3-methyl-butyl)-amide; 4-(2-Trifluoromethyl-benzoyl)-3,4,5,6-tetrahydro-2H-[1, 2′]bipyrazinyl-5′-carboxylic acid (2-phenoxy-ethyl)-amide; and 4-(2-Trifluoromethyl-benzoyl)-3,4,5,6-tetrahydro-2H-[1, 2′]bipyrazinyl-5′-carboxylic acid pentylamide.

The compounds of Formula II also include: 4-trifluoromethyl-2-[4-(2-trifluoromethylbenzoyl)piperazin-1-yl]-pyrimidine-5-carboxylic acid (3-methylbutyl)amide; and 2-[4-(2-Trifluoromethylbenzoyl)piperazin-1-yl]pyrimidine-5-carboxylic acid (3-methylbutyl)amide.

The methods of the present invention also comprise administration of an effective amount of an SCD-inhibiting, Aβ42-lowering, composition, to a patient in need of such treatment, wherein the composition comprises a compound according to Formula III:

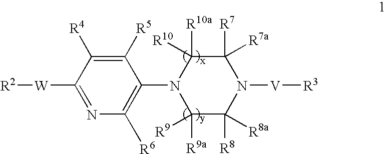

wherein: x and y are each independently 1, 2 or 3;

W is —O—, —N(R¹)—, —C(O)—, —S(O)ₜ—; (where t is 0, 1 or 2), —N(R¹)S(O)₂—, —S(O)₂N(R¹)—, —OS(O)₂N(R¹)—, —C(O)N(R¹)—, —OC(O)N(R¹)—, —C(S)N(R¹)—, —OC(S)N(R¹)—, —N(R¹)C(O)—, or —N(R¹)C(O)N(R¹)—; V is —C(O)—, —C(S)—, —C(O)N(R¹)—, —C(O)O—, —S(O)₂—, —S(O)₂N(R¹)— or —C(R¹¹) H—;

each R¹ is independently selected from the group consisting of hydrogen, C₁-C₁₂alkyl, C₂-C₁₂hydroxyalkyl, C₄-C₁₂cycloalkylalkyl and C₇-C₁₉aralkyl;

R² is selected from the group consisting of C₁-C₁₂alkyl, C₂-C₁₂alkenyl, C₂-C¹²hydroxyalkyl, C₂-C₁₂hydroxyalkenyl, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂heteroarylalkyl;

or R² is a multi-ring structure having 2 to 4 rings wherein the rings are independently selected from the group consisting of cycloalkyl, heterocyclyl, aryl and heteroaryl and where some or all of the rings may be fused to each other;

R³ is selected from the group consisting of C₁-C₁₂alkyl, C₂-C₁₂alkenyl, C₂-C₁₂hydroxyalkyl, C₂-C₁₂hydroxyalkenyl, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl and C₃-C₁₂heteroarylalkyl;

or R³ is a multi-ring structure having 2 to 4 rings wherein the rings are independently selected from the group consisting of cycloalkyl, heterocyclyl, aryl and heteroaryl and where some or all of the rings may be fused to each other;

R⁴, R⁵ and R⁶ are each independently selected from hydrogen, fluoro, chloro, methyl, methoxy, trifluoromethyl, cyano, nitro or —N(R¹³)₂;

R⁷, R^{7a}, R⁸, R^{8a}, R⁹, R^{9a}, R¹⁰ and R^{10a} are each independently selected from hydrogen or C₁-C₃alkyl;

or R⁷ and R^{7a} together, or R⁸ and R^{8a} together, or R⁹ and R^{9a} together, or R¹⁰ and R^{10a} together are an oxo group, provided that when V is —C(O)—, R⁷ and R^{7a} together or R⁸ and R^{8a} together do not form an oxo group, while the remaining R⁷, R^{7a}, R⁸, R^{8a}, R⁹, R^{9a}, R¹⁰, and R^{10a} are each independently selected from hydrogen or C₁-C₃alkyl;

or one of R¹⁰, R^{10a}, R⁷, and R^{7a} together with one of R⁸, R^{8a}, R⁹ and R^{9a} form an alkylen bridge, while the remaining R¹⁰, R^{10a}, R⁷, R^{7a}, R⁸, R^{8a}, R⁹, and R^{9a} are each independently selected from hydrogen or C₁-C₃alkyl;

R¹¹ is hydrogen or C₁-C₃alkyl;

and each R¹³ is independently selected from hydrogen or C₁-C₆alkyl;

a stereoisomer, enantiomer or tautomer thereof, a pharmaceutical acceptable salt thereof, a pharmaceutical composition thereof or a prodrug thereof.

The compounds of Formula III include: 5-[4-(Naphthalene-1-carbonyl)-piperazin-1-yl]-pyridine-2-carboxylic acid (3-phenyl-propyl)-amide; and 5-[4-(Naphthalene-1-carbonyl)piperazin-1-yl]pyridine-2-carboxylic acid phenethylamide.

The compounds of Formula III also include: 4-[2-({5-[4-(2-Trifluoromethyl-benzoyl)-piperazin-1-yl]-pyridine-2-carbonyl}-amino)-ethyl]-piperazine-1-carboxylic acid tert-butyl ester.

The compounds of Formula III also include: 5-[4-(2-Trifluoromethylbenzoyl)piperazin-1-yl]pyridine-2-carboxylic acid (3-cyclohexyl-propyl)amide; 5-[4-(6-Trifluoromethyl-cyclohexa-1,3-dienecarbonyl)-piperazin-1-yl]-pyridine-2-carboxylic acid (2-cyclohexyl-ethyl)-amide; and 5-[4-(2-Trifluoromethyl-benzoyl)-piperazin-1-yl]-pyridine-2-carboxylic acid cyclohexylmethyl-amide.

The compounds of Formula III also include: 5-[4-(2-Trifluoromethyl-benzoyl)-piperazin-1-yl]-pyridine-2-carboxylic acid (3-methyl-butyl)-amide; 5-[4-(2-Trifluoromethyl-benzoyl)-piperazin-1-yl]-pyridine-2-carboxylic acid hexylamide; 5-[4-(2-Trifluoromethyl-benzoyl)-piperazin-1-yl]-pyridine-2-carboxylic acid pentylamide; 5-[4-(4-Fluoro-2-trifluoromethyl-benzoyl)-piperazin-1-yl]-pyridine-2-carboxylic acid (3-methyl-butyl)-amide; and 5-[4-(5-Fluoro-2-trifluoromethyl-benzoyl)-piperazin-1-yl]-pyridine-2-carboxylic acid (3-methyl-butyl)-amide.

The compounds of Formula III also include: 5-[4-(2-Trifluoromethyl-benzoyl)-piperazin-1-yl]-pyridine-2-carboxylic acid (3-phenyl-propyl)-amide; 5-[4-(2-Trifluoromethyl-benzoyl)-piperazin-1-yl]-pyridine-2-carboxylic acid phenethyl-amide; 5-[4-(2-Trifluoromethylbenzoyl)piperazin-1-yl]pyridine-2-carboxylic acid [2-(4-fluoro-phenyl)ethyl]amide; 5-[4-(2-Trifluoromethyl-benzoyl)-piperazin-1-yl]-pyridine-2-carboxylic acid [3-(4-fluoro-phenyl)-propyl]-amide; 5-[4-(2-Trifluoromethyl-benzoyl)-piperazin-1-yl]-pyridine-2-carboxylic acid 4-trifluoromethyl-benzylamide; 5-[4-(2-Trifluoromethyl-benzoyl)-piperazin-1-yl]-pyridine-2-carboxylic acid [3-(4-trifluoromethyl-phenyl)-propyl]-amide; and 5-[4-(2-Trifluoromethyl-benzoyl)-piperazin-1-yl]-pyridine-2-carboxylic acid [2-(4-trifluoromethyl-phenyl)-ethyl]-amide.

The compounds of Formula III also include: 1-[3-(4-Fluoro-phenyl)-propyl]-3-5-[4-(2-trifluoromethyl-benzoyl)-piperazin-1-yl]-pyridin-2-yl}-urea; 1-Phenethyl-3-{5-[4-(2-trifluoromethyl-benzoyl)-piperazin-1-yl]-pyridin-2-yl}-urea; and 1-Benzyl-3-{5-[4-(2-trifluoromethyl-benzoyl)-piperazin-1-yl]-pyridin-2-yl}-urea.

The compounds of Formula IV also include: 1-(3-Methyl-butyl)-3-{5-[4-(2-trifluoromethyl-benzoyl)-piperazin-1-yl]-pyridin-2-yl}-urea; 1-Pentyl-3-{5-[4-(2-trifluoromethyl-benzoyl)-piperazain-1-yl]-pyridin-2-yl}-urea; and 1-Butyl-3-{5-[4-(2-trifluoromethyl-benzoyl)-piperazin-1-yl]-pyridin-2-yl}-urea.

The compounds of Formula III also include: 3-Phenyl-propane-1-sulfonic acid {5-[4-(2-trifluoromethyl-benzoyl)-piperazin-1-yl]-pyridin-2-yl}-amide.

The compounds of Formula III also include: Pentane-1-sulfonic acid {5-[4-(2-trifluoromethyl-benzoyl)-piperazin-1-yl]-pyridin-2-yl}-amide; and Hexane-1-sulfonic acid {5-[4-(2-trifluoromethyl-benzoyl)-piperazin-1-yl]-pyridin-2-yl}-amide.

The compounds of Formula III also include: 4-Fluoro-N-{5-[4-(2-trifluoromethylbenzoyl)piperazin-1-yl]pyridin-2-yl}benzamide.

The compounds of Formula III also include: 3-Pyridin-3-yl-N-{5-[4-(2-trifluoromethyl-benzoyl)-piperazin-1-yl]-pyridin-2-yl}-propionamide.

The compounds of Formula III also include: Hexanoic acid {5-[4-(2-trifluoromethyl-benzoyl)-piperazin-1-yl]-pyridin-2-yl}-amide; Heptanoic acid {5-[4-(2-trifluoromethyl-benzoyl)-piperazin-1-yl]-pyridin-2-yl}-amide; and 5-Methylpentanoic acid {5-[4-(2-trifluoromethyl-benzoyl)-piperazin-1-yl]-pyridin-2-yl}-amide.

The compounds of Formula III also include: 3-(4-Fluoro-phenyl)-N-{5-[4-(2-trifluoromethyl-benzoyl)-piperazin-1-yl]-pyridin-2-yl}-propionamide; 4-Phenyl-N-{5-[4-(2-trifluoromethyl-benzoyl)-piperazin-1-yl]-pyridin-2-yl}-butyramide; 4-(4-Fluoro-phenyl)-N-{5-[4-(2-trifluoromethyl-benzoyl)-piperazin-1-yl]-pyridin-2-yl}-butyramide; and 3-Phenyl-N-{5-[4-(2-trifluoromethyl-benzoyl)-piperazin-1-yl]-pyridin-2-yl}-propionamide.

The methods of the present invention also comprise administration of an effective amount of an SCD-inhibiting, Aβ42-lowering, composition, to a patient in need of such treatment, wherein the composition comprises a compound according to Formula IV(a):

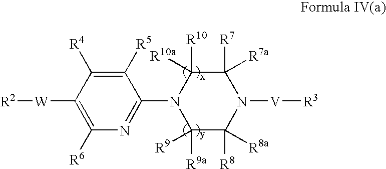

wherein: x and y are each independently 1, 2 or 3;

W is —O—, —N(R¹)—, —C(R¹)₂—, —C(O)—, —OC(O)—, —S(O)ₜ—; (where t is 0, 1 or 2), —N(R¹)S(O)ₜ—(where t is 1 or 2), —S(O)₂N(R¹)—, —C(O)N(R¹)—, —C(S)N(R¹)—, —OS(O)₂N(R¹)—, —OC(O)N(R¹)—, —OC(S)N(R¹)—, —N(R¹)C(O)N(R¹)— or —N(R¹)C(S)N(R¹)—;

V is —C(O)—, —C(S)—, —C(O)N(R¹)—, —C(O)O—, —C(S)O—, —S(O) where t is 1 or 2), —S(O)ₜN(R¹)— (where t is 1 or 2) or —C(R¹¹)H;

each R¹ is independently selected from the group consisting of hydrogen, C₁-C₁₂alkyl, C₂-C₁₂hydroxyalkyl, C₄-C₁₂cycloalkylalkyl and C₇-C₁₉aralkyl;

R² is selected from the group consisting of C₁-C₁₂alkyl, C₂-C₁₂alkenyl, C₂-C₁₂hydroxyalkyl, C₂-C₁₂hydroxyalkenyl, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂heteroarylalkyl;

or R² is a multi-ring structure having 2 to 4 rings wherein the rings are independently selected from the group consisting of cycloalkyl, heterocyclyl, aryl and heteroaryl and where some or all of the rings may be fused to each other;

R³ is selected from the group consisting of C₁-C₁₂alkyl, C₂-C₁₂alkenyl, C₂-C₁₂hydroxyalkyl, C₂-C₁₂hydroxyalkenyl, C₂-C₁₂alkoxyalkyl, C₃-C₁₂cycloalkyl, C₄-C₁₂cycloalkyalkyl, aryl, C₇-C₁₉aralkyl, C₃-C₁₂heterocyclyl, C₃-C₁₂heterocyclylalkyl, C₁-C₁₂heteroaryl and C₃-C₁₂heteroarylalkyl;

or R³ is a multi-ring structure having 2 to 4 rings wherein the rings are independently selected from the group consisting of cycloalkyl, heterocyclyl, aryl and heteroaryl and where some or all of the rings may be fused to each other;

R⁴, R⁵ and R⁶ are each independently selected from hydrogen, bromo, fluoro, chloro, methyl, methoxy, trifluoromethyl, cyano, nitro or —N(R¹³)₂;

R⁷, R^{7a}, R⁸, R^{8a}, R⁹, R^{9a}, R¹⁰, and R^{10a} are each independently selected from hydrogen or C₁-C₃alkyl;

or R⁷ and R^{7a} together, or R⁸ and R^{8a} together, or R⁹ and R^{9a} together, or R¹⁰ and R^{10a} together are an oxo group, provided that when V is —C(O)—, R⁷ and R^{7a} together or R⁸ and R^{8a} together do not form an oxo group, while the remaining R⁷, R^{7a}, R⁸, R^{8a}, R⁹, R^{9a}, R¹⁰, and R^{10a} are each independently selected from hydrogen or C₁-C₃alkyl;

or one of R¹⁰, R^{10a}, R⁷, and R^{7a} together with one of R⁸, R^{8a}, R⁹ and R^{9a} form an alkylen bridge, while the remaining R¹⁰ R^{10a}, R⁷, R^{7a}, R⁸, R^{8a}, R⁹, and R^{9a} are each independently selected from hydrogen or C₁-C₃alkyl;

R¹¹ is hydrogen or C₁-C₃alkyl; and each R¹³ is independently selected from hydrogen or C₁-C₃ alkyl);

a stereoisomer, enantiomer or tautomer thereof, a pharmaceutical acceptable salt thereof, a pharmaceutical composition thereof or a prodrug thereof.

The compounds of Formula IV(a) include: 5-Bromo-6-[4-(5-fluoro-2-trifluoromethylbenzoyl)piperazin-1-yl]-pyridine-3-sulfonic acid (2-cyclopropylethyl)amide; and 6-[4-(5-fluoro-2-trifluoromethylbenzoyl)piperazin-1-yl]pyridine-3-sulfonic acid (2-cyclopropylethyl)amide.

The compounds of Formula IV(a) also include: 1-Pentyl-3-{6-[4-(pyridine-2-carbonyl)piperazin-1-yl]-pyridin-3-yl}urea; and 1-Pentyl-3-{6-[4-(pyridine-4-carbonyl)piperazin-1-yl]-pyridin-3-yl}urea.

The compounds of Formula IV(a) also include: 1-Pentyl-3-{6-[4-(2-trifluoromethylbenzoyl)piperazin-1-yl]-pyridin-3-yl}urea; 1-Butyl-3-{6-[4-(2-trifluoromethylbenzoyl)piperazin-1-yl]-pyridin-3-yl}urea; 1-Phenethyl-3-{6-[4-(2-trifluoromethylbenzoyl)piperazin-1-yl]pyridin-3-yl}urea; 1-Benzyl-3-{6-[4-(2-trifluoromethylbenzoyl)piperazin-1-yl]-pyridin-3-yl}urea; and 1-(4-Fluorobenzyl)-3-{6-[4-(2-trifluoromethylbenzoyl)-piperazin-1-yl]pyridin-3-yl}urea.

The compounds of Formula IV(a) also include: 1-[6-(4-Cyclohexanecarbonylpiperazin-1-yl)pyridin-3-yl]-3-pentylurea; and 1-[6-(4-Cyclopentanecarbonylpiperazin-1-yl)pyridin-3-yl]-3-pentylurea.

The compounds of Formula IV(a) also include: Propane-1-sulfonic acid {6-[4-(2-trifluoromethylbenzoyl)-piperazin-1-yl]pyridin-3-yl}amide; Pentane-1-sulfonic acid {6-[4-(2-trifluoromethylbenzoyl)-piperazin-1-yl]pyridin-3-yl}amide; Butane-1-sulfonic acid {6-[4-(2-trifluoromethylbenzoyl)-piperazin-1-yl]pyridin-3-yl}amide; Hexane-1-sulfonic acid {6-[4-(2-trifluoromethylbenzoyl)-piperazin-1-yl]pyridin-3-y]}amide; Pentane-1-sulfonic acid {6-[4-(2-bromobenzoyl)piperazin-1-yl]pyridin-3-yl}amide; Hexane-1-sulfonic acid {6-[4-(2,5-dichlorobenzoyl)-piperazin-1-yl]pyridin-3-yl}amide; Pentane-1-sulfonic acid {6-[4-(2,5-dichlorobenzoyl)-piperazin-1-yl]pyridin-3-yl}amide; Hexane-1-sulfonic acid {6-[4-(naphthalene-1-carbonyl)-piperazin-1-yl]pyridin-3-yl}amide; Pentane-1-sulfonic acid {6-[4-(naphthalene-1-carbonyl)-piperazin-1-yl]pyridin-3-yl}amide; and 3-Phenylpropane-1-sulfonic acid {6-[4-(2-trifluoromethyl-benzoyl)piperazin-1-yl]pyridin-3-yl}amide.

The compounds of Formula IV(a) also include: 3-Phenyl-N-{6-[4-(2-trifluoromethylbenzoyl)piperazin-1-yl]-pyridin-3-yl}propionamide; 4-Phenyl-N-{6-4-(2-trifluoromethylbenzoyl)piperazin-1-yl]-pyridin-3-ylbutyramide; and N-{6-12-Oxo-4-(2-trifluoromethylbenzoyl)piperazin-1-yl]-pyridin-3-yl}-4-phenylbutyramide.

The compounds of Formula IV(a) also include: Cyclohexanecarboxylic acid {6-[4-(2-trifluoromethylbenzoyl)piperazin-1-yl]pyridin-3-yl}amide.

The compounds of Formula IV(a) also include: 4-Methylpentanoic acid {6-[4-(2-trifluoromethylbenzoyl)piperazin-1-yl]pyridin-3-yl}amide; Hexanoic acid {6-[4-(2-trifluoromethylbenzoyl)piperazin-1-yl]pyridin-3-yl}amide; Heptanoic acid {6-[4-(2-trifluoromethylbenzoyl)piperazin-1-yl]pyridin-3-yl}amide; Heptanoic acid {6-[4-(2,5-dichlorobenzoyl)piperazin-1-yl]pyridin-3-yl}amide; and Hexanoic acid {6-[4-(2,5-dichlorobenzoyl)piperazin-1-yl]pyridin-3-yl}amide.

The methods of the present invention also comprise administration of an effective amount of an SCD-inhibiting, Aβ42-lowering, composition, to a patient in need of such treatment, wherein the composition comprises a compound according to Formula IV(b):

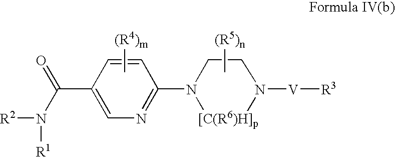

wherein:

m is 1, 2 or 3;

n is 1, 2, 3, or 4;

p is 2, 3, or 4;

V is —C(O)—, —S(O)— or —S(O)₂—;

R¹ is hydrogen, alkyl, alkenyl, aryl, aralkyl, aralkenyl or cycloalkyl;

R² is selected from the group consisting of hydrogen, —R⁷—OR⁸, —R⁷—N(R⁸)₂, —R⁷—S(O)ₜR¹⁰ (where t is 0, 1 or 2), alkyl, alkenyl, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl;

R³ is selected from the group consisting of hydrogen, —R⁹—OR⁸, —R⁹—N(R⁸)₂, alkyl, alkenyl, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl;

each R⁴ is independently hydrogen, alkyl, alkenyl, halo, haloalkyl, aryl, cyano, nitro, —R⁹—OR⁸, —R⁹—N(R⁸)₂ or —S(O)ₜR¹⁰ (where t is 0, 1 or 2);

each R⁵ and R⁶ is independently hydrogen, oxo, alkyl, alkenyl, halo, haloalkyl or aryl; or one R⁵ and one R⁶ may together form an straight or branched alkylene bridge; each R⁷ is independently a straight or branched alkylene or alkenylene chain;

each R⁸ is independently hydrogen, alkyl, alkenyl, haloalkyl, cycloalkyl, cycloalkylalkyl, aryl, aralkyl, heterocyclyl, heterocylylalkyl, heteroaryl or heteroarylalkyl;

each R⁹ is independently a direct bond or a straight or branched alkylene or alkenylene chain;

and R¹⁰ is alkyl, alkenyl, haloalkyl, cycloalkyl, cycloalkylalkyl, aryl, aralkyl, heterocyclyl, heterocylylalkyl, heteroaryl or heteroarylalkyl; as a single stereoisomer, a mixture of stereoisomers, a racemic mixture thereof of stereoisomers, or as a tautomer;

or as a pharmaceutically acceptable salt, prodrug, solvate or polymorph thereof.

Of this group of embodiments, one subgroup of embodiments is directed to the methods wherein the compound is a compound of Formula IV(b) wherein m is 1 or 2; n is 1 or 2; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2), alkyl, alkenyl, optionally substituted cycloalkyl, optionally substituted cycloalkylalkyl or optionally substituted cycloalkylalkenyl; R³ is alkyl; each R⁴ is independently hydrogen, alkyl, halo, or haloalkyl; each R⁵ and R⁶ is independently hydrogen, oxo, alkyl, halo or haloalkyl; each R⁷ is a straight or branched alkylene chain; each R⁸ is independently hydrogen, alkyl, haloalkyl, cycloalkyl, cycloalkylalkyl, aryl and aralkyl; and R¹⁰ is alkyl, aryl or aralkyl.

Of this subgroup of embodiments, one class of embodiments is directed to the methods wherein the compound is a compound of Formula IV(b) wherein m is 1; n is 1; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2) or alkyl; R³ is alkyl; R⁴ is hydrogen; R⁵ is hydrogen; each R⁶ is hydrogen; R⁷ is a straight or branched alkylene chain; R⁸ is hydrogen or alkyl; and R¹⁰ is alkyl, aryl or aralkyl.

Of the group of embodiments set forth above, another subgroup of embodiments is directed to the methods wherein the compound is a compound of Formula IV(b) wherein m is 1 or 2; n is 1 or 2; p is 2 or 3; V is —C(O)— or —S(O)₂—; R¹ is hydrogen or alkyl; R² is selected from the group consisting of optionally substituted aryl, optionally substituted aralkyl, optionally substituted aralkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is alkyl or —R⁷—N(R⁸)₂; each R⁴ is independently hydrogen, alkyl, halo, or haloalkyl; and each R⁵ and R⁶ is independently hydrogen, oxo, alkyl, alkenyl, halo, haloalkyl or aryl; or one R⁵ and one R⁶ may together form an straight or branched alkylene bridge; R⁷ is a direct bond; and each R⁸ is independently hydrogen or alkyl.

Of this subgroup of embodiments, one class of embodiments is directed to the methods wherein the compound is a compound of Formula IV(b) wherein m is 1; n is 1; p is 2 or 3; V is —C(O)— or —S(O)₂—; R¹ is hydrogen or alkyl; R² is selected from the group consisting of optionally substituted aryl, optionally substituted aralkyl, optionally substituted heterocyclyl, optionally substituted heterocyclylalkyl, optionally substituted heteroaryl and optionally substituted heteroarylalkyl; R³ is alkyl or —R⁷—N(R⁸)₂; R⁴ is hydrogen, alkyl, halo or haloalkyl; and each R⁵ and R⁶ is independently hydrogen, oxo, alkyl, halo or haloalkyl; or one R⁵ and one R⁶ may together form a methylene bridge; R⁷ is a direct bond; and each R⁸ is independently hydrogen or alkyl.

Of this group of embodiments, a subgroup of embodiments is directed to the methods wherein the compound of formula IV(b) is a compound of formula IV(b) wherein m is 1 or 2; n is 1 or 2; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2), alkyl, alkenyl, optionally substituted cycloalkyl, optionally substituted cycloalkylalkyl or optionally substituted cycloalkylalkenyl; R³ is optionally substituted cycloalkyl or optionally substituted cycloalkylalkyl; each R⁴ is independently hydrogen, alkyl, halo, or haloalkyl; each R⁵ and R⁶ is independently hydrogen, oxo, alkyl, halo or haloalkyl; each R⁷ is a straight or branched alkylene chain; each R⁸ is independently hydrogen, alkyl, haloalkyl, cycloalkyl, cycloalkylalkyl, aryl and aralkyl; and R¹⁰ is alkyl, aryl or aralkyl.

Of this subgroup of embodiments, a class of embodiments is directed to the methods wherein the compound is a compound of Formula IV(b) wherein m is 1; n is 1; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0 to 2) or alkyl; R³ is optionally substituted cycloalkyl or optionally substituted cycloalkylalkyl; R⁴ is hydrogen; R⁵ is hydrogen; each R⁶ is hydrogen; R⁷ is a straight or branched alkylene chain; R⁸ is hydrogen or alkyl; and R¹⁰ is alkyl, aryl or aralkyl.

Of the group of embodiments set forth above, another subgroup of embodiments is directed to the methods wherein the compound is a compound of Formula IV(b) wherein m is 1 or 2; n is 1 or 2; p is 2; V is —C(O)—; R¹ is hydrogen or alkyl; R² is selected from the group consisting of optionally substituted aryl, optionally substituted aralkyl, optionally substituted aralkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is optionally substituted cycloalkyl or optionally substituted cycloalkylalkyl; each R⁴ is independently hydrogen, alkyl, halo, or haloalkyl; and each R⁵ and R⁶ is independently hydrogen, oxo, alkyl, halo or haloalkyl.

Of this subgroup of embodiments, a class of embodiments is directed to the methods wherein the compound is a compound of Formula IV(b) wherein m is 1; n is 1; p is 2; V is —C(O)—; R¹ is hydrogen or alkyl; R² is selected from the group consisting of optionally substituted aryl, optionally substituted aralkyl, optionally substituted heterocyclyl, optionally substituted heterocyclylalkyl, optionally substituted heteroaryl and optionally substituted heteroarylalkyl; R³ is optionally substituted cycloalkyl or optionally substituted cycloalkylalkyl; R⁴ is hydrogen, alkyl, halo or haloalkyl; R⁵ is independently hydrogen, oxo, alkyl, halo or haloalkyl; and each R⁶ is independently hydrogen, oxo, alkyl, halo or haloalkyl.

Of the various embodiments of the invention as set forth above in the Summary of the Invention, another group of embodiments is directed to the methods of decreasing Aβ42 secretion or condition in a mammal wherein the compound of formula IV(b) is a compound of formula IV(b) wherein m is 1 or 2; n is 1 or 2; p is 2; V is —C(O)—; R¹ is hydrogen or alkyl; R² is selected from the group consisting of hydrogen, —R⁷—OR⁸, —R⁷—N(R⁸)₂, —R⁷—S(O)ₜR¹⁰ (where t is 0, 1 or 2), alkyl, alkenyl, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is optionally substituted aryl; each R⁴ is independently hydrogen, alkyl, alkenyl, halo, haloalkyl, aryl or —R⁹—OR⁸; each R⁵ and R⁶ is independently hydrogen, oxo, alkyl, alkenyl, halo, haloalkyl or aryl; each R⁷ is independently a straight or branched alkylene or alkenylene chain; each R⁸ is independently hydrogen, alkyl, alkenyl, haloalkyl, cycloalkyl, cycloalkylalkyl, aryl, aralkyl, heterocyclyl, heterocylylalkyl, heteroaryl or heteroarylalkyl; R⁹ is a direct bond or a straight or branched alkylene chain; and R¹⁰ is alkyl, aryl or aralkyl.

Of this group of embodiments, a subgroup of embodiments is directed to the methods wherein the compound of formula IV(b) is a compound of formula IV(b) wherein m is 1 or 2; n is 1 or 2; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0 to 2), alkyl, alkenyl, optionally substituted cycloalkyl, optionally substituted cycloalkylalkyl or optionally substituted cycloalkylalkenyl; R³ is optionally substituted aryl; each R⁴ is independently hydrogen, alkyl, halo, haloalkyl or —R⁹—OR⁸; each R⁵ and R⁶ is independently hydrogen, oxo, alkyl, halo or haloalkyl; each R⁷ is a straight or branched alkylene chain; each R⁸ is independently hydrogen, alkyl, haloalkyl, cycloalkyl, cycloalkylalkyl, aryl and aralkyl; R⁹ is a direct bond or a straight or branched alkylene chain; and R¹⁰ is alkyl, aryl or aralkyl.

Of this subgroup of embodiments, a class of embodiments is directed to the methods wherein the compound of formula IV(b) is a compound of formula IV(b) wherein m is 1 or 2; n is 1; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2), alkyl, optionally substituted cycloalkyl or optionally substituted cycloalkylalkyl; R³ is optionally substituted aryl; each R⁴ is independently hydrogen, halo, haloalkyl or —R⁹—OR⁸; R⁵ is hydrogen; each R⁶ is hydrogen; R⁷ is a straight or branched alkylene chain; R⁸ is hydrogen or alkyl; R⁹ is a direct bond or a straight or branched alkylene chain; and R¹⁰ is alkyl, aryl or aralkyl.

Of the group of embodiments set forth above, another subgroup of embodiments is directed to the methods wherein the compound of formula IV(b) is a compound of formula IV(b) wherein m is 1 or 2; n is 1 or 2; p is 2; V is —C(O)—; R¹ is hydrogen or alkyl; R² is selected from the group consisting of optionally substituted aryl, optionally substituted aralkyl, optionally substituted aralkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is optionally substituted aryl; each R⁴ is independently hydrogen, alkyl, halo, or haloalkyl; and each R⁵ and R⁶ is independently hydrogen, oxo, alkyl, halo or haloalkyl.

Of this subgroup of embodiments, a class of embodiments is directed to the methods wherein the compound of formula IV(b) is a compound of formula IV(b) wherein m is 1 or 2; n is 1; p is 2; V is —C(O)—; R¹ is hydrogen or alkyl; R² is selected from the group consisting of optionally substituted aryl, optionally substituted aralkyl, optionally substituted heterocyclyl, optionally substituted heterocyclylalkyl, optionally substituted heteroaryl and optionally substituted heteroarylalkyl; R³ is optionally substituted aryl; each R⁴ is independently hydrogen, alkyl, halo or haloalkyl; R⁵ is hydrogen, oxo, alkyl, halo or haloalkyl; and each R⁶ is independently hydrogen, alkyl, halo or haloalkyl.

Of the various embodiments of the invention as set forth above in the Summary of the Invention, another group of embodiments is directed to the methods of treating mild cognitive impairment, Alzheimer's disease, or dementia in a mammal wherein the compound of formula IV(b) is a compound of formula IV(b) wherein m is 1 or 2; n is 1 or 2; p is 2; V is —C(O)—; R¹ is hydrogen or alkyl; R² is selected from the group consisting of hydrogen, —R⁷—OR⁸, —R⁷—N(R⁸)₂, —R⁷—S(O)ₜR¹⁰ (where t is 0, 1 or 2), alkyl, alkenyl, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is optionally substituted heteroaryl, optionally substituted heteroarylalkyl or optionally substituted heteroarylalkenyl; each R⁴ is independently hydrogen, alkyl, alkenyl, halo, haloalkyl or aryl; each R⁵ and R⁶ is independently hydrogen, oxo, alkyl, alkenyl, halo, haloalkyl or aryl; each R⁷ is independently a straight or branched alkylene or alkenylene chain; each R⁸ is independently hydrogen, alkyl, alkenyl, haloalkyl, cycloalkyl, cycloalkylalkyl, aryl, aralkyl, heterocyclyl, heterocylylalkyl, heteroaryl or heteroarylalkyl; and R¹⁰ is alkyl, aryl or aralkyl.

Of this group of embodiments, a subgroup of embodiments is directed to the methods wherein the compound of formula IV(b) is a compound wherein m is 1 or 2; n is 1 or 2; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2), alkyl, alkenyl, optionally substituted cycloalkyl, optionally substituted cycloalkylalkyl or optionally substituted cycloalkylalkenyl; R³ is optionally substituted heteroaryl, optionally substituted heteroarylalkyl or optionally substituted heteroarylalkenyl; each R⁴ is independently hydrogen, alkyl, halo, or haloalkyl; each R⁵ and R⁶ is independently hydrogen, oxo, alkyl, halo or haloalkyl; each R⁷ is a straight or branched alkylene chain; each R⁸ is independently hydrogen, alkyl, haloalkyl, cycloalkyl, cycloalkylalkyl, aryl and aralkyl; and R¹⁰ is alkyl, aryl or aralkyl.

Of this subgroup of embodiments, a class of embodiments is directed to the methods wherein the compound of formula (I) is a compound wherein m is 1 or 2; n is 1; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2) or alkyl; R³ is optionally substituted heteroaryl, optionally substituted heteroarylalkyl or optionally substituted heteroarylalkenyl; each R⁴ is independently hydrogen, halo or haloalkyl; R⁵ is hydrogen; each R⁶ is hydrogen; R⁷ is a straight or branched alkylene chain; R⁸ is hydrogen or alkyl; and R¹⁰ is alkyl, aryl or aralkyl.

Of the group of embodiments set forth above, another subgroup of embodiments is directed to the methods wherein the compound of formula IV(b) is a compound of formula IV(b) wherein m is 1 or 2; n is 1 or 2; p is 2; V is —C(O)—; R¹ is hydrogen or alkyl; R² is selected from the group consisting of optionally substituted aryl, optionally substituted aralkyl, optionally substituted aralkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is optionally substituted heteroaryl, optionally substituted heteroarylalkyl or optionally substituted heteroarylalkenyl; each R⁴ is independently hydrogen, alkyl, halo, or haloalkyl; and each R⁵ and R⁶ is independently hydrogen, oxo, alkyl, halo or haloalkyl.

Of this subgroup of embodiments, a class of embodiments is directed to the methods wherein the compound of formula IV(b) is a compound of formula IV(b) wherein m is 1 or 2; n is 1; p is 2; V is —C(O)—; R¹ is hydrogen or alkyl; R² is selected from the group consisting of optionally substituted aryl, optionally substituted aralkyl, optionally substituted heterocyclyl, optionally substituted heterocyclylalkyl, optionally substituted heteroaryl and optionally substituted heteroarylalkyl; R³ is optionally substituted heteroaryl, optionally substituted heteroarylalkyl or optionally substituted heteroarylalkenyl; each R⁴ is independently hydrogen, alkyl, halo, or haloalkyl; R⁵ is hydrogen, oxo, alkyl, halo or haloalkyl; and each R⁶ independently hydrogen, oxo, alkyl, halo or haloalkyl.

Of the various embodiments of the invention as set forth above in the Summary of the Invention, another group of embodiments is directed to the methods of treating mild cognitive impairment, Alzheimer's disease, or dementia in a mammal wherein the compound of formula IV(b) is a compound of formula IV(b) wherein m is 1 or 2; n is 1 or 2; p is 2; V is —C(O)—; R¹ is hydrogen or alkyl; R² is selected from the group consisting of hydrogen, —R⁷—OR⁸, —R⁷—N(R⁸)₂, —R⁷—S(O)ₜR¹⁰ (where t is 0, 1 or 2), alkyl, alkenyl, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is optionally substituted aralkyl or optionally substituted aralkenyl; each R⁴ is independently hydrogen, alkyl, alkenyl, halo, haloalkyl, aryl or —R⁹—OR⁸; each R⁵ and R⁶ is independently hydrogen, oxo, alkyl, alkenyl, halo, haloalkyl or aryl; each R⁷ is independently a straight or branched alkylene or alkenylene chain; each R⁸ is independently hydrogen, alkyl, alkenyl, haloalkyl, cycloalkyl, cycloalkylalkyl, aryl, aralkyl, heterocyclyl, heterocylylalkyl, heteroaryl or heteroarylalkyl; R⁹ is a direct bond or a straight or branched alkylene chain; and R¹⁰ is alkyl, aryl or aralkyl.

Of the group of embodiments set forth above, a subgroup of embodiments is directed to the methods wherein the compound of formula IV(b) is a compound of formula IV(b) wherein m is 1 or 2; n is 1 or 2; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2), alkyl, alkenyl, optionally substituted cycloalkyl, optionally substituted cycloalkylalkyl or optionally substituted cycloalkylalkenyl; R³ is optionally substituted aralkyl or optionally substituted aralkenyl; each R⁴ is independently hydrogen, alkyl, halo, or haloalkyl; each R⁵ and R⁶ is independently hydrogen, oxo, alkyl, halo or haloalkyl; each R⁷ is a straight or branched alkylene chain; each R⁸ is independently hydrogen, alkyl, haloalkyl, cycloalkyl, cycloalkylalkyl, aryl and aralkyl; and R¹⁰ is alkyl, aryl or aralkyl.

Of this subgroup of embodiments, a class of embodiments is directed to the methods wherein the compound of formula IV(b) is a compound of formula IV(b) wherein m is 1 or 2; n is 1; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2) or alkyl; R³ is optionally substituted aralkyl or optionally substituted aralkenyl; each R⁴ is independently hydrogen, halo or haloalkyl; R⁵ is hydrogen; each R⁶ is hydrogen; R⁷ is a straight or branched alkylene chain; R⁸ is hydrogen or alkyl; and R¹⁰ is alkyl, aryl or aralkyl.

Of the group of embodiments set forth above, another subgroup of embodiments is directed to the methods wherein the compound of formula IV(b) is a compound wherein m is 1 or 2; n is 1 or 2; p is 2; V is —C(O)—; R¹ is hydrogen or alkyl; R² is selected from the group consisting of optionally substituted aryl, optionally substituted aralkyl, optionally substituted aralkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is optionally substituted aralkyl or optionally substituted aralkenyl; each R⁴ is independently hydrogen, alkyl, halo, or haloalkyl; and each R⁵ and R⁶ is independently hydrogen, oxo, alkyl, halo or haloalkyl.

Of this subgroup of embodiments, a class of embodiments is directed to the methods wherein the compound of formula IV(b) is a compound of formula IV(b) wherein m is 1 or 2; n is 1; p is 2; V is —C(O)—; R¹ is hydrogen or alkyl; R² is selected from the group consisting of optionally substituted aryl, optionally substituted aralkyl, optionally substituted heterocyclyl, optionally substituted heterocyclylalkyl, optionally substituted heteroaryl and optionally substituted heteroarylalkyl; R³ is optionally substituted aralkyl or optionally substituted aralkenyl; each R⁴ is independently hydrogen, alkyl, halo or haloalkyl; and R⁵ is hydrogen, oxo, alkyl, halo or haloalkyl; and each R⁶ is independently hydrogen, oxo, alkyl, halo or haloalkyl.

Of the compounds of formula IV(b) set forth above in the Summary of the Invention, one group of embodiments is directed to the compounds of formula IV(b) wherein m is 1, 2 or 3; n is 1, 2, 3 or 4; p is 2, 3 or 4; V is —C(O)—, —S(O)— or —S(O)₂—; R¹ is hydrogen, alkyl, alkenyl, aryl, aralkyl, aralkenyl or cycloalkyl; R² is selected from the group consisting of hydrogen, —R⁷—OR⁸, —R⁷—N(R⁸)₂, —R⁷—S(O)ₜR¹⁰ (where t is 0, 1 or 2), alkyl, alkenyl, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; R³ is selected from the group consisting of cycloalkyl substituted by one or more substituents independently selected from the group consisting of alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —R⁹—OR⁸, —R⁹—N(R⁸)₂, —R⁹—C(O)R⁸, —R⁹—C(O)OR⁸, —R⁹—C(O)N(R⁸)₂, —R⁹—N(R⁸)C(O)OR¹⁰, —R⁹—N(R⁸)C(O)R¹⁰, —R⁹—N(R⁸)(S(O)ₜR¹⁰) (where t is 1 or 2), —R⁹—S(O)ₜOR¹⁰ (where t is 1 or 2), —R⁹—S(O)ₜR¹⁰ (where t is 0, 1 or 2), and —R⁹—S(O)ₜN(R⁸)₂ (where t is 1 or 2); each R⁴ is independently hydrogen, alkyl, alkenyl, halo, haloalkyl, aryl, cyano, nitro, —R⁹—OR⁸, —R⁹—N(R⁸)₂ or —S(O)ₜR¹⁰ (where t is 0, 1 or 2); each R⁵ and R⁶ is independently hydrogen, oxo, alkyl, alkenyl, halo, haloalkyl or aryl; or one R⁵ and one R⁶ may together form an straight or branched alkylene bridge; each R⁷ is independently a straight or branched alkylene or alkenylene chain; each R⁸ is independently hydrogen, alkyl, alkenyl, haloalkyl, cycloalkyl, cycloalkylalkyl, aryl, aralkyl, heterocyclyl, heterocylylalkyl, heteroaryl or heteroarylalkyl; each R⁹ is independently a direct bond or a straight or branched alkylene or alkenylene chain; and R¹⁰ is alkyl, alkenyl, haloalkyl, cycloalkyl, cycloalkylalkyl, aryl, aralkyl, heterocyclyl, heterocylylalkyl, heteroaryl or heteroarylalkyl.

Of this group of embodiments, a subgroup of embodiments is directed the compounds wherein R³ is cyclopropyl substituted by optionally substituted aryl or optionally substituted heteroaryl.

Of this subgroup of embodiments, a class of embodiments is directed to the compounds wherein m is 1 or 2; n is 1 or 2; p is 2; V is —C(O)—; R¹ is hydrogen or alkyl; R² is selected from the group consisting of hydrogen, —R⁷—OR⁸, —R⁷—N(R⁸)₂, —R⁷—S(O)ₜR¹⁰(where t is 0, 1 or 2), alkyl, alkenyl, optionally substituted aryl, optionally substituted aralkyl, optionally substituted aralkenyl, optionally substituted cycloalkyl, optionally substituted cycloalkylalkyl, optionally substituted cycloalkylalkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; each R⁴ is independently hydrogen, alkyl, alkenyl, halo, haloalkyl, aryl or —R⁹—OR⁸; each R⁵ and R⁶ is independently hydrogen, oxo, alkyl, alkenyl, halo, haloalkyl or aryl; each R⁷ is independently a straight or branched alkylene or alkenylene chain; each R⁸ is independently hydrogen, alkyl, alkenyl, haloalkyl, cycloalkyl, cycloalkylalkyl, aryl, aralkyl, heterocyclyl, heterocylylalkyl, heteroaryl or heteroarylalkyl; R⁹ is a direct bond or a straight or branched alkylene chain; and R¹⁰ is alkyl, aryl or aralkyl.

Of this class of embodiments, a subclass of embodiments is directed to the compounds wherein m is 1 or 2; n is 1 or 2; p is 2; V is —C(O)—; R¹ is hydrogen or alkyl; R² is selected from the group consisting of optionally substituted aryl, optionally substituted aralkyl, optionally substituted aralkenyl, optionally substituted heterocyclyl, optionally substituted heterocyclylalkyl, optionally substituted heterocyclylalkenyl, optionally substituted heteroaryl, optionally substituted heteroarylalkyl and optionally substituted heteroarylalkenyl; each R⁴ is independently hydrogen, alkyl, halo, or haloalkyl; and each R⁵ and R⁶ is independently hydrogen, oxo, alkyl, halo or haloalkyl.

Of this subclass of embodiments, a set of embodiments is directed to the compounds wherein m is 1; n is 1; p is 2; V is —C(O)—; R¹ is hydrogen or alkyl; R² is optionally substituted aralkyl, optionally substituted heteroarylalkyl, or optionally substituted heterocyclylalkyl; R³ is cyclopropyl substituted by phenyl; R⁴ is hydrogen, alkyl, halo or haloalkyl; R⁵ is hydrogen, oxo, alkyl, halo or haloalkyl; and each R⁶ is hydrogen.

Of the class of embodiments set forth above, another subclass of embodiments is directed to the compounds wherein m is 1 or 2; n is 1 or 2; p is 2; V is —C(O)—; R¹ is hydrogen or alkyl; R² is selected from the group consisting of —R⁷—OR⁸, —R⁷—N(R⁸)₂, —R⁷—S(O)ₜR¹⁰ (where t is 0, 1 or 2), alkyl, alkenyl, optionally substituted cycloalkyl, optionally substituted cycloalkylalkyl or optionally substituted cycloalkylalkenyl; each R⁴ is independently hydrogen, alkyl, halo, or haloalkyl; each R⁵ and R⁶ is independently hydrogen, oxo, alkyl, halo or haloalkyl; each R⁷ is a straight or branched alkylene chain; each R⁸ is independently hydrogen, alkyl, haloalkyl, cycloalkyl, cycloalkylalkyl, aryl and aralkyl; and R¹⁰ is alkyl, aryl or aralkyl.

Of this subclass of embodiments, a set of embodiments is directed to the compounds wherein m is 1; n is 1; p is 2; V is —C(O)—; R¹ is hydrogen or alkyl; R² is selected from the group consisting of alkyl, —R⁷—OR⁸, —R⁷—N(R⁸)₂, or —R⁷—S(O)ₜR¹⁰ (where t is 0); R³ is cyclopropyl substituted by phenyl; R⁴ is hydrogen; R⁵ is hydrogen; each R⁶ is hydrogen; R⁷ is a straight or branched alkylene chain; R⁸ is hydrogen or alkyl; and R¹⁰ is alkyl, aryl or aralkyl.

The compounds of Formula IV(b) include: 6-[4-(2-Ethylbutyryl)piperazin-1-yl)-n-(3-phenylpropyl)nicotinamide-; 6-[4-(4-Methyl-hexanoyl)piperazin-1-yl]-n-(3-phenylpropyl)nicotinam-ide; 423.2 (M+1); 6-[4-(2-Ethylbutyryl)-3-methylpiperazin-1-yl]-n-(3-phenylpropyl)nic-otinamide; 6-[4-(2-Phenylcyclopropanecarbonyl)piperazin-1-yl]-n-(3-phenylpropy-1) nicotinamide; 6-[4-(3-Cyclohexylpropionyl)piperazin-1-yl]-n-(3-methylbutyl)nicotinamide; 6-[4-(2-Cyclohexylacetyl)piperazin-1-yl]-n-hexyl-nicotinamide; N-Butyl-6-[4-(3-cyclohexylpropionyl)-piperazin-1-yl]nicotinamide; 6-[4-(2-Cyclohexylacetyl)piperazin-1-yl]-n-pentyl-nicotinamide; 6-[4-(3-Cyclohexylpropionyl)piperazin-1-yl]-n-pentyl-nicotinamide; N-[2-(3-Chlorophenyl)ethyl]-6-[4-(3-cyclohexylpropionyl)piperazin-1-yl]nicotinamide; N-[2-(3-Chlorophenyl)ethyl]-6-[4-(2-cyclohexylacetyl)piperazin-1-yl-]nicotinamide; N-Butyl-6-[4-(2-mercapto-benzoyl)piperazin-1-yl]nicotinamide; N-(3-Methylbutyl)-6-[4-(2-o-tolylacetyl)piperazin-1-yl]nicotinamide; 6-{4-[2-(2,4-Dimethylphenyl)-acetyl]-piperazin-1-yl}-N-(3-methylbut-yl)nicotinamide; 409.2 (M+1); 6-[4-(2-Bromo-benzoyl)piperazin-1-yl]-n-(3-ethoxy-propyl)nicotinamide; 6-{4-[2-(2-Chloro-6-fluorophenyl)-acetyl]-piperazin-1-yl}-N-(3-meth-ylbutyl)nicotinamide; N-(3-Methylbutyl)-6-[4-(2-trifluoromethylbenzoyl)piperazin-1-yl]nicotinamide; N-(3-Methylbutyl)-6-[4-(2-trifluoromethylbenzoyl)piperazin-1-yl)nicotinamide hydrochloride; N-(3-Methylbutyl)-4-trifluoromethyl-6-[4-(2-trifluoromethylbenzoyl)-piperazin-1-yl]nicotinamide; 2-Chloro-5-fluoro-N-(3-methylbutyl)-6-[4-(2-trifluoromethylbenzoyl)-piperazin-1-yl]nicotinamide; 2-Chloro-N-(3-methylbutyl)-6-[4-(2-trifluoromethylbenzoyl)piperazin-1-yl]nicotinamide; N-(3-Methylbutyl)-6-[3-oxo-4-(2-trifluoromethylbenzyl)piperazin-1-yl]nicotinamide; 6-[4-(2,5-Dichloro-benzoyl)piperazin-1-yl]-n-(3-imidazol-1-ylpropyl-)nicotinamide; 6-[4-(2,4-Dichloro-benzoyl)piperazin-1-yl]-n-(3-imidazol-1-ylpropyl-)nicotinamide; 6-[4-(2-Bromo-5-methoxy-benzoyl)piperazin-1-yl]-n-(3-phenylpropyl)nicotinamide; 6-[4-(2-Bromo-benzoyl)piperazin-1-yl]-n-[2-(3H-imidazol-4-yl)ethyl]nicotinamide; N-[2-(3-Chlorophenyl)ethyl]-6-[4-(2,4-dichloro-benzoyl)piperazin-1-yl]nicotinamide; 6-[2-Oxo-4-(2-trifluoromethylbenzoyl)piperazin-1-yl]-n-(3-phenylpropyl)nicotinamide; N-(3-Methylbutyl)-6-[2-oxo-4-(2-trifluoromethylbenzoyl)piperazin-1-yl]nicotinamide; 6-[4-(3-Methyl-3H-1.vertline.4-thiophene-2-carbonyl)piperazin-1-yl]-n-pentyl-nicotinamide; N-[2-(3-Chlorophenyl)ethyl]-6-[4-(3-methyl-thiophene-2-carbonyl)piperazin-1-yl]nicotinamide; 6-{4-[2-(4-Chlorophenyl)propionyl]-piperazin-1-yl}-N-(3-methylbutyl-)nicotinamide; 6-[4-(2-Phenylbutyryl)piperazin-1-yl]-n-(3-phenylpropyl)nicotinamide; N-Pentyl-6-[4-(2-phenylcyclopropanecarbonyl)piperazin-1-yl]nicotinamide; N-(3-Methylbutyl)-6-[4-(naphthalene-1-carbonyl)piperazin-1-yl]nicotinamide; N-(3-Methylbutyl)-6-[4-(naphthalene-1-carbonyl)piperazin-1-yl]nicotinamide; N-[2-(3-Chlorophenyl)ethyl]-6-[4-(2-phenylcyclopropanecarbonyl)piperazin-1-yl]nicotinamide; 6-[5-(2-Ethylbutyryl)-2,5-diaza-bicyclo[2.2.1]hept-2-yl]-n-(3-phenylpropyl)nicotinamide; 6-[4-(2-Ethylbutyl)piperazin-1-yl]-n-(3-phenylpropyl)nicotinamide; 6-[4-(Butane-1-sulfonyl)piperazin-1-yl]-n-(3-phenylpropyl)nicotinamide; 4-[5-(3-Phenylpropylcarbamoyl)pyridin-2-yl]-piperazine-1-carboxylic acid butylamide; N-(3-Ethoxy-propyl)-6-[4-(2-ethylbutyryl)-piperazin-1-yl]-nicotinamideN-(3-Ethoxy-propyl)-6-(4-pentanoyl-piperazin-1-yl)-nicotinamide; N-(3-Ethoxy-propyl)-6-[4-(3-methyl-pentanoyl)-piperazin-1-yl]nicotinamide; N-(3-Butoxy-propyl)-6-(4-pentanoyl-piperazin-1-yl)-nicotinamide; N-(3-Methyl-butyl)-6-[4-(3-methyl-pentanoyl)-piperazin-1-yl]-nicotinamide; 6-[4-(3-Methyl-pentanoyl)-piperazin-1-yl]-N-pentyl-nicotinamide; N-Butyl-6-[4-(3-methyl-pentanoyl)-piperazin-1-yl]-nicotinamide; N-Butyl-6-[4-(2-ethylbutyryl)-piperazin-1-yl]-nicotinamide; N-(3-Methyl-butyl)-6-(4-pentanoyl-piperazin-1-yl)-nicotinamide; N-(2-Ethylsulfanyl-ethyl)-6-(4-pentanoyl-piperazin-1-yl)-nicotinamide; N-(3-Methoxy-propyl)-6-[4-(3-methyl-pentanoyl)-piperazin-1-yl]-nicotinamide; 6-(4-Pentanoyl-piperazin-1-yl)-N-pentyl-nicotinamide; N-Hexyl-6-[4-(3-methyl-pentanoyl)-piperazin-1-yl]-nicotinamide; N-(1,3-Dimethylbutyl)-6-[4-(3-methyl-pentanoyl)-piperazin-1-yl]-nicotinamide; N-(1-Methyl-butyl)-6-(4-pentanoyl-piperazin-1-yl)-nicotinamide; N-(3-Dimethylamino-propyl)-6-[4-(2-ethylbutyryl)-piperazin-1-yl]-nicotinamide; N-(3-Methoxy-propyl)-6-(4-pentanoyl-piperazin-1-yl)-nicotinamide; N-(1,3-Dimethylbutyl)-6-(4-pentanoyl-piperazin-1-yl)-nicotinamide; N-(2-Methyl-butyl)-6-(4-pentanoyl-piperazin-1-yl)-nicotinamide; N-(3-Butoxy-propyl)-6-[4-(2-ethylbutyryl)-piperazin-1-yl]-nicotinamide; 6-[4-(2-Ethylbutyryl)-piperazin-1-yl]-N-(3-methylbutyl)-nicotinamide; N-Butyl-6-(4-pentanoyl-piperazin-1-yl)-nicotinamide; 6-[4-(2-Ethylbutyryl)-piperazin-1-yl]-N-(2-methylbutyl)-nicotinamide; N-(3-Isopropoxy-propyl)-6-(4-pentanoyl-piperazin-1-yl)-nicotinamide; N-(2-Ethylsulfanyl-ethyl)-6-[4-(3-methyl-pentanoyl)-piperazin-1-yl]-nicotinamide; 6-[4-(2-Ethylbutyryl)-piperazin-1-yl]-N-(2-ethylsulfanyl-ethyl)-nicotinamide; 6-[4-(2-Ethylbutyryl)-piperazin-1-yl]-N-hexyl-nicotinamide; 6-[4-(2-Ethylbutyryl)-piperazin-1-yl]-N-(1-methylbutyl)-nicotinamide; N-(2-Methyl-butyl)-6-[4-(3-methyl-pentanoyl)-piperazin-1-yl]-nicotinamide; N-(1,3-Dimethylbutyl)-6-[4-(2-ethylbutyryl)-piperazin-1-yl]-nicotinamide; 6-[4-(2-Ethylbutyryl)-piperazin-1-yl]-N-pentyl-nicotinamide; N-(3-Isopropoxy-propyl)-6-[4-(3-methyl-pentanoyl)-piperazin-1-yl]-nicotinamide; N-(3-Dimethylamino-propyl)-6-[4-(3-methyl-pentanoyl)-piperazin-1-yl-]-nicotinamide; 6-[4-(2-Ethylbutyryl)-piperazin-1-yl]-N-(3-methoxy-propyl)-nicotinamide; 6-[4-(2-Ethylbutyryl)-piperazin-1-yl]-N-(3-isopropoxy-propyl)-nicotinamide; N-(3-Butoxy-propyl)-6-[4-(3-methyl-pentanoyl)-piperazin-1-yl]-nicotinamide; N-(1-Methyl-butyl)-6-[4-(3-methyl-pentanoyl)-piperazin-1-yl]-nicotinamide; N-Hexyl-6-(4-pentanoyl-piperazin-1-yl)-nicotinamide; N-(3-Dimethylamino-propyl)-6-(4-pentanoyl-piperazin-1-yl)-nicotinamide; N-[2-(3-Chlorophenyl)-1-methylethyl]-6-[4-(2-ethylbutyryl)-piperazi-n-1-yl]-nicotinamide; N-[2-(3-Chlorophenyl)-ethyl]-6-[4-(3-methyl-pentanoyl)-piperazin-1-yl]-nicotinamide; N-[2-(3-Chlorophenyl)-ethyl]-6-[4-(2-ethylbutyryl)-piperazin-1-yl]-nicotinamide; 6-(4-Pentanoyl-piperazin-1-yl)-N-(4-phenyl-butyl)-nicotinamide; N-[2-(3-Chlorophenyl)-ethyl]-6-(4-pentanoyl-piperazin-1-yl)-nicotinamide; 6-[4-(2-Ethylbutyryl)-piperazin-1-yl]-N-phenethyl-nicotinamide; 6-[4-(3-Methyl-pentanoyl)-piperazin-1-yl]-N-phenethyl-nicotinamide; 6-(4-Pentanoyl-piperazin-1-yl)-N-(3-phenyl-propyl)nicotinamide; N-(1-Methyl-3-phenyl-propyl)-6-(4-pentanoyl-piperazin-1-yl)-nicotinamide; 6-[4-(3-Methyl-pentanoyl)-piperazin-1-yl]-N-(tetrahydro-furan-2-yl-methyl)-nicotinamide; 6-[4-(2-Ethylbutyryl)-piperazin-1-yl]-N-(tetrahydro-furan-2-yl-methyl)-nicotinamide; 6-(4-Pentanoyl-piperazin-1-yl)-N-(tetrahydro-furan-2-ylmethyl)-nicotinamide; 6-[4-(2-Ethylbutyryl)-piperazin-1-yl]-N-(4-phenyl-butyl)-nicotinamide; N-(3-Imidazol-1-yl-propyl)-6-(4-pentanoyl-piperazin-1-yl)-nicotinamide; 6-[4-(2-Ethylbutyryl)-piperazin-1-yl]-N-(3-imidazol-1-yl-propyl)-nicotinamide; N-[2-(3H-Imidazol-4-yl)-ethyl]-6-(4-pentanoyl-piperazin-1-yl)-nicotinamide; N-[2-(3H-Imidazol-4-yl)-ethyl]-6-[4-(3-methyl-pentanoyl)-piperazin-1-yl]-nicotinamide; 6-(4-Pentanoyl-piperazin-1-yl)-N-phenethyl-nicotinamide; 6-[4-(2-Ethylbutyryl)-piperazin-1-yl]-N-[2-(3H-imidazol-4-yl)-ethyl-]-nicotinamide; N-(3-Imidazol-1-yl-propyl)-6-[4-(3-methyl-pentanoyl)-piperazin-1-yl-]-nicotinamide; 6-[4-(2-Cyclohexyl-acetyl)-piperazin-1-yl]-N-(3-ethoxy-propyl)-nicotinamide; N-Butyl-6-[4-(2-cyclohexyl-acetyl)-piperazin-1-yl]-nicotinamide; 6-[4-(3-Cyclohexyl-propionyl)-piperazin-1-yl]-N-(3-methoxy-propyl)-nicotinamide; N-Pentyl-6-[4-(3-trifluoromethyl-benzoyl)-piperazin-1-yl]-nicotinamide; 6-[4-(2-Cyclohexyl-acetyl)-piperazin-1-yl]-N-(2-methylbutyl)-nicotinamide; 6-[4-(3-Cyclohexyl-propionyl)-piperazin-1-yl]-N-(3-isopropoxy-propyl)-nicotinamide; 6-[4-(2-Cyclohexyl-acetyl)-piperazin-1-yl]-N-(1,3-dimethylbutyl)-nicotinamide; 6-[4-(2-Cyclohexyl-acetyl)-piperazin-1-yl]-N-(1-methylbutyl)-nicotinamide; 6-[4-(2-Cyclohexyl-acetyl)-piperazin-1-yl]-N-(3-methylbutyl)-nicotinamide; 6-[4-(2-Cyclohexyl-acetyl)-piperazin-1-yl]-N-(2-ethylsulfanyl-ethyl-)-nicotinamide; N-(3-Butoxy-propyl)-6-[4-(2-cyclohexyl-acetyl)-piperazin-1-yl]-nicotinamide; 6-[4-(3-Cyclohexyl-propionyl)-piperazin-1-yl]-N-(2-ethylsulfanyl-et-hyl)-nicotinamide; 6-[4-(2-Cyclohexyl-acetyl)-piperazin-1-yl]-N-(3-methoxy-propyl)-nicotinamide; 6-[4-(3-Cyclohexyl-propionyl)-piperazin-1-yl]-N-(1-methylbutyl)-nicotinamide; 6-[4-(2-Cyclohexyl-acetyl)-piperazin-1-yl]-N-(3-isopropoxy-propyl)-nicotinamide; 6-[4-(3-Cyclohexyl-propionyl)-piperazin-1-yl]-N-(3-ethoxy-propyl)-nicotinamide; 6-[4-(3-Cyclohexyl-propionyl)-piperazin-1-yl]-N-(1,3-dimethylbutyl)-nicotinamide; 6-[4-(3-Cyclohexyl-propionyl)-piperazin-1-yl]-N-(2-methylbutyl)-nicotinamide; 6-[4-(3-Cyclohexyl-propionyl)-piperazin-1-yl]-N-hexyl-nicotinamide; 6-[4-(3-Cyclohexyl-propionyl)-piperazin-1-yl]-N-(3-dimethylamino-pr-opyl)-nicotinamide; N-(3-Ethoxy-propyl)-6-[4-(2-phenyl-cyclopropanecarbonyl)-piperazin-1-yl]-nicotinamide; N-(2-Ethylsulfanyl-ethyl)-6-[4-(2-phenyl-cyclopropanecarbonyl)-pipe-razin-1-yl]nicotinamide; N-(1,3-Dimethylbutyl)-6-[4-(2-phenyl-cyclopropanecarbonyl)-piperazin-1-yl]-nicotinamide; N-(3-Methyl-butyl)-6-[4-(2-phenyl-cyclopropanecarbonyl)-piperazin-1-yl]-nicotinamide; N-(3-Methoxy-propyl)-6-[4-(2-phenyl-cyclopropanecarbonyl)-piperazin-1-yl]-nicotinamide; N-(3-Butoxy-propyl)-6-[4-(2-phenyl-cyclopropanecarbonyl)-piperazin-1-yl]-nicotinamide; N-(3-Dimethylamino-propyl)-6-[4-(2-phenyl-cyclopropanecarbonyl)-piperazin-1-yl]-nicotinamide; N-(3-Isopropoxy-propyl)-6-[4-(2-phenyl-cyclopropanecarbonyl)-pipera-zin-1-yl]-nicotinamide; N-(1-Methyl-butyl)-6-[4-(2-phenyl-cyclopropanecarbonyl)-piperazin-1-yl]-nicotinamide; N-Butyl-6-[4-(2-phenyl-cyclopropanecarbonyl)-piperazin-1-yl]-nicotinamide; N-Hexyl-6-[4-(2-phenyl-cyclopropanecarbonyl)-piperazin-1-yl]-nicotinamide; N-(2-Methyl-butyl)-6-[4-(2-phenyl-cyclopropanecarbonyl)-piperazin-1-yl]-nicotinamide; 6-[4-(2-Cyclohexyl-acetyl)-piperazin-1-yl]-N-(3-dimethylamino-propyl)-nicotinamide; 6-[4-(2-Phenyl-cyclopropanecarbonyl)-piperazin-1-yl]-N-(3-phenyl-propyl)-nicotinamide; N-(4-Phenyl-butyl)-6-[4-(2-phenyl-cyclopropanecarbonyl)-piperazin-1-yl]-nicotinamide; 6-[4-(2-Cyclohexyl-acetyl)-piperazin-1-yl]-N-phenethyl-nicotinamide-; N-[2-(3-Chlorophenyl)-ethyl]-6-[4-(2-phenyl-cyclopropanecarbonyl)-p-piperazin-1-yl]-nicotinamide; N-(1-Methyl-3-phenyl-propyl)-6-[4-(2-phenyl-cyclopropanecarbonyl)-p-piperazin-1-yl]-nicotinamide; N-Phenethyl-6-[4-(2-phenyl-cyclopropanecarbonyl)-piperazin-1-yl]-nicotinamide; 6-[4-(2-Phenyl-cyclopropanecarbonyl)-piperazin-1-yl]-N-(tetrahydrofuran-2-ylmethyl)-nicotinamide; N-[2-(3H-Imidazol-4-yl)-ethyl]-6-[4-(2-phenyl-cyclopropanecarbonyl)-piperazin-1-yl]-nicotinamide; N-(3-Imidazol-1-yl-propyl)-6-[4-(2-phenyl-cyclopropanecarbonyl)-piperazin-1-yl]-nicotinamide; 6-[4-(2-Cyclohexyl-acetyl)-piperazin-1-yl]-N-[2-(3H-imidazol-4-yl)-ethyl]-nicotinamide; 6-[4-(2-Cyclohexyl-acetyl)-piperazin-1-yl]-N-(3-phenyl-propyl)-nicotinamide; 6-[4-(3-Cyclohexyl-propionyl)-piperazin-1-yl]-N-phenethyl-nicotinamide; 6-[4-(3-Cyclohexyl-propionyl)-piperazin-1-yl]-N-(3-phenyl-propyl)-nicotinamide; 6-[4-(3-Methyl-pentanoyl)-piperazin-1-yl]-N-(4-phenyl-butyl)-nicotinamide; 6-[4-(2-Cyclohexyl-acetyl)-piperazin-1-yl]-N-(4-phenyl-butyl)-nicotinamide; 6-[4-(2-Cyclohexyl-acetyl)-piperazin-1-yl]-N-(1-methyl-3-phenyl-propyl)-nicotinamide; 6-[4-(3-Cyclohexyl-propionyl)-piperazin-1-yl]-N-[2-(3H-imidazol-4-yl)-ethyl]-nicotinamide; 6-[4-(3-Cyclohexyl-propionyl)-piperazin-1-yl]-N-(tetrahydro-furan-2-ylmethyl)-nicotinamide; 6-[4-(2-Cyclohexyl-acetyl)-piperazin-1-yl]-N-(3-imidazol-1-yl-propyl)-nicotinamide; 6-[4-(3-Cyclohexyl-propionyl)-piperazin-1-yl]-N-(3-imidazol-1-yl-propyl)-nicotinamide; 6-[4-(2-Cyclohexyl-acetyl)-piperazin-1-yl]-N-(tetrahydro-furan-2-yl-methyl)-nicotinamide; 6-[4-(3,5-Dichloro-benzoyl)-piperazin-1-yl]-N-(3-methoxy-propyl)-nicotinamide; 6-[4-(2-Bromo-5-methoxy-benzoyl)-piperazin-1-yl]-N-(3-dimethylamino-propyl)-nicotinamide; 6-[4-(2,5-Dichloro-benzoyl)-piperazin-1-yl]-N-(3-methylbutyl)-nicotinamide; 6-[4-(2,5-Dichloro-benzoyl)-piperazin-1-yl]-N-(3-ethoxy-propyl)-nicotinamide; N-Butyl-6-[4-(3,5-dichloro-benzoyl)-piperazin-1-yl]-nicotinamide; 6-[4-(2,4-Dichloro-benzoyl)-piperazin-1-yl]-N-(3-ethoxy-propyl)-nicotinamide; 6-[4-(2,4-Dichloro-benzoyl)-piperazin-1-yl]-N-(2-ethylsulfanyl-ethyl)-nicotinamide; N-(3-Ethoxy-propyl)-6-[4-(4-trifluoromethyl-benzoyl)-piperazin-1-yl-]-nicotinamide; N-(3-Dimethylamino-propyl)-6-[4-(2,3,4,5-tetrafluoro-benzoyl)-piperazin-1-yl]-nicotinamide; 6-[4-(2-Bromo-5-methoxy-benzoyl)-piperazin-1-yl]-N-(2-ethylsulfanyl-ethyl)-nicotinamide; N-(2-Ethylsulfanyl-ethyl)-6-[4-(3-trifluoromethyl-benzoyl)-piperazin-1-yl]-nicotinamide; 6-[4-(2-Bromo-benzoyl)-piperazin-1-yl]-N-(3-methylbutyl)-nicotinamide; N-Butyl-6-[4-(2,4-dimethyl-benzoyl)-piperazin-1-yl]-nicotinamide; 6-[4-(3,5-Dichloro-benzoyl)-piperazin-1-yl]-N-(3-isopropoxy-propyl)-nicotinamide; 6-[4-(2-Bromo-5-methoxy-benzoyl)-piperazin-1-yl]-N-(3-methylbutyl)-nicotinamide; N-(3-Butoxy-propyl)-6-[4-(2,5-dichloro-benzoyl)-piperazin-1-yl]-nicotinamide; N-(3-Methyl-butyl)-6-[4-(3-trifluoromethyl-benzoyl)-piperazin-1-yl]-nicotinamide; 6-[4-(2-Bromo-benzoyl)-piperazin-1-yl]-N-(3-butoxy-propyl)-nicotinamide; 6-[4-(2,5-Dichloro-benzoyl)-piperazin-1-yl]-N-pentyl-nicotinamide; N-(3-Butoxy-propyl)-6-[4-(2,4-dimethyl-benzoyl)-piperazin-1-yl]-nicotinamide; 6-[4-(2-Bromo-5-methoxy-benzoyl)-piperazin-1-yl]-N-(3-ethoxy-propyl-)-nicotinamide; 6-[4-(2,4-Dichloro-benzoyl)-piperazin-1-yl]-N-(3-isopropoxy-propyl)-nicotinamide; N-(3-Ethoxy-propyl)-6-[4-(2-trifluoromethyl-benzoyl)-piperazin-1-yl-]-nicotinamide; N-Pentyl-6-[4-(3-trifluoromethyl-benzoyl)-piperazin-1-yl]-nicotinamide; N-Butyl-6-[4-(2-trifluoromethyl-benzoyl)-piperazin-1-yl]-nicotinamide; N-Butyl-6-[4-(2,5-dichloro-benzoyl)-piperazin-1-yl]-nicotinamide; 6-[4-(2-Bromo-benzoyl)-piperazin-1-yl]-N-(3-isopropoxy-propyl)-nicotinamide; 6-[4-(2,5-Dichloro-benzoyl)-piperazin-1-yl]-N-(3-isopropoxy-propyl)-nicotinamide; N-(3-Butoxy-propyl)-6-[4-(4-trifluoromethyl-benzoyl)-piperazin-1-yl-]-nicotinamide; 6-[4-(2,5-Dichloro-benzoyl)-piperazin-1-yl]-N-(2-ethylsulfanyl-ethyl)-nicotinamide; N-(3-Butoxy-propyl)-6-[4-(2-trifluoromethyl-benzoyl)-piperazin-1-yl-]-nicotinamide; 6-[4-(2,5-Dichloro-benzoyl)-piperazin-1-yl]-N-(3-methoxy-propyl)-nicotinamide; 6-[4-(2,4-Dimethyl-benzoyl)-piperazin-1-yl]-N-(1,3-dimethylbutyl)-nicotinamide; 6-[4-(2,4-Dichloro-benzoyl)-piperazin-1-yl]-N-(2-methylbutyl)-nicotinamide; 6-[4-(3,5-Dichloro-benzoyl)-piperazin-1-yl]-N-(2-methylbutyl)-nicotinamide; 6-[4-(2,4-Dimethyl-benzoyl)-piperazin-1-yl]-N-(2-ethylsulfanyl-ethyl)-nicotinamide; N-(1,3-Dimethylbutyl)-6-[4-(3-trifluoromethyl-benzoyl)-piperazin-1-yl]-nicotinamide; 6-[4-(2-Bromo-5-methoxy-benzoyl)-piperazin-1-yl]-N-(2-methylbutyl)-nicotinamide; N-(3-Dimethylamino-propyl)-6-[4-(2-trifluoromethyl-benzoyl)-piperazin-1-yl]-nicotinamide; N-Hexyl-6-[4-(3-trifluoromethyl-benzoyl)-piperazin-1-yl]-nicotinamide; 6-[4-(2,4-Dichloro-benzoyl)-piperazin-1-yl]-N-(1,3-dimethylbutyl)-nicotinamide; N-(3-Isopropoxy-propyl)-6-[4-(3-trifluoromethyl-benzoyl)-piperazin-1-yl]-nicotinamide; 6-[4-(2-Bromo-5-methoxy-benzoyl)-piperazin-1-yl]-N-hexyl-nicotinamide; 6-[4-(2-Bromo-benzoyl)-piperazin-1-yl]-N-hexyl-nicotinamide; N-(3-Isopropoxy-propyl)-6-[4-(2-trifluoromethyl-benzoyl)-piperazin-1-yl]-nicotinamide; 6-[4-(2-Bromo-5-methoxy-benzoyl)-piperazin-1-yl]-N-(3-methoxy-propy-1)-nicotinamide; 6-[4-(2-Bromo-benzoyl)-piperazin-1-yl]-N-(1-methylbutyl)-nicotinamide; 6-[4-(3,5-Dichloro-benzoyl)-piperazin-1-yl]-N-hexyl-nicotinamide; N-(3-Methoxy-propyl)-6-[4-(2-trifluoromethyl-benzoyl)-piperazin-1-y-l]-nicotinamide; 6-[4-(2,4-Dichloro-benzoyl)-piperazin-1-yl]-N-hexyl-nicotinamide; N-(2-Ethylsulfanyl-ethyl)-6-[4-(2-trifluoromethyl-benzoyl)-piperazi-n-1-yl]-nicotinamide; N-Pentyl-6-[4-(2-trifluoromethyl-benzoyl)-piperazin-1-yl]-nicotinamide; N-(2-Methyl-butyl)-6-[4-(3-trifluoromethyl-benzoyl)-piperazin-1-yl]-nicotinamide; N-(2-Ethylsulfanyl-ethyl)-6-[4-(4-trifluoromethyl-benzoyl)-piperazi-n-1-yl]-nicotinamide; 6-[4-(2,4-Dichloro-benzoyl)-piperazin-1-yl]-N-pentyl-nicotinamide; 6-[4-(2-Bromo-benzoyl)-piperazin-1-yl]-N-(2-methylbutyl)-nicotinamide; 6-[4-(2-Bromo-5-methoxy-benzoyl)-piperazin-1-yl]-N-(3-butoxy-propyl-)-nicotinamide; N-(1,3-Dimethylbutyl)-6-[4-(4-trifluoromethyl-benzoyl)-piperazin-1-yl]-nicotinamide; 6-[4-(3,5-Dichloro-benzoyl)-piperazin-1-yl]-N-pentyl-nicotinamide; N-(3-Butoxy-propyl)-6-[4-(3,5-dichloro-benzoyl)-piperazin-1-yl]-nicotinamide; 6-[4-(2-Bromo-5-methoxy-benzoyl)-piperazin-1-yl]-N-butyl-nicotinamide; 6-[4-(2-Bromo-benzoyl)-piperazin-1-yl]-N-pentyl-nicotinamide; N-(1,3-Dimethylbutyl)-6-[4-(2-trifluoromethyl-benzoyl)-piperazin-1-yl]-nicotinamide; N-(3-Methoxy-propyl)-6-[4-(4-trifluoromethyl-benzoyl)-piperazin-1-yl]-nicotinamide; 6-[4-(2,4-Dimethyl-benzoyl)-piperazin-1-yl]-N-(1-methylbutyl)-nicotinamide; 6-[4-(2,4-Dichloro-benzoyl)-piperazin-1-yl]-N-(3-methoxy-propyl)-nicotinamide; N-(1-Methyl-butyl)-6-[4-(2-trifluoromethyl-benzoyl)-piperazin-1-yl]-nicotinamide; N-(3-Dimethylamino-propyl)-6-[4-(3-trifluoromethyl-benzoyl)-piperazin-1-yl]-nicotinamide; 6-[4-(2,4-Dichloro-benzoyl)-piperazin-1-yl]-N-(1-methylbutyl)-nicotinamide; 6-[4-(2,5-Dichloro-benzoyl)-piperazin-1-yl]-N-(1-methylbutyl)-nicotinamide; N-(1-Methyl-butyl)-6-[4-(3-trifluoromethyl-benzoyl)-piperazin-1-yl]-nicotinamide; 6-[4-(2,5-Dichloro-benzoyl)-piperazin-1-yl]-N-hexyl-nicotinamide; 6-[4-(2,4-Dimethyl-benzoyl)-piperazin-1-yl]-N-(3-methoxy-propyl)-nicotinamide; 6-[4-(3,5-Dichloro-benzoyl)-piperazin-1-yl]-N-(1-methylbutyl)-nicotinamide; 6-[4-(2,4-Dichloro-benzoyl)-piperazin-1-yl]-N-(3-methylbutyl)-nicotinamide; N-(3-Butoxy-propyl)-6-[4-(3-trifluoromethyl-benzoyl)-piperazin-1-yl-]-nicotinamide; 6-[4-(2,4-Dimethyl-benzoyl)-piperazin-1-yl]-N-(3-isopropoxy-propyl)-nicotinamide; N-(1-Methyl-butyl)-6-[4-(4-trifluoromethyl-benzoyl)-piperazin-1-yl]-nicotinamide; N-Butyl-6-[4-(2,4-dichloro-benzoyl)-piperazin-1-yl]-nicotinamide; 6-[4-(3,5-Dichloro-benzoyl)-piperazin-1-yl]-N-(1,3-dimethylbutyl)-nicotinamide; N-Butyl-6-[4-(3-trifluoromethyl-benzoyl)-piperazin-1-yl]-nicotinamide; 6-[4-(2-Bromo-5-methoxy-benzoyl)-piperazin-1-yl]-N-(1,3-dimethylbutyl)-nicotinamide; 6-[4-(2,4-Dimethyl-benzoyl)-piperazin-1-yl]-N-hexyl-nicotinamide; 6-[4-(2,5-Dichloro-benzoyl)-piperazin-1-yl]-N-(1,3-dimethylbutyl)-nicotinamide; N-(3-Butoxy-propyl)-6-[4-(2,4-dichloro-benzoyl)-piperazin-1-yl]-nicotinamide; N-(3-Methoxy-propyl)-6-[4-(3-trifluoromethyl-benzoyl)-piperazin-1-y-1]-nicotinamide; 6-[4-(2,4-Dimethyl-benzoyl)-piperazin-1-yl]-N-(3-ethoxy-propyl)-nicotinamide; 6-[4-(2,5-Dichloro-benzoyl)-piperazin-1-yl]-N-(2-methylbutyl)-nicotinamide; N-Hexyl-6-[4-(4-trifluoromethyl-benzoyl)-piperazin-1-yl]-nicotinamide; 6-[4-(2,4-Dimethyl-benzoyl)-piperazin-1-yl]-N-pentyl-nicotinamide; 6-[4-(2-Bromo-benzoyl)-piperazin-1-yl]-N-(1,3-dimethylbutyl)-nicotinamide; N-Hexyl-6-[4-(2-trifluoromethyl-benzoyl)-piperazin-1-yl]-nicotinamide; 6-[4-(2-Bromo-benzoyl)-piperazin-1-yl]-N-(3-methoxy-propyl)-nicotinamide; N-(2-Methyl-butyl)-6-[4-(4-trifluoromethyl-benzoyl)-piperazin-1-yl]-nicotinamide; 6-[4-(2-Bromo-5-methoxy-benzoyl)-piperazin-1-yl]-N-(1-methylbutyl)-nicotinamide; 6-[4-(2,4-Dimethyl-benzoyl)-piperazin-1-yl]-N-(2-methylbutyl)-nicotinamide; 6-[4-(2-Bromo-5-methoxy-benzoyl)-piperazin-1-yl]-N-pentyl-nicotinamide; N-(2-Methyl-butyl)-6-[4-(2-trifluoromethyl-benzoyl)-piperazin-1-yl]-nicotinamide; 6-[4-(3,5-Dichloro-benzoyl)-piperazin-1-yl]-N-(3-methylbutyl)-nicotinamide; 6-[4-(2-Bromo-benzoyl)-piperazin-1-yl]-N-(2-ethylsulfanyl-ethyl)-nicotinamide; 6-[4-(3,5-Dichloro-benzoyl)-piperazin-1-yl]-N-(3-methoxy-propyl)-nicotinamide; 6-[4-(2-Bromo-5-methoxy-benzoyl)-piperazin-1-yl]-N-(3-isopropoxy-pr-opyl)-nicotinamide; N-(3-Butoxy-propyl)-6-[4-(2,3,4,5-tetrafluoro-benzoyl)-piperazin-1-yl]-nicotinamide; N-(1,3-Dimethylbutyl)-6-[4-(2,3,4,5-tetrafluoro-benzoyl)-piperazin-1-yl]-nicotinamide; N-Butyl-6-[4-(2,3,4,5-tetrafluoro-benzoyl)-piperazin-1-yl]-nicotinamide; N-(1-Methyl-butyl)-6-[4-(2,3,4,5-tetrafluoro-benzoyl)-piperazin-1-y-1]-nicotinamide; N-(3-Methoxy-propyl)-6-[4-(2,3,4,5-tetrafluoro-benzoyl)-piperazin-1-yl]-nicotinamide; N-(3-Isopropoxy-propyl)-6-[4-(2,3,4,5-tetrafluoro-benzoyl)-piperazin-1-yl]-nicotinamide; N-Pentyl-6-[4-(2,3,4,5-tetrafluoro-benzoyl)-piperazin-1-yl]-nicotinamide; N-(2-Methyl-butyl)-6-[4-(2,3,4,5-tetrafluoro-benzoyl)-piperazin-1-y-1]-nicotinamide; N-Hexyl-6-[4-(2,3,4,5-tetrafluoro-benzoyl)-piperazin-1-yl]-nicotinamide; N-(3-Methyl-butyl)-6-[4-(2,3,4,5-tetrafluoro-benzoyl)-piperazin-1-y-l]-nicotinamide; N-(2-Ethylsulfanyl-ethyl)-6-[4-(2,3,4,5-tetrafluoro-benzoyl)-piperazin-1-yl]-nicotinamide; N-(3-Ethoxy-propyl)-6-[4-(2,3,4,5-tetrafluoro-benzoyl)-piperazin-1-yl]-nicotinamide; N-(3-Dimethylamino-propyl)-6-[4-(4-trifluoromethyl-benzoyl)-piperazin-1-yl]-nicotinamide; N-(3-Ethoxy-propyl)-6-[4-(3-trifluoromethyl-benzoyl)-piperazin-1-yl]-nicotinamide; 6-[4-(3,5-Dichloro-benzoyl)-piperazin-1-yl]-N-(2-ethylsulfanyl-ethyl)-nicotinamide; N-(3-Methyl-butyl)-6-[4-(4-trifluoromethyl-benzoyl)-piperazin-1-yl]-nicotinamide; N-Butyl-6-[4-(4-trifluoromethyl-benzoyl)-piperazin-1-yl]-nicotinamide; N-(3-Isopropoxy-propyl)-6-[4-(4-trifluoromethyl-benzoyl)-piperazin-1-yl]-nicotinamide; N-Pentyl-6-[4-(4-trifluoromethyl-benzoyl)-piperazin-1-yl]-nicotinamide; 6-[4-(3,5-Dichloro-benzoyl)-piperazin-1-yl]-N-(3-dimethylamino-propyl)-nicotinamide; 6-[4-(2-Bromo-benzoyl)-piperazin-1-yl]-N-(3-dimethylamino-propyl)-nicotinamide; 6-[4-(2,4-Dichloro-benzoyl)-piperazin-1-yl]-N-(3-dimethylamino-propyl)-nicotinamide; 6-[4-(2,5-Dichloro-benzoyl)-piperazin-1-yl]-N-(3-dimethylamino-propyl)-nicotinamide; N-(3-Dimethylamino-propyl)-6-[4-(2,4-dimethyl-benzoyl)-piperazin-1-yl]-nicotinamide; N-(3-Ethoxy-propyl)-6-[4-(2-naphthalen-2-yl-acetyl)-piperazin-1-yl]-nicotinamide; 6-[4-(2-Naphthalen-2-yl-acetyl)-piperazin-1-yl]-N-pentyl-nicotinamide; N-Butyl-6-[4-(naphthalene-1-carbonyl)-piperazin-1-yl]-nicotinamide; N-(3-Butoxy-propyl)-6-[4-(2-naphthalen-2-yl-acetyl)-piperazin-1-yl]-nicotinamide; N-Butyl-6-[4-(2-naphthalen-2-yl-acetyl)-piperazin-1-yl]-nicotinamide; N-(3-Butoxy-propyl)-6-[4-(naphthalene-1-carbonyl)-piperazin-1-yl]-nicotinamide; N-(3-Methoxy-propyl)-6-[4-(naphthalene-1-carbonyl)-piperazin-1-yl]-nicotinamide; N-(1-Methyl-butyl)-6-[4-(naphthalene-1-carbonyl)-piperazin-1-yl]-nicotinamide; 6-[4-(Naphthalene-1-carbonyl)-piperazin-1-yl]-N-pentyl-nicotinamide; N-(3-Isopropoxy-propyl)-6-[4-(2-naphthalen-2-yl-acetyl)-piperazin-1-yl]-nicotinamide; N-(1-Methyl-butyl)-6-[4-(2-naphthalen-2-yl-acetyl)-piperazin-1-yl]-nicotinamide; N-(3-Methyl-butyl)-6-[4-(2-naphthalen-2-yl-acetyl)-piperazin-1-yl]-nicotinamide; N-(3-Dimethylamino-propyl)-6-[4-(2-naphthalen-2-yl-acetyl)-piperazi-n-1-yl]-nicotinamide; N-(3-Ethoxy-propyl)-6-[4-(naphthalene-1-carbonyl)-piperazin-1-yl]-nicotinamide; N-Hexyl-6-[4-(naphthalene-1-carbonyl)-piperazin-1-yl]-nicotinamide; N-(2-Ethylsulfanyl-ethyl)-6-[4-(2-naphthalen-2-yl-acetyl)-piperazin-1-yl]-nicotinamide; N-Hexyl-6-[4-(2-naphthalen-2-yl-acetyl)-piperazin-1-yl]-nicotinamide; N-(1,3-Dimethylbutyl)-6-[4-(naphthalene-1-carbonyl)-piperazin-1-yl]-nicotinamide; N-(3-Isopropoxy-propyl)-6-[4-(naphthalene-1-carbonyl)-piperazin-1-yl]-nicotinamide; N-(1,3-Dimethylbutyl)-6-[4-(naphthalene-2-carbonyl)-piperazin-1-yl]-nicotinamide; N-(1,3-Dimethylbutyl)-6-[4-(2-naphthalen-2-yl-acetyl)-piperazin-1-yl]-nicotinamide; N-(2-Methyl-butyl)-6-[4-(naphthalene-1-carbonyl)-piperazin-1-yl]-nicotinamide; N-(2-Ethylsulfanyl-ethyl)-6-[4-(naphthalene-1-carbonyl)-piperazin-1-yl]-nicotinamide; N-(2-Methyl-butyl)-6-[4-(2-naphthalen-2-yl-acetyl)-piperazin-1-yl]-nicotinamide; N-(3-Ethoxy-propyl)-6-[4-(naphthalene-2-carbonyl)-piperazin-1-yl]-nicotinamide; N-(2-Methyl-butyl)-6-[4-(naphthalene-2-carbonyl)-piperazin-1-yl]-nicotinamide; N-(3-Methyl-butyl)-6-[4-(naphthalene-2-carbonyl)-piperazin-1-yl]-nicotinamide; N-(3-Methoxy-propyl)-6-[4-(naphthalene-2-carbonyl)-piperazin-1-yl]-nicotinamide; N-Butyl-6-[4-(naphthalene-2-carbonyl)-piperazin-1-yl]-nicotinamide; N-(3-Butoxy-propyl)-6-[4-(naphthalene-2-carbonyl)-piperazin-1-yl]nicotinamide; N-(1-Methyl-butyl)-6-[4-(naphthalene-2-carbonyl)-piperazin-1-yl]-nicotinamide; N-(3-Isopropoxy-propyl)-6-[4-(naphthalene-2-carbonyl)-piperazin-1-y-l]-nicotinamide; N-Hexyl-6-[4-(naphthalene-2-carbonyl)-piperazin-1-yl]-nicotinamide; N-(3-Dimethylamino-propyl)-6-[4-(naphthalene-1-carbonyl)-piperazin-1-yl]-nicotinamide; N-(2-Ethylsulfanyl-ethyl)-6-[4-(naphthalene-2-carbonyl)-piperazin-1-yl]-nicotinamide; N-(3-Dimethylamino-propyl)-6-[4-(naphthalene-2-carbonyl)-piperazin-1-yl]-nicotinamide; N-(3-Methoxy-propyl)-6-[4-(2-naphthalen-2-yl-acetyl)-piperazin-1-yl-]-nicotinamide; N-(2-Cyclopropyl-ethyl)-6-[4-(3-trifluoromethyl-benzoyl)-piperazin-1-yl]-nicotinamide; N-(2-Cyclopropyl-ethyl)-6-[4-(5-fluoro-2-trifluoromethyl-benzoyl)-piperazin-1-yl]-nicotinamide; N-(2-Cyclopropyl-ethyl)-2-hydroxy-6-[4-(2-trifluoromethyl-benzoyl)-piperazin-1-yl]-nicotinamide; N-(2-Cyclobutyl-ethyl)-6-[4-(5-fluoro-2-trifluoromethyl-benzoyl)-piperazin-1-yl]-nicotinamide; N-(3-Cyclopropyl-propyl)-6-[4-(5-fluoro-2-trifluoromethyl-benzoyl)-piperazin-1-yl]-nicotinamide; 6-[4-(5-Fluoro-2-trifluoromethyl-benzoyl)-piperazin-1-yl]-N-(4-methyl-pentyl)-nicotinamide; N-(3,3-Dimethylbutyl)-6-[4-(5-fluoro-2-trifluoromethyl-benzoyl)-piperazin-1-yl]-nicotinamide; N-(1-Methyl-2-phenyl-ethyl)-6-[4-(2-trifluoromethyl-benzoyl)-piperazin-1-yl]-nicotinamide; N-(1-Methyl-2-phenyl-ethyl)-6-[4-(4-trifluoromethyl-benzoyl)-piperazin-1-yl]-nicotinamide; 6-[4-(Naphthalene-1-carbonyl)-piperazin-1-yl]-N-(4-phenyl-butyl)-nicotinamideN-[2-(3-Chlorophenyl)-ethyl]-6-[4-(2-naphthalen-2-yl-acetyl)-piperazin-1-yl]-nicotinamide; 6-[4-(Naphthalene-2-carbonyl)-piperazin-1-yl]-N-(3-phenyl-propyl)-nicotinamide; N-(3-Imidazol-1-yl-propyl)-6-[4-(2-naphthalen-2-yl-acetyl)-piperazin-1-yl]-nicotinamide; 6-[4-(Naphthalene-1-carbonyl)-piperazin-1-yl]-N-(3-phenyl-propyl)-nicotinamide; N-(1-Methyl-3-phenyl-propyl)-6-[4-(2-naphthalen-2-yl-acetyl)-piperazin-1-yl]-nicotinamide; 6-[4-(2-Naphthalen-2-yl-acetyl)-piperazin-1-yl]-N-phenethyl-nicotinamide; N-[2-(3-Chlorophenyl)-ethyl]-6-[4-(naphthalene-1-carbonyl)-piperazin-1-yl]-nicotinamide; N-(1-Methyl-3-phenyl-propyl)-6-[4-(naphthalene-1-carbonyl)-piperazin-1-yl]-nicotinamide; 6-[4-(Naphthalene-1-carbonyl)-piperazin-1-yl]-N-phenethyl-nicotinamide; N-[2-(3H-Imidazol-4-yl)-ethyl]-6-[4-(2-naphthalen-2-yl-acetyl)-pipe-razin-1-yl]-nicotinamide; 6-[4-(2-Naphthalen-2-yl-acetyl)-piperazin-1-yl]-N-(4-phenyl-butyl)-nicotinamide; 6-[4-(Naphthalene-2-carbonyl)-piperazin-1-yl]-N-(3-phenyl-propyl)-nicotinamide; N-(1-Methyl-3-phenyl-propyl)-6-[4-(naphthalene-2-carbonyl)-piperazin-1-yl]-nicotinamide; N-[2-(3H-Imidazol-4-yl)-ethyl]-6-[4-(naphthalene-1-carbonyl)-piperazin-1-yl]-nicotinamide; 6-[4-(Naphthalene-2-carbonyl)-piperazin-1-yl]-N-(tetrahydro-furan-2-ylmethyl)-nicotinamide; 6-[4-(Naphthalene-1-carbonyl)-piperazin-1-yl]-N-(tetrahydro-furan-2-ylmethyl)-nicotinamide; N-(3-Imidazol-1-yl-propyl)-6-[4-(naphthalene-2-carbonyl)-piperazin-1-yl]-nicotinamide; N-[2-(3H-imidazol-4-yl)-ethyl]-6-[4-(naphthalene-2-carbonyl)-piperazin-1-yl]-nicotinamide; 6-[4-(Naphthalene-2-carbonyl)-piperazin-1-yl]-N-phenethyl-nicotinamide; 6-[4-(Naphthalene-2-carbonyl)-piperazin-1-yl]-N-(4-phenyl-butyl)-nicotinamide; N-[2-(3-Chlorophenyl)-ethyl]-6-[4-(naphthalene-2-carbonyl)-piperazin-1-yl]-nicotinamide; N-(3-Imidazol-1-yl-propyl)-6-[4-(naphthalene-1-carbonyl)-piperazinl-yl]-nicotinamide; 6-[4-(2-Naphthalen-2-yl-acetyl)-piperazin-1-yl]-N-(tetrahydro-furan-2-ylmethyl)-nicotinamide; 6-[4-(2-Bromo-benzoyl)-piperazin-1-yl]-N-(3-phenyl-propyl)-nicotinamide; 6-[4-(2,4-Dimethyl-benzoyl)-piperazin-1-yl]-N-(4-phenyl-butyl)-nicotinamide;]N-Phenethyl-6-[4-(2-p-tolyl-acetyl)-piperazin-1-yl]-nicotinamide; 6-{4-[2-(2,5-Dichloro-phenyl)-acetyl]-piperazin-1-yl}-N-(2-methyl-3-phenyl-propyl)-nicotinamide; 6-[4-(2,4-Dimethyl-benzoyl)-piperazin-1-yl]-N-(3-phenyl-propyl)-nic-otinamide; N-[2-(3-Chlorophenyl)-ethyl]-6-[4-(2-fluoro-4-methyl-benzoyl)-piper-azin-1-yl]-nicotinamide; N-[2-(3-Chlorophenyl)-ethyl]-6-[4-(2,5-dichloro-benzoyl)-piperazin-1-yl]-nicotinamide; N-(3-Phenyl-propyl)-6-[4-(4-trifluoromethyl-benzoyl)-piperazin-1-yl-]-nicotinamide; 6N-(4-Phenyl-butyl)-6-[4-(2-trifluoromethyl-benzoyl)-piperazin-1-yl]-nicotinamide; N-(3-Phenyl-propyl)-6-[4-(3-trifluoromethyl-benzoyl)-piperazin-1-yl-]-nicotinamide; 6-[4-(2-Bromo-benzoyl)-piperazin-1-yl]-N-[2-(3-chloro-phenyl)-ethyl-]-nicotinamide; 6-[4-(2-Bromo-benzoyl)-piperazin-1-yl]-N-(2-methyl-3-phenyl-propyl)-nicotinamide; 6-[4-(2-Bromo-5-methoxy-benzoyl)-piperazin-1-yl]-N-(5-phenyl-pentyl)-nicotinamide; 6-[4-(2-Bromo-5-methoxy-benzoyl)-piperazin-1-yl]-N-[2-(3-chloro-phe-nyl)-ethyl]-nicotinamide; N-[2-(3-Chlorophenyl)-ethyl]-6-[4-(2-trifluoromethyl-benzoyl)-piperazin-1-yl]-nicotinamide; N-(3-Phenyl-propyl)-6-[4-(2-trifluoromethyl-benzoyl)-piperazin-1-yl-]-nicotinamide; N-(4-Phenyl-butyl)-6-[4-(3-trifluoromethyl-benzoyl)-piperazin-1-yl]-nicotinamide; 6-[4-(2,4-Dichloro-benzoyl)-piperazin-1-yl]-N-phenethyl-nicotinamide; 6-[4-(2-Bromo-benzoyl)-piperazin-1-yl]-N-(4-phenyl-butyl)-nicotinamide; 6-[4-(2-Bromo-benzoyl)-piperazin-1-yl]-N-phenethyl-nicotinamide; 6-[4-(2-Bromo-5-methoxy-benzoyl)-piperazin-1-yl]-N-(1-methyl-3-phenyl-propyl)-nicotinamide; 6-[4-(3,5-Dichloro-benzoyl)-piperazin-1-yl]-N-(3-phenyl-propyl)-nicotinamide; 6-[4-(3,5-Dichloro-benzoyl)-piperazin-1-yl]-N-(1-methyl-3-phenyl-propyl)-nicotinamide; N-Phenethyl-6-[4-(4-trifluoromethyl-benzoyl)-piperazin-1-yl]-nicotinamide; 6-[4-(2,4-Dimethyl-benzoyl)-piperazin-1-yl]-N-phenethyl-nicotinamide; N-Phenethyl-6-[4-(2-trifluoromethyl-benzoyl)-piperazin-1-yl]-nicotinamide; 6-[4-(2,4-Dichloro-benzoyl)-piperazin-1-yl]-N-(1-methyl-3-phenyl-pr-opyl)-nicotinamide; 6-[4-(2,5-Dichloro-benzoyl)-piperazin-1-yl]-N-(3-phenyl-propyl)-nicotinamide; 6-[4-(2-Bromo-5-methoxy-benzoyl)-piperazin-1-yl]-N-phenethyl-nicotinamide; 6-[4-(2-Bromo-5-methoxy-benzoyl)-piperazin-1-yl]-N-(3-imidazol-1-yl-propyl)-nicotinamide; 6-[4-(3,5-Dichloro-benzoyl)-piperazin-1-yl]-N-(3-imidazol-1-yl-propyl)-nicotinamide; N-[2-(3H-Imidazol-4-yl)-ethyl]-6-[4-(2,3,4,5-tetrafluoro-benzoyl)-piperazin-1-yl]-nicotinamide; N-Phenethyl-6-[4-(3-trifluoromethyl-benzoyl)-piperazin-1-yl]-nicotinamide; 6-[4-(2,5-Dichloro-benzoyl)-piperazin-1-yl]-N-phenethyl-nicotinamide; 6-[4-(3-Methyl-pentanoyl)-piperazin-1-yl]-N-(1-methyl-3-phenyl-propyl)-nicotinamide; 6-[4-(2,3,4,5-Tetrafluoro-benzoyl)-piperazin-1-yl]-N-(tetrahydro furan-2-ylmethyl)-nicotinamide; 6-[4-(2-Bromo-5-methoxy-benzoyl)-piperazin-1-yl]-N-(tetrahydrofuran-2-ylmethyl)-nicotinamide; N-(Tetrahydro furan-2-ylmethyl)-6-[4-(4-trifluoromethyl-benzoyl)-piperazin-1-yl]-nicotinamide; 6-[4-(3,5-Dichloro-benzoyl)-piperazin-1-yl]-N-(4-phenyl-butyl)-nicotinamide; 6-[4-(2,4-Dimethyl-benzoyl)-piperazin-1-yl]-N-(1-methyl-3-phenyl-pr-opyl)-nicotinamide; 6-[4-(2,4-Dimethyl-benzoyl)-piperazin-1-yl]-N-(tetrahydro-furan-2-ylmethyl)-nicotinamide; 6-[4-(2-Bromo-benzoyl)-piperazin-1-yl]-N-(tetrahydro-furan-2-ylmethyl)-nicotinamide; N-(1-Methyl-3-phenyl-propyl)-6-[4-(3-trifluoromethyl-benzoyl)-piperazin-1-yl]-nicotinamide; N-Phenethyl-6-[4-(2,3,4,5-tetrafluoro-benzoyl)-piperazin-1-yl]-nicotinamide; N-(4-Phenyl-butyl)-6-[4-(2,3,4,5-tetrafluoro-benzoyl)-piperazin-1-yl]-nicotinamide; N-(1-Methyl-3-phenyl-propyl)-6-[4-(2,3,4,5-tetrafluoro-benzoyl)-piperazin-1-yl]-nicotinamide; N-[2-(3-Chlorophenyl)-ethyl]-6-[4-(2,3,4,5-tetrafluoro-benzoyl)-piperazin-1-yl]-nicotinamide; N-(3-Phenyl-propyl)-6-[4-(2,3,4,5-tetrafluoro-benzoyl)-piperazin-1-yl]-nicotinamide; 6-[4-(2,5-Dichloro-benzoyl)-piperazin-1-yl]-N-(tetrahydro-furan-2-ylmethyl)-nicotinamide; 6-[4-(2,4-Dimethyl-benzoyl)-piperazin-1-yl]-N-[2-(3H-imidazol-4-yl)-ethyl]-nicotinamide; N-[2-(3H-Imidazol-4-yl)-ethyl]-6-[4-(3-trifluoromethyl-benzoyl)-piperazin-yl]-nicotinamide; N-(3-Imidazol-1-yl-propyl)-6-[4-(3-trifluoromethyl-benzoyl)-piperazin-1-yl]-nicotinamide; 6-[4-(2,4-Dimethyl-benzoyl)-piperazin-1-yl]-N-(3-imidazol-1-yl-propyl)-nicotinamide; 6-[4-(2,5-Dichloro-benzoyl)-piperazin-1-yl]-N-[2-(3H-imidazol-4-yl)-ethyl]-nicotinamide; N-[2-(3H-Imidazol-4-yl)-ethyl]-6-[4-(4-trifluoromethyl-benzoyl)-piperazin-1-yl]-nicotinamide; 6-[4-(2,4-Dichloro-benzoyl)-piperazin-1-yl]-N-[2-(3H-imidazol-4-yl)-ethyl]-nicotinamide; 6-[4-(3,5-Dichloro-benzoyl)-piperazin-1-yl]-N-(tetrahydro-furan-2-ylmethyl)-nicotinamide; N-(3-Imidazol-1-yl-propyl)-6-[4-(2-trifluoromethyl-benzoyl)-piperazin-1-yl]-nicotinamide; N-(Tetrahydro-furan-2-ylmethyl)-6-[4-(3-trifluoromethyl-benzoyl)-piperazin-1-yl]-nicotinamide; N-(Tetrahydro-furan-2-ylmethyl)-6-[4-(2-trifluoromethyl-benzoyl)-piperazin-1-yl]-nicotinamide; 6-[4-(2,4-Dichloro-benzoyl)-piperazin-1-yl]-N-(tetrahydro-furan-2-ylmethyl)-nicotinamide; N-[2-(3H-Imidazol-4-yl)-ethyl]-6-[4-(2-trifluoromethyl-benzoyl)-piperazin-1-yl]-nicotinamide; 6-[4-(2-Bromo-5-methoxy-benzoyl)-piperazin-1-yl]-N-[2-(3H-imidazol-4-yl)-ethyl]-nicotinamide; N-[2-(3-Chlorophenyl)-ethyl]-6-[4-(3-trifluoromethyl-benzoyl)-piperazin-1-yl]-nicotinamide; N-[2-(3-Chlorophenyl)-ethyl]-6-[4-(4-trifluoromethyl-benzoyl)-piperazin-1-yl]-nicotinamide; N-[2-(3-Chlorophenyl)-ethyl]-6-[4-(3,5-dichloro-benzoyl)-piperazin-1-yl]-nicotinamide; N-(4-Phenyl-butyl)-6-[4-(4-trifluoromethyl-benzoyl)-piperazin-1-yl]-nicotinamide; 6-[4-(2,4-Dichloro-benzoyl)-piperazin-1-yl]-N-(4-phenyl-butyl)-nicotinamide; 6-[4-(2,4-Dichloro-benzoyl)-piperazin-1-yl]-N-(3-phenyl-propyl)-nicotinamide; 6-[4-(2,5-Dichloro-benzoyl)-piperazin-1-yl]-N-(4-phenyl-butyl)-nicotinamide; 6-[4-(3,5-Dichloro-benzoyl)-piperazin-1-yl]-N-phenethyl-nicotinamide; N-(3-Imidazol-1-yl-propyl)-6-[4-(4-trifluoromethyl-benzoyl)-piperazin-1-yl]-nicotinamide; 6-[4-(3,5-Dichloro-benzoyl)-piperazin-1-yl]-N-[2-(3H-imidazol-4-yl)-ethyl]-nicotinamide; N-(3-Imidazol-1-yl-propyl)-6-[4-(2,3,4,5-tetrafluoro-benzoyl)-piperazin-1-yl]-nicotinamide; N-(3-Dimethylamino-propyl)-6-[4-(3-methyl-thiophene-2-carbonyl)-piperazin-1-yl]-nicotinamide; N-(3-Dimethylamino-propyl)-6-[4-(thiophene-2-carbonyl)-piperazin-1-yl]-nicotinamide; 6-[4-(2-Chloro-pyridine-3-carbonyl)-piperazin-1-yl]-N-(3-ethoxy-propyl)-nicotinamide; 6-[4-(2-Chloro-pyridine-3-carbonyl)-piperazin-1-yl]-N-(2-ethylsulfanyl-ethyl)-nicotinamide; 6-[4-(2-Chloro-pyridine-3-carbonyl)-piperazin-1-yl]-N-(3-methylbutyl)-nicotinamide; 6-[4-(2-Chloro-pyridine-3-carbonyl)-piperazin-1-yl]-N-(3-isopropoxy-propyl)-nicotinamide; N-(2-Ethylsulfanyl-ethyl)-6-[4-(thiophene-2-carbonyl)-piperazin-1-yl]-nicotinamide; N-(3-Ethoxy-propyl)-6-[4-(thiophene-2-carbonyl)-piperazin-1-yl]-nicotinamide; N-(3-Methyl-butyl)-6-[4-(thiophene-2-carbonyl)-piperazin-1-yl]-nicotinamide; 6-[4-(2-Chloro-pyridine-3-carbonyl)-piperazin-1-yl]-N-(3-methoxy-propyl)-nicotinamide; N-Pentyl-6-[4-(thiophene-2-carbonyl)-piperazin-1-yl]-nicotinamide; 6-[4-(2-Chloro-pyridine-3-carbonyl)-piperazin-1-yl]-N-hexyl-nicotinamide; N-(3-Isopropoxy-propyl)-6-[4-(thiophene-2-carbonyl)-piperazin-1-yl]-nicotinamide; N-(1-Methyl-butyl)-6-[4-(thiophene-2-carbonyl)-piperazin-1-yl]-nicotinamide; N-Hexyl-6-[4-(thiophene-2-carbonyl)-piperazin-1-yl]-nicotinamide; N-(1,3-Dimethylbutyl)-6-[4-(thiophene-2-carbonyl)-piperazin-1-yl]-nicotinamide; N-Butyl-6-[4-(thiophene-2-carbonyl)-piperazin-1-yl]-nicotinamide; N-(3-Methoxy-propyl)-6-[4-(thiophene-2-carbonyl)-piperazin-1-yl]-nicotinamide; 6-[4-(2-Chloro-pyridine-3-carbonyl)-piperazin-1-yl]-N-(1,3-dimethyl-butyl)-nicotinamide; N-Butyl-6-[4-(2-chloro-pyridine-3-carbonyl)-piperazin-1-yl]-nicotinamide; 6-[4-(2-Chloro-pyridine-3-carbonyl)-piperazin-1-yl]-N-(2-methylbuty-1)-nicotinamide; N-(2-Methyl-butyl)-6-[4-(thiophene-2-carbonyl)-piperazin-1-yl]-nicotinamide; 6-[4-(2-Chloro-pyridine-3-carbonyl)-piperazin-1-yl]-N-(1-methylbutyl)-nicotinamide; N-(1,3-Dimethylbutyl)-6-[4-(3-methyl-thiophene-2-carbonyl)-piperazin-1-yl]-nicotinamide; N-Butyl-6-[4-(3-methyl-thiophene-2-carbonyl)-piperazin-1-yl]-nicotinamide; N-(3-Butoxy-propyl)-6-[4-(3-methyl-thiophene-2-carbonyl)-piperazin-1-yl]-nicotinamide; N-(3-isopropoxy-propyl)-6-[4-(3-methyl-thiophene-2-carbonyl)-piperazin-1-yl]-nicotinamide; N-(3-Ethoxy-propyl)-6-[4-(3-methyl-thiophene-2-carbonyl)-piperazin-1-yl]-nicotinamide; N-(2-Methyl-butyl)-6-[4-(3-methyl-thiophene-2-carbonyl)-piperazin-1-yl]-nicotinamide; N-(2-Ethylsulfanyl-ethyl)-6-[4-(3-methyl-thiophene-2-carbonyl)-piperazin-1-yl]-nicotinamide; N-(3-Methoxy-propyl)-6-[4-(3-methyl-thiophene-2-carbonyl)-piperazin-1-yl]-nicotinamide; N-(1-Methyl-butyl)-6-[4-(3-methyl-thiophene-2-carbonyl)-piperazin-1-yl]-nicotinamide; N-(3-Methyl-butyl)-6-[4-(3-methyl-thiophene-2-carbonyl)-piperazin-1-yl]-nicotinamide; N-(3-Butoxy-propyl)-6-[4-(2-chloro-pyridine-3-carbonyl)-piperazin-1-yl]-nicotinamide; N-(3-Butoxy-propyl)-6-[4-(thiophene-2-carbonyl)-piperazin-1-yl]-nicotinamide; 6-[4-(2-Chloro-pyridine-3-carbonyl)-piperazin-1-yl]-N-pentyl-nicotinamide; N-Hexyl-6-[4-(3-methyl-thiophene-2-carbonyl)-piperazin-1-yl]-nicotinamide; 6-[4-(2-Chloro-pyridine-3-carbonyl)-piperazin-1-yl]-N-(3-dimethylaminopropyl)-nicotinamide; N-[2-(3-Chlorophenyl)-ethyl]-6-{4-[2-(2-chloro-pyridin-3-yl)-acetyl-]-piperazin-1-yl}-nicotinamide; 6-[4-(2-Chloro-pyridine-3-carbonyl)-piperazin-1-yl]-N-(4-phenyl-butyl)-nicotinamide; N-[2-(3-Chlorophenyl)-ethyl]-6-[4-(thiophene-2-carbonyl)-piperazin-1-yl]-nicotinamide; 6-[4-(2-Chloro-pyridine-3-carbonyl)-piperazin-1-yl]-N-(3-phenyl-propyl)-nicotinamide; N-(3-Phenyl-propyl)-6-[4-(thiophene-2-carbonyl)-piperazin-1-yl]-nicotinamide; N-Phenethyl-6-[4-(thiophene-2-carbonyl)-piperazin-1-yl]-nicotinamide; N-(1-Methyl-3-phenyl-propyl)-6-[4-(thiophene-2-carbonyl)-piperazin-1-yl]-nicotinamide; N-(4-Phenyl-butyl)-6-[4-(thiophene-2-carbonyl)-piperazin-1-yl]-nicotinamide; 6-[4-(2-Chloro-pyridine-3-carbonyl)-piperazin-1-yl]-N-(1-methyl-3-phenyl-propyl)-nicotinamide; 6-[4-(2-Chloro-pyridine-3-carbonyl)-piperazin-1-yl]-N-phenethyl-nicotinamide; 6-[4-(2-Chloro-pyridine-3-carbonyl)-piperazin-1-yl]-N-(tetrahydrofuran-2-ylmethyl)-nicotinamide; 6-[4-(3-Methyl-thiophene-2-carbonyl)-piperazin-1-yl]-N-(3-phenyl-propyl)-nicotinamide; N-(1-Methyl-3-phenyl-propyl)-6-[4-(3-methyl-thiophene-2-carbonyl)-piperazin-1-yl]-nicotinamide; 6-[4-(3-Methyl-thiophene-2-carbonyl)-piperazin-1-yl]-N-(4-phenyl-butyl)-nicotinamide; 6-[4-(3-Methyl-thiophene-2-carbonyl)-piperazin-1-yl]-N-phenethyl-nicotinamide; 6-[4-(3-Methyl-thiophene-2-carbonyl)-piperazin-1-yl]-N-(tetrahydrofuran-2-ylmethyl)-nicotinamide; N-(3-Imidazol-1-yl-propyl)-6-[4-(thiophene-2-carbonyl)-piperazin-1-yl]-nicotinamide; N-(Tetrahydro-furan-2-ylmethyl)-6-[4-(thiophene-2-carbonyl)-piperazin-1-yl]-nicotinamide; N-[2-(3H-Imidazol-4-yl)-ethyl]-6-[4-(thiophene-2-carbonyl)-piperazin-1-yl]-nicotinamide; N-(3-Imidazol-1-yl-propyl)-6-[4-(3-methyl-thiophene-2-carbonyl)-piperazin-1-yl]-nicotinamide; N-[2-(3H-Imidazol-4-yl)-ethyl]-6-[4-(3-methyl-thiophene-2-carbonyl)-piperazin-1-yl]-nicotinamide; 6-[4-(2-Chloro-pyridine-3-carbonyl)-piperazin-1-yl]-N-(3-imidazol-1-yl-propyl)-nicotinamide; 6-[4-(2-Chloro-pyridine-3-carbonyl)-piperazin-1-yl]-N-[2-(3H-imidazol-4-yl)-ethyl]-nicotinamide; 6-{4-[2-(2-Chloro-6-fluoro-phenyl)-acetyl]-piperazin-1-yl}-N-(3-ethoxy-propyl)-nicotinamide; 6-{4-[3-(3,4-Difluoro-phenyl)-propionyl]-piperazin-1-yl}-N-(3-dimethylamino-propyl)-nicotinamide; 6-{4-[3-(3,4-Difluoro-phenyl)-propionyl]-piperazin-1-yl}-N-(3-ethoxy-propyl)-nicotinamide; 6-{4-[2-(4-Chlorophenyl)-propionyl]-piperazin-1-yl}-N-(3-ethoxy-propyl)-nicotinamide; N-(3-Ethoxy-propyl)-6-[4-(2-phenyl-butyryl)-piperazin-1-yl]-nicotinamide; N-(2-Ethylsulfanyl-ethyl)-6-[4-(2-o-tolyl-acetyl)-piperazin-1-yl]-nicotinamide; 6-[4-(3-Methyl-pentanoyl)-piperazin-1-yl]-N-(4-phenyl-butyl)-nicotinamide; N-Butyl-6-{4-[3-(3,4-difluoro-phenyl)-propionyl]-piperazin-1-yl}-nicotinamide; 6-{4-[2-(2-Chloro-6-fluoro-phenyl)-acetyl]-piperazin-1-yl}-N-(3-dimethylamino-propyl)-nicotinamide; 6-{4-[3-(3,4-Difluoro-phenyl)-propionyl]-piperazin-1-yl}-N-(2-ethylsulfanyl-ethyl)-nicotinamide; N-(3-Methoxy-propyl)-6-[4-(2-phenyl-butyryl)-piperazin-1-yl]-nicotinamide; N-Butyl-6-[4-(2-phenyl-butyryl)-piperazin-1-yl]-nicotinamide; N-(3-Butoxy-propyl)-6-{4-[2-(4-chloro-phenyl)-propionyl]-piperazin-1-yl}-nicotinamide; N-(3-Methoxy-propyl)-6-[4-(2-o-tolyl-acetyl)-piperazin-1-yl]-nicotinamide; N-(3-Methyl-butyl)-6-[4-(2-phenyl-butyryl)-piperazin-1-yl]-nicotinamide; N-(1-Methyl-butyl)-6-[4-(2-p-tolyl-acetyl)-piperazin-1-yl]-nicotinamide; N-Butyl-6-{4-[2-(2-chloro-6-fluoro-phenyl)-acetyl]-piperazin-1-yl}-nicotinamide; N-(2-Methyl-butyl)-6-[4-(2-o-tolyl-acetyl)-piperazin-1-yl]-nicotinamide; 6-{4-[3-(3,4-Difluoro-phenyl)-propionyl]-piperazin-1-yl}-N-(2-methylbutyl)-nicotinamide; N-(3-Isopropoxy-propyl)-6-[4-(2-o-tolyl-acetyl)-piperazin-1-yl]-nicotinamide; 6-{4-[3-(3,4-Difluoro-phenyl)-propionyl]-piperazin-1-yl}-N-(3-methylbutyl)-nicotinamide; 6-{4-[3-(3,4-Difluoro-phenyl)-propionyl]-piperazin-1-yl}-N-(3-methoxy-propyl)-nicotinamide; N-(1-Methyl-butyl)-6-[4-(2-o-tolyl-acetyl)-piperazin-1-yl]-nicotinamide; 6-{4-[2-(2-Chloro-6-fluoro-phenyl)-acetyl]-piperazin-1-yl}-N-(2-methylbutyl)-nicotinamide; 6-{4-[2-(2-Chloro-6-fluoro-phenyl)-acetyl]-piperazin-1-yl}-N-(1-methylbutyl)-nicotinamide; N-(2-Methyl-butyl)-6-[4-(2-phenyl-butyryl)-piperazin-1-yl]-nicotinamide; N-(1,3-Dimethylbutyl)-6-[4-(2-phenyl-butyryl)-piperazin-1-yl]-nicotinamide; 6-{4-[2-(4-Chlorophenyl)-propionyl]-piperazin-1-yl}-N-(2-methylbutyl)-nicotinamide; 6-{4-[2-(2-Chloro-6-fluoro-phenyl)-acetyl]-piperazin-1-yl}-N-(3-iso-propoxy-propyl)-nicotinamide; N-(1,3-Dimethylbutyl)-6-[4-(2-p-tolyl-acetyl)-piperazin-1-yl]-nicotinamide; 6-{4-[3-(3,4-Difluoro-phenyl)-propionyl]-piperazin-1-yl}-N-hexyl-nicotinamide; N-(1-Methyl-butyl)-6-[4-(2-phenyl-butyryl)-piperazin-1-yl]-nicotinamide; 6-{4-[2-(4-Chlorophenyl)-propionyl]-piperazin-1-yl}-N-(1-methylbutyl)-nicotinamide; 6-{4-[2-(4-Chlorophenyl)-propionyl]-piperazin-1-yl}-N-(1,3-dimethyl-butyl)-nicotinamide; N-Butyl-6-[4-(2-p-tolyl-acetyl)-piperazin-1-yl]-nicotinamide; N-(1,3-Dimethylbutyl)-6-[4-(2-o-tolyl-acetyl)-piperazin-1-yl]-nicotinamide; 6-{4-[2-(2-Chloro-6-fluoro-phenyl)-acetyl]-piperazin-1-yl}-N-(1,3-dimethylbutyl)-nicotinamide; N-(3-Methyl-butyl)-6-[4-(2-p-tolyl-acetyl)-piperazin-1-yl]-nicotinamide; N-Butyl-6-[4-(2-o-tolyl-acetyl)-piperazin-1-yl]-nicotinamide; N-(3-Butoxy-propyl)-6-[4-(2-p-tolyl-acetyl)-piperazin-1-yl]-nicotinamide; N-Pentyl-6-[4-(2-p-tolyl-acetyl)-piperazin-1-yl]-nicotinamide; N-(3-Butoxy-propyl)-6-[4-(2-phenyl-butyryl)-piperazin-1-yl]-nicotinamide; N-Pentyl-6-[4-(2-phenyl-butyryl)-piperazin-1-yl]-nicotinamide; N-(2-Ethylsulfanyl-ethyl)-6-[4-(2-p-tolyl-acetyl)-piperazin-1-yl]-nicotinamide; 6-{4-[2-(4-Chlorophenyl)-propionyl]-piperazin-1-yl}-N-(2-ethylsulfanyl-ethyl)-nicotinamide; N-(3-Ethoxy-propyl)-6-[4-(2-o-tolyl-acetyl)-piperazin-1-yl]-nicotinamide; 6-{4-[2-(2-Chloro-6-fluoro-phenyl)-acetyl]-piperazin-1-yl}-N-(3-methoxy-propyl)-nicotinamide; 6-{4-[3-(3,4-Difluoro-phenyl)-propionyl]-piperazin-1-yl}-N-(3-isopropoxy-propyl)-nicotinamide; 6-{4-[2-(4-Chlorophenyl)-propionyl]-piperazin-1-yl}-N-pentyl-nicotinamide; N-(3-Butoxy-propyl)-6-{4-[2-(2-chloro-6-fluoro-phenyl)-acetyl]-piperazin-1-yl}-nicotinamide; 6-{4-[2-(2-Chloro-6-fluoro-phenyl)-acetyl]-piperazin-1-yl}-N-hexyl-nicotinamide; 6-{4-[2-(4-Chlorophenyl)-propionyl]-piperazin-1-yl}-N-(3-methoxy-propyl)-nicotinamide; N-Hexyl-6-[4-(2-o -tolyl-acetyl)-piperazin-1-yl]-nicotinamide; 6-{4-[3-(3,4-Difluoro-phenyl)-propionyl]-piperazin-1-yl}-N-(4-phenyl-butyl)-nicotinamide; N-(3-Isopropoxy-propyl)-6-[4-(2-p-tolyl-acetyl)-piperazin-1-yl]-nicotinamide; 6-{4-[3-(3,4-Difluoro-phenyl)-propionyl]-piperazin-1-yl}-N-(1-methylbutyl)-nicotinamide; 6-{4-[3-(3,4-Difluoro-phenyl)-propionyl]-piperazin-1-yl}-N-pentyl-nicotinamide; 6-{4-[2-(4-Chlorophenyl)-propionyl]-piperazin-1-yl}-N-hexyl-nicotinamide; N-Butyl-6-{4-[2-(4-chloro-phenyl)-propionyl]-piperazin-1-yl}-nicotinamide; 6-{4-[2-(2-Chloro-6-fluoro-phenyl)-acetyl]-piperazin-1-yl}-N-(2-ethylsulfanyl-ethyl)-nicotinamide; 6-{4-[2-(2-Chloro-6-fluoro-phenyl)-acetyl]-piperazin-1-yl}-N-pentyl-nicotinamide; N-Hexyl-6-[4-(2-phenyl-butyryl)-piperazin-1-yl]-nicotinamide; N-(3-Isopropoxy-propyl)-6-[4-(2-phenyl-butyryl)-piperazin-1-yl]-nicotinamide; N-Pentyl-6-[4-(2-o-tolyl-acetyl)-piperazin-1-yl]-nicotinamide; N-Hexyl-6-[4-(2-p-tolyl-acetyl)-piperazin-1-yl]-nicotinamide; 6-{4-[3-(3,4-Difluoro-phenyl)-propionyl]-piperazin-1-yl}-N-(1,3-dimethylbutyl)-nicotinamide; 6-[4-(Naphthalene-2-carbonyl)-piperazin-1-yl]-N-pentyl-nicotinamide; 6-{4-[2-(4-Chlorophenyl)-propionyl]-piperazin-1-yl}-N-(3-dimethylamino-propyl)-nicotinamide; N-(3-Dimethylamino-propyl)-6-[4-(2-phenyl-butyryl)-piperazin-1-yl]-nicotinamide; N-(3-Dimethylamino-propyl)-6-[4-(2-p-tolyl-acetyl)-piperazin-1-yl]-nicotinamide; N-(3-Dimethylamino-propyl)-6-[4-(2-o-tolyl-acetyl)-piperazin-1-yl]-nicotinamide; N-(3-Butoxy-propyl)-6-{4-[3-(3,4-difluoro-phenyl)-propionyl]-piperazin-1-yl}-nicotinamide; N-(3-Methoxy-propyl)-6-[4-(2-p-tolyl-acetyl)-piperazin-1-yl]-nicotinamide; N-(2-Methyl-butyl)-6-[4-(2-p-tolyl-acetyl)-piperazin-1-yl]-nicotinamide; N-(3-Butoxy-propyl)-6-[4-(2-o-tolyl-acetyl)-piperazin-1-yl]-nicotinamide; 6-{4-[2-(4-Chlorophenyl)-propionyl]-piperazin-1-yl}-N-(3-isopropoxy-propyl)-nicotinamide; 6-{4-[3-(3,4-Difluoro-phenyl)-propionyl]-piperazin-1-yl}-N-phenethyl-nicotinamide; N-[2-(3-Chlorophenyl)-ethyl]-6-[4-(2-o-tolyl-acetyl)-piperazin-1-yl-]-nicotinamide; 6-{4-[2-(2-Chloro-6-fluoro-phenyl)-acetyl]-piperazin-1-yl}-N-(3-phenyl-propyl)-nicotinamide; N-(1-Methyl-3-phenyl-propyl)-6-[4-(2-p-tolyl-acetyl)-piperazin-1-yl-]-nicotinamide; N-[2-(3-Chlorophenyl)-ethyl]-6-[4-(2-phenyl-butyryl)-piperazin-1-yl-]-nicotinamide; 6-{4-[2-(2-Chloro-6-fluoro-phenyl)-acetyl]-piperazin-1-yl}-N-phenethyl-nicotinamide; N-(3-Phenyl-propyl)-6-[4-(2-o-tolyl-acetyl)-piperazin-1-yl]-nicotinamide; 6-{4-[2-(2-Chloro-6-fluoro-phenyl)-acetyl]-piperazin-1-yl}-N-[2-(3-chloro-phenyl)-ethyl]-nicotinamide; 6-{4-[2-(4-Chlorophenyl)-propionyl]-piperazin-1-yl}-N-(3-phenyl-propyl)-nicotinamide; 6-{4-[2-(4-Chlorophenyl)-propionyl]-piperazin-1-yl}-N-(1-methyl-3-phenyl-propyl)-nicotinamide; N-[2-(3-Chlorophenyl)-ethyl]-6-{4-[2-(4-chloro-phenyl)-propionyl]-piperazin-1-yl}-nicotinamide; N-[2-(3-Chlorophenyl)-ethyl]-6-[4-(2-p-tolyl-acetyl)-piperazin-1-yl-]-nicotinamide; N-Phenethyl-6-[4-(2-p-tolyl-acetyl)-piperazin-1-yl]-nicotinamide; N-(3-Phenyl-propyl)-6-[4-(2-p-tolyl-acetyl)-piperazin-1-yl]-nicotinamide; N-(1-Methyl-3-phenyl-propyl)-6-[4-(2-phenyl-butyryl)-piperazin-1-yl]-nicotinamide; 6-{4-[2-(4-Chlorophenyl)-propionyl]-piperazin-1-yl}-N-(4-phenyl-butyl)-nicotinamide; N-(4-Phenyl-butyl)-6-[4-(2-phenyl-butyryl)-piperazin-1-yl]-nicotinamide; 6-{4-[2-(2-Chloro-6-fluoro-phenyl)-acetyl]-piperazin-1-yl}-N-(1-methyl-3-phenyl-propyl)-nicotinamide; 6-{4-[3-(3,4-Difluoro-phenyl)-propionyl]-piperazin-1-yl}-N-(3-pheny-1-propyl)-nicotinamide; N-Phenethyl-6-[4-(2-o-tolyl-acetyl)-piperazin-1-yl]-nicotinamide; 6-{4-[3-(3,4-Difluoro-phenyl)-propionyl]-piperazin-1-yl}-N-(1-methy-1-3-phenyl-propyl)-nicotinamide; N-(4-Phenyl-butyl)-6-[4-(2-o-tolyl-acetyl)-piperazin-1-yl]-nicotinamide; N-(3-Imidazol-1-yl-propyl)-6-[4-(2-phenyl-butyryl)-piperazin-1-yl]-nicotinamide; 6-{4-[2-(2-Chloro-6-fluoro-phenyl)-acetyl]-piperazin-1-yl}-N-(4-phenyl-butyl)-nicotinamide; 6-{4-[2-(4-Chlorophenyl)-propionyl]-piperazin-1-yl}-N-[2-(3H-imidazol-4-yl)-ethyl]-nicotinamide; 6-{4-[2-(2-Chloro-6-fluoro-phenyl)-acetyl]-piperazin-1-yl}-N-(3-imi-dazol-1-yl-propyl)-nicotinamide; N-[2-(3H-Imidazol-4-yl)-ethyl]-6-[4-(2-p-tolyl-acetyl)-piperazin-1-yl]-nicotinamide; 6-{4-[2-(4-Chlorophenyl)-propionyl]-piperazin-1-yl}-N-(3-imidazol-1-yl-propyl)-nicotinamide; N-(2-Ethylsulfanyl-ethyl)-6-[4-(2-phenyl-butyryl)-piperazin-1-yl]-nicotinamide; N-(1-Methyl-3-phenyl-propyl)-6-[4-(2-o-tolyl-acetyl)-piperazin-1-yl-]-nicotinamide; 6-{4-[3-(3,4-Difluoro-phenyl)-propionyl]-piperazin-1-yl}-N-phenethy-1-nicotinamide; N-Phenethyl-6-[4-(2-phenyl-butyryl)-piperazin-1-yl]-nicotinamide; N-(Tetrahydrofuran-2-ylmethyl)-6-[4-(2-p-tolyl-acetyl)-piperazin-1-yl]-nicotinamide; 6-{4-[2-(4-Chlorophenyl)-propionyl]-piperazin-1-yl}-N-(tetrahydrofuran-2-ylmethyl)-nicotinamide; N-(4-Phenyl-butyl)-6-[4-(2-p-tolyl-acetyl)-piperazin-1-yl]-nicotinamide; 6-{4-[3-(3,4-Difluoro-phenyl)-propionyl]-piperazin-1-yl}-N-(tetrahydro-furan-2-ylmethyl)-nicotinamide; N-(Tetrahydrofuran-2-ylmethyl)-6-[4-(2-o-tolyl-acetyl)-piperazin-1-yl]-nicotinamide; 6-[4-(2-Phenyl-butyryl)-piperazin-1-yl]-N-(tetrahydrofuran-2-ylmethyl)-nicotinamide; 6-{4-[3-(3,4-Difluoro-phenyl)-propionyl]-piperazin-1-yl}-N-[2-(3H-1-midazol-4-yl)-ethyl]-nicotinamide; N-[2-(3H-Imidazol-4-yl)-ethyl]-6-[4-(2-o-tolyl-acetyl)-piperazin-1-yl]-nicotinamide; N-(3-Imidazol-1-yl-propyl)-6-[4-(2-o-tolyl-acetyl)-piperazin-1-yl]-nicotinamide; 6-{4-[3-(3,4-Difluoro-phenyl)-propionyl]-piperazin-1-yl}-N-(3-imidazol-1-yl-propyl)-nicotinamide; N-[2-(3H-Imidazol-4-yl)-ethyl]-6-[4-(2-phenyl-butyryl)-piperazin-1-yl]-nicotinamide; N-(3-Imidazol-1-yl-propyl)-6-[4-(2-p-tolyl-acetyl)-piperazin-1-yl]-nicotinamide; 6-{4-[2-(2-Chloro-6-fluoro-phenyl)-acetyl]-piperazin-1-yl}-N-(tetra-hydro-furan-2-ylmethyl)-nicotinamide; 6-{4-[2-(2-Chloro-6-fluoro-phenyl)-acetyl]-piperazin-1-yl}-N-[2-(3H-imidazol-4-yl)-ethyl]-nicotinamide; N-(4-Phenyl-butyl)-6-[4-(2-phenyl-cyclopropanecarbonyl)-piperazin-1-yl]-nicotinamide; N-(1-Methyl-3-phenyl-propyl)-6-[4-(2-phenyl-cyclopropanecarbonyl)-piperazin-1-yl]-nicotinamide; N-Phenethyl-6-[4-(2-phenyl-cyclopropanecarbonyl)-piperazin-1-yl]-nicotinamide; 6-[4-(2-Phenyl-cyclopropanecarbonyl)-piperazin-1-yl]-N-(tetrahydro furan-2-ylmethyl)-nicotinamide; N-[2-(3H-Imidazol-4-yl)-ethyl]-6-[4-(2-phenyl-cyclopropanecarbonyl)-piperazin-1-yl]-nicotinamide; N-(3-Imidazol-1-yl-propyl)-6-[4-(2-phenyl-cyclopropanecarbonyl)-piperazin-1-yl]-nicotinamide; N-(3-Ethoxy-propyl)-6-[4-(2-phenyl-cyclopropanecarbonyl)-piperazin-1-yl]-nicotinamide; N-(2-Ethylsulfanyl-ethyl)-6-[4-(2-phenyl-cyclopropanecarbonyl)-piperazin-1-yl]-nicotinamide; N-(1,3-Dimethylbutyl)-6-[4-(2-phenyl-cyclopropanecarbonyl)-piperazin-1-yl]-nicotinamide; N-(3-Methyl-butyl)-6-[4-(2-phenyl-cyclopropanecarbonyl)-piperazin-1-yl]-nicotinamide; N-(3-Methoxy-propyl)-6-[4-(2-phenyl-cyclopropanecarbonyl)-piperazin-1-yl]-nicotinamide; N-(3-Butoxy-propyl)-6-[4-(2-phenyl-cyclopropanecarbonyl)-piperazin-1-yl]-nicotinamide; N-Pentyl-6-[4-(2-phenyl-cyclopropanecarbonyl)-piperazin-1-yl]-nicotinamide; N-(3-Dimethylamino-propyl)-6-[4-(2-phenyl-cyclopropanecarbonyl)-piperazin-yl]-nicotinamide; N-(3-Isopropoxy-propyl)-6-[4-(2-phenyl-cyclopropanecarbonyl)-piperazin-1-yl]-nicotinamide; N-(1-Methyl-butyl)-6-[4-(2-phenyl-cyclopropanecarbonyl)-piperazin-1-yl]-nicotinamide; N-Butyl-6-[4-(2-phenyl-cyclopropanecarbonyl)-piperazin-1-yl]-nicotinamide; N-Hexyl-6-[4-(2-phenyl-cyclopropanecarbonyl)-piperazin-1-yl]-nicotinamide; and N-(2-Methyl-butyl)-6-[4-(2-phenyl-cyclopropanecarbonyl)-piperazin-1-yl]-nicotinamide.

Of the compounds described above, the most preferred are selected from the group consisting of the following:
6-[4-(2-Phenyl-cyclopropanecarbonyl)-piperazin-1-yl]-N-(3-phenyl-propyl)-nicotinamide;
N-(4-Phenyl-butyl)-6-[4-(2-phenyl-cyclopropanecarbonyl)-piperazin-1-yl]-nicotinamide;
N-(1-Methyl-3-phenyl-propyl)-6-[4-(2-phenyl-cyclopropanecarbonyl)-piperazin-1-yl]-nicotinamide;
N-Phenethyl-6-[4-(2-phenyl-cyclopropanecarbonyl)-piperazin-1-yl]-nicotinamide;
6-[4-(2-Phenyl-cyclopropanecarbonyl)-piperazin-1-yl]-N-(tetrahydrofuran-2-ylmethyl)-nicotinamide;
N-[2-(3H-imidazol-4-yl)-ethyl]-6-[4-(2-phenyl-cyclopropanecarbonyl)-piperazin-1-yl]-nicotinamide;
N-(3-Imidazol-1-yl-propyl)-6-[4-(2-phenyl-cyclopropanecarbonyl)-piperazin-1-yl]-nicotinamide;
N-(3-Ethoxy-propyl)-6-[4-(2-phenyl-cyclopropanecarbonyl)-piperazin-1-yl]-nicotinamide;
N-(2-Ethylsulfanyl-ethyl)-6-[4-(2-phenyl-cyclopropanecarbonyl)-piperazin-1-yl]-nicotinamide;
N-(1,3-Dimethylbutyl)-6-[4-(2-phenyl-cyclopropanecarbonyl)-piperazi-n-1-yl]-nicotinamide;
N-(3-Methyl-butyl)-6-[4-(2-phenyl-cyclopropanecarbonyl)-piperazin-1-yl]-nicotinamide;
N-(3-Methoxy-propyl)-6-[4-(2-phenyl-cyclopropanecarbonyl)-piperazin-1-yl]-nicotinamide;
N-(3-Butoxy-propyl)-6-[4-(2-phenyl-cyclopropanecarbonyl)-piperazin-1-yl]-nicotinamide;
N-Pentyl-6-[4-(2-phenyl-cyclopropanecarbonyl)-piperazin-1-yl]-nicotinamide;
N-(3-Dimethylamino-propyl)-6-[4-(2-phenyl-cyclopropanecarbonyl)-piperazin-1-yl]-nicotinamide;
N-(3-Isopropoxy-propyl)-6-[4-(2-phenyl-cyclopropanecarbonyl)-piperazin-1-yl]-nicotinamide;
N-(1-Methyl-butyl)-6-[4-(2-phenyl-cyclopropanecarbonyl)-piperazin-1-yl]-nicotinamide;
N-Butyl-6-[4-(2-phenyl-cyclopropanecarbonyl)-piperazin-1-yl]-nicotinamide;
N-Hexyl-6-[4-(2-phenyl-cyclopropanecarbonyl)-piperazin-1-yl]-nicotinamide; and
N-(2-Methyl-butyl)-6-[4-(2-phenyl-cyclopropanecarbonyl)-piperazin-1-yl]-nicotinamide.

The methods of the present invention also comprise administration of an effective amount of an SCD-inhibiting, Aβ42-lowering, composition, to a patient in need of such treatment, wherein the composition comprises a compound according to Formula V(a):

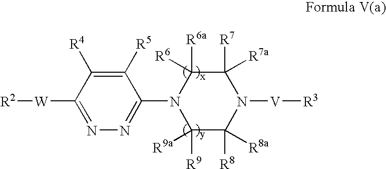

wherein: x and y are each independently 1, 2 or 3;

W is —O—, —C(O)O—, —N(R¹)—, —S(O)ₜ— (where t is 0, 1 or 2), —N(R¹)S(O)₂—, —OC(O)—, or —C (O)—, V is —C(O)—, —C(S)—, —C(O)N(R¹)—, —C(O)O—, —S(O)₂—, —S(O)₂N(R)—, or —C(R¹¹)H—;

each R¹ is independently selected from the group consisting of hydrogen, C₁-C₁₂alkyl, C₂-C₁₂hydroxyalkyl, C₄-C₁₂cycloalkylalkyl and C₇-C₁₉aralkyl;

R² is selected from the group consisting of C₁-C₁₂alkyl, C₂-C₁₂alkenyl, C₂-C₁₂hydroxyalkyl, C₂-C₁₂hydroxyalkenyl, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂heteroarylalkyl;

or R² is a multi-ring structure having 2 to 4 rings wherein the rings are independently selected from the group consisting of cycloalkyl, heterocyclyl, aryl and heteroaryl and where some or all of the rings may be fused to each other;

R³ is selected from the group consisting of C₁-C₁₂alkyl, C₂-C₁₂alkenyl, C₂-C₁₂hydroxyalkyl, C₂-C₁₂hydroxyalkenyl, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂heteroarylalkyl;

or R³ is a multi-ring structure having 2 to 4 rings wherein the rings are independently selected from the group consisting of cycloalkyl, heterocyclyl, aryl and heteroaryl and where some or all of the rings may be fused to each other;

R⁴ and R⁵ are each independently selected from hydrogen, fluoro, chloro, methyl, methoxy, trifluoromethyl, cyano, nitro, or —N(R¹³)₂;

R⁶, R^{6a}, R⁷, R^{7a}, R⁸, R^{8a}, R⁹, and R^{9a} are each independently selected from hydrogen or C₁-C₃alkyl;

or R⁷ and R^{7a} together, or R⁸ and R^{8a} together, or R⁹ and R^{9a} together, or R⁶ and R^{6a} together are an oxo group, provided that when V is —C(O)—, R⁷ and R^{7a} together or R⁸ and R^{8a} together do not form an oxo group, while the remaining R⁷, R^{7a}, R⁸, R^{8a}, R⁹, R^{9a}, R⁶ and R^{6a} are each independently selected from hydrogen or C₁-C₃alkyl;

or one of R⁶, R^{6a}, R⁷, and R^{7a} together with one of R⁸, R^{8a}, R⁹ and R^{9a} form an alkylen bridge, while the remaining R⁶, R^{6a}, R⁷, R^{7a}, R⁸, R⁹, and R^{9a} are each independently selected from hydrogen or C₁-C₃alkyl;

R¹¹ is hydrogen or C₁-C₃alkyl;

and each R¹³ is independently selected from hydrogen or C₁-C₆alkyl;

a stereoisomer, enantiomer or tautomer thereof, a pharmaceutically acceptable salt thereof, a pharmaceutical composition thereof or a prodrug thereof.

The compounds of Formula V(a) include: [4-(6-Phenethylamino-pyridazin-3-yl)-piperazin-1-yl]-(2-trifluoromethyl-phenyl)-methanone; and {4-[6-(Methyl-phenethyl-amino)-pyridazin-3-yl]-piperazin-1-yl}-(2-trifluoromethyl phenyl)-methanone.

The compounds of Formula V(a) also include: {4-[6-(3-Methyl-butylsulfanyl)-pyridazin-3-yl]-piperazin-1-yl}-(2-trifluoromethyl-phenyl)-methanone.

The compounds of Formula V(a) also include: [4-(6-Phenethylsulfanyl-pyridazin-3-yl)-piperazin-1-yl]-(2-trifluoromethyl-phenyl)-methanone; {4-[6-(2-Phenyl-ethanesulfinyl)-pyridazin-3-yl]-piperazin-1-yl}-(2-trifluoromethyl-phenyl)-methanone; and {4-[6-(2-Phenyl-ethanesulfonly)-pyridazin-3-yl]-piperazin-1-yl}-(2-trifluoromethyl-phenyl)-methanone.

The compounds of Formula V(a) also include: {4-[6-(2-Cyclopropyl-ethoxy}-pyridazin-3-yl]-piperazin-1-yl}-(2-trifluoromethyl-phenyl)-methanone. A compound, namely, [4-(6-Phenethyloxy_pyridazin-3-yl)-piperazin-1-y]-(2-trifluoromethyl-phenyl)-methanone.

The methods of the present invention also comprise administration of an effective amount of an SCD-inhibiting, Aβ42-lowering, composition, to a patient in need of such treatment, wherein the composition comprises a compound according to Formula V(b):

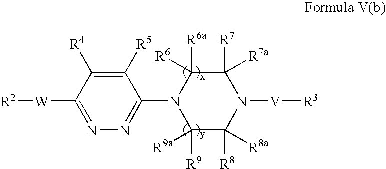

wherein: x and y are each independently 1, 2 or 3;

W is —C(O)N(R¹)—; —C(O)N[C(O)R^{1a}]—, —N(R¹)C(O)N(R¹)— or —N(R¹)C(O)—; V is —C(O)—, —C(S)—, or —C(R¹⁰)H;

each R¹ is independently selected from the group consisting of hydrogen;

C₁-C₆alkyl optionally substituted with one or more substituents selected from the group consisting of halo, methyl, or trifluoromethyl;

and C₂-C₆alkyl optionally substituted with one or more substituents selected from the group consisting of methoxy and hydroxyl;

R^{1a} is selected from the group consisting of hydrogen, C₁-C₆alkyl, and cycloalkyl;

R² is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkyalkyl, aryl, C₇-C₁₂aralkyl, C₃-C₁₂heterocyclyl, C₃-C₁₂heterocylylalkyl, C₁-C₁₂heterorayl, and C₃-C₁₂heteroarylalkyl;

or R² is a multi-ring structure having 2 to 4 rings wherein the rings are independently selected from the group consisting of cycloalkyl, heterocyclyl, aryl and heteroaryl and where some or all of the rings may be fused to each other;

R³ is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₁-C₁₂heteroaryl and C₃-C₁₂heteroarlalkyl;

or R³ is a multi-ring structure having 2 to 4 rings wherein the rings are independently selected from the group consisting of cycloalkyl, heterocyclyl, aryl and heteroaryl and where some or all of the rings may be fused to each other;

R⁴ and R⁵ are each independently selected from hydrogen, fluoro, chloro, methyl, methoxy, trifluoromethyl, cyano, nitro, or —N(R¹²)₂;

R⁶, R^{6a}, R⁷, R^{7a}, R⁸, R^{8a}, R⁹, and R^{9a} are each independently selected from hydrogen or C₁-C₃ alkyl;

or R⁶ and R^{6a} together, or R⁷ and R^{7a} together, or R⁸ and R^{8a} together, or R⁹ and R^{9a} together are an oxo group, provided that when V is —C(O)—, R⁷ and R⁷, together or R⁸ and R^{8a} together do not form an oxo group, while the remaining R⁶, R^{6a}, R⁷, R^{7a}, R⁸, R^{8a}, R⁹, and R^{9a} are each independently selected from hydrogen or C₁-C₃alkyl;

or one of R⁶, R^{6a}, R⁷, and R^{7a} together with one of R⁸, R^{8a}, R⁹ and R^{9a} form an alkylen bridge, while the remaining R⁶, R^{6a}, R⁷, R^{7a}, R⁸, R^{8a}, R⁹, and R^{9a} are each independently selected from hydrogen or C₁-C₃alkyl;

R¹⁰ is hydrogen or C₁-C₃alkyl; and each R¹² is independently selected from hydrogen or C₁-C₆alkyl;

a stereoisomer, enantiomer or tautomer thereof, a pharmaceutical acceptable salt thereof, a pharmaceutical composition thereof or a prodrug thereof.

The compounds of Formula V(b) include: 1-Pentyl-3-{6-[4-(2-trifluoromethylbenzoyl)piperazin-1-yl]pyridazin-3-yl}urea.

The compounds of Formula V(b) also include: 1-Benzyl-3-{6-[4-(2-trifluoromethylbenzoyl)piperazin-1-yl]pyridazin-3-yl}urea.

The compounds of Formula V(b) also include: 3-(3-{6-[4-(2-Trifluoromethylbenzoyl)piperazin-1-yl]-pyridazin-3-yl}ureido) propionic acid ethyl ester; 1-Butyl-3-{6-[4-(2-trifluoromethylbenzoyl)piperazin-1-yl]pyridazin-3-yl}urea; 1-(2-chloroethyl)-3-{6-[4-(2-trifluoromethylbenzoyl)-piperazin-1-yl]pyridazin-3-yl}urea; 1-{6-[4-(2,6-Difluorobenzoyl)piperazin-1-yl]pyridazin-3-yl}-3-(3-methylbutyl) urea; 1-(3,3-Dimethylbutyl)-3-{6-[4-(2-trifluoromethylbenzoyl)piperazin-1-yl]pyridazin-3-yl}urea; 1-(2-Isopropoxyethyl)-3-{6-[4-(2-trifluoromethylbenzoyl)-piperazin-1-yl]pyridazin-3-yl}urea; 1-(3-Hydroxy-4,4-dimethylpentyl)-3-{6-[4-(2-trifluoromethylbenzoyl)piperazin-1-yl]pyridazin-3-yl}urea; 1-Hexyl-3-{6-[4-(2-trifluoromethylbenzoyl)piperazin-1-yl]pyridazin-3-yl}urea; 1-Heptyl-3-{6-[4-(2-trifluoromethylbenzoyl)piperazin-1-yl]pyridazin-3-yl}urea; and 1-(4-Methylpentyl)-3-{6-[4-(2-trifluoromethylbenzoyl)piperazin-1-yl]pyridazin-3-yl}urea.

The compounds of Formula V(b) also include: 1-(4-Fluorophenyl)-3-{6-[4-(2-trifluoromethylbenzoyl)piperazin-1-yl]-pyridazin-3-yl}urea; and 1-(2-Fluorophenyl)-3-{6-[4-(2-trifluoromethylbenzoyl)piperazin-1-yl]pyridazin-3-yl}urea.

The compounds of Formula V(b) also include: 1-[1-4(-Fluorophenyl)ethyl]3-{6-[4-(2-trifluoromethylbenzoyl)piperazin-1-yl]pyridazin-3-yl}urea; 1-[1-(4-Fluorophenyl)ethyl]-3-{6-[4-(2-trifluoromethylbenzoyl)piperazin-1-yl]pyridazin-3-yl)urea; 1-(3-(4-Fluorophenyl)propyl]-3-{6-[4-(2-trifluoromethylbenzoyl)piperazin-1-yl]pyridazin-3-yl}urea; 1-Phenethyl-3-{6-[4-(2-trifluoromethylbenzoyl)piperazin-1-yl]-pyridazin-3-yl}urea; 1-(4-Fluorobenzyl)-3-{6-[4-(2-trifluoromethylbenzoyl)piperazin-1-yl]pyridazin-3-yl}urea; and 1-(3,4-Dichlorobenzyl)-3-{6-[4-(2-trifluoromethylbenzoyl)piperazin-1-yl]pyridazin-3-yl}urea.

The compounds of Formula V(b) also include: 1-(2-Phenylcyclopropyl)-3-{6-[4-(2-trifluoromethylbenzoyl)piperazin-1-yl]pyridazin-3-yl}urea; 1-Cycoopentyl-3-{6-[4-(2-trifluoromethylbenzoyl)piperazin-1-yl]-pyridazin-3-yl}urea; 1-(3-Cyclopropylpropyl)-3-{6-[4-(2-trifluoromethylbenzoyl)piperazin-1-yl]pyridazin-3-yl}urea; 1-Cyclopropylmethyl-3-{6-[4-(2-trifluoromethylbenzoyl)piperazin-1-yl]pyridazin-3-yl}urea; 1-(2-Cyclopropylethyl)-3-{6-[4-(2-trifluoromethylbenzoyl)piperazin-1-yl}pyridazin-3-yl}urea; 1-(2-Cyclopropylethyl)-3-{6-[4-(2-fluoro-6-trifluoromethylbenzoyl)piperazin-1-yl]pyridazin-3-yl}urea; 1-(2-Cyclopropylethyl)-3-{6-[4-(5-fluoro-2-trifluoromethylbenzoyl)piperazin-1-yl]pyridazin-3-yl}urea; 1-Cyclohexyl-3-{6-[4-(2-trifluoromethylbenzoyl)piperazin-1-yl]pyridazin-3-yl urea 1-(2-Cyclopropylethyl)-3-{6-[4-(2,6-difluorobenzoyl)piperazin-1-yl]pyridazin-3-yl}urea; and 1-(3-Cyclopropylpropyl)-3-{6-[4-(5-fluoro-2-trifluoromethylbenzoyl)piperazin-1-yl]pyridazin-3-yl}urea.

The compounds of Formula V(b) also include: 4-Phenyl-N-{6-[4-(2-trifluoromethylbenzoyl)piperazin-1-yl]pyridazin-3-y}butyramide.

The compounds of Formula V(b) also include: 4-Methylpentanoic acid {6-[4-(2-trifluoromethylbenzoyl)-piperazin-1-yl]pyridazin-3-yl]amide.

The compounds of Formula V(b) also include: 3-Cyclopentyl-N-{6-[4-(2-trifluoromethylbenzoyl)piperazin-1-yl]pyridazin-3-yl}propionamide.

The compounds of Formula V(b) also include: 6-[4-(2-Trifluoromethylbenzoyl)piperazin-1-yl]pyridazine-3-carboxylic acid phenethylamide; 6-[4-(2-Trifluoromethylbenzoyl)piperazin-1-yl]pyridazine-3-carboxylic acid [2-(4-methoxyphenyl)ethyl]amide; 6-[4-(2-Trifluoromethylbenzoyl)piperazin-1-yl]pyridazine-3-carboxylic acid [2-(3-fluorophenyl)ethyl]amide; 6-[4-(2-Trifluoromethylbenzoyl)piperazin-1-yl]pyridazine-3-carboxylic acid (3-phenylpropyl)amide; and 6-[4-(2-Trifluoromethylbenzoyl)piperazin-1-yl]pyridazine-3-carboxylic acid [2-(4-fluorophenyl)ethyl]amide.

The compounds of Formula V(b) also include: 4-Methyl-2-({6-[4-(2-trifluoromethylbenzoyl)piperazin-1-yl]-pyridazine-3-carbonyl}amino)pentanoic acid methyl ester; 6-[4-(2-Trifluoromethylbenzoyl)piperazin-1-yl]pyridazine-3-carboxylic acid (3-methylbutyl)amide; 6-[4-(5-Fluoro-2-trifluoromethylbenzoyl)piperazin-1-yl]pyridazine-3-carboxylic acid (3-methylbutyl)amide; 6-[4-(4-Fluoro-2-trifluoromethylbenzoyl)piperazin-1-yl]pyridazine-3-carboxylic acid (3-methylbutyl)amide; and 4-Methyl-2-({6-[4-(2-trifluoromethylbenzoyl)piperazin-1-yl]pyridazine-3-carbonyl}amino)pentanoic acid.

The compounds of Formula V(b) also include: 6-[4-(2-Trifluoromethylbenzoyl)piperazin-1-yl]pyridazine-3-carboxylic acid (2-cyclopropylethyl)amide; 6-[4-(5-Fluoro-2-trifluoromethylbenzoyl)piperazin-1-yl]pyridazine-3-carboxylic acid (2-cyclopropylethyl)amide; and 6-[4-(2-Trifluoromethylbenzoyl)piperazin-1-yl]pyridazine-3-carboxylic acid cyclopropylmethylamide.

The compounds of Formula V(b) also include: 4-(4-Methoxyphenyl)-N-{6-[4-(2-trifluoromethylbenzoyl)-piperazin-1-yl]pyridazin-3-yl}butyramide; and 3-(4-Fluorophenyl)-N-{6-[4-(2-trifluoromethylbenzoyl)-piperazin-1-yl]-pyridazin-3-yl}propionamide.

The compounds of Formula V(b) also include: 2-Benzyloxy-N-{6-[4-(2-trifluoromethylbenzoyl)piperazin-1-yl]pyridazin-3-yl}acetamide; 2-Ethoxy-N-{6-[4-(2-trifluoromethylbenzoyl)piperazin-1-yl]pyridazin-3-yl}acetamide; 2-Cyclopropylmethoxy-N-{6-[4-(2-trifluoromethylbenzoyl)-piperazin-1-yl]pyridazin-3-yl}acetamide; 2-(2-Methoxyethoxy)-N-{6-[4-(2-trifluoromethylbenzoyl)-piperazin-1-yl]pyridazin-3-yl}acetamide; N-{6-[4-(2-Trifluoromethylbenzoyl)piperazin-1-yl]pyridazin-3-yl}-2-(3,3,3-trifluoropropoxy)acetamide; 3-Methoxy-N-{6-[4-(2-trifluoromethylbenzoyl)piperazin-1-yl]pyridazin-3-yl}propionamide; 3-Phenoxy-N-{6-[4-(2-trifluoromethylbenzoyl)piperazin-1-yl]-pyridazin-3-yl}propionamide; 2-Butoxy-N-{6-[4-(2-trifluoromethylbenzoyl)piperazin-1-yl]pyridazin-3-yl}acetamide; 2-Methyl-1-{6-[4-(2-trifluoromethylbenzoyl)piperazin-1-yl]pyridazin-3-yl carbamoyl}propylamine; 2-Phenoxy-N-{6-[4-(2-trifluoromethylbenzoyl)piperazin-1-yl]pyridazin-3-yl}acetamide; {6-[4-(2-Trifluoromethylbenzoyl)piperazin-1-yl]pyridazin-3-yl}carbamic acid butyl ester; {6-[4-(2-Trifluoromethylbenzoyl)piperazin-1-yl]pyridazin-3-yl}carbarnic acid propyl ester; {6-[4-(2-Trifluoromethylbenzoyl)piperazin-1-yl]pyridazin-3-yl}carbamic acid isobutyl ester; {6-[4-(2-Trifluoromethylbenzoyl)piperazin-1-yl]pyridazin-3-yl}carbamic acid ethyl ester; Hexanoic Acid {6-[4-(2-trifluoromethylbenzoyl)piperazin-1-yl]-pyridazin-3-yl}amide; 4-Fluoro-N-{6-[4-(2-trifluoromethylbenzoyl)piperazin-1-yl]-pyridazin-3-yl benzamide; {6-[4-(2-Trifluoromethylbenzoyl)piperazin-1-yl]pyridazin-3-yl}carbamic acid 3,3-dimethylbutyl ester; and {6-[4-(2-Trifluoromethylbenzoyl)piperazin-1-yl]pyridazin-3-yl}carbamic acid 2-cyclopropylethyl ester.

The compounds of Formula V(b) also include: 4-Cyclohexyl-N-{6-[4-(2-trifluoromethylbenzoyl)piperazin-1-yl]pyridazin-3-yl}butyramide; 2,2,3,3-Tetramethylcyclopropanecarboxylic acid {6-[4-(2-trifluoromethylbenzoyl)piperazin-1-yl]pyridazin-3-yl}amide; Cyclopropanecarboxylic acid {6-[4-(2-trifluoromethyl-benzoyl)piperazin-1-yl]pyridazin-3-yl)amide; 1-Trifluoromethylcyclopropanecarboxylic acid {6-[4-(2-trifluoromethylbenzoyl)piperazin-1-yl]pyridazin-3-yl}amide; and 2-Phenylcyclopropanecarboxylic acid {6-[4-(2-trifluoromethylbenzoyl)piperazin-1-yl]pyridazin-3-yl}amide.

The compounds of Formula V(b) also include: 6-[4-(2-Trifluoromethylbenzoyl)piperazin-1-yl]-pyridazine-3-carboxylic acid indan-1-yl amide; 6-[4-(2-Trifluoromethylbenzoyl)piperazin-1-yl]-pyridazine-3-carboxylic acid (2-oxo-1,3-diaza-bicyclo[3.1.0]hex-3-en-4-yl)amide; 6-[4-(2-Trifluoromethylbenzoyl)piperazin-1-yl]-pyridazine-3-carboxylic acid (5-oxo-4,5-dihydro-1H-pyrazol-3-yl)amide; 6-[4-(2-Trifluoromethylbenzoyl)piperazin-1-yl]-pyridazine-3-carboxylic acid indan-5-yl amide; 5-[1, 2]Dithiolan-3-yl-pentanoic acid {6-[4-(2-Trifluoromethylbenzoyl)-1-yl]-pyridazin-3-yl}-amide; 6-[4-(2-Trifluoromethyl-benzoyl)-piperazin-1-yl]-pyridazine-3-carboxylic acid (2-thiophen-2-yl-ethyl)-amide; 6-[4-(2-Trifluoromethylbenzoyl)piperazin-1-yl]pyridazine-3-carboxylic acid (2-benzo[1,3]dioxol-5-yl-ethyl)amide; 6-[4-(2-Trifluoromethylbenzoyl)piperazin-1-yl]pyridazine-3-carboxylic acid (2,2-difluoro-2-pyridin-2-ylethyl)amide; 6-[4-(2-Trifluoromethylbenzoyl)piperazin-1-yl]pyridazine-3-carboxylic acid (2-pyridin-2-yl ethyl)amide, and 6-[4-(2-Trifluoromethylbenzoyl)piperazin-1-yl]pyridazine-3-carboxylic acid (pyridin-2-yl-methyl)amide.

The compounds of Formula V(b) also include: 6-[4-(2-Trifluoromethylbenzoyl)piperazin-1-yl]pyridazine-3-carboxylic acid (5-chloro-pyridin-2-yl)amide; 6-[4-(2-Trifluoromethylbenzoyl)piperazin-1-yl]pyridazine-3-carboxylic acid (5-trifluoromethylpyridin-2-yl)amide; 6-[4-(2-Trifluoromethylbenzoyl)piperazin-1-yl]pyridazine-3-carboxylic acid (7H-purin-6-yl)amide; 6-[4-(2-Trifluoromethylbenzoyl)piperazin-1-yl]pyridazine-3-carboxylic acid pyrazin-2-yl amide; 6-[4-(2-Trifluoromethylbenzoyl)piperazin-1-yl]-pyridazine-3-carboxylic acid (1H-tetrazol-5-yl)amide; 6-[4-(2-Trifluoromethylbenzoyl)piperazin-1-yl]pyridazine-3-carboxylic acid (2H-[1,2,4]triazol-3-yl)amide; 6-[4-(2-Trifluoromethylbenzoyl)piperazin-1-yl]pyridazine-3-carboxylic acid (3-methyl-isoxazol-5-yl)amide; 6-[4-(2-Trifluoromethylbenzoyl)piperazin-1-yl]-pyridazine-3-carboxylic acid (5-methyl-isoxazol-3-yl)amide; 6-[4-(2-Trifluoromethylbenzoyl)piperazin-1-yl]-pyridazine-3-carboxylic acid (1H-pyrazol-3-yl)amide; 6-[4-(2-Trifluoromethylbenzoyl)piperazin-1-yl]-pyridazine-3-carboxylic acid (5-methyl-1H-pyrazol-3-yl)amide; 6-[4-(2-Trifluoromethylbenzoyl)piperazin-1-yl]-pyridazine-3-carboxylic acid pyrimidin-2-yl amide; 6-[4-(2-Trifluoromethylbenzoyl)piperazin-1-yl]-pyridazine-3-carboxylic acid pyrazin-2-yl amide; 6-[4-(2-Trifluoromethylbenzoyl)piperazin-1-yl]-pyridazine-3-carboxylic acid (4-methyl-pyrimidin-2-yl)amide; 6-[4-(2-Trifluoromethylbenzoyl)piperazin-1-yl]pyridazine-3-carboxylic acid (2-oxo-2,3-dihydro-pyrimidin-4-yl)amide; 6-[4-(2-Trifluoromethylbenzoyl)piperazin-1-yl]pyridazine-3-carboxylic acid (6-oxo-1,6-dihydro-pyrimidin-2-yl)amide; 6-[4-(2-Trifluoromethylbenzoyl)piperazin-1-yl]-pyridazine-3-carboxylic acid [1,3,4]thiadiazol-2-yl amide; 6-[4-(2-Trifluoromethylbenzoyl)piperazin-1-yl]-pyridazine-3-carboxylic acid thiazol-2-yl amide; 6-[4-(2-Trifluoromethylbenzoyl)piperazin-1-yl]pyridazine-3-carboxylic acid pyridin-2-yl amide; 6-[4-(2-Trifluoromethyl-benzoyl)piperazin-1-yl]pyridazine-3-carboxylic acid pyridazin-3-ylamide; 6-[4-(2-Trifluoromethyl-benzoyl)piperazin-1-yl]pyridazine-3-carboxylic acid pyridin-3-yl amide; 6-[4-(2-Trifluoromethylbenzoyl)piperazin-1-yl)pyridazine-3-carboxylic acid pyridin-4-yl)amide; 6-[4-(2-Trifluoromethylbenzoyl)piperazin-1-yl]pyridazine-3-carboxylic acid (6-oxo-1,6-dihydro-[1,3,5]triazin-2-yl)amide; 6-[4-(2-Trifluoromethylbenzoyl)piperazin-1-yl]pyridazine-3-carboxylic acid (5-fluor-pyridin-2-yt)-amide; 6-[4-(2-Trifluoromethyl-benzoyl)-piperazin-1-yl]-pyridazine-3-carboxylic acid (5-cyano-pyridin-2-yl)-amide; 6-[4-(2-Trifluoromethylbenzoyl)piperazin-1-yl]pyridazine-3-carboxylic acid (4,6-dimethyl-pyrimidin-2-yl)-amide; 6-[4-(2-Trifluoromethylbenzoyl)piperazin-1-yl]pyridazine-3-carboxylic acid (2-chloro-pyridin-4-yl)-amide; 6-[4-(2-Trifluoromethyl-benzoyl)-piperazin-1-yl]-pyridazine-3-carboxylic acid (1H-indol-6-yl)-amide; 6-[4-(2-Trifluoromethyl-benzoyl)-piperazin-1-yl]-pyridazine-3-carboxylic acid (1H-indol-4-yl)-amide; 6-[4-(2-Trifluoromethyl-benzoyl)-piperazin-1-yl]-pyridazine-3-carboxylic acid (1H-indazol-5-yl)-amide; 6-[4-(2-Trifluoromethyl-benzoyl)-piperazin-1-yl]-pyridazine-3-carboxylic acid (1H-indazol-6-yl)-amide; 6-[4-(2-Trifluoromethylbenzoyl)piperazin-1-yl]pyridazine-3-carboxylic acid (4-methyl-thiazol-2-yl)-amide; 6-[4-(2-Tri fluoromethyl-benzoyl)-piperazin-1-yl]-pyridazine-3-carboxylic acid (5-methyl-thiazol-2-yl)-amide; 6-[4-(2-Trifluoromethyl-benzoyl)-piperazin-1-yl]-pyridazine-3-carboxylic acid (5-thioxo-4,5-dihydro-1H-[1, 2,4]triazol-3-yl)-amide; 6-[4-(2-Trifluoromethylbenzoyl)piperazin-1-yl]-pyridazine-3-carboxylic acid (1H-benzoimidazol-2-yl)-amide; 6-[4-(2-Trifluoromethylbenzoyl)piperazin-1-yl]pyridazine-3-carboxylic acid (6-methylpyridazin-3-yl)amide; 6-[4-(2-Trifluoromethylbenzoyl)piperazin-1-yl]pyridazine-3-carboxylic acid (6-methoxypyridazin-3-yl)amide; and 6-[4-(2-Trifluoromethylbenzoyl)-piperazin-1-yl]-pyridazine-3-carboxylic acid (6-chloro-pyridazin-3-yl)-amide.

The compounds of Formula V(b) also include: 6-[4-(2-Trifluoromethylbenzoyl)piperazin-1-yl]pyridazine-3-carboxylic acid (4-chloro-phenyl)amide; 6-[4-(2-Trifluoromethylbenzoyl)piperazin-1-yl]pyridazine-3-carboxylic acid (4-carbamoyl-phenyl)amide; 6-[4-(2-Trifluoromethylbenzoyl)piperazin-1-yl]pyridazine-3-carboxylic acid (3-carbamoyl-phenyl)amide; 6-[4-(2-Trifluoromethylbenzoyl)piperazin-1-yl]pyridazine-3-carboxylic acid m-tolylamide; 6-[4-(2-Trifluoromethylbenzoyl)piperazin-1-yl]pyridazine-3-carboxylic acid p-tolylamide; 6-[4-(2-Trifluoromethylbenzoyl)piperazin-1-yl]pyridazine-3-carboxylic acid o-tolylamide; 6-14-(2-Trifluoromethylbenzoyl)piperazin-1-yl]pyridazine-3-carboxylic acid (2-propylphenyl)amide; 6-[4-(2-Trifluoromethylbenzoyl)piperazin-1-yl]-pyridazine-3-carboxylic acid (4-propylphenyl)amide; 6-[4-(2-Trifluoromethylbenzoyl)piperazin-1-yl]pyridazine-3-carboxylic acid (4-isopropylphenyl)amide; 6-[4-(2-Trifluoromethyl-benzoyl)piperazin-1-yl]pyridazine-3-carboxylic acid (2-isopropylphenyl)amide; 6-[4-(2-Trifluoromethylbenzoyl)piperazin-1-yl]pyridazine-3-carboxylic acid (2-chloro-phenyl)amide; 6-[4-(2-Trifluoromethylbenzoyl)piperazin-1-yl]pyridazine-3-carboxylic acid (2-cyano-3-fluorophenyl)amide; 6-[4-(2-Trifluoromethylbenzoyl)piperazin-1-yl]pyridazine-3-carboxylic acid (2,4-dimethyl-phenyl)amide; 6-[4-(2-Trifluoromethylbenzoyl)piperazin-1-yl]pyridazine-3-carboxylic acid (2,5-dimethyl-phenyl)amide; 6-[4-(2-Trifluoromethylbenzoyl)piperazin-1-yl]pyridazine-3-carboxylic acid (2,6-dimethyl-phenyl)amide; 6-[4-(2-Trifluoromethylbenzoyl)piperazin-1-yl]pyridazine-3-carboxylic acid (2,3-dimethyl-phenyl)amide; 6-[4-(2-Trifluoromethylbenzoyl)piperazin-1-yl]pyridazine-3-carboxylic acid (3,5-dimethyl-phenyl)amide; 6-[4-(2-Trifluoromethylbenzoyl)piperazin-1-yl]pyridazine-3-carboxylic acid (3,4-dimethyl-phenyl)amide; 6-[4-(2-Trifluoromethyl-benzoyl)piperazin-1-yl]pyridazine-3-carboxylic acid (4-ethyl-phenyl)amide; 6-[4-(2-Trifluoromethyl-benzoyl)piperazin-1-yl]pyridazine-3-carboxylic acid (2-ethyl-phenyl)amide; 6-[4-(2-Trifluoromethylbenzoyl)piperazin-1-yl]pyridazine-3-carboxylic acid (3-fluoro-2-methylphenyl)amide; 6-[4-(2-Trifluoromethylbenzoyl)piperazin-1-yl]pyridazine-3-carboxylic acid (2-fluoro-4-methylphenyl)amide; 6-[4-(2-Trifluoromethylbenzoyl)piperazin-1-yl]pyridazine-3-carboxylic acid (4-fluoro-2-methylphenyl)amide; 6-[4-(2-Trifluoromethylbenzoyl)piperazin-1-yl]pyridazine-3-carboxylic acid (2-fluoro-5-methylphenyl)amide; 6-[4-(2-Trifluoromethylbenzoyl)piperazin-1-yl]-pyridazine-3-carboxylic acid (3-fluoro-5-methylphenyl)amide; 6-[4-(2-Trifluoromethylbenzoyl)piperazin-1-yl]-pyridazine-3-carboxylic acid (3-fluor-phenyl)-amide; 6-[4-(2-Trifluoromethylbenzoyl)piperazin-1-yl]-pyridazine-3-carboxylic acid (2-fluor-phenyl)amide; 6-[4-(2-Trifluoromethylbenzoyl)piperazin-1-yl]pyridazine-3-carboxylic acid (4-fluoro-phenyl)amide; 6-[4-(2-Trifluoromethylbenzoyl)piperazin-1-yl]pyridazine-3-carboxylic acid (2,4-difluoro-phenyl)amide; 6-[4-(2-Trifluoromethylbenzoyl)piperazin-1-yl]pyridazine-3-carboxylic acid (2,5-difluoro-phenyl)amide; 6-[4-(2-Trifluoromethylbenzoyl)piperazin-1-yl]-pyridazine-3-carboxylic acid (3,4-difluoro-phenyl)amide; 6-[4-(2-Trifluoromethylbenzoyl)piperazin-1-yl]-pyridazine-3-carboxylic acid (2,3-difluoro-phenyl)amide; 6-[4-(2-Trifluoromethylbenzoyl)piperazin-1-yl]pyridazine-3-carboxylic acid (2,6-difluoro-phenyl)amide; 6-[4-(2-Trifluoromethylbenzoyl)piperazin-1-yl]pyridazine-3-carboxylic acid (4-cyano-phenyl)-amide; 6-[4-(2-Trifluoromethylbenzoyl)piperazin-1-yl]pyridazine-3-carboxylic acid (2-cyano-phenyl)-amide; 6-[4-(2-Trifluoromethylbenzoyl)piperazin-1-yl]pyridazine-3-carboxylic acid (3-cyano-phenyl)amide; 6-[4-(2-Trifluoromethylbenzoyl)piperazin-1-yl]pyridazine-3-carboxylic acid (3-chloro-phenyl)amide; 6-[4-(2-Trifluoromethylbenzoyl)piperazin-1-yl]pyridazine-3-carboxylic acid (3-chloro-2-methylphenyl)amide; 6-[4-(2-Trifluoromethylbenzoyl)piperazin-1-yl]pyridazine-3-carboxylic acid (2-chloro-3-methylphenyl)amide; 6-[4-(2-Trifluoromethylbenzoyl)piperazin-1-yl]pyridazine-3-carboxylic acid (2,5-dichlorophenyl)amide; 6-[4-(2-Trifluoromethylbenzoyl)piperazin-1-yl]pyridazine-3-carboxylic acid (2-chloro-5-methylphenyl)amide; 6-[4-(2-Trifluoromethylbenzoyl)piperazin-1-yl]pyridazine-3-carboxylic acid (2-chloro-6-methylphenyl)amide; 6-[4-(2-Trifluoromethylbenzoyl)piperazin-1-yl]pyridazine-3-carboxylic acid (4-chloro-2-methylphenyl)amide; 6-[4-(2-Trifluoromethylbenzoyl)piperazin-1-yl]pyridazine-3-carboxylic acid (4-chloro-3-methylphenyl)amide; 6-[4-(2-Trifluoromethylbenzoyl)piperazin-1-yl]pyridazine-3-carboxylic acid (3-chloro-4-methylphenyl)amide; 6-[4-(2-Trifluoromethylbenzoyl)piperazin-1-yl]pyridazine-3-carboxylic acid (2-chloro-4-methylphenyl)amide; 6-[4-(2-Trifluoromethylbenzoyl)piperazin-1-yl]pyridazine-3-carboxylic acid (2-chloro-5-fluorophenyl)amide; 6-[4-(2-Trifluoromethylbenzoyl)piperazin-1-yl]pyridazine-3-carboxylic acid (5-chloro-2-fluorophenyl)amide; 6-[4-(2-Trifluoromethylbenzoyl)piperazin-1-yl]pyridazine-3-carboxylic acid (2,5-difluorophenyl)amide; 6-[4-(2-Trifluoromethylbenzoyl)piperazin-1-yl]pyridazine-3-carboxylic acid (2,6-dichlorophenyl)amide; 6-[4-(2-Trifluoromethylbenzoyl)piperazin-1-yl]pyridazine-3-carboxylic acid (2-trifluoromethylphenyl)amide; 6-[4-(2-Trifluoromethylbenzoyl)piperazin-1-yl]pyridazine-3-carboxylic acid (4-trifluoromethylphenyl)amide; 6-[4-(2-Tri fluoromethylbenzoyl)-piperazin-1-yl]pyridazine-3-carboxylic acid (3-trifluoromethylphenyl)amide; 6-[4-(2-Trifluoromethylbenzoyl)piperazin-1-yl]pyridazine-3-carboxylic acid phenylamide; 6-[4-(2-Trifluoromethylbenzoyl)piperazin-1-yl]pyridazine-3-carboxylic acid (5-chloro-2-methoxyphenyl)amide; 6-[4-(2-Trifluoromethylbenzoyl)piperazin-1-yl]pyridazine-3-carboxylic acid (2,5-dimethoxyphenyl)amide; 6-[4-(2-Trifluoromethylbenzoyl)piperazin-1-yl]pyridazine-3-carboxylic acid (2-chloro-4-methoxyphenyl)amide; 6-[4-(2-Trifluoromethylbenzoyl)-piperazin-1-yl]pyridazine-3-carboxylic acid (4-methoxy-phenyl)amide; 6-[4-(2-Trifluoromethylbenzoyl)piperazin-1-yl]pyridazine-3-carboxylic acid (2-methoxyphenyl)amide; 6-[4-(2-Trifluoromethylbenzoyl)piperazin-1-yl]pyridazine-3-carboxylic acid (3-methoxyphenyl)amide; 4-({6-[4-(2-Trifluoromethylbenzoyl)piperazin-1-yl]pyridazine-3-carbonyl}amino)-benzoic acid methyl ester; 4-({6-[4-(2-Trifluoromethylbenzoyl)piperazin-1-yl]pyridazine-3-carbonyl}amino)-benzoic acid; 2-({6-[4-(2-Trifluoromethylbenzoyl)piperazin-1-yl]pyridazine-3-carbonyllamino)-benzoic acid methyl ester; 2-({6-[4-(2-Trifluoromethylbenzoyl)piperazin-1-yl]pyridazine-3-carbonyl}amino)-benzoic acid; 6-[4-(2-Trifluoromethylbenzoyl)piperazin-1-yl]pyridazine-3-carboxylic acid (3,4-dichlorophenyl)amide;

The compounds of Formula V(b) also include: 6-[4-(2-Trifluoromethylbenzoyl)piperazin-1-yl]pyridazine-3-carboxylic acid (2-ethoxyethyl)amide; 6-[4-(2-Trifluoromethylbenzoyl)piperazin-1-yl]pyridazine-3-carboxylic acid (2-methoxy-3,3-dimethylbutyl)amide; and 2-(2-Cyclopropyl-ethoxy)-N-{6-[4-(2-trifluoromethyl-benzoyl)-piperazin-1-yl]-pyridazin-3-yl}-acetamide.

The compounds of Formula V(b) also include: 6-[4-(2-Trifluoromethylbenzoyl)piperazin-1-yl]pyridazine-3-carboxylic acid [2-(2,4-fluorophenyl)ethyl]amide; 6-[4-(2-Trifluoromethylbenzoyl)piperazin-1-yl]pyridazine-3-carboxylic acid [2-(2-fluorophenyl)ethyl]amide; 6-[4-(2-Trifluoromethylbenzoyl)piperazin-1-yl]pyridazine-3-carboxylic acid [2-(4-chlorophenyl)ethyl]amide; 6-[4-(2-Trifluoromethylbenzoyl)piperazin-1-yl]pyridazine-3-carboxylic acid [2-(3-chlorophenyl)ethyl]amide; 6-[4-(2-Trifluoromethylbenzoyl)piperazin-1-yl]pyridazine-3-carboxylic acid (2-phenylpropyl)amide; 6-[4-(2-Trifluoromethylbenzoyl)piperazin-1-yl]pyridazine-3-carboxylic acid (2-biphenyl-4-ylethyl)amide; (R)-6-[4-(2-Trifluoromethylbenzoyl)piperazin-1-yl]pyridazine-3-carboxylic acid (2-hydroxy-2-phenylethyl)-amide; (S)-6-[4-(2-Trifluoromethylbenzoyl)piperazin-1-yl]pyridazine-3-carboxylic acid (2-hydroxy-2-phenylethyl)-amide; Acetic acid 1-phenyl-2-({6-[4-(2-trifluoromethyl-benzoyl)-piperazin-1-yl]pyridazine-3-carbonyl}amino)ethyl ester; 6-[4-(2-Trifluoromethylbenzoyl)piperazin-1-yl]pyridazine-3-carboxylic acid [3-(4-fluorophenyl)propyl]amide; 6-[4-(2-Trifluoromethylbenzoyl)piperazin-1-yl]pyridazine-3-carboxylic acid (2,2-difluoro-2-phenylethyl)amide; and 6-[4-(2-Trifluoromethylbenzoyl)piperazin-1-yl]pyridazine-3-carboxylic acid [2-(3-fluorophenyl)-2-hydroxyethyl]amide.

The compounds of Formula V(b) also include: 6-[4-(2-Trifluoromethylbenzoyl)piperazin-1-yl]pyridazine-3-carboxylic acid (4-hydroxybutyl)amide; 6-[4-(2-Trifluoromethylbenzoyl)piperazin-1-yl]pyridazine-3-carboxylic acid (3-hydroxy-4,4-dimethylpentyl)amide; 6-[4-(2-Trifluoromethylbenzoyl)piperazin-1-yl]pyridazine-3-carboxylic acid (3-hydroxy-3-methylbutyl)amide; 6-[4-(2-Trifluoromethylbenzoyl)piperazin-1-yl]pyridazine-3-carboxylic acid (2-hydroxy-3,3-dimethylbutyl)amide; and 6-[4-(5-Fluoro-2-trifluoromethylbenzoyl)piperazin-1-yl]pyridazine-3-carboxylic acid (2-hydroxy-3,3-dimethylbutyl)amide.

The compounds of Formula V(b) also include: 6-[4-(2-Nitrobenzoyl)piperazin-1-yl]pyridazine-3-carboxylic acid (3-methylbutyl)amide; 6-[4-(2-Chlorobenzoyl)piperazin-1-yl]pyridazine-3-carboxylic acid (3-methylbutyl)amide; 6-[4-(2,4-Dichlorobenzoyl)piperazin-1-yl]pyridazine-3-carboxylic acid (3-methylbutyl)amide; 6-[4-(2-Aminobenzoyl)piperazin-1-yl]pyridazine-3-carboxylic acid (3-methylbutyl)amide; 6-[4-(2-Trifluoromethylbenzoyl)piperazin-1-yl]pyridazine-3-carboxylic acid [2-(4-chlorophenoxy)ethyl]amide; 6-[4-(2-Trifluoromethylbenzoyl)piperazin-1-yl]pyridazine-3-carboxylic acid [2-(4-fluorophenoxy)ethyl]amide; 6-[4-(2-Trifluoromethylbenzoyl)piperazin-1-yl]pyridazine-3-carboxylic acid (3,3-dimethylbutyl)amide; 6-[4-(2-Trifluoromethylbenzoyl)piperazin-1-yl]pyridazine-3-carboxylic acid pentylamide; 4-({6-[4-(2-Trifluoromethylbenzoyl)piperazin-1-yl]pyridazine-3-carbonyl)amino)butyric acid ethyl ester; 6-[4-(5-Fluoro-2-trifluoromethylbenzoyl)piperazin-1-yl]pyridazine-3-carboxylic acid pentylamide; 6-[4-(2-Trifluoromethylbenzoyl)piperazin-1-yl]pyridazine-3-carboxylic acid (4-methylpentyl)amide; 6-[4-(2-Fluoro-6-trifluoromethylbenzoyl)piperazin-1-yl]pyridazine-3-carboxylic acid (3-methylbutyl)amide; 6-[4-(2,6-Difluorobenzoyl)piperazin-1-yl]pyridazine-3-carboxylic acid (3-methylbutyl)amide; 6-[4-(2-Trifluoromethylbenzoyl)piperazin-1-yl]pyridazine-3-carboxylic acid (2-oxo-2-phenylethyl)amide; Acetic acid 1,1-DIMETHYL-3-({6-[4-(2-trifluoromentyl-benzoyl)piperazin-1-yl]pyridazine-3-carbonyl}amino)propyl ester; 6-[4-(2-Trifluoromethylbenzoyl)piperazin-1-yl]pyridazine-3-carboxylic acid (2-phenoxyethyl)amide; 6-[4-(2-Trifluoromethylbenzoyl)piperazin-1-yl]pyridazine-3-carboxylic acid hexylamide; 6-[4-(2-Trifluoromethylbenzoyl)piperazin-1-yl]pyridazine-3-carboxylic acid (4-methylpentyl)amide; 6-[4-(5-Fluoro-2-trifluoromethylbenzoyl)piperazin-1-yl]pyridazine-3-carboxylic acid (4-methylpentyl)amide; 6-[2,5-Dimethyl-4-(2-trifluoromehtylbenzoyl)piperazine-1-yl]pyridazine-3-carboxylic acid pentylamide; 6-[4-(2-Trifluoromethyl-benzoyl)-piperazin-1-yl]-pyridazine-3-carboxylic acid heptylamide; 6-[4-(2-Sulfamoyl-benzoyl)-piperazin-1-yl]-pyridazine-3-carboxylic acid (3-methyl-butyl)-amide; 6-[4-(5-Chloro-2-trifluoromethyl-benzoyl)-piperazin-1-yl]-pyridazine-3-carboxylic acid hexylamide; 6-[4-(2-Trifluoromethyl-benzoyl)-piperazin-1-yl]-pyridazine-3-carboxylic acid (2-cyclopropyl-2-oxo-ethyl)-amide; 4-Trifluoromethyl-6-[4-(2-trifluoromethyl-benzoyl)-piperazin-1-yl]-pyridazine-3-carboxylic acid (3-methyl-butyl)-amide; and 6-[4-(5-Fluoro-2-trifluoromethylbenzoyl)piperazin-1-yl]pyridazine-3-carboxylic acid pentyl-4-enylamide.

The compounds of Formula V(b) also include: 6-(4-Benzoylpiperazin-1-yl)pyridazine-3-carboxylic acid (2-cyclopropylethyl)amide; 6-[4-(2-Chloro-5-fluorobenzoyl)piperazin-1-yl]pyridazine-3-carboxylic acid (2-cyclopropylethyl)amide; 6-[4-(5-Chloro-2-trifluoromethylbenzoyl)piperazin-1-yl]pyridazine-3-carboxylic acid (2-cyclopropylethyl)amide; 6-[4-(2,6-Difluorobenzoyl)piperazin-1-yl]pyridazine-3-carboxylic acid (2-cyclopropylethyl)amide; 6-[4-(2,5-Bis-trifluoromethylbenzoyl)piperazin-1-yl}pyridzine-3-carboxylic acid (2-cyclopropylethyl)amide 6-[4-(2,4-Bis-trifluoromethylbenzoyl)piperazin-1-yl]pyridazine-3-carboxylic acid (2-cyclopropylethyl)amide; 6-[4-(2,5-Difluorobenzoyl)piperazin-1-yl]pyridazine-3-carboxylic acid (2-cyclopropylethyl)amide; 6-[4-(5-Fluoro-2-trifluoromethylbenzoyl)piperazin-1-yl]pyridazine-3-carboxylic acid (3-cyclopropylpropyl)amide; 6-[4-(2-Fluorobenzoyl)piperazin-1-yl]pyridazine-3-carboxylic acid (2-cyclopropylethyl)amide; 6-[4-(3-Fluoro-2-trifluoromethylbenzoyl)piperazin-1-yl]pyridazine-3-carboxylic acid (2-cyclopropylethyl)amide; 6-[4-(4-Fluoro-2-trifluoromethylbenzoyl)piperazin-1-yl]pyridazine-3-carboxylic acid (3-cyclopropylpropyl)amide; 6-[4-(2-Trifluoromethylbenzoyl)piperazin-1-yl]pyridazine-3-carboxylic acid (2-methylcyclopropylmethyl)amide; 6-[4-(5-Fluoro-2-methoxybenzoyl)piperazin-1-yl]pyridazine-3-carboxylic acid (2-cyclopropylethyl)amide; 6-[4-(2-Dimethylaminobenzoyl)piperazin-1-yl] pyridazine-3-carboxylic acid (2-cyclopropylethyl)amide; 6-[4-(2-Chloro-5-dimethylaminobenzoyl)piperazin-1-yl]pyridazine-3-carboxylic acid (2-cyclopropylethyl)amide; 6-[4-(2,5-Dimethylbenzoyl)piperazin-1-yl] pyridazine-3-carboxylic acid (2-cyclopropylethyl)amide; 6-[4-(2,5-Dichlorobenzoyl)piperazin-1-yl] pyridazine-3-carboxylic acid (2-cyclopropylethyl)amide; 6-[4-(2-Trifluoromethylbenzoyl)piperazin-1-yl] pyridazine-3-carboxylic acid cyclobutylmethylamide; Acetic acid 2-4-[6-(2-cyclopropylethylcarbamoyl)-pyridazin-3-yl] piperazine-1-carbonyl}phenyl ester; 6-[4-(2-Trifluoromethylbenzoyl)piperazin-1-yl] pyridazine-3-carboxylic acid (2-cyclopropyl-2-hydroxyethyl)amide; 6-[4-(2-Trifluoromethylbenzoyl)piperazin-1-yl pyridazine-3-carboxylic acid (2-phenylcyclopropylmethyl)amide; 6-[4-(2-Tri fluoromethylbenzoyl)piperazin-1-yl]pyridazine-3-carboxylic acid (3-cyclopropylpropyl)amide; 6-[4-(2-Cyanobenzoyl)piperazin-1-yl] pyridazine-3-carboxylic acid (2-cyclopropylethyl)amide; 6-{4-[2-(2-Trifluoromethylphenyl)acetyl]piperazin-1-yl}pyridazine-3-carboxylic acid (2-cyclopropylethyl)amide; 6-[4-(4-Fluoro-2-trifluoromethylbenzoyl)piperazin-1-yl]pyridazine-3-carboxylic acid (2-cyclopropylethyl)amide; 6-[4-(5-Chloro-2-trifluoromethylbenzoyl)piperazin-1-yl]pyridazine-3-carboxylic acid (3-cyclopropylpropyl)amide; 6-[3,5-Dimethyl-4-(2-trifluoromethylbenzoyl)piperazin-1-yl] pyridazine-3-carboxylic acid (2-cyclopropylethyl)amide; 2-{4-[6-(2-Cyclopropylethylcarbamoyl)pyridazin-3-yl]piperazine-1-carbonyl}benzoic acid methyl ester; 6-[4-(2-Trifluoromethyl-benzoyl)-piperazin-1-yl]-pyridazine-3-carboxylic acid (2-cyclobutyl-ethyl)-amide; 2-{4-[6-(2-Cyclopropyl-ethylcarbamoyl)-pyridazin-3-yl]-piperazine-1-carbonyl}-benzoic acid; 6-[4-(5-Chloro-2-trifluoromethyl-benzoyl)-piperazin-1-yl]-pyridazine-3-carboxylic acid (2-cyclobutyl-ethyl)-amide; 6-[4-(5-Fluoro-2-trifluoromethyl-benzoyl)-piperazin-1-yl]-pyridazine-3-carboxylic acid (2-cyclobutyl-ethyl)-amide; 6-[4-(2-Trifluoromethyl-benzoyl)-piperazin-1-yl]-pyridazine-3-carboxylic acid (3-cyclobutyl-propyl)-amide; 6-[4-(5-Fluoro-2-trifluoromethyl-benzoyl)-[1,4] diazepan-1-yl]-pyridazine-3-carboxylic acid (2-cyclopropyl-ethyl)-amide; 6-[4-(2-Trifluoromethyl-thiobenzoyl)-piperazin-1-yl]-pyridazine-3-carboxylic acid (2-cyclopropyl-ethyl)-amide; 6-[4-(2-Trifluoromethyl-benzoyl)-piperazin-1-yl]-pyridazine-3-carboxylic acid (4-cyclopropyl-butyl)-amide; and 6-[4-(2-Trifluoromethyl-benzoyl)-piperazin-1-yl]-pyridazine-3-carboxylic acid (2,2-dimethyl-cyclopropylmethyl)-amide.

The compounds of Formula V(b) also include: 6-[4-(Pyridine-2-carbonyl)piperazin-1-yl] pyridazine-3-carboxylic acid (2-cyclopropylethyl)amide; 6-[4-(2-Ttrifluoromenthylfuran-3-carbonyl)piperazin-1-yl]pyridazine-3-carboxylic acid (2-cyclopropylethyl)amide; 6-[4-(2-Chloro-4-trifluoromethylpyrimidine-5-carbonyl)piperazin-1-yl]pyridazine-3-carboxylic acid (2-cyclopropylethyl)amide; 6-[4-(5-Methyl-2-trifluoromethylfuran-3-carbonyl)piperazin-1-yl]pyridazine-3-carboxylic acid (2-cyclopropylethyl)amide; 6-[4-(2-Chloropyridine-3-carbonyl)piperazin-1-yl]pyridazine-3-carboxylic acid (2-cyclopropylethyl)amide; 6-[4-(2-Methyl-5-trifluoromethyloxazole-4-carbonyl)piperazin-1-yl]-pyridazine-3-carboxylic acid (2-cyclopropylethyl)amide; 6-[4-(2,6-Dichloropyridine-3-carbonyl)piperazin-1-yl]pyridazine-3-carboxylic acid (2-cyclopropylethyl)amide; 6-[4-(Pyrrolidine-1-carbonyl)piperazin-1-yl] pyridazine-3-carboxylic acid (2-cyclopropylethyl)amide; 6-[4-(1-Methyl-1H-pyrrole-2-carbonyl)piperazin-1-yl] pyridazine-3-carboxylic acid (2-cyclopropylethyl)amide; and 6-[4-(Tetrahydrofuran-2-carbonyl)piperazin-1-yl]pyridazine-3-carboxylic acid (2-cyclopropylethyl)amide.

The compounds of Formula V(b) also include: 6-{4-[2-(2-Trifluoromethylphenyl)acetyl]piperazin-1-yl}pyridazine-3-carboxylic acid (2-cyclopropylethyl)amide.

The compounds of Formula V(b) also include: 4-[6-(3-Methylbutylcarbamoyl)pyridazin-3-yl]piperazine-1-carboxylic acid t-butyl ester; and 4-[6-(2-Cyclopropylethylcarbamoyl)pyridazin-3-yl]piperazine-1-carboxylic acid t-butyl ester.

The compounds of Formula V(b) also include: 6-[4-(4,4,4-Trifluoro-3-hydroxy-3-trifluoromethylbutyryl)piperazin-1-yl] pyridazine-3-carboxylic acid (2-cyclopropylethyl)amide; 6-[4-(4,4,4-Trifluoro-3-hydroxy-3-methylbutyryl)piperazin-1-yl] pyridazine-3-carboxylic acid (2-cyclopropylethyl)amide; and 6-[4-(3,3,3-Trifluoro-2-hydroxy-2-methylpropionyl)piperazin-1-yl] pyridazine-3-carboxylic acid (2-cyclopropylethyl)amide.

The compounds of Formula V(b) also include: 6-[4-(1-Hydroxycyclopropanecarbonyl)piperazin-1-yl] pyridazine-3-carboxylic acid (2-cyclopropylethyl)amide; 6-(4-Cyclobutanecarbonylpiperazin-1-yl)pyridazine-3-carboxylic acid (2-cyclopropylethyl)amide; 6-[4-(2-Trifluoromethylcyclopropanecarbonyl)piperazin-1-yl] pyridazine-3-carboxylic acid (2-cyclopropylethyl)amide; 6-(4-Cyclohexanecarbonylpiperazin-1-yl)pyridazine-3-carboxylic acid (2-cyclopropylethyl)amide; 6-[4-(2-Methylcyclohexanecarbonyl)piperazin-1-yl] pyridazine-3-carboxylic acid (2-cyclopropylethyl)amide; 6-[4-(3-Methylcyclohexanecarbonyl)piperazin-1-yl] pyridazine-3-carboxylic acid (2-cyclopropylethyl)amide; 6-[4-(4-Methylcyclohexanecarbonyl)piperazin-1-yl]pyridazine-3-carboxylic acid (2-cyclopropylethyl)amide; 6-[4-(2-Methylcyclopropanecarbonyl)piperazin-1-yl] pyridazine-3-carboxylic acid (2-cyclopropylethyl)amide; and 6-[4-(2,2,3,3-Tetramethylcyclopropanecarbonyl)piperazin-1-yl] pyridazine-3-carboxylic acid (2-cyclopropylethyl)amide.

The compounds of Formula V(b) also include: 6-[4-(2-Ethylbutyryl)piperazin-1-yl] pyridazine-3-carboxylic acid (2-cyclopropylethyl)amide; 6-[4-(3,3,3-Trifluoro-2-methyl-2-trifluoromehtylpropionyl)piperazin-1-yl] pyridazine-3-carboxylic acid (2-cyclopropylethyl)amide; 6-[4-(2,2-Dimethylpropionyl)piperazin-1-yl]pyridazine-3-carboxylic acid (2-cyclopropylethyl)amide; 6-[4-(2,2-Dimethylbutyryl)piperazin-1-yl] pyridazine-3-carboxylic acid (2-cyclopropylethyl)amide; 6-[4-(2,2-Dimethylpentanoyl)piperazin-1-yl] pyridazine-3-carboxylic acid (2-cyclopropylethyl)amide; 6-[4-(4,4,4-Trifluorobut-2-enoyl)piperazin-1-yl] pyridazine-3-carboxylic acid (2-cyclopropylethyl)amide; and 6-[4-(4,4,4-Trifluoro-3-trifluoromethylbut-2-enoyl)-piperazin-1-yl] pyridazine-3-carboxylic acid (2-cyclopropyl-ethyl)amide.

The methods of the present invention comprise administration of an effective amount of an SCD-inhibiting, Aβ42-lowering, composition to a patient in need of such treatment. Such therapeutic composition comprises a compound according to Formula V(c)(1):

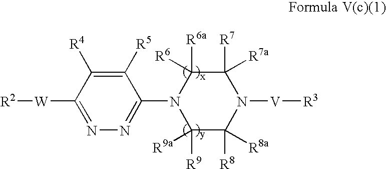

wherein:

x and y are each independently 1, 2 or 3;

W is —C(O)N(R¹)—; —C(O)N[C(O)R^{1a}]—, —N(R¹)C(O)N(R¹)— or —N(R¹)C(O)—;

V is —C(O)—, —C(S)—, or —C(R¹⁰)H;

each R¹ is independently selected from the group consisting of hydrogen; C₁-C₆alkyl optionally substituted with one or more substituents selected from the group consisting of halo, methyl or trifluoromethyl; and C₂-C₆alkyl optionally substituted with one or more substituents selected from the group consisting of methoxy and hydroxyl;

R^{1a} is selected from the group consisting of hydrogen, C₁-C₆alkyl and cycloalkyl;

R² is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₁-C₁₂heteroaryl, and C₃-C₁₂heteroarylalkyl;

or R² is a multi-ring structure having 2 to 4 rings wherein the rings are independently selected from the group consisting of cycloalkyl, heterocyclyl, aryl and heteroaryl and where some or all of the rings may be fused to each other;

R³ is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₁-C₁₂heteroaryl and C₃-C₁₂heteroarylalkyl;

or R³ is a multi-ring structure having 2 to 4 rings wherein the rings are independently selected from the group consisting of cycloalkyl, heterocyclyl, aryl and heteroaryl and where some or all of the rings may be fused to each other;

R⁴ and R⁵ are each independently selected from hydrogen, fluoro, chloro, methyl, methoxy, trifluoromethyl, cyano, nitro or —N(R¹²)₂;

R⁶, R^{6a}, R⁷, R^{7a}, R⁸, R^{8a}, R⁹, and R^{9a} are each independently selected from hydrogen or C₁-C₃alkyl;

or R⁶ and R^{6a} together, or R⁷ and R^{7a} together, or R⁸ and R^{8a} together, or R⁹ and R^{9a} together are an oxo group, provided that when V is —C(O)—, R⁷ and R^{7a} together or R⁸ and R^{8a} together do not form an oxo group, while the remaining R⁶, R^{6a}, R⁷, R^{7a}, R⁸, R^{8a}, R⁹, and R^{9a} are each independently selected from hydrogen or C₁-C₃alkyl;

or one of R⁶, R^{6a}, R⁷, and R^{7a} together with one of R⁸, R^{8a}, R⁹ and R^{9a} form an alkylene bridge, while the remaining R6, R^{6a}, R⁷, R^{7a}, R8, R^{8a}, R⁹, and R^{9a} are each independently selected from hydrogen or C₁-C₃alkyl;

R¹⁰ is hydrogen or C₁-C₃alkyl; and

each R¹² is independently selected from hydrogen or C₁-C₆alkyl;

a stereoisomer, enantiomer or tautomer thereof, a pharmaceutically acceptable salt thereof, a pharmaceutical composition thereof or a prodrug thereof.

Other compounds include those of Formula V(c)(2):

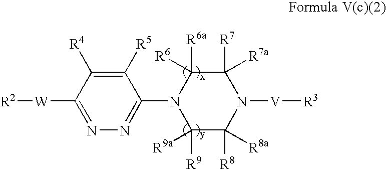

wherein:

x and y are each independently 1, 2 or 3;

W is a direct bond, —C(O)N(R¹)—; —C(O)N[C(O)R^{1a}]—, —N(R¹)C(O)N(R¹)—(R¹)C(O)—, —OC(O)N(R¹)—, —N(R¹)S(O)ₚ— (where p is 1 or 2), —S(O)ₚN(R¹)— (where p is 1 or 2), —C(O)—, —OS(O)₂N(R¹)—, —OC(O)—, —C(O)O—, —N(R¹)C(O)O—, —N(R¹)C(═NR^{1a})N(R¹)—, —N(R¹)C(═S)N(R¹—)—, —N(R¹)C(═NR^{1a})—, or —C(═NR^{1a})N(R¹)—;

V is —C(O)—, —C(O)O—, —C(S)—, —C(O)N(R¹)—, —S(O)ₜ— (where t is 0, 1 or 2), —S(O)ₚN(R¹)— (where p is 1 or 2), —C(R¹⁰)H—, or —C(═NR^{1a})—;

each R¹ is independently selected from the group consisting of hydrogen; C₁-C₆alkyl optionally substituted with one or more substituents selected from the group consisting of halo, methyl or trifluoromethyl; and C₂-C₆alkyl optionally substituted with one or more substituents selected from the group consisting of methoxy and hydroxyl;

R^{1a} is selected from the group consisting of hydrogen, —OR¹, cyano, C¹-C₆alkyl and cycloalkylalkyl;

R² is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₁-C₁₂heteroaryl, and C₃-C₁₂heteroarylalkyl;

or R² is a multi-ring structure having 2 to 4 rings wherein the rings are independently selected from the group consisting of cycloalkyl, heterocyclyl, aryl and heteroaryl and where some or all of the rings may be fused to each other;

R³ is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₁-C₁₂heteroaryl and C₃-C₁₂heteroarylalkyl;

or R³ is a multi-ring structure having 2 to 4 rings wherein the rings are independently selected from the group consisting of cycloalkyl, heterocyclyl, aryl and heteroaryl and where some or all of the rings may be fused to each other;

R⁴ and R⁵ are each independently selected from hydrogen, fluoro, chloro, methyl, methoxy, trifluoromethyl, cyano, nitro or —N(R¹²)₂;

R⁶, R^{6a}, R⁷, R^{7a}, R⁸, R^{8a}, R⁹, and R^{9a} are each independently selected from hydrogen or C₁-C₃alkyl;

or R⁶ and R⁶, together, or R⁷ and R^{7a} together, or R⁸ and R^{8a} together, or R⁹ and R^{9a} together are an oxo group, provided that when V is —C(O)—, R⁷ and R^{7a} together or R⁸ and R^{8a} together do not form an oxo group, while the remaining R⁵, R^{6a}, R⁷, R^{7a}, R⁸, R^{8a}, R⁹, and R^{9a} are each independently selected from hydrogen or C₁-C₃alkyl;

or one of R⁶, R^{6a}, R⁷, and R^{7a} together with one of R⁸, R^{8a}, R⁹ and R^{9a} form an alkylene bridge, while the remaining R⁶, R^{6a}, R⁷, R^{7a}, R⁸, R^{8a}, R⁹, and R^{9a} are each independently selected from hydrogen or C₁-C₃alkyl;

R¹⁰ is hydrogen or C₁-C₃alkyl; and

each R¹² is independently selected from hydrogen or C₁-C₆alkyl;

a stereoisomer, enantiomer or tautomer thereof, a pharmaceutically acceptable salt thereof, a pharmaceutical composition thereof or a prodrug thereof.

The methods of the present invention also comprise administration of an effective amount of an SCD-inhibiting, Aβ42-lowering, composition, to a patient in need of such treatment, wherein the composition comprises a compound according to Formula V(d):

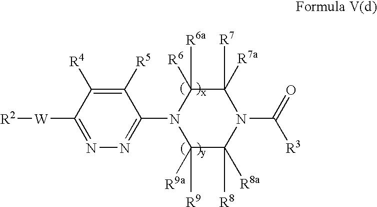

wherein:

x and y are each independently 1, 2 or 3;

W is selected from —C(O)N(R¹)— and —N(R¹)C(O)—; each R² is independently selected from the group consisting of hydrogen; C₁-C₆alkyl optionally substituted with one or more substituents selected from the group consisting of halo, methyl or trifluoromethyl; and C₂-C₆alkyl optionally substituted with one or more substituents selected from the group consisting of methoxy and hydroxy;

R² is selected from the group consisting of C₇-C₁₂alkyl, C₃-C₁₂alkenyl, C₇-C₁₂hydroxyalkyl, C₂-C₁₂alkoxyalkyl, C₃-C₁₂hydroxyalkenyl, C₃-C₁₂cycloalkyl, C₄-C₁₂cycloalkylalkyl, C₁₃-C₁₉aralkyl, C₃-C₁₂heterocyclylalkyl, and C₃-C₁₂heteroarylalkyl;

or R² is a multi-ring structure having 2 to 4 rings wherein the rings are independently selected from the group consisting of cycloalkyl, heterocyclyl, aryl and heteroaryl, where some or all of the rings may be fused to each other;

R³ is selected from the group consisting of C₃-C₁₂alkyl, C₃-C₁₂alkenyl, C₃-C₁₂hydroxyalkyl, C₃-C₁₂hydroxyalkenyl, C₃-C₁₂alkoxy, C₃-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₅-C₁₂ heteroaryl and C₃-C₁₂heteroarylalkyl;

or R³ is a multi-ring structure having 2 to 4 rings wherein the rings are independently selected from the group consisting of cycloalkyl, heterocyclyl, aryl and heteroaryl and where some or all of the rings may be fused to each other;

R⁴ and R⁵ are each independently selected from hydrogen, fluoro, chloro, methyl, methoxy and trifluoromethyl; and

R⁶, R^{6a}, R⁷, R^{7a}, R⁸, R^{8a}, R⁹, and R^{9a} are each independently selected from hydrogen or C₁-C₃alkyl;

or R⁶ and R^{6a} together, or R⁷ and R^{7a} together, or R⁸ and R^{8a} together, or R⁹ and R^{9a} together are an oxo group, provided that when V is —C(O)—, R⁷ and R^{7a} together or R⁸ and R^{8a} together do not form an oxo group, while the remaining R⁶, R^{6a}, R⁷, R^{7a}, R⁸, R^{8a}, R⁹, and R^{9a} are each independently selected from hydrogen or C₁-C₃alkyl;

or one of R⁶, R^{6a}, R⁷, and R^{7a} together with one of R⁸, R^{8a}, R⁹ and R^{9a} form an alkylene bridge, while the remaining R⁶, R^{6a}, R⁷, R^{7a}, R⁸, R^{8a}, R⁹, and R^{9a} are each independently selected from hydrogen or C₁-C₃alkyl;

including a stereoisomer, enantiomer or tautomer thereof, a pharmaceutically acceptable salt thereof, a pharmaceutical composition thereof or a prodrug thereof.

The methods of the present invention also comprise administration of an effective amount of an SCD-inhibiting, Aβ42-lowering, composition, to a patient in need of such treatment, wherein the composition comprises a compound according to Formula V(e):

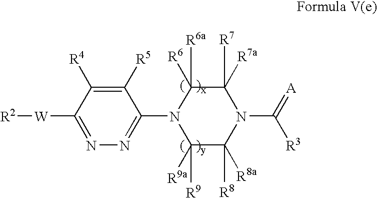

wherein:

x and y are each independently 1, 2 or 3;

A is oxygen or sulfur;

W is selected from —C(O)N(R¹)— and —N(R¹)C(O)—;

each R¹ is independently selected from the group consisting of hydrogen; C₁-C₆alkyl optionally substituted with one or more substituents selected from the group consisting of halo, methyl or trifluoromethyl; and C₂-C₆alkyl optionally substituted with one or more substituents selected from the group consisting of methoxy and hydroxy;

R² is selected from the group consisting of C₁-C₁₂alkyl, C₂-C₁₂alkenyl, C₂-C₁₂hydroxyalkyl, C₂-C₁₂hydroxyalkenyl, C₁-C₆alkoxy, C₃-C₁₂alkoxyalkyl, C₃-C₁₂cycloalkyl, C₄-C₁₂cycloalkylalkyl, aryl, C7-C₁₂aralkyl, C₃-C₁₂ heterocyclyl, C₃-C₁₂heterocyclylalkyl, C₁-C₁₂heteroaryl and C₃-C₁₂heteroarylalkyl;

or R² is a multi-ring structure having 2 to 4 rings wherein the rings are independently selected from the group consisting of cycloalkyl, heterocyclyl, aryl and heteroaryl, where some or all of the rings may be fused to each other;

R³ is phenyl optionally substituted by one or more substituents selected from the group consisting of halo, cyano, nitro, hydroxy, C₁-C₆alkyl, C₁-C₆trihaloalkyl, C₁-C₆trihaloalkoxy, C₁-C₆alkylsulfonyl, —N(R¹¹)₂, —OC(O)R¹¹, —C(O)OR¹¹, —S(O)₂N(R¹¹)₂, cycloalkyl, heterocyclyl, heteroaryl and heteroarylcycloalkyl, provided that R³ is not phenyl substituted with optionally substituted thienyl;

R⁴ and R⁵ are each independently selected from hydrogen, fluoro, chloro, methyl, methoxy and trifluoromethyl;

R⁶, R^{6a}, R⁷, R^{7a}, R⁸, R^{8a}, R⁹, and R^{9a} are each independently selected from hydrogen or C₁-C₃alkyl;

or R⁶ and R^{6a} together, or R⁷ and R^{7a} together, or R⁸ and R^{8a} together, or R⁹ and R^{9a} together are an oxo group, provided that when V is —C(O)—, R⁷ and R^{7a} together or R³ and R^{8a} together do not form an oxo group, while the remaining R⁶, R^{6a}, R⁷, R^{7a}, R⁸, R^{8a}, R⁹, and R^{9a} are each independently selected from hydrogen or C₁-C₃alkyl;

or one of R⁶, R^{6a}, R⁷, and R^{7a} together with one of R⁸, R^{8a}, R⁹ and R^{9a} form an alkylene bridge, while the remaining R⁶, R^{6a}, R⁷, R^{7a}, R⁸, R^{8a}, R⁹, and R^{9a} are each independently selected from hydrogen or C₁-C₃alkyl; and

each R¹ is independently selected from hydrogen, C₁-C₆alkyl, C₃-C₆cycloalkyl, aryl or aralkyl;

a stereoisomer, enantiomer or tautomer thereof, a pharmaceutically acceptable salt thereof, a pharmaceutical composition thereof or a prodrug thereof.

The methods of the present invention also comprise administration of an effective amount of an SCD-inhibiting, Aβ42-lowering, composition, to a patient in need of such treatment, wherein the composition comprises a compound according to Formula V(f):

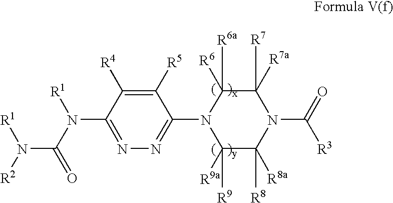

wherein:

x and y are each independently 1, 2 or 3;

each R¹ is independently selected from the group consisting of hydrogen; C₁-C₆alkyl optionally substituted with one or more substituents selected from the group consisting of halo, methyl or trifluoromethyl; and C₂-C₆alkyl optionally substituted with one or more substituents selected from the group consisting of methoxy and hydroxy;

R² is selected from the group consisting of C₁-C₁₂alkyl, C₂-C₁₂alkenyl, C₂-C₁₂hydroxyalkyl, C₂-C₁₂hydroxyalkenyl, C₂-C₁₂alkoxyalkyl, C₃-C₁₂cycloalkyl, C₄-C₁₂cycloalkylalkyl, C₃-C₁₂heterocyclyl, C₃-C₁₂heterocyclylalkyl, aryl, C₇-C₁₂aralkyl, C₁-C₁₂heteroaryl, and C₃-C₁₂heteroarylalkyl;

or R² is a multi-ring structure having 2 to 4 rings wherein the rings are independently selected from the group consisting of cycloalkyl, heterocyclyl, aryl and heteroaryl and where some or all of the rings may be fused to each other;

R³ is selected from the group consisting of C₁-C₁₂alkyl, C₂-C₁₂alkenyl, C₂-C₁₂hydroxyalkyl, C₂-C₁₂ hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₁-C₁₂heteroaryl and C₃-C₁₂heteroarylalkyl;

or R³ is a multi-ring structure having 2 to 4 rings wherein the rings are independently selected from the group consisting of cycloalkyl, heterocyclyl, aryl and heteroaryl and where some or all of the rings may be fused to each other;

R⁴ and R⁵ are each independently selected from hydrogen, fluoro, chloro, methyl, methoxy and trifluoromethyl; and

R⁶, R^{6a}, R⁷, R^{7a}, R⁸, R₈ₐ, R⁹, and R^{9a} are each independently selected from hydrogen or C₁-C₃alkyl;

or R⁶ and R^{6a} together, or R⁷ and R^{7a} together, or R⁸ and R^{8a} together, or R⁹ and R^{9a} together are an oxo group, provided that when V is —C(O)—, R⁷ and R⁷, together or R⁸ and R^{8a} together do not form an oxo group, while the remaining R⁶, R^{6a}, R⁷, R^{7a}, R⁸, R^{8a}, R⁹, and R^{9a} are each independently selected from hydrogen or C₁-C₃alkyl; or one of R⁶, R^{6a}, R⁷, and R^{7a} together with one of R⁸, Rₐ, R⁹ and R^{9a} form an alkylene bridge, while the remaining R⁶, R^{6a}, R⁷, R^{7a}, R⁸, R^{8a}, R⁹, and R^{9a} are each independently selected from hydrogen or C₁-C₃alkyl;

a stereoisomer, enantiomer or tautomer thereof, a pharmaceutically acceptable salt thereof, a pharmaceutical composition thereof or a prodrug thereof.

The methods of the present invention also comprise administration of an effective amount of an SCD-inhibiting, Aβ42-lowering, composition, to a patient in need of such treatment, wherein the composition comprises a compound according to Formula V(g)(1):

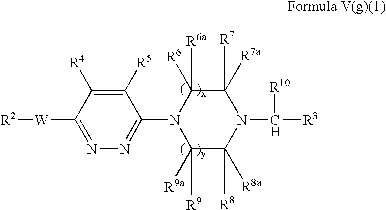

wherein:

x and y are each independently 1, 2 or 3;

W is —C(O)N(R¹)—; —N(R¹)C(O)N(R¹)— or —N(R¹)C(O)—;

each R¹ is independently selected from the group consisting of hydrogen; C₁-C₆alkyl optionally substituted with one or more substituents selected from the group consisting of halo, methyl or trifluoromethyl; and C₂-C₆alkyl optionally substituted with one or more substituents selected from the group consisting of methoxy and hydroxy;

R² is selected from the group consisting of C₇-C₁₂alkyl, C₂-C₁₂alkenyl, C₇-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, C₁₃-C₁₉aralkyl, C₃-C₁₂heterocyclyl, C₃-C₁₂heterocyclylalkyl, C₁-C₁₂heteroaryl, and C₃-C₁₂heteroarylalkyl;

R³ is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₁-C₁₂heteroaryl and C₃-C₁₂heteroarylalkyl;

R⁴ and R⁵ are each independently selected from hydrogen, fluoro, chloro, methyl, methoxy, trifluoromethyl, cyano, nitro or —N(R¹²)₂;

R⁶, R^{6a}, R⁷, R^{7a}, R⁸, R^{8a}, R⁹, and R^{9a} are each independently selected from hydrogen or C₁-C₃alkyl;

or R⁶ and R^{6a} together, or R⁷ and R^{7a} together, or R⁸ and R^{8a} together, or R⁹ and R^{9a} together are an oxo group, provided that when V is —C(O)—, R⁷ and R^{7a} together or R⁸ and R^{8a} together do not form an oxo group, while the remaining R⁶, R^{6a}, R⁷, R^{7a}, R⁸, R^{8a}, R⁹, and R^{9a} are each independently selected from hydrogen or C₁-C₃alkyl;

or one of R⁶, R^{6a}, R⁷, and R^{7a} together with one of R⁸, R^{8a}, R⁹ and R^{9a} form an alkylene bridge, while the remaining R⁶, R^{6a}, R⁷, R^{7a}, R⁸, R^{8a}, R⁹, and R^{9a} are each independently selected from hydrogen or C₁-C₃alkyl;

R¹⁰ is hydrogen or C₁-C₃alkyl; and
each R¹² is independently selected from hydrogen or C₁-C₆alkyl;
provided, however, that R² can not be pyrazinyl, pyridinonyl, pyrrolidinonyl or imidazolyl;

a stereoisomer, enantiomer or tautomer thereof, a pharmaceutically acceptable salt thereof, a pharmaceutical composition thereof or a prodrug thereof.

The methods of the present invention also comprise administration of an effective amount of an SCD-inhibiting, Aβ42-lowering, composition, to a patient in need of such treatment, wherein the composition comprises a compound according to Formula V(g)(2):

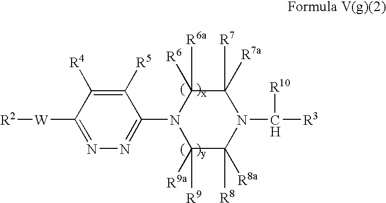

wherein:

x and y are each independently 1, 2 or 3;

W is —C(O)N(R¹)—; —N(R¹)C(O)N(R¹)— or —N(R¹)C(O)—;

each R¹ is independently selected from the group consisting of hydrogen; C₁-C₆alkyl optionally substituted with one or more substituents selected from the group consisting of halo, methyl or trifluoromethyl; and C₂-C₆alkyl optionally substituted with one or more substituents selected from the group consisting of methoxy and hydroxy;

R² is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₂aralkyl, C₃-C₁₂heterocyclyl, C₃-C₁₂heterocyclylalkyl, C₁-C₁₂heteroaryl, and C₃-C₁₂heteroarylalkyl;

R³ is selected from the group consisting of C₇-C₁₂alkyl, C₂-C₁₂alkenyl, C₂-C₁₂hydroxyalkyl, C₂-C₁₂hydroxyalkenyl, C₁-C₁₂alkoxy or C₂-C₁₂alkoxyalkyl

R⁴ and R⁵ are each independently selected from hydrogen, fluoro, chloro, methyl, methoxy, trifluoromethyl, cyano, nitro or —N(R¹²)₂;

R⁶, R^{6a}, R⁷, R^{7a}, R⁸, R^{8a}, R⁹, and R^{9a} are each independently selected from hydrogen or C₁-C₃alkyl;

or R⁶ and R^{6a} together, or R⁷ and R^{7a}, together, or R⁸ and R^{8a} together, or R⁹ and R^{9a} together are an oxo group, provided that when V is —C(O)—, R⁷ and R^{7a} together or R⁸ and R^{8a} together do not form an oxo group, while the remaining R⁶, R^{6a}, R⁷, R^{7a}, R⁸, R^{8a}, R⁹, and R^{9a} are each independently selected from hydrogen or C₁-C₃alkyl;

or one of R⁶, R^{6a}, R⁷, and R^{7a} together with one of R⁸, R^{8a} , R⁹ and R^{9a} form an alkylene bridge, while the remaining R⁶, R^{6a}, R⁷, R^{7a}, R⁸, R^{8a}, R⁹, and R^{9a} are each independently selected from hydrogen or C₁-C₃alkyl;

R¹⁰ is hydrogen or C₁-C₃alkyl; and

each R¹² is independently selected from hydrogen or C₁-C₆alkyl; as a stereoisomer, enantiomer or tautomer thereof, a pharmaceutically acceptable salt thereof, a pharmaceutical composition thereof or a prodrug thereof.

The compounds of Formulae V(c)(1)-V(g)(2) include: 4-methylpentanoic acid {6-[4-(2-trifluoromethylbenzoyl)-piperazin-1-yl]-pyridazin-3-yl}-amide; 4-phenyl-N-{6-[4-(2-trifluoromethylbenzoyl)-piperazin-1-yl]-pyridazin-3-yl}-butyramide; 4-(4-methoxyphenyl)-N-{6-[4-(2-trifluoromethylbenzoyl)-piperazin-1-yl]-pyridazin-3-yl}-butyramide; 2-benzyloxy-N-{6-[4-(2-trifluoromethylbenzoyl)-piperazin-1-yl]-pyridazin-3-yl}-; 4-cyclohexyl-N-{6-[4-(2-trifluoromethylbenzoyl)-piperazin-1-yl]-pyridazin-3-yl}-butyramide; 2-ethoxy-N-{6-[4-(2-trifluoromethylbenzoyl)-piperazin-1-yl]-pyridazin-3-yl}-acetamide; 2-cyclopropylmethoxy-N-{6-[4-(2-trifluoromethylbenzoyl)-piperazin-1-yl]-pyridazin-3-yl}-acetamide; 2-(2-methoxyehtoxy)-N-{6-[4-(2-trifluoromethylbenzoyl)-piperazin-1-yl]-pyridazin-3-yl}-acetamide; 2,2,3,3-tetramethylcyclopropanecarboxylic acid {6-[4-(2-trifluoromethylbenzoyl)-piperazin-1-yl]-pyridazin-3-yl}-amide; cyclopropanecarboxylic acid {6-[4-(2-trifluoromethylbenzoyl)-piperazin-1-yl]-pyridazin-3-yl}-amide; 1-trifluoromethylcyclopropanecarboxylic acid {6-[4-(2-trifluoromethylbenzoyl)-piperazin-1-yl]-pyridazin-3-yl}-amide; N-{6-[4-(2-trifluoromethylbenzoyl)-piperazin-1-yl]-pyridazin-3-yl}-2-(3,3,3-trifluoropropoxy)-acetamide; 3-methoxy-N-{6-[4-(2-trifluoromethylbenzoyl)-piperazin-1-yl]-pyridazin-3-yl}-propionamide; 3-phenoxy-N-{6-[4-(2-trifluoromethylbenzoyl)-piperazin-1-yl]-pyridazin-3-yl}-propionamide; 3-(4-fluorophenyl)-N-{6-[4-(2-trifluoromethylbenzoyl)-piperazin-1-yl]-pyridazin-3-yl}-propionamide; 2-butoxy-N-{6-[4-(2-trifluoromethylbenzoyl)-piperazin-1-yl]-pyridazin-3-yl}-acetamide; 2-methyl-1-{6-[4-(2-trifluoromethylbenzoyl)-piperazin-1-yl]-pyridazin-3-ylcarbamoyl}-propylammonium chloride; 5-[1,2]-dithiolan-3-yl-pentanoic acid {6-[4-(2-trifluoromethylbenzoyl)-piperazin-1-yl]-pyridazin-3-yl}-amide; 2-(2-cyclopropylethoxy)-N-{6-[4-(2-trifluoromethylbenzoyl)-piperazin-1-yl]-pyridazin-3-yl}-acetamide; 6-[4-(isoxazole-5-carbonyl)-piperazin-1-yl]-pyridazine-3-carboxylic acid (3-methylbutyl)-amide; 6-[4-(1-methyl-5-trifluoromethyl-1 h-pyrazole-4-carbonyl)-piperazin-1-yl]-pyridazine-3-carboxylic acid (3-methylbutyl)-amide; 6-[4-(4-methylpiperazine-1-carbonyl)-piperazin-1-yl]-pyridazine-3-carboxylic acid (3-methylbutyl)-amide; 6-(4-benzoylpiperazin-1-yl)-pyridazine-3-carboxylic acid (2-cyclopropylethyl)-amide; 6-[4-(2-ethylbutyryl)-piperazin-1-yl]-pyridazine-3-carboxylic acid (2-cyclopropylethyl)-amide; 6-(4-cyclohexanecarbonylpiperazin-1-yl)-pyridazine-3-carboxylic acid (2-cyclopropylethyl)-amide; 6-[4-(2-trifluoromethoxybenzoyl)-piperazin-1-yl]-pyridazine-3-carboxylic acid (3-methylbutyl)-amide; 6-[4-(5-chloro-2-trifluoromethylbenzoyl)-piperazin-1-yl]-pyridazine-3-carboxylic acid (3-methylbutyl)-amide; 6-[4-(2-trifluoromethylbenzoyl)-piperazin-1-yl]-pyridazine-3-carboxylic acid (2-cyclopropylethyl)-amide; 6-[4-(2-chloro-5-fluorobenzoyl)-piperazin-1-yl]-pyridazine-3-carboxylic acid (2-cyclopropylethyl)-amide; 6-[4-(3,3,3-trifluoro-2-methyl-2-trifluoromethylpropionyl)-piperazin-1-yl]-pyridazine-3-carboxylic acid (2-cyclopropylethyl)-amide; 6-[4-(2,2-dimethylpropionyl)-piperazin-1-yl]-pyridazine-3-carboxylic acid (2-cyclopropylethyl)-amide; 6-[4-(5-chloro-2-trifluoromethylbenzoyl)-piperazin-1-yl]-pyridazine-3-carboxylic acid (2-cyclopropylethyl)-amide; 6-[4-(5-fluoro-2-trifluoromethylbenzoyl)-piperazin-1-yl]-pyridazine-3-carboxylic acid (2-cyclopropylethyl)-amide; 6-[4-(2,6-difluorobenzoyl)-piperazin-1-yl]-pyridazine-3-carboxylic acid (2-cyclopropylethyl)-amide; 6-[4-(pyrrolidine-1-carbonyl)-piperazin-1-yl]-pyridazine-3-carboxylic acid (2-cyclopropylethyl)-amide; 6-[4-(2,5-bis-trifluoromethylbenzoyl)-piperazin-1-yl]-pyridazine-3-carboxylic acid (2-cyclopropylethyl)-amide; 6-[4-(2,4-bis-trifluoromethylbenzoyl)-piperazin-1-yl]-pyridazine-3-carboxylic acid (2-cyclopropylethyl)-amide; 6-[4-(2,5-difluorobenzoyl)-piperazin-1-yl]-pyridazine-3-carboxylic acid (2-cyclopropylethyl)-amide; 6-[4-(5-fluoro-2-trifluoromethylbenzoyl)-piperazin-1-yl]-pyridazine-3-carboxylic acid (3-cyclopropylpropyl)-amide; 6-[4-(2-chloro-4-trifluoromethylpyrimidine-5-carbonyl)-piperazin-1-yl]-pyridazine-3-carboxylic acid (2-cyclopropylethyl)-amide; 6-[4-(2-fluorobenzoyl)-piperazin-1-yl]-pyridazine-3-carboxylic acid (2-cyclopropylethyl)-amide; 6-[4-(3-fluoro-2-trifluoromethylbenzoyl)-piperazin-1-yl]-pyridazine-3-carboxylic acid (2-cyclopropylethyl)-amide; 6-[4-(4-fluoro-2-trifluoromethylbenzoyl)-piperazin-1-yl]-pyridazine-3-carboxylic acid (3-cyclopropylpropyl)-amide; 6-[4-(5-chloro-2-trifluoromethylbenzoyl)-piperazin-1-yl]-pyridazine-3-carboxylic acid (3-cyclopropylpropyl)-amide; 6-[4-(5-fluoro-2-trifluoromethylbenzoyl)-piperazin-1-yl]-pyridazine-3-carboxylic acid (4-methylpentyl)-amide; 6-[4-(2-trifluoromethylbenzoyl)-piperazin-1-yl]-pyridazine-3-carboxylic acid (4-methylpentyl)-amide; 6-[4-(4-fluoro-2-trifluoromethylbenzoyl)-piperazin-1-yl]-pyridazine-3-carboxylic acid (2-cyclopropylethyl)-amide; 6-[4-(2-nitrobenzoyl)-piperazin-1-yl]-pyridazine-3-carboxylic acid (3-methylbutyl)-amide; 6-[4-(2-chlorobenzoyl)-piperazin-1-yl]-pyridazine-3-carboxylic acid (3-methylbutyl)-amide; 6-[4-(2,4-dichlorobenzoyl)-piperazin-1-yl]-pyridazine-3-carboxylic acid (3-methylbutyl)-; acetic acid 2-{4-[6-(2-cyclopropylethylcarbamoyl)-pyridazin-3-yl]-piperazine-1-carbonyl}-phenyl ester; 6-[4-(5-chloro-2-trifluoromethylbenzoyl)-piperazin-1-yl]-pyridazine-3-carboxylic acid (2-cyclobutylethyl)-amide; 6-[4-(5-fluoro-2-trifluoromethylbenzoyl)-piperazin-1-yl]-pyridazine-3-carboxylic acid (2-cyclobutylethyl)-amide; 6-[4-(5-chloro-2-trifluoromethylbenzoyl)-piperazin-1-yl]-pyridazine-3-carboxylic acid hexylamide; 6-[4-(5-fluoro-2-trifluoromethylbenzoyl)-[1,4]-diazepan-1-yl}-pyridazine-3-carboxylic acid (2-cyclopropylethyl)-amide; 6-(4-benzylpiperazin-1-yl)-pyridazine-3-carboxylic acid (3-methylbutyl)-; 1-(2-phenylcyclopropyl)-3-{6-[4-(2-trifluoromethylbenzoyl)-piperazin-1-yl]-pyridazin-3-yl}-urea; 3-(3-{6-[4-(2-trifluoromethylbenzoyl)-piperazin-1-yl]-pyridazin-3-yl}-ureido)-propionic acid ethyl ester; 1-pentyl-3-{6-[4-(2-trifluoromethylbenzoyl)-piperazin-1-yl]-pyridazin-3-yl}-urea; 1-benzyl-3-{6-[4-(2-trifluoromethylbenzoyl)-piperazin-1-yl]-pyridazin-3-yl}-urea; 1-(4-fluorophenyl)-3-{6-[4-(2-trifluoromethylbenzoyl)-piperazin-1-yl]-pyridazin-3-yl}-urea; 1-(2-fluorophenyl)-3-{6-[4-(2-trifluoromethylbenzoyl)-piperazin-1-yl]-pyridazin-3-yl}-urea; 1-phenethyl-3-{6-[4-(2-trifluoromethylbenzoyl)-piperazin-1-yl]-pyridazin-3-yl}-urea; 1-(4-fluorobenzyl)-3-{6-[4-(2-trifluoromethylbenzoyl)-piperazin-1-yl]-pyridazin-3-yl}-urea; 1-butyl-3-{6-[4-(2-trifluoromethylbenzoyl)-piperazin-1-yl]-pyridazin-3-yl}-urea; 1-cyclopentyl-3-{6-[4-(2-trifluoromethylbenzoyl)-piperazin-1-yl]-pyridazin-3-yl}-urea; 1-hexyl-3-{6-[4-(2-trifluoromethylbenzoyl)-piperazin-1-yl]-pyridazin-3-yl}-urea; 1-heptyl-3-{6-[4-(2-trifluoromethylbenzoyl)-piperazin-1-yl]-pyridazin-3-yl}-urea; 1-(3,4-dichlorobenzoyl)-3-{6-[4-(2-trifluoromethylbenzoyl)-piperazin-1-yl]-pyridazin-3-yl}-urea; 1-cyclohexyl-3-{6-[4-(2-trifluoromethylbenzoyl)-piperazin-1-yl]-pyridazin-3-yl}-urea; 2-phenoxy-N-{6-[4-(2-trifluoromethylbenzoyl)-piperazin-1-yl]-pyridazin-3-yl}-acetamide; 2-phenylcyclopropanecarboxylic acid (2-phenylcyclopropanecarbonyl)-{6-[4-(2-trifluoromethylbenzoyl)-piperazin-1-yl]-pyridazin-3-yl}-amide; 2-phenylcyclopropanecarboxylic acid {6-[4-(2-trifluoromethylbenzoyl)-piperazin-1-yl]-pyridazin-3-yl}-amide; hexanoic acid {6-[4-(2-trifluoromethylbenzoyl)-piperazin-1-yl]-pyridazin-3-y-l}-amide; 4-fluoro-N-{6-[4-(2-trifluoromethylbenzoyl)-piperazin-1-yl]-pyridazin-3-yl}-benzamide; {6-[4-(2-trifluoromethylbenzoyl)-piperazin-1-yl]-pyridazin-3-yl}-carbamic acid butyl ester; {6-[4-(2-trifluoromethylbenzoyl)-piperazin-1-yl]-pyridazin-3-yl}-carbamic acid propyl ester; {6-[4-(2-trifluoromethylbenzoyl)-piperazin-1-yl]-pyridazin-3-yl}-carbamic acid isobutyl ester; {6-[4-(2-trifluoromethylbenzoyl)-piperazin-1-yl]-pyridazin-3-yl}-carbamic acid ethyl ester; 1-(3-cyclopropylpropyl)-3-{6-[4-(2-trifluoromethylbenzoyl)-piperazin-1-yl]-pyridazin-3-yl}-urea; 1-{6-[4-(2,6-difluorobenzoyl)-piperazin-1-yl]-pyridazin-3-yl}-3-(3-methylbutyl)-urea; 1-cyclopropylmethyl-3-{6-[4-(2-trifluoromethylbenzoyl)-piperazin-1-yl]-pyridazin-3-yl}-urea; 1-(3,3-dimethylbutyl)-3-{6-[4-(2-trifluoromethylbenzoyl)-piperazin-1-yl]-pyridazin-3-yl}-urea; 1-(2-cyclopropylethyl)-3-{6-[4-(2-trifluoromethylbenzoyl)-piperazin-1-yl]-pyridazin-3-yl}-urea; 1-(2-isopropoxyethyl)-3{6-[4-(2-trifluoromethylbenzoyl)-piperazin-1-yl]-pyridazin-3-yl}-urea; 1-(3-hydroxy-4,4-dimethylpentyl)-3-{6-[4-(2-trifluoromethylbenzoyl)-piperazin-1-yl]-pyridazin-3-yl}-urea; 1-(2-cyclopropylethyl)-3-{6-[4-(2-fluoro-6-trifluoromethylbenzoyl)-piperazin-1-yl]-pyridazin-3-yl}-urea; 1-(2-cyclopropylethyl)-3-{6-[4-(5-fluoro-2-trifluoromethylbenzoyl)-piperazin-1-yl]-pyridazin-3-yl}-urea; 1-(2-cyclopropylethyl)-3-{6-[4-(2,6-difluorobenzoyl)-piperazin-1-yl]-pyridazin-3-yl}-urea; 1-(3-cyclopropylpropyl)-3-{6-[4-(5-fluoro-2-trifluoromethylbenzoyl)-piperazin-1-yl]-pyridazin-3-yl}-urea; 1-(4-methylpentyl)-3-{6-[4-(2-trifluoromethylbenzoyl)-piperazin-1-yl]-pyridazin-3-yl}-urea; 6-[4-(2,5-dichlorobenzoyl)-piperazin-1-yl]-pyridazine-3-carboxylic acid (3-methylbutyl)-; 6-[4-(5-methyl-2-trifluoromethylfuran-3-carbonyl)-piperazin-1-yl]-pyridazine-3-carboxylic acid (2-cyclopropylethyl)-amide; 6-[4-(2-chloropyridine-3-carbonyl)-piperazin-1-yl]-pyridazine-3-carboxylic acid (2-cyclopropylethyl)-amide; 6-[4-(2-methyl-5-trifluoromethyloxazole-4-carbonyl)-piperazin-1-yl]-pyridazine-3-carboxylic acid (2-cyclopropylethyl)-amide; 6-[4-(2,6-dichloropyridine-3-carbonyl)-piperazin-1-yl]-pyridazine-3-carboxylic acid (2-cyclopropylethyl)-amide; 6-[4-(1-benzyl-5-trifluoromethyl-1h-[1,2,3]-triazole-4-carbonyl)-piperazin-1-yl]-pyridazine-3-carboxylic acid (3-methylbutyl)-amide; 6-[4-(3-benzyl-5-trifluoromethyl-3h-[1,2,3]-triazole-4-carbonyl)-piperazin-1-yl]-pyridazine-3-carboxylic acid (3-methylbutyl)-amide; 6-[4-(2-methyl-5-trifluoromethyl-2h-[1,2,3]-triazole-4-carbonyl)-piperazin-1-yl]-pyridazine-3-carboxylic acid (3-methylbutyl)-amide; 6-[4-(5-trifluoromethyl-3h-imidazole-4-carbonyl)-piperazin-1-yl]-pyridazine-3-carboxylic acid (3-methylbutyl)-amide; 6-[4-(2-methanesulfonylbenzoyl)-piperazin-1-yl]-pyridazine-3-carboxylic acid (3-methylbutyl)-amide; 6-[4-(2,2-dimethylbutyryl)-piperazin-1-yl]-pyridazine-3-carboxylic acid (2-cyclopropylethyl)-amide; 6-[4-(2,2-dimethylpentanoyl)-piperazin-1-yl]-pyridazine-3-carboxylic acid (2-cyclopropylethyl)-amide; 6-[4-(5-fluoro-2-methoxybenzoyl)-piperazin-1-yl]-pyridazine-3-carboxylic acid (2-cyclopropylethyl)-amide; 6-[4-(2-dimethylaminobenzoyl)-piperazin-1-yl]-pyridazine-3-carboxylic acid (2-cyclopropylethyl)-amide; 6-[4-(2-chloro-5-dimethylaminobenzoyl)-piperazin-1-yl]-pyridazine-3-carboxylic acid (2-cyclopropylethyl)-amide; 6-[4-(2,5-dimethylbenzoyl)-piperazin-1-yl]-pyridazine-3-carboxylic acid (2-cyclopropylethyl)-amide; 6-[4-(2,5-dichlorobenzoyl)-piperazin-1-yl]pyridazine-3-carboxylic acid (2-cyclopropylethyl)-amide; 6-[4-(1-methyl-1h-pyrrole-2-carbonyl)-piperazin-1-yl]-pyridazine-3-carboxylic acid (2-cyclopropylethyl)-amide; 6-[4-(4,4,4-trifluorobut-2-enoyl)-piperazin-1-yl]-pyridazine-3-carboxylic acid (2-cyclopropylethyl)-amide; 6-[4-(1-hydroxycyclopropanecarbonyl)-piperazin-1-yl]-pyridazine-3-carboxylic acid (2-cyclopropylethyl)-amide; 6-[4-(4,4,4-trifluoro-3-hydroxy-3-trifluoromethylbutyryl)-piperazin-1-yl]-pyridazine-3-carboxylic acid (2-cyclopropylethyl)-amide; 6-[4-(4,4,4-trifluoro-3-hydroxy-3-methylbutyryl)-piperazin-1-yl]-pyridazine-3-carboxylic acid (2-cyclopropylethyl)-amide; 6-(4-cyclobutanecarbonylpiperazin-1-yl)-pyridazine-3-carboxylic acid (2-cyclopropylethyl)-amide; 6-[4-(2-trifluoromethylcyclopropanecarbonyl)-piperazin-1-yl]-pyridazine-3-carboxylic acid (2-cyclopropylethyl)-amide; 6-[4-(4,4,4-trifluoro-3-trifluoromethylbut-2-enoyl)-piperazin-1-yl]-pyridazine-3-carboxylic acid (2-cyclopropylethyl)-amide; 6-[4-(2-trifluoromethylbenzoyl)-piperazin-1-yl]-pyridazine-3-carboxylic acid cyclobutylmethyl amide; 6-{4-[2-(2-trifluoromethylphenyl)-acetyl]-piperazin-1-yl}-pyridazine-3-carbox-ylic acid (2-cyclopropylethyl)-amide; 6-[4-(2-cyanobenzoyl)-piperazin-1-yl]-pyridazine-3-carboxylic acid (2-cyclopropylethyl)-amide; 6-[4-(4-trifluoromethylpyridine-3-carbonyl)-piperazin-1-yl]-pyridazine-3-carboxylic acid (3-methylbutyl)-amide; 6-[4-(4,4,4-trifluoro-3-methylbut-2-enoyl)-piperazin-1-yl]-pyridazine-3-carboxylic acid (3-methylbutyl)-amide; 6-[4-(1-trifluoromethylcyclopropanecarbonyl)-piperazin-1-yl]-pyridazine-3-carboxylic acid (3-methylbutyl)-amide; 6-[4-(pyridine-2-carbonyl)-piperazin-1-yl]-pyridazine-3-carboxylic acid (2-cyclopropylethyl)-amide; 6-[4-(2-trifluoromethylfuran-3-carbonyl)-piperazin-1-yl]-pyridazine-3-carboxylic acid (2-cyclopropylethyl)-amide; 6-[4-(5-trifluoromethyl-3h-[1,2,3]-triazole-4-carbonyl)-piperaz-in-1-yl]-pyridazine-3-carboxylic acid (3-methylbutyl)-amide; 6-[4-(2-trifluoromethylbenzyl)-piperazin-1-yl]-pyridazine-3-carboxylic acid (3-methylbutyl)-amide; 6-[4-(2-trifluoromethylbenzyl)-piperazin-1-yl]-pyridazine-3-carboxylic acid (2-cyclopropylethyl)-amide; 6-[4-(5-fluoro-2-trifluoromethylbenzyl)-piperazin-1-yl]-pyridazine-3-carboxylic acid (2-cyclopropylethyl)-amide; 6-[4-(4-fluoro-2-trifluoromethylbenzyl)-piperazin-1-yl]-pyridazine-3-carboxylic acid (2-cyclopropylethyl)-amide; 6-[4-(5-chloro-2-trifluoromethylbenzyl)-piperazin-1-yl]-pyridazine-3-carboxylic acid (2-cyclopropylethyl)-amide; 6-[4-(2-chloro-4-fluorobenzyl)-piperazin-1-yl]-pyridazine-3-carboxylic acid (2-cyclopropylethyl)-amide; 6-[4-(2,5-dichlorobenzyl)-piperazin-1-yl]-pyridazine-3-carboxylic acid (2-cyclopropylethyl)-amide; 6-[4-(5-fluoro-2-trifluoromethylbenzyl)-piperazin-1-yl]-pyridazine-3-carboxylic acid (3-methylbutyl)-amide; 6-[4-(2,4-dichlorobenzyl)-piperazin-1-yl]-pyridazine-3-carboxylic acid (2-cyclopropylethyl)-amide; 6-[4-(5-fluoro-2-trifluoromethylbenzyl)-piperazin-1-yl]-pyridazine-3-carboxylic acid (3-cyclopropylpropyl)-amide; 6-[4-(5-fluoro-2-trifluoromethylbenzoyl)-piperazin-1-yl]-pyridazine-3-carboxylic acid pent-4-enylamide; 6-[4-(2-aminobenzoyl)-piperazin-1-yl]-pyridazine-3-carboxylic acid (3-methylbutyl)-amide; {6-[4-(2-trifluoromethylbenzoyl)-piperazin-1-yl]-pyridazin-3-yl-}-carbamic acid 3,3-dimethylbutyl ester; {6-[4-(2-trifluoromethylbenzoyl)-piperazin-1-yl]-pyridazin-3-yl}-carbamic acid 2-cyclopropylethyl ester; 6-[4-(4,4,4-trifluoro-2-methylbutyryl)-piperazin-1-yl]-pyridazine-3-carboxylic acid (3-methylbutyl)-amide; 6-[4-(4,4,4-trifluoro-3-methylbutyryl)-piperazin-1-yl]-pyridazine-3-carboxylic acid (3-methylbutyl)-amide; 6-[4-(4,4,4-trifluorobutyryl)-piperazin-1-yl]-pyridazine-3-carboxylic acid (3-methylbutyl)-amide; 6-[4-(6-chloropyridine-2-carbonyl)-piperazin-1-yl]-pyridazine-3-carboxylic acid (3-methylbutyl)-amide; 6-[4-(2-methylcyclohexanecarbonyl)-piperazin-1-yl]-pyridazine-3-carboxylic acid (2-cyclopropylethyl)-amide; 6-[4-(3-methylcyclohexanecarbonyl)-piperazin-1-yl]-pyridazine-3-carboxylic acid (2-cyclopropylethyl)-amide; 6-[4-(4-methylcyclohexanecarbonyl)-piperazin-1-yl]-pyridazine-3-carboxylic acid (2-cyclopropylethyl)-amide; 2-{4-[6-(2-cyclopropylethylcarbamoyl)-pyridazin-3-yl]-piperazine-1-carbonyl}-benzoic acid methyl ester; 6-[4-(3,3,3-trifluoro-2-hydroxy-2-methylpropionyl)-piperazin-1-yl]-pyridazine-3-carboxylic acid (2-cyclopropylethyl)-amide; 6-[4-(2-trifluoromethylbenzoyl)-piperazin-1-yl]-pyridazine-3-carboxylic acid (2-cyclopropyl-2-hydroxyethyl)-amide; 4-methyl-2-({6-[4-(2-trifluoromethylbenzoyl)-piperazin-1-yl]-pyridazine-3-carbonyl}-amino)-pentanoic acid methyl ester; 6-[4-(2-trifluoromethylbenzoyl)-piperazin-1-yl]-pyridazine-3-carboxylic acid cyclopropylmethyl amide; 6-[4-(2-trifluoromethylbenzoyl)-piperazin-1-yl]-pyridazine-3-carboxylic acid [2-(4-metoxyphenyl)-ethyl]-amide; 6-[4-(2-trifluoromethylbenzoyl)-piperazin-1-yl]-pyridazine-3-carboxylic acid (3-phenylpropyl)-amide; 6-[4-(2-trifluoromethylbenzoyl)-piperazin-1-yl]-pyridazine-3-carboxylic acid [2-(4-chlorophenoxy)-ethyl]-amide; 6-[4-(2-trifluoromethylbenzoyl)-piperazin-1-yl]-pyridazine-3-carboxylic acid [2-(4-fluorophenoxy)-ethyl]-amide; 6-[4-(2-trifluoromethylbenzoyl)-piperazin-1-yl]-pyridazine-3-carboxylic acid [2-(2,4-difluorophenyl)-ethyl]-amide; 6-[4-(2-trifluoromethylbenzoyl)-piperazin-1-yl]-pyridazine-3-carboxylic acid (3,3-dimethylbutyl)-amide; 6-[4-(2-trifluoromethylbenzoyl)-piperazin-1-yl]-pyridazine-3-carboxylic acid (2-phenylcyclopropylmethyl)-amide; 6-[4-(2-trifluoromethylbenzoyl)-piperazin-1-yl]-pyridazine-3-carboxylic acid (3-cyclopropylpropyl)-amide; 4-[6-(2-cyclopropylethylcarbamoyl)-pyridazin-3-yl]-piperazine-1-carboxylic acid t-butyl ester; 6-[4-(tetrahydrofuran-2-carbonyl)-piperazin-1-yl]-pyridazine-3-carboxylic acid (2-cyclopropylethyl)-amide; 6-[4-(2-trifluoromethylbenzoyl)-piperazin-1-yl]-pyridazine-3-carboxylic acid [2-(3-fluorophenyl)-ethyl]-amide; 6-[4-(2-trifluoromethylbenzoyl)-piperazin-1-yl]-pyridazine-3-carboxylic acid [2-(4-fluorophenyl)-ethyl]-amide; 6-[4-(2-trifluoromethylbenzoyl)-piperazin-1-yl]-pyridazine-3-carboxylic acid [2-(2-fluorophenyl)-ethyl]-amide; 6-[4-(2-trifluoromethylbenzoyl)-piperazin-1-yl]-pyridazine-3-carboxylic acid [2-(4-chlorophenyl)-ethyl]-amide; 6-[4-(2-trifluoromethylbenzoyl)-piperazin-1-yl]-pyridazine-3-carboxylic acid [2-(3-chlorophenyl)-ethyl]-amide; 6-[4-(2-trifluoromethylbenzoyl)-piperazin-1-yl]-pyridazine-3-carboxylic acid (2-phenylpropyl)-amide; 6-[4-(2-trifluoromethylbenzoyl)-piperazin-1-yl]-pyridazine-3-carboxylic acid (2-biphenyl-4-yl-ethyl)-amide; 6-[4-(2-trifluoromethylbenzoyl)-piperazin-1-yl]-pyridazine-3-carboxylic acid (3-methylbutyl)-amide; 6-[4-(2-trifluoromethylbenzoyl)-piperazin-1-yl]-pyridazine-3-carboxylic acid (4-hydroxybutyl)-amide; (r)-6-[4-(2-trifluoromethylbenzoyl)-piperazin-1-yl]-pyridazine-3-carboxylic acid (2-hydroxy-2-phenylethyl)-amide; (s)-6-[4-(2-trifluoromethylbenzoyl)-piperazin-1-yl]-pyridazine-3-carboxylic acid (2-hydroxy-2-phenylethyl)-amide; 4-({6-[4-(2-trifluoromethylbenzoyl)-piperazin-1-yl]-pyridazine-3-carbonyl}-am-ino)-butyric acid ethyl ester; 6-[4-(2-trifluoromethylbenzoyl)-piperazin-1-yl]-pyridazine-3-carboxylic acid (3-hydroxy-4,4-dimethylpentyl)-amide; 6-[4-(2-trifluoromethylbenzoyl)-piperazin-1-yl]-pyridazine-3-carboxylic acid (3-hydroxy-3-methylbutyl)-amide; 6-[4-(2-trifluoromethylbenzoyl)-piperazin-1-yl]-pyridazine-3-carboxylic acid (2-ethoxyethyl)-amide; 6-[4-(2-trifluoromethylbenzoyl)-piperazin-1-yl]-pyridazine-3-carboxylic acid pentylamide; 6-[4-(2-trifluoromethylbenzoyl)-piperazin-1-yl]-pyridazine-3-carboxylic acid (2-hydroxy-3,3-dimethylbutyl)-amide; 6-[4-(5-fluoro-2-trifluoromethylbenzoyl)-piperazin-1-yl]-pyridazine-3-carboxylic acid (2-hydroxy-3,3-dimethylbutyl)-amide; 6-[4-(2-methylcyclopropanecarbonyl)-piperazin-1-yl]-pyridazine-3-carboxylic acid (2-cyclopropylethyl)-amide; 6-[4-(5-fluoro-2-trifluoromethylbenzoyl)-piperazin-1-yl]-pyridazine-3-carboxylic acid pentylamide; 6-[4-(2-trifluoromethylbenzoyl)-piperazin-1-yl]-pyridazine-3-carboxylic acid (4-methylpentyl)-amide; 6-[4-(5-fluoro-2-trifluoromethylbenzoyl)-piperazin-1-yl]-pyridazine-3-carboxylic acid (3-methylbutyl)-amide; 6-[4-(4-fluoro-2-trifluoromethylbenzoyl)-piperazin-1-yl]-pyridazine-3-carboxylic acid (3-methylbutyl)-amide; 6-[4-(2-fluoro-6-trifluoromethylbenzoyl)-piperazin-1-yl]-pyridazine-3-carboxylic acid (3-methylbutyl)-amide; 6-[4-(2,6-difluorobenzoyl)-piperazin-1-yl]-pyridazine-3-carboxylic acid (3-methylbutyl)-amide; 6-[4-(2,2,3,3-tetramethylcyclopropanecarbonyl)-piperazin-1-yl]-pyridazine-3-carboxylic acid (2-cyclopropylethyl)-amide; 6-[4-(2-trifluoromethylbenzoyl)-piperazin-1-yl]-pyridazine-3-carboxylic acid (2-methylcyclopropylmethyl)-amide; 4-[6-(3-methylbutylcarbamoyl)-pyridazin-3-yl]-piperazine-1-carboxylic acid t-butyl ester; 6-[4-(2-trifluoromethylbenzoyl)-piperazin-1-yl]-pyridazine-3-carboxylic acid (2-cyclobutylethyl)-amide; 6-[4-(2-trifluoromethylbenzoyl)-piperazin-1-yl]-pyridazine-3-carboxylic acid hexylamide; 6-[4-(2-trifluoromethylbenzoyl)-piperazin-1-yl]-pyridazine-3-carboxylic acid (3-cyclobutylpropyl)-amide; 6-[4-(2-trifluoromethylbenzoyl)-piperazin-1-yl]-pyridazine-3-carboxylic acid heptylamide; 6-[4-(2-trifluoromethylbenzoyl)-piperazin-1-yl]-pyridazine-3-carboxylic acid (4-cyclopropylbutyl)-amide; 4-methyl-2-({6-[4-(2-trifluoromethylbenzoyl)-piperazin-1-yl]-pyridazine-3-carbonyl}-amino)-pentanoic acid; 6-{4-[1-(2-trifluoromethylphenyl)-ethyl]-piperazin-1-yl}-pyridazine-3-carboxylic acid (2-cyclopropylethyl)-amide hydrochloride; 6-[4-(2-trifluoromethylbenzoyl)-piperazin-1-yl]-pyridazine-3-carboxylic acid (2-oxo-2-phenylethyl)-amide; acetic acid 1-phenyl-2-({6-[4-(2-trifluoromethyl-benzoyl)-pipe-razin-1-yl]-pyridazine-3-carbonyl}-amino)-ethyl ester; acetic acid 1,1-dimethyl-3-({6-[4-(2-trifluoromethylbenzoyl)-piperazin-1-yl]-pyridazine-3-carbonyl}amino)-propyl ester; 6-[4-(2-trifluoromethylbenzoyl)-piperazin-1-yl]-pyridazine-3-carboxylic acid (2-methoxy-3,3-dimethylbutyl)-amide; 6-[3,5-dimethyl-4-(2-trifluoromethylbenzoyl)-piperazin-1-yl]-pyridazine-3-carboxylic acid (2-cyclopropylethyl)-amide; 6-[2,5-dimethyl-4-(2-trifluoromethylbenzoyl)-piperazin-1-yl]-pyridazine-3-carboxylic acid pentylamide; 2-{4-[6-(2-cyclopropylethylcarbamoyl)-pyridazin-3-yl]-piperazine-1-carbonyl}-benzoic acid; 6-[4-(2-trifluoromethylbenzoyl)-piperazin-1-yl]-pyridazine-3-carboxylic acid 2,2-(dimethylcyclopropylmethyl)-amide; 6-[4-(2-trifluoromethylbenzoyl)-piperazin-1-yl]-pyridazine-3-carboxylic acid (2-thiophen-2-yl-ethyl)-amide; 6-[4-(2-trifluoromethylbenzoyl)-piperazin-1-yl]-pyridazine-3-carboxylic acid (6-chloropyridazin-3-yl)-amide; 6-[4-(2-trifluoromethylbenzoyl)-piperazin-1-yl]-pyridazine-3-carboxylic acid (2-cyclopropyl-2-oxoethyl)-amide; 6-[4-(2-sulfamoylbenzoyl)-piperazin-1-yl]-pyridazine-3-carboxylic acid (3-methylbutyl)-amide; 6-[4-(2-trifluoromethylbenzoyl)-piperazin-1-yl]-pyridazine-3-carboxylic acid (4-chlorophenyl)-amide; 6-[4-(2-trifluoromethylbenzoyl)-piperazin-1-yl]-pyridazine-3-carboxylic acid (5-chloropyridin-2-yl)-amide; 6-[4-(2-trifluoromethylbenzoyl)-piperazin-1-yl]-pyridazine-3-carboxylic acid (2,2-difluoro-2-pyridin-2-ylethyl)-amide; 6-[4-(2-trifluoromethylbenzoyl)-piperazin-1-yl]-pyridazine-3-carboxylic acid (2,2-difluoro-2-phenylethyl)-amide; 6-[4-(2-trifluoromethylbenzoyl)-piperazin-1-yl]-pyridazine-3-carboxylic acid [2-(3-fluorophenyl)-2-hydroxyethyl]-amide; 6-[4-(2-trifluoromethylbenzoyl)-piperazin-1-yl]-pyridazine-3-carboxylic acid pyridin-2-ylamide; 6-[4-(2-trifluoromethylbenzoyl)-piperazin-1-yl]-pyridazine-3-carboxylic acid pyridazin-3-ylamide; 6-[4-(2-trifluoromethylbenzoyl)-piperazin-1-yl]-pyridazine-3-carboxylic acid (2-pyridin-2-ylethyl)-amide; 6-[4-(2-trifluoromethylbenzoyl)-piperazin-1-yl]-pyridazine-3-carboxylic acid (benzo[1,3]-dioxol-5-yl-methyl)-amide; 6-[4-(2-trifluoromethylbenzoyl)-piperazin-1-yl]-pyridazine-3-carboxylic acid (pyridin-2-yl-methyl)-amide; 6-[4-(2-trifluoromethylbenzoyl)-piperazin-1-yl]-pyridazine-3-carboxylic acid (2-benzo[1,3]-dioxol-5-yl-ethyl)-amide; 6-[4-(2-trifluoromethylthiobenzoyl)-piperazin-1-yl]-pyridazine-3-carboxylic acid (2-cyclopropylethyl)-amide; 6-[4-(2-trifluoromethylbenzoyl)-piperazin-1-yl]-pyridazine-3-carboxylic acid (2-phenoxyethyl)-amide; 6-[4-(2-trifluoromethylbenzoyl)-piperazin-1-yl]-pyridazine-3-carboxylic acid [3-(4-fluorophenyl)-propyl]-amide; 1-[1-(4-fluorophenyl)-ethyl]-3-{6-[4-(2-trifluoromethylbenzoyl)-piperazin-1-yl]-pyridazin-3-yl}-urea; 1-[3-(4-fluorophenyl)-propyl]-3-{6-[4-(2-trifluoromethylbenzoyl)-piperazin-1-yl]-pyridazin-3-yl}-urea; 3-cyclopentyl-N-{6-[4-(2-trifluoromethylbenzoyl)-piperazin-1-yl]-pyridazin-3-yl}-propionamide; 6-[4-(2-trifluoromethylbenzoyl)-piperazin-1-yl]-pyridazine-3-carboxylic acid phenethylamide; 6-[4-(2-trifluoromethylbenzoyl)-piperazin-1-yl]-pyridazine-3-carboxylic acid (5-trifluoromethylpyridin-2-yl)-amide; 6-[4-(2-trifluoromethylbenzoyl)-piperazin-1-yl]-pyridazine-3-carboxylic acid (4-carbamoylphenyl)-amide; 6-[4-(2-trifluoromethylbenzoyl)-piperazin-1-yl]-pyridazine-3-carboxylic acid (3-carbamoylphenyl)-amide; 6-[4-(2-trifluoromethylbenzoyl)-piperazin-1-yl]-pyridazine-3-carboxylic acid m-tolylamide; 6-[4-(2-trifluoromethylbenzoyl)-piperazin-1-yl]-pyridazine-3-carboxylic acid p-tolylamide; 6-[4-(2-trifluoromethylbenzoyl)-piperazin-1-yl]-pyridazine-3-carboxylic acid o-tolylamide; 6-[4-(2-trifluoromethylbenzoyl)-piperazin-1-yl]-pyridazine-3-carboxylic acid (2-propylphenyl)-amide; 6-[4-(2-trifluoromethylbenzoyl)-piperazin-1-yl]-pyridazine-3-carboxylic acid (4-propylphenyl)-amide; 6-[4-(2-trifluoromethylbenzoyl)-piperazin-1-yl]-pyridazine-3-carboxylic acid (4-isopropylphenyl)-amide; 6-[4-(2-trifluoromethylbenzoyl)-piperazin-1-yl]-pyridazine-3-carboxylic acid (2-isopropylphenyl)-amide; 6-[4-(2-trifluoromethylbenzoyl)-piperazin-1-yl]-pyridazine-3-carboxylic acid (2-chloro-phenyl)-amide; 6-[4-(2-trifluoromethylbenzoyl)-piperazin-1-yl]-pyridazine-3-carboxylic acid (2-cyano-3-fluorophenyl)-amide; 6-[4-(2-trifluoromethylbenzoyl)-piperazin-1-yl]-pyridazine-3-carboxylic acid (2,4-dimethylphenyl)-amide; 6-[4-(2-trifluoromethylbenzoyl)-piperazin-1-yl]-pyridazine-3-carboxylic acid (2,5-dimethylphenyl)-amide; 6-[4-(2-trifluoromethylbenzoyl)-piperazin-1-yl]-pyridazine-3-carboxylic acid (2,6-dimethylphenyl)-amide; 6-[4-(2-trifluoromethylbenzoyl)-piperazin-1-yl]-pyridazine-3-carboxylic acid (2,3-dimethylphenyl)-amide; 6-[4-(2-trifluoromethylbenzoyl)-piperazin-1-yl]-pyridazine-3-carboxylic acid (3,5-dimethylphenyl)-amide; 6-[4-(2-trifluoromethylbenzoyl)-piperazin-1-yl]-pyridazine-3-carboxylic acid (3,4-dimethyl-phenyl)-amide; 6-[4-(2-trifluoromethylbenzoyl)-piperazin-1-yl]-pyridazine-3-carboxylic acid (4-ethyl-phenyl)-amide; 6-[4-(2-trifluoromethylbenzoyl)-piperazin-1-yl]-pyridazine-3-carboxylic acid (2-ethyl-phenyl)-amide; 6-[4-(2-trifluoromethylbenzoyl)-piperazin-1-yl]-pyridazine-3-carboxylic acid (3-fluoro-2-methylphenyl)-amide; 6-[4-(2-trifluoromethylbenzoyl)-piperazin-1-yl]-pyridazine-3-carboxylic acid (2-fluoro-4-methylphenyl)-amide; 6-[4-(2-trifluoromethylbenzoyl)-piperazin-1-yl]-pyridazine-3-carboxylic acid (4-fluoro-2-methylphenyl)-amide; 6-[4-(2-trifluoromethylbenzoyl)-piperazin-1-yl]-pyridazine-3-carboxylic acid (2-fluoro-5-methylphenyl)-amide; 6-[4-(2-trifluoromethylbenzoyl)-piperazin-1-yl]-pyridazine-3-carboxylic acid (3-fluoro-5-methylphenyl)-amide; 6-[4-(2-trifluoromethylbenzoyl)-piperazin-1-yl]-pyridazine-3-carboxylic acid (3-fluoro-phenyl)-amide; 6-[4-(2-trifluoromethylbenzoyl)-piperazin-1-yl]-pyridazine-3-carboxylic acid (2-fluoro-phenyl)-amide; 6-[4-(2-trifluoromethylbenzoyl)-piperazin-1-yl]-pyridazine-3-carboxylic acid (4-fluoro-phenyl)-amide; 6-[4-(2-trifluoromethylbenzoyl)-piperazin-1-yl]-pyridazine-3-carboxylic acid (2,4-difluorophenyl)-amide; 6-[4-(2-trifluoromethylbenzoyl)-piperazin-1-yl]-pyridazine-3-carboxylic acid (2,5-difluorophenyl)-amide; 6-[4-(2-trifluoromethylbenzoyl)-piperazin-1-yl]-pyridazine-3-carboxylic acid (3,4-difluorophenyl)-amide; 6-[4-(2-trifluoromethylbenzoyl)-piperazin-1-yl]-pyridazine-3-carboxylic acid (2,3-difluorophenyl)-amide; 6-[4-(2-trifluoromethylbenzoyl)-piperazin-1-yl]-pyridazine-3-carboxylic acid (2,6-difluorophenyl)-amide; 6-[4-(2-trifluoromethylbenzoyl)-piperazin-1-yl]-pyridazine-3-carboxylic acid (7h-purin-6-yl)-amide; 6-[4-(2-trifluoromethylbenzoyl)-piperazin-1-yl]-pyridazine-3-carboxylic acid pyrazin-2-ylamide; 6-[4-(2-trifluoromethylbenzoyl)-piperazin-1-yl]-pyridazine-3-carboxylic acid indan-1-ylamide; 6-[4-(2-trifluoromethylbenzoyl)-piperazin-1-yl]-pyridazine-3-carboxylic acid (1 h-tetrazol-5-yl)-amide; 6-[4-(2-trifluoromethylbenzoyl)-piperazin-1-yl]-pyridazine-3-carboxylic acid (2h-[[0665]-1,2,4]-triazol-3-yl)-amide; 6-[4-(2-trifluoromethylbenzoyl)-piperazin-1-yl]-pyridazine-3-carboxylic acid (3-methyl-isoxazol-5-yl)-amide; 6-[4-(2-trifluoromethylbenzoyl)-piperazin-1-yl]-pyridazine-3-carboxylic acid (5-methyl-isoxazol-3-yl)-amide; 6-[4-(2-trifluoromethylbenzoyl)-piperazin-1-yl]-pyridazine-3-carboxylic acid (1 h-pyrazol-3-yl)-amide; 6-[4-(2-trifluoromethylbenzoyl)-piperazin-1-yl]-pyridazine-3-carboxylic acid (5-methyl-1h-pyrazol-3-yl)-amide; 6-[4-(2-trifluoromethylbenzoyl)-piperazin-1-yl]-pyridazine-3-carboxylic acid pyrimidin-2-ylamide; 6-[4-(2-trifluoromethylbenzoyl)-piperazin-1-yl]-pyridazine-3-carboxylic acid pyrazin-2-ylamide; 6-[4-(2-trifluoromethylbenzoyl)-piperazin-1-yl]-pyridazine-3-carboxylic acid (4-methyl-pyrimidin-2-yl)-amide; 6-[4-(2-trifluoromethylbenzoyl)-piperazin-1-yl]-pyridazine-3-carboxylic acid (2-oxo-2,3-dihydropyrimidin-4-yl)-amide; 6-[4-(2-trifluoromethylbenzoyl)-piperazin-1-yl]-pyridazine-3-carboxylic acid (6-oxo-1,6-dihydropyrimidin-2-yl)-amide; 6-[4-(2-trifluoromethylbenzoyl)-piperazin-1-yl]-pyridazine-3-carboxylic acid (2-oxo-1,3-diazabicyclo[3.1.0]-hex-3-en-4-yl)-amide; 6-[4-(2-trifluoromethylbenzoyl)-piperazin-1-yl]-pyridazine-3-carboxylic acid (5-oxo-4,5-dihydro-1h-pyrazol-3-yl)-amide; 6-[4-(2-trifluoromethylbenzoyl)-piperazin-1-yl]-pyridazine-3-carboxylic acid [1,3,4]-thiadiazol-2-ylamide; 6-[4-(2-trifluoromethylbenzoyl)-piperazin-1-yl]-pyridazine-3-carboxylic acid thiazol-2-ylamide; 6-[4-(2-trifluoromethylbenzoyl)-piperazin-1-yl]-pyridazine-3-carboxylic acid indan-5-ylamide; 6-[4-(2-trifluoromethyl-benzoyl)-piperazin-1-yl]-pyridazine-3-carboxylic acid pyridin-2-ylamide; 6-[4-(2-trifluoromethylbenzoyl)-piperazin-1-yl]-pyridazine-3-carboxylic acid pyridin-3-ylamide; 6-[4-(2-trifluoromethylbenzoyl)-piperazin-1-yl]-pyridazine-3-carboxylic acid pyridin-4-ylamide; 6-[4-(2-trifluoromethylbenzoyl)-piperazin-1-yl]-pyridazine-3-carboxylic acid (6-oxo-1,6-dihydro[1,3,5]-triazin-2-yl)-amide; 6-[4-(2-trifluoromethylbenzoyl)-piperazin-1-yl]-pyridazine-3-carboxylic acid (5-fluoro-pyridin-2-yl)-amide; 6-[4-(2-trifluoromethylbenzoyl)-piperazin-1-yl]-pyridazine-3-carboxylic acid (4-cyano-phenyl)-amide; 6-[4-(2-trifluoromethylbenzoyl)-piperazin-1-yl]-pyridazine-3-carboxylic acid (2-cyano-phenyl)-amide; 6-[4-(2-trifluoromethylbenzoyl)-piperazin-1-yl]-pyridazine-3-carboxylic acid (3-cyano-phenyl)-amide; 6-[4-(2-trifluoromethylbenzoyl)-piperazin-1-yl]-pyridazine-3-carboxylic acid (5-cyano-pyridin-2-yl)-amide; 6-[4-(2-trifluoromethylbenzoyl)-piperazin-1-yl]-pyridazine-3-carboxylic acid (4,6-dimethylpyrimidin-2-yl)-amide; 6-[4-(2-trifluoromethylbenzoyl)-piperazin-1-yl]-pyridazine-3-carboxylic acid (2-chloro-pyridin-4-yl)-amide; 6-[4-(2-trifluoromethylbenzoyl)-piperazin-1-yl]-pyridazine-3-carboxylic acid (1 h-indol-6-yl)-amide; 6-[4-(2-trifluoromethylbenzoyl)-piperazin-1-yl]-pyridazine-3-carboxylic acid (1 h-indol-4-yl)-amide; 6-[4-(2-trifluoromethylbenzoyl)-piperazin-1-yl]-pyridazine-3-carboxylic acid (1 h-indazol-5-yl)-amide; 6-[4-(2-trifluoromethylbenzoyl)-piperazin-1-yl]-pyridazine-3-carboxylic acid (1 h-indazol-6-yl)-amide; 6-[4-(2-trifluoromethylbenzoyl)-piperazin-1-yl]-pyridazine-3-carboxylic acid (4-methyl-thiazol-2-yl)-amide; 6-[4-(2-trifluoromethylbenzoyl)-piperazin-1-yl]-pyridazine-3-carboxylic acid (5-methyl-thiazol-2-yl)-amide; 6-[4-(2-trifluoromethylbenzoyl)-piperazin-1-yl]-pyridazine-3-carboxylic acid (5-thioxo-4,5-dihydro-1h-[1,2,4]-triazol-3-yl)-amide; 6-[4-(2-trifluoromethylbenzoyl)-piperazin-1-yl]-pyridazine-3-carboxylic acid (1 h-benzoimidazol-2-yl)-amide; 6-[4-(2-trifluoromethylbenzoyl)-piperazin-1-yl]-pyridazine-3-carboxylic acid (6-methylpyridazin-3-yl)-amide; 6-[4-(2-trifluoromethylbenzoyl)-piperazin-1-yl]-pyridazine-3-carboxylic acid (6-methoxypyridazin-3-yl)-amide; 6-[4-(2-trifluoromethylbenzoyl)-piperazin-1-yl]-pyridazine-3-carboxylic acid (3-chloro-phenyl)-amide; 6-[4-(2-trifluoromethylbenzoyl)-piperazin-1-yl]-pyridazine-3-carboxylic acid (3-chloro-2-methylphenyl)-amide; 6-[4-(2-trifluoromethylbenzoyl)-piperazin-1-yl]-pyridazine-3-carboxylic acid (2-chloro-3-methylphenyl)-amide; 6-[4-(2-trifluoromethylbenzoyl)-piperazin-1-yl]-pyridazine-3-carboxylic acid (2,5-dichlorophenyl)-amide; 6-[4-(2-trifluoromethylbenzoyl)-piperazin-1-yl]-pyridazine-3-carboxylic acid (2-chloro-5-methylphenyl)-amide; 6-[4-(2-trifluoromethylbenzoyl)-piperazin-1-yl]-pyridazine-3-carboxylic acid (2-chloro-6-methylphenyl)-amide; 6-[4-(2-trifluoromethylbenzoyl)-piperazin-1-yl]-pyridazine-3-carboxylic acid (4-chloro-2-methylphenyl)-amide; 6-[4-(2-trifluoromethylbenzoyl)-piperazin-1-yl]-pyridazine-3-carboxylic acid (4-chloro-3-methylphenyl)-amide; 6-[4-(2-trifluoromethylbenzoyl)-piperazin-1-yl]-pyridazine-3-carboxylic acid (3-chloro-4-methylphenyl)-amide; 6-[4-(2-trifluoromethylbenzoyl)-piperazin-1-yl]-pyridazine-3-carboxylic acid (2-chloro-4-methylphenyl)-amide; 6-[4-(2-trifluoromethylbenzoyl)-piperazin-1-yl]-pyridazine-3-carboxylic acid (2-chloro-5-fluorophenyl)-amide; 6-[4-(2-trifluoromethylbenzoyl)-piperazin-1-yl]-pyridazine-3-carboxylic acid (5-chloro-2-fluorophenyl)-amide; 6-[4-(2-trifluoromethylbenzoyl)-piperazin-1-yl]-pyridazine-3-carboxylic acid (2,5-difluorophenyl)-amide; 6-[4-(2-trifluoromethylbenzoyl)-piperazin-1-yl]-pyridazine-3-carboxylic acid (2,6-dichlorophenyl)-amide; 6-[4-(2-trifluoromethylbenzoyl)-piperazin-1-yl]-pyridazine-3-carboxylic acid (2-trifluoromethylphenyl)-amide; 6-[4-(2-trifluoromethylbenzoyl)-piperazin-1-yl]-pyridazine-3-carboxylic acid (4-trifluoromethylphenyl)-amide; 6-[4-(2-trifluoromethylbenzoyl)-piperazin-1-yl]-pyridazine-3-carboxylic acid (3-trifluoromethylphenyl)-amide; 6-[4-(2-trifluoromethylbenzoyl)-piperazin-1-yl]pyridazine-3-carboxylic acid phenylamide; 6-[4-(2-trifluoromethylbenzoyl)-piperazin-1-yl]-pyridazine-3-carboxylic acid (5-chloro-2-methoxyphenyl)-amide; 6-[4-(2-trifluoromethylbenzoyl)-piperazin-1-yl]-pyridazine-3-carboxylic acid (2,5-dimethoxyphenyl)-amide; 6-[4-(2-trifluoromethylbenzoyl)-piperazin-1-yl]-pyridazine-3-carboxylic acid (2-chloro-4-methoxyphenyl)-amide; 6-[4-(2-trifluoromethylbenzoyl)-piperazin-1-yl]-pyridazine-3-carboxylic acid (4-methoxyphenyl)-amide; 6-[4-(2-trifluoromethylbenzoyl)-piperazin-1-yl]-pyridazine-3-carboxylic acid (2-methoxyphenyl)-amide; 6-[4-(2-trifluoromethylbenzoyl)-piperazin-1-yl]-pyridazine-3-carboxylic acid (3-methoxyphenyl)-amide; 4-({6-[4-(2-trifluoromethylbenzoyl)-piperazin-1-yl]-pyridazine-3-carbonyl}-amino)-benzoic acid methyl ester; 4-({6-[4-(2-trifluoromethylbenzoyl)-piperazin-1-yl]-pyridazine-3-carbonyl}-amino)-benzoic acid; 2-({6-[4-(2-trifluoromethylbenzoyl)-piperazin-1-yl]-pyridazine-3-carbonyl}-amino)-benzoic acid methyl ester; 2-({6-[4-(2-trifluoromethylbenzoyl)-piperazin-1-yl]-pyridazine-3-carbonyl}-amino)-benzoic acid; 6-[4-(2-trifluoromethylbenzoyl)-piperazin-1-yl]-pyridazine-3-carboxylic acid (3,4-dichlorophenyl)-amide; 1-[1-(4-fluorophenyl)-ethyl]-3-{6-[4-(2-trifluoromethylbenzoyl)-piperazin-1-yl]-pyridazin-3-yl}-urea.

Without wishing to be bound by theory, it is believed that the compounds and methods of the present invention can reduce the secretion of Aβ42 peptide from cells within the brains of patients and thereby lower Aβ42 peptide production in the patients' brains. In so doing, it is believed that the compounds of the present invention are useful for treating and/or preventing neurodegenerative diseases according to the methods of the invention. Thus, in one aspect of this invention, which is described in detail below, methods of treating AD, MCI, CAA, or dementia are provided comprising identifying a patient in need of such treatment, and administering to that patient an Aβ42-lowering, effective amount of a compound of the present invention. Preferably, the compound that is used in the methods of the invention is capable of reducing Aβ42 secretion by neurons by at least 10, 20, 30, 40, or 50 percent, at a concentration of 10 μM in an assay of Aβ42 secretion.

Preferred compounds for use in the methods of the invention are those that have an IC₅₀, in assays of Aβ42 secretion, of 100 μM or less, more preferably 10 μM or less, and even more preferably 1 μM or less.

Additionally, the Formulae presented above are intended to cover solvated as well as unsolvated forms of the identified compounds. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

It is understood that while the compounds for use in the invention may exhibit the phenomenon of tautomerism, the formula drawings within this specification expressly depict only one of the possible tautomeric forms. It is therefore to be understood that within this specification the formulae are intended to represent any tautomeric form of the depicted compound, and the depicted compounds are not to be limited merely to a specific tautomeric form depicted by a formula drawing.

Some of the compounds for use in the invention may exist as single stereoisomers (i.e., essentially free of other stereoisomers), racemates, and/or mixtures of enantiomers and/or diastereomers. All such single stereoisomers, racemates, and mixtures thereof are intended to be within the scope of the present invention. Preferably, the inventive compounds that are optically active are used in an optically pure form.

As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the Formulae presented above are intended to cover solvated as well as unsolvated forms of the identified compounds. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of the Formulae I-V(g)(2), the invention includes pharmaceutically acceptable prodrugs, pharmaceutically active metabolites, and pharmaceutically acceptable salts of such compounds.

“A pharmaceutically acceptable prodrug” is a compound that may be converted under physiological conditions or by solvolysis to the specified compound or to a pharmaceutically acceptable salt of such compound.

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

Prodrugs and active metabolites of compound may be identified using routine techniques known in the art. See, e.g., Bertolini, G et al., J. Med. Chem., 40, 2011-2016 (1997); Shan, D. et al., J. Pharm. Sci., 86 (7), 756-767; Bagshawe K., Drug Dev. Res., 34, 220-230 (1995); Bodor N, Advance in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

“A pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma.-hydroxybutyrates, glycollates, tartrates, methane-sulfonates, propanesulfonates, naphthalene-1-sulfonates, naphthalene-2-sulfonates, and mandelates.

If the compound for use in the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. These substituents may optionally be further substituted with a substituent selected from such groups.

The compounds of the present invention can have asymmetric centers and/or can exist in the form of cis or trans derivatives. The invention covers the racemates, mixtures of cis and trans compounds, and also covers optically active products with the cis derivatives and the trans derivatives taken independently. These pure products will be obtained by the methods known to those skilled in the art, in particular by chromatography, especially on chiral columns in the case of optical isomers.

### Pharmaceutical Compositions

The present invention also provides pharmaceutical compositions comprising a therapeutic SCD-reducing, Aβ42-lowering compound according to the present invention and a pharmaceutically acceptable excipient or carrier. Such pharmaceutical compositions are formulated so as to deliver a therapeutically or prophylactically effective amount of the therapeutic compound to a patient in need of such treatment.

When the composition having a compound of Formulae I-V(g)(2) is administered, according to the treatment regimens of the invention, to an individual desiring or needing such treatment, it provides an improvement or lessening of a decline in cognitive function, a biochemical disease marker, and/or amyloid plaque morphology and pathology associated with a neurodegenerative disorder characterized by the formation or accumulation of amyloid plaques. The pharmaceutical composition of the invention is formulated with one or more pharmaceutically acceptable salts, excipients, or carriers. The pharmaceutical composition can be delivered orally, preferably in a tablet or capsule dosage form, or by any other effective route. The pharmaceutical composition having a compound of Formulae I-V(g)(2) can be used in methods for treating or preventing neurodegenerative diseases or disorders characterized by the formation or accumulation of amyloid plaques, or in the prophylaxis of such diseases or disorders in patients having increased risk of developing such diseases or disorders.

In a specific embodiment of this aspect of the invention, the dosage is provided as a pharmaceutical composition that is composed of an effective amount of a compound of Formulae I-V(g)(2), a pharmaceutically acceptable salt, a release agent, a carrier or excipient, and additional optional ingredients. In another specific embodiment of this aspect of the invention, the dosage is provided as a pharmaceutical composition that is a tablet composed of a compound of Formulae I-V(g)(2), microcrystalline cellulose, colloidal silicon dioxide, and magnesium stearate. In another specific embodiment of this aspect of the invention, the dosage is provided as a pharmaceutical composition comprising a compound of Formulae I-V(g)(2), microcrystalline cellulose, colloidal silicon dioxide, and magnesium stearate, all encapsulated in a pharmaceutically acceptable capsule, optionally including lactose monohydrate, hydroxylpropyl methyl cellulose, titanium dioxide, tracetin/glycerol triacetate, and iron oxide.

### Combination Therapy

The invention further provides a combination therapy strategy for treating or preventing AD, MCI, and CAA. According to this aspect of the invention, an individual in need of treatment is administered a therapeutic amount of a compound of the present invention according to Formula I, and a compound selected from the group consisting of NSAIDs (non-steroidal anti-inflammatory drugs), COX-2 inhibitors (cyclooxygenase-2), β-secretase inhibitors, R-flurbiprofen, and γ-secretase inhibitors.

The methods of combination therapy provided are thought to provide a synergistic effect in reducing Aβ42 levels and are thought to be especially effective for preventing AD, MCI, and CAA. The treatment regimens used in the combination therapy can involve administration of pharmaceutical compositions comprising a combination of active ingredients, or the concomitant administration of separate compositions, each comprising at least one active ingredient. Furthermore, the administration of the active ingredients can be performed at different times and/or via different routes. For example, a composition comprising at least one active ingredient can be administered in the morning, and a composition comprising at least one different active ingredient can be administered in the evening. Another example would involve the administration of a composition having at least one active ingredient orally while the second composition having at least on other active ingredient is administered intravenously.

In addition to the advantages described above, while not wishing to be bound by theory, it is believed that therapeutic compounds of Formula I are capable of slowing the rate of death of neurons. Accordingly, it is also believed that the compounds of Formula I act in vivo to treat and/or prevent AD, MCI, and CAA by slowing the rate of death of neurons that is present, or would be present, in the absence of such treatment.

### Example Compounds, Synthesis, Effects and Effective Dosages

Specific compounds of Formula I for use in the methods of the present invention are described in detail in U.S. patent application Ser. Nos. 10/901,563, 10/885,901, and 10/566,856, and international patent application publications WO 2005/011653, WO 2005/011654, WO 2005/011655, WO 2005/011656, and WO 2005/011657. The specific compounds described in these patent applications are all incorporated by reference herein. Furthermore, the compounds for use in the methods of the present invention, and described in the aforementioned patent applications, can be synthesized by a skilled artisan according to the techniques described in U.S. patent application Ser. Nos. 10/901,563, 10/885,901, and 10/566,856, and international patent application publications WO 2005/011653, WO 2005/011654, WO 2005/011655, WO 2005/011656, and WO 2005/011657. The synthetic protocols and schemes provided in these patent applications are all incorporated by reference herein. The aforementioned patent applications also refer to dosages, formulations, and routes of delivery, all of which can be used in the methods of the present invention.

In one set of embodiments, preferred compounds for use in the methods of the present invention are those disclosed compounds that effectively inhibit SCD when present at μM concentrations, or, more preferably, at nM concentrations. Without wishing to be bound by theory, it is believed that by inhibiting human SCD with compounds such as: 6-[4-(2-Ethylbutyryl)piperazin-1-yl]pyridazine-3-carboxylic acid (2-cyclopropylethyl)amide; 6-[4-(3,3,3-Trifluoro-2-methyl-2-trifluoromethylpropionyl)piperazin-1-yl]pyridazine-3-carboxylic acid (2-cyclopropylethyl)amide; 6-[4-(2,2-Dimethylpropionyl)piperazin-1-yl]pyridazine-3-carboxylic acid (2-cyclopropylethyl)amide; 6-[4-(2,2-Dimethylbutyryl)piperazin-1-yl]pyridazine-3-carboxylic acid (2-cyclopropylethyl)amide; 6-[4-(2,2-Dimethylpentanoyl)piperazin-1-yl]pyridazine-3-carboxylic acid (2-cyclopropylethyl)amide; 6-[4-(4,4,4-Trifluorobut-2-enoyl)piperazin-1-yl]pyridazine-3-carboxylic acid (2-cyclopropylethyl)amide; or 6-[4-(4,4,4-Trifluoro-3-trifluoromethylbut-2-enoyl)-piperazin-1-yl]pyridazine-3-carboxylic acid (2-cyclopropyl-ethyl)amide; one can modulate amyloid precursor protein processing, thereby lowering Aβ42 secretion by neurons, and subsequently lowering levels of Aβ42 peptide in a patient, and thus treat and/or prevent AD or MCI according to the methods of the invention. Thus, methods of treating AD or MCI are provided comprising identifying a patient in need of such treatment, and administering to that patient a SCD-inhibiting, Aβ42-lowering, effective amount of a compound of Formula I. Preferably, the compound of Formula I that is used in the methods of the present invention is capable of inhibiting at least 10, 20, 30, 40, or 50 percent or more of the enzymatic activity of SCD, at a concentration of 1 μM (compound of Formula I). Additionally, preferred compounds for use in the methods of the present invention are those compounds according to Formula I that have a Kᵢ for SCD of 50 μM or less, more preferably 10 μM or less, and even more preferably 1 μM or less. Further, preferred compounds for use in the methods of the present invention are those according to Formula I that have an IC₅₀ for SCD of 50 μM or less, more preferably 10 μM or less, and even more preferably 1 μM or less. The efficacy of the compounds of Formula I in inhibiting SCD can be assessed by any of the methods provided in U.S. patent application Ser. Nos. 10/901,563, 10/885,901, and 10/566,856, and international patent application publications WO 2005/011653, WO 2005/011654, WO 2005/011655, WO 2005/011656, and WO 2005/011657. These methods for determining the efficacy of compounds in inhibiting SCD are incorporated by reference herein. The efficacy of the compounds of Formula I for lowering levels of Aβ42 peptide in cells, tissues or organs in a patient, and treating, delaying the onset of symptoms, slowing the progression of symptoms, or reversing the symptoms of MCI, AD, CAA, or dementia associated with DS, according to the methods of the present invention, can be assessed using one of the several assays presented in the Examples Section below.

### Formulations

The pills, tablets, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the tablet-forming machine are selected by the skilled artisan.

### Therapeutic Methods

As noted above, the present invention provides methods for treating, or preventing, or delaying the onset of, or reversing the symptoms of neurodegenerative diseases and disorders characterized by the deposition or accumulation of amyloid plaques comprising the Aβ42 peptide. These methods can be applied in any such neurodegenerative disease or disorder, but have clear application in AD, at all stages of its progression, and can also potentially be applied in MCI, CAA and dementia associated with DS. Such, methods have in common the lowering of Aβ42 levels in the brains of patients in need of such treatment through the reduction in cellular secretion of the Aβ42 peptide. While not wishing to be bound by theory, it is believed that by lowering the amounts of Aβ42 in the brains of an individual by administering an effective amount of a composition described herein, neurodegenerative diseases such as AD, MCI, CAA, and dementia associated with DS can be treated or prevented, or the symptoms of such diseases can be alleviated or even eliminated.

Generally, the invention relates to the concept that compounds which reduce SCD activity in cells lower the amount of Aβ42 produced by the cells. Thus, diseases characterized by increased levels cellular production or secretion of Aβ42, or by the accumulation or deposition of amyloid plaques comprising the Aβ42 peptide, can be treated or prevented with the methods of the invention, which are specifically designed to lower the amount of Aβ42 produced by cells, prevent an increase in Aβ42 secretion by cells, and/or reduce the rate of increase of Aβ42 levels in the brain of a patient.

Importantly, however, the methods in the present invention may also be used prophylactically in patients at risk of developing neurodegenerative diseases and disorders characterized by the deposition or accumulation of amyloid plaques comprising the Aβ42 peptide. Such patients may be identified by any acceptable method in the art, such as through genotyping by any suitable method, or by analysis of their family's history of disease, or through pedigree analysis. Methods of determining the genotype of an individual include nucleic acid sequencing, selective hybridization, allele-specific amplification, and the like. For patients found to be at risk by such methods, the methods of the present invention may be used to prevent or delay the onset of symptoms of neurodegenerative diseases and disorders characterized by the deposition or accumulation of amyloid plaques comprising the Aβ42 peptide.

As noted above, the present invention also provides therapeutic methods for use in treating patients in need of such treatments. These methods generally comprise administration of an effective amount of an SCD activity reducing, Aβ42-lowering, compound of the present invention to a patient in need of such treatment, through the administration of a pharmaceutical composition of the present invention.

As a first step, the therapeutic methods of present invention require the identification of patients in need of such treatment. This first step can be achieved by way of any of the appropriate techniques known in the art, including assessment of cognitive function, assays for biochemical markers, and/or determination of amyloid plaque number, density, size and morphology.

The decline in cognitive function observed in neurodegenerative diseases such as AD can be characterized by cognition tests. It is preferred that the lessening in decline in cognitive function is at least 25% as compared to individuals treated with placebo, more preferably at least 40%, and even more preferably at least 60%. For example, an individual treated with placebo having probable mild-to-moderate AD is expected to score approximately 5.5 points higher on the ADAS-cog test (higher scores indicate more impairment) after a specified period of time (e.g., 1 year) whereas an individual treated with the composition of the invention for the same period of time will score approximately 3.3 points higher on the ADAS-cog scale with a 60% decrease in decline in cognitive function or 2.2 points higher with a 40% decrease in decline cognitive function when treated for the same specified period of time.

In certain embodiments, the present invention relates to a method of preventing AD. According to this embodiment, a method for preventing AD is provided which comprises administering, to an individual in need of such treatment, a composition comprising a therapeutically effective amount of a compound according to Formula I. The method of this embodiment is useful for preventing or delaying the onset of the symptoms of AD, the onset of AD, and/or the progression of the disease. In these embodiments the patient in need of such treatment may be one who has yet to exhibit symptoms of AD, but is at risk of developing the disease. Alternatively, the patient to be treated may suffer from MCI or CAA, but who has yet to be clinically diagnosed with AD. Individuals at risk of developing AD can be identified by any acceptable method in the art. As noted above, such methods can include genotyping by any suitable method, analysis of family history of the disease, or through pedigree analysis. Methods of determining risk through genotyping include determining genotype by nucleic acid sequencing, selective hybridization, allele-specific amplification, and the like. Additionally, various biomarkers, such as Aβ42 peptide concentrations in plasma or serum or cerebrospinal fluid (CF), or amyloid plaque number, density, size and morphology, can be used to assess whether an individual is at risk of developing a neurodegenerative disease that can be treated or prevented using the methods of the present invention.

### Patient Population

Any individual having, or suspected of having, a neurodegenerative disorder, such as AD, MCI, CAA, or dementia can be treated using the methods of the present invention. Individuals who would particularly benefit from the methods of the invention include those individuals diagnosed as having mild to moderate AD according to a medically-accepted diagnosis, such as, for example the NINCDS-ADRDA criteria. Progression of the disease may be followed by medically accepted measure of cognitive function, such as, for example, the Mini-Mental State Exam (MMSE; see Mohs et al. Int. Psychogeriatr. 8:195-203 (1996)); ADAS-Cog (Alzheimer Disease Assessment Scale-Cognitive; see Galasko et al. Alzheimer Dis. Assoc. Disord. 11 suppl 2:S33-9 (1997)); Behavioral Pathology in Alzheimer's Disease Rating Scale (BEHAVE-AD); Blessed Test; CANTAB—Cambridge Neuropsychological Test Automated Battery; CERAD (The Consortium to Establish a Registry for Alzheimer's Disease) Clinical and Neuropsychological Tests (includes MMSE); Clock Draw Test; Cornell Scale for Depression in Dementia (CSDD); Geriatric Depression Scale (GDS); Neuropsychiatric Inventory (NPI); the 7 Minute Screen; the Alzheimer's Disease Cooperative Study Activities of Daily Living scale (ADCS-ADL; see McKhann et al. Neurology 34:939-944 (1984)); the DSM-IV (Diagnostic and Statistical Manual of Mental Disorders—Fourth Edition (DSM-IV), published by the American Psychiatric Association, Washington D.C., 1994); or the NINCDS-ADRDA criteria (see Folstein et al. J. Psychiatr. Res. 12:189-198 (1975)). Individuals diagnosed as having probable AD can be identified as having a mild-to-moderate form of the disease by an accepted measure of cognitive function such as the MMSE. In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993). The severity of AD is generally determined by one of the initial tests provided above. For example, MMSE scores of 26-19 indicate mild AD, while scores from 18-10 indicate moderate AD.

Diagnoses of AD based on these tests are recorded as presumptive or probable, and may optionally be supported by one or more additional criteria. For example, a diagnosis of AD may be supported by evidence of a family history of AD; non-specific changes in EEG, such as increased slow-wave activity; evidence of cerebral atrophy on CT with progression documented by serial observation; associated symptoms such as depression, insomnia, incontinence, delusions, illusions, hallucinations, catastrophic verbal, emotional or physical outbursts, sexual disorders, weight loss, and/or attendant neurologic abnormalities, such as increased muscle tone, myoclonus or gait disorder, etc.

Additionally, amyloid deposits, generally associated with AD, may be detected through the use of positron emission tomography (PET) using an amyloid-specific tracer such as Pittsburgh Compound-B (PIB). See Klunk et al., Ann. Neurol. 55(3):306-309 (2004). Increased amyloid deposits in the frontal, parietal, temporal and occipital cortices, and in the striatum, relative to normal brain tissue, as visualized, for example by PIB, support a diagnosis of AD. Generally, a greater number and density of amyloid deposits indicates more advanced AD.

Additionally, the invention, is some embodiments, relates to identifying an individual who is experiencing a decrease in the ratio of Aβ42/Aβ40 ratio in cerebral spinal fluids (CSF) levels and treating said individual with a combination of the acetylcholine esterase inhibitor donepezil and the one or more second compounds, as described elsewhere in this application. Methods of monitoring CSF levels of Aβ42 and Aβ40 are known to the skilled artisan and described herein.

The invention encompasses the treatment of an individual having mild to moderate AD, to the extent that individual has AD, whether or not one or more non-AD neurodegenerative diseases or conditions are previously, concurrently or subsequently diagnosed.

The compounds and methods of the present invention are useful for individuals who have received prior medication for AD, as well as individuals who have received no prior medication for AD, and is useful for individuals currently receiving medication for AD other than a compound of the present invention, and for individuals not receiving medication for AD other than a compound of the present invention.

Individuals of any age may be treated by the methods of the invention, with the pharmaceutical compositions of the invention; however, the invention encompasses specific embodiments for treating or preventing AD in individuals between the ages of 45 and 100. In other various specific embodiments, individuals treated by the therapeutic or prophylactic methods of the invention may be from 55 to 70 years of age, 60 to 80 years of age, 55 to 65 years of age, 60 to 75 years of age, 65 to 80 years of age, 55 to 60 years of age, 60 to 65 years of age, 65 to 70 years of age, 70 to 75 years of age, 75 to 80 years of age, or 80 years old and older.

Thus, in one embodiment, the invention provides a method of treating an individual known or suspected of having AD comprising administering a therapeutically effective amount of a compound according to Formula I. In a specific embodiment, said individual is diagnosed as having mild to moderate AD. In another specific embodiment, the individual is diagnosed by a cognitive test as having mild-to-moderate AD. In yet another embodiment, said cognitive test is the Mini-Mental State Exam (MMSE). In another specific embodiment, said individual has a score in said MMSE of from 26 to 19, inclusive. In another more specific embodiment, said individual has a score in said MMSE of from 18 to 10, inclusive. In another specific embodiment, said individual has a score in said MMSE of from 26 to 10, inclusive. In another specific embodiment, said individual has a score in said MMSE of from 18 or more, 19 or more, 20 or more, 21 or more, 22 or more, 23 or more, 24 or more, or 25 or more.

In yet another embodiment, the invention provides a method of slowing cognitive decline in an individual suspected of having mild cognitive impairment (MCI) comprising administering to the individual a therapeutically effective amount of a compound according to Formula I. MCI is a clinical condition between normal aging and AD characterized by memory loss greater than expected for the particular age of the individual yet the individual does not meet the currently accepted definition for probable AD. See, e.g., Petersen et al. Arch. Neurol. 58:1985-1992 (2001); Petersen Nature Rev. 2:646-653 (2003); and Morris et al. J. Mol. Neuro. 17:101-118 (2001). Thus, according to one aspect of the invention, an individual suspected of having or diagnosed with MCI is treated twice daily with a composition having from 400 mg to about 1200 mg per dose of a compound of the present invention, either alone, or in combination with a therapeutically effective amount of another suitable therapeutic compound, for at least 4 weeks, at least 4 months, preferably at least 8 months, and more desirably at least 1 year. Typically, patients having MCI first complain of or have a loss of memory. Preferably, a healthy individual personally associated with the patient can corroborate the memory deficit. Furthermore, general cognition is not sufficiently impaired to cause concern about more widespread cognitive disorder and although daily living activities may be affected that are not significantly impaired and the patients are not demented. Individuals having or suspected of having MCI that are not treated according to this embodiment can expect to experience a slow cognitive decline and/or progression to probable AD, mild AD, and or mild-to-moderate AD. When such individuals are treated according to the methods of the present invention, they can expect a lessening of the rate of progression of their cognitive decline, or even a stopping of their cognitive decline.

The decline in cognitive function in human patients can be characterized by cognition tests. It is preferred that the lessening in decline in cognitive function is at least 25% as compared to individuals treated with placebo, at least 40%, or at least 60%. For example, an individual treated with placebo having probably mild-to-moderate AD is expected to score approximately 5.5 points higher on the ADAS-cog test after a specified period of time (e.g., 1 year) whereas an individual treated with a composition of the invention for the same period of time will score only approximately 3.3 points higher on the ADAS-cog scale, i.e., will show 60% of the decline in cognitive function relative to untreated individuals, or 2.2 points higher i.e., will show 40% of the decline in cognitive function relative to untreated individuals, when treated for the same specified period of time.

In other embodiments, the invention provides a method of treating an individual known or suspected of having AD or MCI comprising administering an effective amount of a therapeutic compound of the present invention, wherein said individual is concurrently taking a second drug for the treatment of AD. In a further embodiment, said individual has been diagnosed as having mild to moderate AD. In a specific embodiment, said second drug being taken by that individual is an acetylcholinesterase (AChE) inhibitor. In a more specific embodiment, said AChE inhibitor is Galanthamine (galantamine, Reminyl); E2020 (Donepezil, Aricept); Physostigmine; Tacrine (tetrahydroaminoacridine, THA); Rivastigmine; Phenserine; Metrifonate (Promem); or Huperazine, or a combination of any of the foregoing. In another embodiment, said second drug is a drug other than an acetylcholinesterase inhibitor. In a preferred embodiment, the method or compositions of the invention are used in patients or individuals undergoing therapy with Aricept. The invention also encompasses methods of treating patients refractory to, or who no longer show improvement with, conventional AD therapy.

In another embodiment, the individual to be treated with a pharmaceutical composition of the present invention is concurrently taking a non-pharmaceutical substance for the treatment of AD along with a therapeutic compound of the present invention. In a specific embodiment, said non-pharmaceutical substance is an anti-oxidant. In a more specific example, said anti-oxidant is vitamin C or vitamin E. In an even more specific embodiment, said vitamin C is taken in a dose of 500-1000 mg per dose. In another even more specific embodiment, said vitamin E is taken in a dose of 400-800 IU per dose. In this regard, the invention encompasses the use of one or more such anti-oxidants as an adjunct to therapy for AD, and not primarily as a nutritional supplement.

In another embodiment, the invention provides a method of treating an individual diagnosed as having mild to moderate AD comprising administering an effective amount of a therapeutic compound of the present invention, wherein said individual has, prior to taking a therapeutic compound of the present invention, taken a second drug for the treatment of AD. In a specific embodiment, said second drug is an acetylcholinesterase (AChE) inhibitor. In a more specific embodiment, said ACE inhibitor is Galanthamine (galantamine, Reminyl); E2020 (Donepezil, Aricept); Physostigmine; Tacrine (tetrahydroaminoacridine, THA); Rivastigmine; Phenserine; Metrifonate (Promem); or Huperazine, or a combination of any of the foregoing. In another embodiment, said second drug is a drug other than an acetylcholinesterase inhibitor.

In another embodiment, said individual has, prior to taking a therapeutic compound of the present invention, taken a non-pharmaceutical substance for the treatment of AD. In a specific embodiment, said non-pharmaceutical substance is an anti-oxidant. In a more specific example, said anti-oxidant is vitamin C or vitamin E. In an even more specific embodiment, said vitamin C is taken in a dose of 500-1000 mg per dose. In another even more specific embodiment, said vitamin E is taken in a dose of 400-800 IU per dose. In this regard, the invention encompasses the use of one or more such anti-oxidants as an adjunct to therapy for AD, and not primarily as a nutritional supplement.

In yet another embodiment, the invention provides a method of slowing cognitive decline in an individual suspected of having mild cognitive impairment (MCI) comprising administering to the individual an effective amount of a therapeutic compound of the present invention. Mild cognitive impairment is a clinical condition between normal aging and AD characterized by memory loss greater than expected for the particular age of the individual yet the individual does not meet the currently accepted definition for probable AD. See, e.g., Petersen et al. Arch. Neurol. 58:1985-1992 (2001); Petersen Nature Rev. 2:646-653 (2003); and Morris et al. J. Mol. Neuro. 17:101-118 (2001). Thus, according to this embodiment an individual suspected of having or diagnosed with MCI is treated twice daily with a composition having from 400 mg to about 800 mg of a therapeutic compound of the present invention per dose for at least 4 weeks, at least 4 months, preferably at least 8 months, and more desirably at least 1 year. Typically, patients having MCI first complain of or have a loss of memory. Preferably a healthy individual associated with the patient can corroborate the memory deficit. Furthermore, general cognition is not sufficiently impaired to cause concern about more widespread cognitive disorder and although daily living activities may be affected that are not significantly impaired and the patients are not demented. Individuals having or suspected of having MCI that are treated according to this embodiment can expect to slow cognitive decline and/or progression to probable AD.

Administration of a pharmaceutical composition of the present invention can be via any route, and the pharmaceutical compositions of the present invention can correspond to any compositions envisioned by one of skill in the art, appropriate to the route of delivery.

### EXAMPLES

### Example 1

### The Calcium- and Integrin-Binding Protein (CIB) Interacts with Stearoyl-CoA Desatursase (SCD)

The principles and methods of the yeast two-hybrid system have been described in detail in The Yeast Two-Hybrid System, Bartel and Fields, eds., pages 183-196, Oxford University Press, New York, N.Y., 1997. The following is thus a description of the particular procedure that we used to identify the interaction discovered between CIB and SCD.

The cDNA encoding the bait protein was generated by PCR from cDNA prepared from a desired tissue. The cDNA product was then introduced by recombination into the yeast expression vector pGBT.Q, which is a close derivative of pGBT.C (See Bartel et al., Nat. Genet., 12:72-77 (1996)) in which the polylinker site has been modified to include M13 sequencing sites. The new construct was selected directly in the yeast strain PNY200 for its ability to drive tryptophane synthesis (genotype of this strain: MATαtrpl-901 leu2-3,112 ura3-52 his3-200 ade2 gal4Δgal80). In these yeast cells, the bait was produced as a C-terminal fusion protein with the DNA binding domain of the transcription factor Gal4 (amino acids 1 to 147). Prey libraries were transformed into the yeast strain BK100 (genotype of this strain: MATαtrpl-901 leu2-3,112 ura3-52 his3-200 gal4Δgal80 LYS2::GAL-HIS3 GAL2-ADE2 met2::GAL7-lacZ), and selected for the ability to drive leucine synthesis. In these yeast cells, each cDNA was expressed as a fusion protein with the transcription activation domain of the transcription factor Gal4 (amino acids 768 to 881) and a 9 amino acid hemagglutinin epitope tag. PNY200 cells (MATα mating type), expressing the bait, were then mated with BK100 cells (MATa mating type), expressing prey proteins from a prey library. The resulting diploid yeast cells expressing proteins interacting with the bait protein were selected for the ability to synthesize tryptophan, leucine, histidine, and adenine. DNA was prepared from each clone, transformed by electroporation into E. coli strain KC8 (Clontech KC8 electrocompetent cells, Catalog No. C2023-1), and the cells were selected on ampicillin-containing plates in the absence of either tryptophane (selection for the bait plasmid) or leucine (selection for the library plasmid). DNA for both plasmids was prepared and sequenced by the dideoxynucleotide chain termination method. The identity of the bait cDNA insert was confirmed and the cDNA insert from the prey library plasmid was identified using the BLAST program to search against public nucleotide and protein databases. Plasmids from the prey library were then individually transformed into yeast cells together with a plasmid driving the synthesis of lamin and 5 other test proteins, respectively, fused to the Gal4 DNA binding domain. Clones that gave a positive signal in the β-galactosidase assay were considered false-positives and discarded. Plasmids for the remaining clones were transformed into yeast cells together with the original bait plasmid. Clones that gave a positive signal in the β-galactosidase assay were considered true positives.

Experiments conducted in this fashion revealed that a bait comprising amino acids 1 through 191 of CIB (as defined in Entrez Nucleotide Accession No. U82226.1 [GI:1848270]) isolated a prey comprising comprising amino acids 320 through 359 of SCD (as defined in Entrez Nucleotide Accession No. Y13647.1 [GI:2190403]). This interaction, which linked SCD to the presenilins PS1 and PS2 thorough their mutual association with CIB, suggested that the activity of SCD may somehow be related to the processing of APP, the production of Aβ42, and the pathoetiology of Alzheimer's disease. This interaction was previously reported by the inventors in U.S. patent application Ser. Nos. 10/776,013, 09/975,072 and 60/240,790.

### Example 2

### Overexpression of SCD Results in Increased Aβ42 Production in H4 Cells Expressing Wild Type APP695 (wtAPP695)

Since Aβ production can be modulated by the local membrane lipid composition and fluidity (for review, see Puglielli et al., Nat. Neurosci. 6:345-351 (2003)), and since SCD interacts with CIB, and thus indirectly interacts with, or is localized near PS1 and PS2, the inventors reasoned that SCD activity might somehow regulate the processing of APP and production of Aβ42.

To investigate this possibility further, SCD and SCD-myc cDNAs were cloned into the pCMV vector and sequence verified. H4 cells expressing wtAPP695 were transfected with the SCD constructs. Initially, protein expression of SCD-myc could not be identified unambiguously because the anti-myc antibody reacted non-specifically with a protein of same size as SCD-myc. To overcome this problem, the SCD cDNA was cloned into a V5 tag expression vector and the new construct was transfected into H4 cells expressing wtAPP695. Expression of SCD-V5 was easily detected by anti-V5 antibody (data not shown).

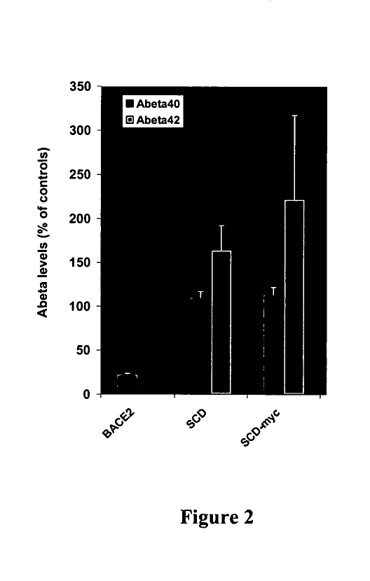

To conduct the over expression experiment, H4 cells expressing wtAPP695 were transfected with the indicated expression vectors. After 6 hours, the cell medium was replaced with fresh medium. After another 48 hours, Aβ40 or Aβ42 levels were measured in the medium by sandwich enzyme-linked immunosorbant assays (ELISAs). Results (FIG. 2) are expressed as a % of the Aβ40 or Aβ42 levels observed in cells transfected by empty vector (control cells). (These results were previously described by the inventors in U.S. patent application Ser. No. 10/776,013.)

As expected from previous experiments, transfection of cells with a BACE2 expression vector resulted in a marked decrease of Aβ40 and Aβ42 secretion (Aβ42 is decreased below the detectable level using the current ELISA kit). Interestingly, overexpression of SCD selectively stimulated Aβ42 secretion, but had no significant effect on Aβ40 secretion.

This result is consistent with a report by Fukumoto and colleagues (Fukumoto et al., J. Biol. Chem. 277:48508-48513 (2002)) showing that treatment of mouse neuroblastoma Neuro2A cells with T0901317 (a transcriptional activator of LXR) produced a modest increase (25%) in Aβ40 secretion and a large increase (126%) of Aβ42 secretion. Activation of LXR transcription is known to increase the expression of many genes, including ABCA1 and SCD.

### Example 3

### Inhibition of SCD with Specific Conjugated Linoleic Acids (CLA) Isomers Results in Lower Levels of Aβ42 Production

Reports in the literature indicate that various lipids including sterculic acid and conjugated linoleic acid (CLA) isomers inhibit SCD activity (See, Gomez et al., Biochem. Biophys. Res. Comm. 300:316-326 (2003); Park et al., Biochim. Biophys. Acta 1486:285-292 (2000); Choi et al., J. Nutr. 130:1920-1924 (2000)). In various mouse models, CLA treatment has been shown to have beneficial health effects such as decreased carcinogenesis, decreased artherogenesis, improved glucose intolerance and improved insulin action in diabetic models.

Cell-based assay models have shown that CLA generally reduces the levels of cellular monounsaturated fatty acids by reducing SCD activity. The trans-10, cis-12 isomer of CLA specifically inhibits SCD, while other isomers including the trans-9, trans-11 isomer do not (Park et al., Biochim. Biophys. Acta 1486:285-292 (2000)). To further test the hypothesis that SCD activity is involved in Aβ42 production, HEK293/swAPP cells were treated with the CLA isoforms, trans-10, cis-12 (active form) and trans-9, trans-11 (inactive form), for 24 hours, and levels Aβ42 were measured in the conditioned medium by ELISA.

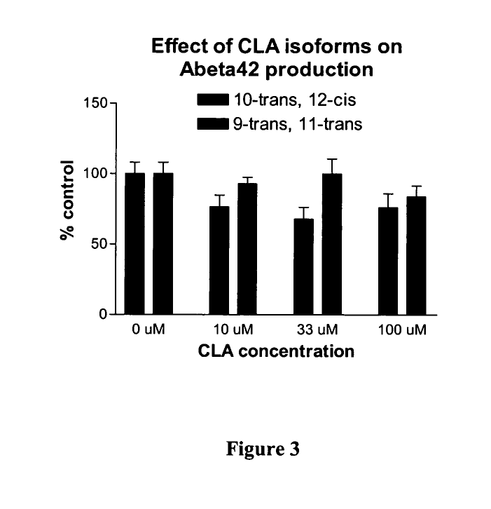

FIG. 3 shows that treatment of cells with 10, 33, or 100 μM of the trans-10, cis-12 isoform of CLA decreases Aβ42 levels by as much as 32%, while the trans-9, trans-11 isoform of CLA only decreased Aβ42 by 14% at 100 μM, the highest concentration tested. Overall, the cells treated with either CLA isoform at 100 μM appeared normal, but there were some bare patches of tissue culture plastic in these wells, indicating that this concentration of CLA may be somewhat toxic. Thus, the reduction of Aβ42 secretion observed at this highest treatment concentration used may not reflect specific inhibition of SCD, but toxicity of the CLA isomers to the cell.

On the other hand, since only the trans-10 cis-12 CLA isoform of CLA reduces Aβ42 secretion, and since this isoform is known to inhibit SCD activity (while the 9-trans 11-trans isoform does not), the observed reduction of Aβ42 secretion elicited by trans-10 cis-12 CLA at 10 and 30 μM was most likely mediated by SCD inhibition. (These results were previously described by the inventors in U.S. patent application Ser. No. 10/776,013.)

In summary, SCD overexpression in H4 cells increased Aβ42 secretion, while a known SCD inhibitor (trans-10 cis-12 CLA isoform of CLA) reduced Aβ42 secretion by HEK293 cells. Moreover, a compound very similar to the inhibitor, the 9-trans 11-trans CLA, which differs from the inhibitor only in the position and geometry of its double bonds, does not affect Aβ42 secretion at sub-toxic concentrations. In combination, these results suggest that SCD is an attractive target that, when inhibited, results in a reduction in Aβ42 production and secretion by cells.

### Example 4

### Knock-down of SCD Expression by RNA Interference Results in Lower Levels of Aβ42 Production and Secretion

HEK293 cells expressing wild type APP were treated with different concentrations (0.3, 3.0, and 30 nM) of a pool of four siRNAs targeting SCD (SCD siRNA), or a control (unrelated) pool of four siRNAs, for 24 hours or 48 hours, at which time conditioned medium was collected and RNA was extracted from the treated cells. SCD mRNA was measured by Northern blot analysis, and Aβ42 and Aβ40 levels were measured by ELISA. The Northern blot (not shown) demonstrated that treatment of cells with 30 nM of SCD siRNA resulted in an almost complete knockdown of SCD mRNA at both 24 hours and 48 hours, treatment with 3 nM SCD siRNA resulted in a more moderate effect, but treatment with 0.3 nM SCD siRNA had no discernable effect. The two bands on the blot (3.9 kb and 5.2 kb), which represent alternative transcripts with differing poly-adenylation sites as described by Zhang et al. (Biochem J. 340:255, 1999), were diminished to similar degrees. The Northern blot was stripped and reprobed for β-actin mRNA (data not shown) in order to confirm that all lanes contained similar amounts of RNA (data not shown).

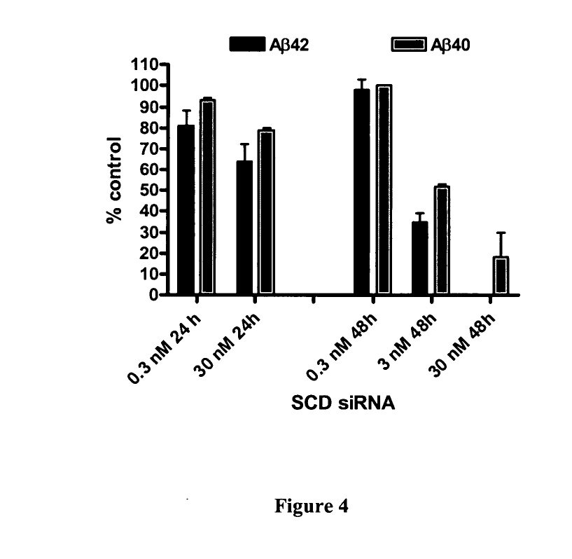

Aβ42 and Aβ40 levels were then measured by ELISA and found to correlate with the SCD mRNA levels (FIG. 4). After 24 hours of SCD siRNA treatment, cells treated with 0.3 nM SCD siRNA produced slightly less Aβ42 and Aβ40 compared to control. Cells treated with 30 nM SCD siRNA at 24 hours produced 35% less Aβ42 and 20% less Aβ140 compared to control. After 48 hours of SCD siRNA treatment, the results were more dramatic. Again, treatment with 0.3 nM SCD siRNA had no effect on Aβ levels compared to control. However, treatment with 3 nM SCD siRNA caused a 65% decrease in Aβ42 and a 50% decrease in Aβ40, and treatment with 30 nM SCD siRNA decreased production of Aβ42 to less than 1% of control, while Aβ40 was decreased to 20% of control. (These results were previously mentioned by the inventors in U.S. patent application Ser. No. 10/776,013.)

Although changes in β-amyloid production were not limited just to decreases in Aβ142, in all cases, Aβ40 did not decrease to the same extent as Aβ42. There also appears to be a delay in altered β-amyloid production in the cells treated with SCD siRNA. According to the Northern blots, SCD mRNA was knocked down after only 24 hours, but β-amyloid levels did not significantly drop in these cells until 48 hours of SCD siRNA treatment. These results fit the hypothesis that inhibiting SCD function results in increased levels of sphingosine at the endoplasmic reticulum (the place of action for APP processing by the γ-secretase complex). Localized increases in sphingosine alter the lipid environment, resulting in the formation of lipid rafts, and anchoring of certain protein complexes. Although SCD mRNA levels drop after 24 hours, it probably takes additional time for changes in the local lipid environment to occur, which could explain the 48 hour requirement for changes in Aβ production.

During the course of siRNA experiments, including those above, it was noticed that cell density in the culture dish can greatly affect the efficiency of a particular siRNA. To examine the effect of cell density on the efficacy of SCD siRNA treatment, HEK293/wtAPP cells were grown to low density (40% confluent) or high density (95% confluent), before being transfected with 0, 2 or 20 nM SCD siRNA for 24 or 48 hours. Following the treatment period conditioned medium was collected and total RNA was isolated from the cells. Northern blot analysis (data not shown) revealed that siRNA treatment of cells grown to the lower density had a greater effect compared to equivalent treatment of cells grown to a high density. When cells grown to the low density were treated with SCD siRNA at 2 and 20 nM, endogenous SCD mRNA levels were lowered by as much as 90%, after only 24 hours of treatment, and that treatment effect was not found to increase after 48 hours. Equivalent treatment with SCD siRNA had a smaller effect on cells grown to high density: the treatment with 2 nM SCD siRNA only decreased SCD mRNA by 50%, whereas the treatment with 2 nM SCD siRNA 20 nM decreased SCD mRNA by approximately 75% (data not shown).

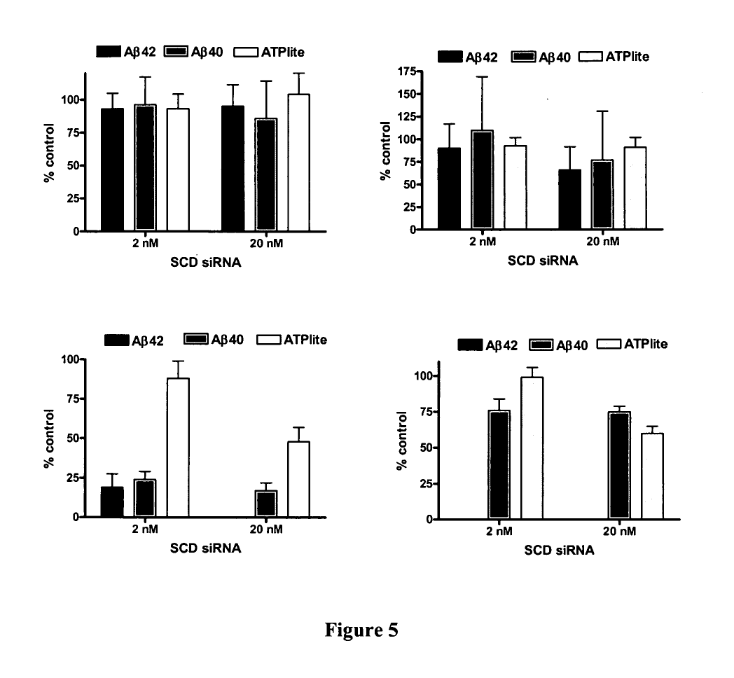

Aβ42 and Aβ40 peptides in the conditioned medium from the cell cultures in this study were quantified by ELISA. The results of these analyses are depicted in the four panels of FIG. 5, which also show the relative survival of the cells by the “ATPlite” assay. The results of assays conducted on cells plated and grown at low densities are shown in left side panels, while those plated and grown at high densities are shown on the right. Assays conducted on conditioned medium harvested after only 24 hours of treatment are shown in the top panels, while assays conducted on conditioned medium harvested after 48 hours of treatment are shown on the bottom.

The results (FIG. 5) indicate that cells treated with SCD siRNA for 24 hours (top panels) shows little or no reduction in Aβ peptide production, whereas cells treated for 48 hours (bottom panels) exhibit a marked reduction in Aβ peptide production. Additionally, cells plated and grown at low densities, and treated with 2 and 20 nM of siRNA for 48 hours (bottom left), exhibit levels of Aβ42 secretion that are decreased by 80% and 100%, respectively, and levels of Aβ140 secretion that are reduced by 75% and 83%, respectively. Cells plated and grown at high densities under the same treatments for 48 hours (bottom right) show a 100% reduction in Aβ42 secretion at both concentrations of siRNA, but only a 25% reduction in Aβ40 secretion.

These results suggest that Aβ42 production is somewhat more sensitive to SCD modulation than is Aβ40 production. These data also show a delay between the knock-down of SCD expression by siRNA treatment, and the observed decrease in Aβ peptide production. This delay is consistent with the hypothesis that changes in local lipid composition at the site of γ-secretase cleavage are necessary for altering APP processing and Aβ peptide production.

### Example 5

### Screening Assays of Oshino et al. for Identifying and Evaluating Inhibitors of SCD

The instant invention provides screening assays designed to detect inhibition of SCD activity that can be used to identify, evaluate, or compare compounds that can be used in the therapeutic methods of the invention for treating, delaying the onset of symptoms, slowing the progression of symptoms, or reversing the symptoms of MCI, AD, CAA, or dementia associated with DS, by inhibiting the enzymatic activity of SCD. Such screening assays measure the conversion of [1-¹⁴C] stearoyl-CoA to [1-¹⁴C]oleate by the SCD activity present in liver microsomes, and are conducted as described in Oshino et al. Biochim. Biophys. Acta 128:13-27 (1966), which is incorporated herein by reference in its entirety. Briefly, the assays are conducted as follows: Rat livers are homogenized in 10 volumes of buffer A (0.25 M sucrose, 1 mM EDTA, 10 mM Tris-HCl, 1 mM PMSF, pH 7.4). The microsomal membrane fractions (100,000×g pellet) are isolated by sequential centrifugation. Reactions are performed at 37° C. for 5 min with 400 μg protein homogenate and 27 nM of [1-¹⁴C] stearoyl-CoA (60,000 dpm), 1 μM NADH, 50 μM Tris-HCl, pH 7.4. After the reaction, fatty acids are extracted and then methylated with 10% acetic chloride/methanol. Saturated fatty acid and monounsaturated fatty acid methyl esters are separated by 10% AgNO₃-impregnated thin-layer chromatography using hexane:diethyl ether (9:1) as the developing solution. The plates are sprayed with 0.2% 2′,7′-dichloroflourescein in 95% ethanol, and the lipids are identified under UV light. The separated fractions are scraped off of the plate, and radioactivity is measured using a liquid scintillation counter. Enzyme activity is expressed as nmol min⁻¹ mg⁻¹ protein.

### Example 6

### Alternative Assay for SCD Enzymatic Activity

To determine the effect of test compounds on human SCD enzymatic activity, a reaction mix is prepared containing 10 μM test compound and 100 μg of human liver microsomes in the presence of 2 μCi of ³H-stearoyl-CoA and 2 mM NADH, in a total volume of 300 μl in PBS at pH 7.2. The reaction mix is incubated for 30 min at 25° C. The reactions are stopped by the addition of 30 μl of 7 N HCl, and the remaining free ³H-stearoyl-CoA is removed by two sequential extractions using Norit-A activated charcoal. ³H in the residual aqueous phase, which is liberated as result of the desaturase reaction, is measured by scintillation counting. Negative controls are run with the same reagents, except that either NADH, or microsomes are omitted. Test compounds to be tested for SCD inhibitory activity can be added to the reaction mix at concentrations other than 10 μM, however, the final concentration of DMSO (the test compound solvent) in all reactions (including controls) is kept at 1%. Reactions are run in triplicate. Microsomes prepared from yeast expressing human SCD can substitute for human liver microsomes.

### Example 7

### Detection of Amyloid Beta with Biosource Elisa Kit (Camarillo, Calif.)

The present invention provides compositions and methods for lowering Aβ42 levels. To test whether compounds and compositions are capable of modulating Aβ levels in cultured cells, sandwich enzyme-linked immunosorbent assay (ELISA) are employed to measure secreted A# (Aβ42 and/or Aβ40) levels. In this example, H4 cells expressing wide type APP695 are seeded at 200,000 cells per well in 6 well plates, and incubated at 37° C. with 5% CO₂ overnight. Cells are treated with 1.5 ml medium containing vehicle (DMSO) or a test compound at 1.25 μM, 2.5 μM, 5.0 μM and 10.0 μM (as well as other concentration if desirable) for 24 hours or 48 hours. The supernatant from treated cells is collected into eppendorf tubes and frozen at −80° C. for future analysis.

The amyloid peptide standard is reconstituted and frozen samples are thawed. The samples and standards are diluted with appropriate diluents and the plate is washed 4 times with Working Wash Buffer and patted dry on a paper towel. 100 μL per well of peptide standards, controls, and dilutions of samples to be analyzed is added. The plate is incubated for 2 hours while shaking on an orbital plate shaker at RT. The plate is then washed 4 times with Working Wash Buffer and patted dry on a paper towel. Detection Antibody Solution is poured into a reservoir and 100 μL/well of Detection Antibody Solution is immediately added to the plate. The plate is incubated at RT for 2 hours while shaking and then washed four times with Working Wash Buffer and patted dry on a paper towel. Secondary Antibody Solution is then poured into a reservoir and 100 μL/well of Secondary Antibody Solution is immediately added to the plate. The plate is incubated at RT for 2 hours with shaking, washed 5 times with Working Wash Buffer, and patted dry on a paper towel.

100 μL of stabilized chromogen is added to each well and the liquid in the wells begins to turn blue. The plate is incubated for 30 minutes at room temperature and in the dark. 100 μL of stop solution is added to each well and the plate is tapped gently to mix resulting in a change of solution color from blue to yellow. The absorbance of each well is read at 450 nm having blanked the plate reader against a chromogen blank composed of 100 μL each of stabilized chromogen and stop solution. The plate is read within 2 hours of adding the stop solution. The absorbance of the standards is plotted against the standard concentration and the concentrations of unknown samples and controls are calculated.

### Example 8

### Detection of Amyloid Beta with Innogenetic Elisa Kit (Gent, Belgium)

The present invention provides compositions and methods for lowering Aβ42 levels. To test whether compounds and compositions are capable of modulating Aβ levels, sandwich enzyme-linked immunosorbent assay (ELISA) is employed to measure secreted Aβ (Aβ42 and/or Aβ40) levels. In this example, H4 cells expressing wide type APP695 are seeded at 200,000 cells per well in 6 well plates, and incubated at 37° C. with 5% CO₂ overnight. Cells are treated with 1.5 mL medium containing vehicle (DMSO) or a test compound at 1.25 μM, 2.5 μM, 5.0 μM and 10.0 μM concentration for 24 hours or 48 hours. The supernatant from treated cells is collected into eppendorf tubes and frozen at −80° C. for future analysis.

130 μL per well of samples, standards, and blanks is added to a 96-well polypropylene plate. 200 μL of samples, standards, and blanks from the polypropylene plate is added to the antibody-coated plates. The strip-holder with the appropriate number of strips is applied to the antibody-coated plates and the strips are covered with an adhesive sealer. The plate is then incubated 3 hours at room temperature while shaking on an orbital plate shaker.

The first antibody solution is prepared with Conjugate Diluent 1 at 1:100 ratio. Each well of the antibody-coated plates is washed 5 times with 400 μL washing solution and 100 μL of the prepared first antibody solution is added to each well. The strips are applied to the plate, covered with an adhesive sealer, and the plate is incubated for 1 hour at room temperature while shaking on an orbital plate shaker.

The second antibody (conjugate 2) solution is prepared with Conjugate Diluent 2 at 1:100 ratio. Each well of the antibody-coated plates are washed for 5 times with 400 μL washing solution and 100 μL of the prepared second antibody solution is added to each well. The strips are applied, covered with an adhesive sealer, and the plate is incubated 30 min at room temperature while shaking on an orbital plate shaker. Each well of the antibody-coated plates is then are washed for 5 times with 400 μL washing solution.

A substrate solution is prepared by diluting substrate 100× with HRP Substrate Buffer. 100 μL of the prepared substrate solution is added to each well of the antibody-coated plate. The strips are applied, covered with an adhesive sealer, and the plate is incubated for 30 min at room temperature. 100 μL Stop Solution is then added to each well to stop the reaction. The strip-holder is carefully taped to ensure through mixing. The reader is blanked and the absorbance of the solution in the wells is read at 450 nm. The absorbance of the standards is plotted against the standard concentration and the concentration of samples is calculated using the standard curve.

### Example 9

### Neuroprotection Assay

The present invention provides compositions and methods for slowing the death or decline of neurons. To test the ability of compositions and methods of the present invention to protect against neurotoxicity, adult female Sprague Dawley rats are obtained and injected intraperitoneally with various doses of a composition of the present invention. At the same time, the test animals also receive a subcutaneous injection of MK-801 (0.5 mg/kg), which has been shown to consistently induce, in all treated rats, a fully developed neurotoxic reaction consisting of acute vacuole formation in the majority of pyramidal neurons in layers III and IV of the posterior cingulate and retrosplenial (PC/RS) cortices.

Control animals are administered the liquid which was used to dissolve the test agent and the same dosage of MK-801 (0.5 mg/kg sc). The animals are sacrificed four hours after treatment and the number of vacuolated PC/RS neurons are counted on each side of the brain, at a rostrocaudal level immediately posterior to where the corpus callosum ceases decussating across the midline (approximately 5.6 mm caudal to bregma). The toxic reaction approaches maximal severity at this level and shows very little variability between different animals.

Percentage reduction in neurotoxicity is calculated by dividing the mean number of vacuolated neurons in a given treatment group, by the mean number of vacuolated neurons in control animals that were treated with MK-801 but not the protective agent. The result is subtracted from one and multiplied by 100, to calculate a percentage. Linear regression analysis can be used to determine an ED₅₀ (i.e., the dosage of a given compound that reduces the mean number of vacuolated neurons to 50% of the value in control animals), with the 25th and 75th percentiles defining the confidence limits.

### Example 10

### Treatment of Animals with a Compound to Determine the Compound's Effect on Levels of Aβ42 and Alzheimer's Disease

To determine the effect of a composition or method of the present invention on levels of Aβ42 and AD, an animal is treated with the compound and the levels of Aβ42 in the brain are measured. Three month-old TG2576 mice that overexpress APP(695) with the “Swedish” mutation (APP695NL) are used. Mice overexpressing APP(695) with the “Swedish” mutation have high levels of soluble Aβ in the their brains and develop memory deficits and plaques with age, making them suitable for examining the effect of compounds on levels of Aβ42 and AD. “Test” TG25276 mice are treated with the compound and “control” TG25276 mice are not. The brain levels of SDS-soluble Aβ40 and Aβ42 for “test” mice are compared to “control” mice using ELISA. Test mice that have a reduction in Aβ42 levels suggest that treatment with the compound could prevent amyloid pathology by decreasing the ratio of Aβ42 to Aβ40 in the brain.

### Example 11

### Treatment of Animals with a Compound to Determine the Compound's Effect on Memory and Alzheimer's Disease

The present invention provides compositions and methods for treating or preventing AD. To test the effect of compositions and methods of the present invention on memory and AD, TG2576 mice that overexpress APP(695) with the “Swedish” mutation (APP695NL) are used. Mice overexpressing APP(695) with the “Swedish” mutation develop memory deficits and plaques with age, making them suitable for examining the effect of compounds on memory and AD. The test compound is administered daily for two weeks to test groups of the TG2576 mice in age groups of: 1) 4-5 months, 2) 6-11 months, 3) 12-18 months, and 4) 20-25 months. Groups of control TG2576 mice of corresponding ages are not administered the compound. Both control and test groups then have memory tested in a version of the Morris water maze (Morris, J. Neurosci. Methods 11:47-60 (1984)) that is modified for mice. The water maze contains a metal circular pool of about 40 cm in height and 75 cm in diameter. The walls of the pool have fixed spatial orientation clues of distinct patterns or shelves containing objects. The pool is filled with room temperature water to a depth of 25 cm and an escape platform is hidden 0.5 cm below the surface of the 25-cm-deep water at a fixed position in the center of one of the southwest quadrant of pool. The test and control mice are trained for 10 days in daily sessions consisting of four trials in which the mouse starts in a different quadrant of the pool for each trial. The mice are timed and given 60 seconds to find the escape platform in the pool. If the mice have not found the escape platform after 60 seconds, they are guided into it. The mice are then allowed to rest on the platform for 30 seconds and the amount of time it takes the mice to find the platform is recorded. Probe trials are run at the end of the trials on the 4^{th}, 7^{th}, and 10^{th} days of training, in which the platform is removed and the mice are allowed to search for the platform for 60 sec. The percentage of time spent in the quadrant where the platform was in previous trials is calculated.

In training trials, the time it takes test group mice to reach the escape platform is compared to the time taken by control group mice of corresponding ages. In probe trials, the percentage of time spent by test group mice in the quadrant where the platform was in previous trials is compared to the percentage time spent by control mice. Quicker location of the escape platform in training trials and/or an increased percentage time spent in the previous quadrant of the maze during probe trials is indicative of spatial learning and memory. Because memory loss is a hallmark of AD, test mice that have better learning and memory when compared to control mice indicate that the compound may be effecting in treating or slowing AD and/or its symptoms.

### Example 12

### Synthesis of Compounds of Formula I-V(g)(2)

Protocols and methods describing the synthesis of the compounds of Formulae I-V(g)(2) are described in detail in U.S. patent application Ser. Nos. 10/901,563, 10/885,901, and 10/566,856, and international patent application publications WO 2005/011653, WO 2005/011654, WO 2005/011655, WO 2005/011656, and WO 2005/011657. The protocols and methods of synthesis disclosed in these patent applications are incorporated by reference herein in their entirety.

### Example 13

### Treatment of MCI with a compound of Formulae I-V(g)(2)

Compounds of Formulae I-V(g)(2) can be used to treat MCI by administering tablets containing 50 mg of the compound, and/or oral gel capsules containing 50 mg of the compound. The typical dosage may be 50, 100, 300 or 600 mg of active ingredients daily. A typical dosage regimen may have 100 mg of the compound taken daily (50 mg twice daily). Another typical dosage may have 50 mg of the compound taken once daily. These dosages can also be divided or modified, and taken with or without food.

### Example 14

### Treatment of Alzheimer's Disease with a Compound of Formulae I-V(g)(2)

The compounds of Formulae I-V(g)(2) can be administered once daily as a tablet containing 1-200 mg of active ingredient or as a capsule containing 1-200 mg of the active ingredient. Typically, for the treatment of mild-to-moderate AD, an individual is diagnosed by a doctor as having the disease using a suitable combination of observations. One criterion indicating a likelihood of mild-to-moderate AD is a score of about 15 to about 26 on the MMSE test. Another criteria indicating mild-to-moderate AD is an observed decline in cognitive function. The compound can also be administered in liquid or other dosage forms. The dosages can also be divided or modified, and taken with or without food. For example, the 200 mg dose can be divided into two 100 mg tablets or capsules.

Depending on the stage of the disease, the compounds of Formula I can also be administered once daily in liquid, capsule, or tablet dosage forms where the dose has various amounts of compound (i.e., 300 mg, 250 mg, 200 mg, 175 mg, 150 mg, 125 mg, 100 mg, 75 mg, 50 mg, 40 mg, 30 mg, 25 mg, 15 mg, 10 mg and 1 mg). Again, the dosages can also be divided or modified, and taken with or without food. The doses can be taken during treatment with other medications for treating AD or symptoms thereof. For example, the compounds of Formula I can be administered in the morning as a tablet containing 100 mg of active ingredient and an acetylcholine esterase inhibitor (i.e., tacrine (Cognex®), donepezil (Aricept®), rivastigmine (Exelon®), and galantamine (Reminyl®)), and/or an NMDA antagonist (i.e., memantine). It may be desirable to lower the amount of acetylcholine esterase inhibitor (and/or NMDA antagonist) to avoid adverse side effects associated with higher doses of these compounds. Alternatively, the acetylcholine esterase inhibitor (and/or NMDA antagonist) can be co-formulated into a single dosage form, i.e., liquid, tablet, capsule, etc.

Patients having mild-to-moderate AD undergoing the treatment regimen of this example with compounds of Formulae I-V(g)(2) in doses of about 1 mg to 400 mg can experience a lessening in decline of cognitive function (as measured by the ADAS-cog or CDR sum of boxes), plaque pathology, and/or biochemical disease marker progression.

### Example 15

### Prevention of Alzheimer's Disease

Prior to the onset of symptoms of AD or just at the very beginning stages of the disease, patients desiring prophylaxis against AD can be treated with a prophylactically effective amount of compounds of Formulae I-V(g)(2)compounds of Formulae I-V(g)(2). Those needing prophylaxis can be assessed by monitoring assayable disease markers, detection of genes conferring a predisposition to the disease, other risks factors such as age, diet, other disease conditions associated with Alzheimer's. The patient can also be treated with a combination of NMDA, and/or R-flurbiprofen, and compounds of Formulae I-V(g)(2) compounds of Formulae I-V(g)(2) to delay or prevent the onset of AD or symptoms thereof.

The patient desiring prophylaxis against AD or prophylaxis of a worsening of the symptoms of AD can be treated with compounds of Formulae I-V(g)(2) in an amount sufficient to delay the onset or progression of symptoms of AD. For example, a patient can be treated with 100 mg of compounds of Formulae I-V(g)(2) once daily. Another preventive regimen involves administering to the patient 50 mg of compounds of Formulae I-V(g)(2) once daily. These amounts of these active ingredients can be modified to lessen side-effects and/or produce the most therapeutic benefit. For example, 25 mg of compounds of Formulae I-V(g)(2) twice daily can be administered to reduce side-effects associated with the use of higher levels of the active ingredient. The preventive treatment can also be, e.g., treatment on alternating days with compounds of Formulae I-V(g)(2) or alternating weeks. Other preventive treatment regimens include, but are not limited to, treatment with compounds of Formulae I-V(g)(2) for 3 weeks out of every 4 weeks, or for several months followed by no treatment for a month and then treatment for several months in an alternating on/off schedule to reduce side effects or toxicity problems.

Patients desiring or in need of prophylaxis against AD undergoing the preventive regimen of this example with compounds of Formulae I-V(g)(2) doses of about 1 mg to 400 mg can decelerate or delay the onset of AD or prevent the occurrence of AD. It can be advantageous to utilize a low dosage prevention regimen that involves administration of pharmaceutical doses of 50 mg compounds of Formulae I-V(g)(2) once daily.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A method of identifying compounds useful for the treatment of mild cognitive impairment (MCI), Alzheimer's disease (AD), cerebral amyloid angiopathy (CAA) and dementia associated with Down syndrome (DS) comprising selecting compounds that reduce the stearoyl-coA desaturase (SCD) activity in cells, wherein the selected compounds that reduce SCD activity in cells are useful for the treatment of MCI, AD, CAA and dementia associated with DS.

2. The method of claim 1, wherein the selecting step comprises contacting test compounds with SCD protein or with a cell expressing SCD.

3. The methods of claim 2 wherein the selecting step comprises identifying compounds that reduce the expression of SCD in cells.

4. The method of claim 3, wherein said compounds that reduce the expression of SCD in cells, reduce the amount of SCD-encoding transcripts in cells.

5. The method of claim 4, wherein said compounds that reduce the amount of SCD-encoding transcripts in the cells are small interfering nucleic acids that induce RNA interference.

6. The method of claim 3, wherein said compounds that reduce the expression of SCD in cells, reduce the amount of translation of SCD-encoding transcripts in the cells.

7. The method of claim 2, wherein said compounds that reduce the expression of SCD in cells are antisense nucleic acids.

8. The method of claim 2, wherein the contacting step comprises identifying compounds that inhibit the enzymatic activity of SCD.

9. The method of claim 8 further comprising determining whether the identified compounds lower the amount of Aβ42 produced or secreted by a test cell, when contacted with said test cell.

10. The method of claim 9 wherein said determining step comprises determining whether the identified compounds lower the amount of Aβ42 produced or secreted by a test cell in cell culture.

11. The method of claim 9 wherein said determining step comprises determining the amount of Aβ42 produced or secreted by a test cell, in a cell-free assay.

12. The method of claim 9 wherein said determining step further comprises testing the compounds that lower the amount of Aβ42 produced or secreted by a test cell in an animal model for MCI, AD, CAA or dementia associated with DS.

13. A method of treating, delaying the onset of symptoms, slowing the progression of symptoms, or reversing the symptoms, of MCI, AD, CAA or dementia associated with DS, comprising:
identifying a patient in need of such treatment,
administering to the patient a therapeutically effective amount of a compound that reduces SCD activity in cells.

14. The method of claim 13, wherein said compound that reduces SCD activity in cells, reduces the expression of SCD.

15. The method of claim 14, wherein the compound that reduces the expression of SCD is an antisense nucleic acid, or a small interfering nucleic acid, that hybridizes to the SCD transcript.

16. The method of claim 13, wherein said compound that reduces SCD activity in cells inhibits the enzymatic activity of SCD.

17. The method of claim 16, wherein the compound that inhibits the enzymatic activity of SCD is selected from the compounds of Formula I: 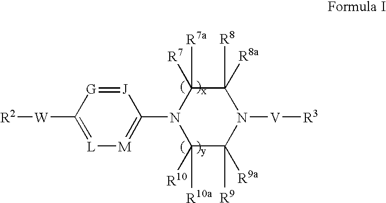
wherein:
x and y are each independently 1, 2 or 3;
W is —N(R¹)C(O)—, —C(O)N(R¹)—, —C(O)N[C(O)R^{1a}]—, —OC(O)N(R¹)—, —N(R¹)C(O)N(R¹)—, —O—, —N(R¹)—, —S(O)ₜ— (where t is 0, 1 or 2), —N(R¹)S(O)ₜ— (where t is 1 or 2), —S(O)₂N(R¹)—, —C(O)—, —OS(O)₂N(R¹)—, —OC(O)—, —C(O)O—, —C(S)N(R¹)—, —OC(S)N(R¹)—, —C(R¹)₂, —N(R¹)C(S)N(R¹)— or —N(R¹)C(O)O—;
V is —C(O)—, —C(O)O—, —C(S)—, —C(S)O—, —C(O)N(R¹)—, —S(O)ₜ— (where t is 1 or 2), —S(O)ₜN(R¹)— (where t is 1 or 2) or —C(R¹¹)H—;
G, J, L and M are each independently selected from —N— or —C(R⁴)—;
each R¹ is independently selected from the group consisting of H, C₁-C₆alkyl optionally substituted with one or more substituents selected from the group consisting of halo, methyl and trifluoromethyl, C₂-C₆alkenyl optionally substituted with one or more substituents selected from the group consisting of methoxy and hydroxyl, C₇-C₁₂alkyl, C₂-C₁₂hydroxyalkyl, C₄-C₁₂cycloalkylalkyl and C₇-C₁₉aralkyl;
R^{1a} is selected from the group consisting of H, C₁-C₆alkyl and cycloalkyl;
R² is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂ heteroarylalkyl; optionally substituted with one or more halo, cyano, oxo, thioxo, nitro, hydroxy, C₁-C₆alkyl, C₁-C₆trihaloalkyl, C₁-C₆trihaloalkoxy, C₁-C₆alkylsulfonyl, —OR¹², —C(O)R¹², N(R²)₂, —OC(O)R², —C(O)OR¹², —C(O)N(R¹²)₂, or —S(O)₂N(R¹²)₂, cycloalkyl, heterocyclyl, aryl, aralkyl, heteroaryl, and hetreoarylcycloalkyl;
or R² is a multi-ring structure having 2 to 4 rings wherein the rings are independently selected from the group consisting of cycloalkyl, heterocyclyl, aryl and heteroaryl and where some or all of the rings may be fused to each other;
R³ is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂heteroarylalkyl;
or R³ is an aryl optionally substituted with one or more substituents chosen from halo, cyano, nitro, hydroxy, C₁-C₆alkyl, C₁-C₆trihaloalkyl, C₁-C₆trihaloalkoxy, C₁-C₆alkylsulfonyl, —N(R¹²)₂, —OC(O)R², —C(O)OR¹², —S(O)₂N(R¹²)₂, cycloalkyl, heterocyclyl, aryl, aralkyl, heteroaryl, and hetreoarylcycloalkyl;
or R³ is a multi-ring structure having 2 to 4 rings wherein the rings are independently selected from the group consisting of cycloalkyl, heterocyclyl, aryl and heteroaryl and where some or all of the rings may be fused to each other;
each R⁴ is independently selected from H, fluoro, chloro, bromo, methyl, methoxy, trifluoromethyl, cyano, nitro, or —N(R¹³)₂;
R⁷, R^{7a}, R⁸, R^{8a}, R⁹, R^{9a}, R¹⁰, R^{10a} are each independently selected from H or C₁-C₃alkyl;
or R⁷ and R^{7a} together, or R⁸ and R^{8a} together, or R⁹ and R^{9a} together, or R¹⁰ and R^{10a} together are an oxo group, provided that when V is —C(O)—, R⁸ and R^{8a} together or R⁹ and R^{9a} together do not form an oxo group, while the remaining R⁷, R^{7a}, R⁸, R^{8a}, R⁹, R^{9a}, R¹⁰ and R^{10a} are each independently selected from H or C₁-C₃alkyl;
or one of R⁷, R^{7a}, R⁸, and R^{8a} together with one of R⁹, R^{9a}, R¹⁰ and R^{10a} form an alkylene bridge, while the remaining R⁷, R^{7a}, R⁸, R^{8a}, R⁹, R^{9a}, R¹⁰, and R^{10a} are each independently selected from H or C₁-C₃alkyl;
R¹¹ is H or C₁-C₃alkyl;
R¹² is H, C₁-C₆alkyl, C₃-C₆cycloalkyl, aryl, or aralkyl; and
each R¹³ is independently selected from H or C₁-C₆alkyl; or
a stereoisomer, enantiomer or tautomer thereof, a pharmaceutically acceptable salt thereof, or a prodrug thereof.

18. A method of altering the amount of Aβ42 in a tissue or organ of a human patient comprising:
identifying a patient in need of such treatment,
administering to the patient a therapeutically effective amount of a compound that reduces SCD activity in cells,
wherein said compound alters the amount of Aβ42 in said tissue or organ of said patient.

19. The method of claim 18, wherein the altering step is selected from raising the amount of Aβ42 in cerebrospinal fluid, or lowering the amount of Aβ42 in plasma, or the brain.

20. The method of claim 19, wherein lowering the amount of Aβ42 in the brain of said patent results in a decrease in the density, number or size of amyloid plaques in the brain.

21. The method of claim 20, wherein the decrease in the density, number or size of amyloid plaques in the brain is determined through the use of positron emission tomography and a tracer that selectively binds or accumulates in amyloid plaques.

22. The method of claim 18, wherein said compound that reduces SCD activity in cells inhibits the enzymatic activity of SCD.

23. The method of claim 22, wherein the compound that inhibits the enzymatic activity of SCD is selected from the compounds of Formula I: 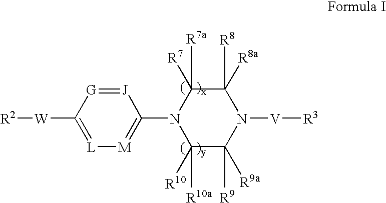
wherein:
x and y are each independently 1, 2 or 3;
W is —N(R¹)C(O)—, —C(O)N(R¹)—, —C(O)N[C(O)R^{1a}]—, —OC(O)N(R¹)—, —N(R¹)C(O)N(R¹)—, —O—, —N(R¹)—, —S(O)ₜ— (where t is 0, 1 or 2), —N(R¹)S(O)ₜ— (where t is 1 or 2), —S(O)₂N(R¹)—, —C(O)—, —OS(O)₂N(R¹)—, —OC(O)—, —C(O)O—, —C(S)N(R¹)—, —OC(S)N(R¹)—, —C(R¹)₂, —N(R¹)C(S)N(R¹)— or —N(R¹)C(O)O—;
V is —C(O)—, —C(O)O—, —C(S)—, —C(S)O—, —C(O)N(R₁)—, —S(O)ₜ— (where t is 1 or 2), —S(O)ₜN(R¹)—(where t is 1 or 2) or —C(R¹¹)H—;
G, J, L and M are each independently selected from —N— or —C(R⁴)—;
each R¹ is independently selected from the group consisting of H, C₁-C₆alkyl optionally substituted with one or more substituents selected from the group consisting of halo, methyl and trifluoromethyl, C₂-C₆alkenyl optionally substituted with one or more substituents selected from the group consisting of methoxy and hydroxyl, C₇-C₁₂alkyl, C₂-C₁₂hydroxyalkyl, C₄-C₁₂cycloalkylalkyl and C₇-C₁₉aralkyl;
R^{1a} is selected from the group consisting of H, C₁-C₆alkyl and cycloalkyl;
R² is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₁₂ heteroarylalkyl; optionally substituted with one or more halo, cyano, oxo, thioxo, nitro, hydroxy, C₁-C₆alkyl, C₁-C₆trihaloalkyl, C₁-C₆trihaloalkoxy, C₁-C₆alkylsulfonyl, —OR¹², —C(O)R¹², N(R¹²)₂, —OC(O)R¹², —C(O)OR¹², —C(O)N(R²)₂, or —S(O)₂N(R²)₂, cycloalkyl, heterocyclyl, aryl, aralkyl, heteroaryl, and hetreoarylcycloalkyl;
or R² is a multi-ring structure having 2 to 4 rings wherein the rings are independently selected from the group consisting of cycloalkyl, heterocyclyl, aryl and heteroaryl and where some or all of the rings may be fused to each other;
R³ is selected from the group consisting of C₁-C₁₂alkyl, C₂-C₁₂alkenyl, C₂-C₁₂hydroxyalkyl, C₂-C₁₂hydroxyalkenyl, C₁-C₁₂alkoxy, C₂-C₁₂alkoxyalkyl, C₃-C₁₂cycloalkyl, C₄-C₁₂cycloalkylalkyl, aryl, C₇-C₁₉aralkyl, C₃-C₁₂heterocyclyl, C₃-C₁₂heterocyclylalkyl, C₁-C₁₂heteroaryl, and C₃-C₂heteroarylalkyl;
or R³ is an aryl optionally substituted with one or more substituents chosen from halo, cyano, nitro, hydroxy, C₁-C₆alkyl, C₁-C₆trihaloalkyl, C₁-C₆trihaloalkoxy, C₁-C₆alkylsulfonyl, —N(R¹²)₂, —OC(O)R², —C(O)OR¹², —S(O)₂N(R¹²)₂, cycloalkyl, heterocyclyl, aryl, aralkyl, heteroaryl, and hetreoarylcycloalkyl;
or R³ is a multi-ring structure having 2 to 4 rings wherein the rings are independently selected from the group consisting of cycloalkyl, heterocyclyl, aryl and heteroaryl and where some or all of the rings may be fused to each other;
each R⁴ is independently selected from H, fluoro, chloro, bromo, methyl, methoxy, trifluoromethyl, cyano, nitro, or —N(R¹³)₂;
R⁷, R^{7a}, R⁸, R^{8a}, R⁹, R^{9a}, R¹⁰, R^{10a} are each independently selected from H or C₁-C₃alkyl;
or R⁷ and R^{7a} together, or R⁸ and R^{8a} together, or R⁹ and R^{9a} together, or R¹⁰ and R^{10a} together are an oxo group, provided that when V is —C(O)—, R⁸ and R^{8a} together or R⁹ and R^{9a} together do not form an oxo group, while the remaining R⁷, R^{7a}, R⁸, R^{8a}, R⁹, R^{9a}, R¹⁰ and R^{10a} are each independently selected from H or C₁-C₃alkyl;
or one of R⁷, R^{7a}, R⁸, and R^{8a} together with one of R⁹, R^{9a}, R¹⁰ and R^{10a} form an alkylene bridge, while the remaining R⁷, R^{7a}, R⁸, Rₐ, R⁹, R^{9a}, R¹⁰, and R^{10a} are each independently selected from H or C₁-C₃alkyl;
R¹¹ is H or C₁-C₃alkyl;
R¹² is H, C₁-C₆alkyl, C₃-C₆cycloalkyl, aryl, or aralkyl; and
each R¹³ is independently selected from H or C₁-C₆alkyl; or
a stereoisomer, enantiomer or tautomer thereof, a pharmaceutically acceptable salt thereof, or a prodrug thereof.


SEQUENCE LISTING (32 sequences)

SEQ ID NO: 1 - DNA, 5473 bp, Homo sapiens
ggcaggacgaggtggcaccaaattcccttcggccaatgacgagccggagtttacagaagcctcattagcatttccccagaggcaggggcaggggcagaggccgggtggtgtggtgtcggtgtcggcagcatccccggcgccctgctgcggtcgccgcgagcctcggcctctgtctcctccccctcccgcccttacctccacgcgggaccgcccgcgccagtcaactcctcgcactttgcccctgcttggcagcggataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaacaactctgcctttatgatgctaagctgatattatttcttctcttatcctctctctcttctaggcccattgtcctccttttcactttattgctatcgccctcctttcccttattgcctcccaggcaagcagctggtcagtctttgctcagtgtccagcttccaaagcctagacaacctttctgtagcctaaaacgaatggtctttgctccagataactctctttccttgagctgttgtgagctttgaagtaggtggcttgagctagagataaaacagaatcttctgggtagtcccctgttgattatcttcagcccaggcttttgctagatggaatggaaaagcaacttcatttgacacaaagcttctaaagcaggtaaattgtcgggggagagagttagcatgtatgaatgtaaggatgagggaagcgaagcaagaggaacctctcgccatgatcagacatacagctgcctacctaatgaggacttcaagccccaccacatagcatgcttcctttctctcctggctcggggtaaaaagtggctgcggtgtttggcaatgctaattcaatgccgcaacatatagttgaggccgaggataaagaaaagacattttaagtttgtagtaaaagtggtctctgctggggaagggttttcttttctttttttctttaataacaaggagatttcttagttcatatatcaagaagtcttgaagttgggtgtttccagaattggtaaaaacagcagctcatagaattttgagtattccatgagctgctcattacagttctttcctctttctgctctgccatcttcaggatattggttcttcccctcatagtaataagatggctgtggcatttccaaacatccaaaaaaagggaaggatttaaggaggtgaagtcgggtcaaaaataaaatatatatacatatatacattgcttagaacgttaaactattagagtatttcccttccaaagagggatgtttggaaaaaactctgaaggagaggaggaattagttgggatgccaatttcctctccactgctggacatgagatggagaggctgagggacaggatctataggcagcttctaagagcgaacttcacataggaagggatctgagaacacgttgccaggggcttgagaaggttactgagtgagttattgggagtcttaataaaataaactagatattaggtccattcattaattagttccagtttctccttgaaatgagtaaaaactagaaggcttctctccacagtgttgtgccccttcactcatttttttttgaggagaagggggtctctgttaacatctagcctaaagtatacaactgcctggggggcagggttaggaatctcttcactaccctgattcttgattcctggctctaccctgtctgtcccttttctttgaccagatctttctcttccctgaacgttttcttctttccctggacaggcagcctcctttgtgtgtattcagaggcagtgatgacttgctgtccaggcagctccctcctgcacacagaatgctcagggtcactgaaccactgcttctcttttgaaagtagagctagctgccactttcacgtggcctccgcagtgtctccacctacacccctgtgctcccctgccacactgatggctcaagacaaggctggcaaaccctcccagaaacatctctggcccagaaagcctctctctccctccctctctcatgaggcacagccaagccaagcgctcatgttgagccagtgggccagccacagagcaaaagagggtttattttcagtcccctctctctgggtcagaaccagagggcatgctgaatgccccctgcttacttggtgagggtgccccgcctgagtcagtgctctcagctggcagtgcaatgcttgtagaagtaggaggaaacagttctcactgggaagaagcaagggcaagaacccaagtgcctcacctcgaaaggaggccctgttccctggagtcagggtgaactgcaaagctttggctgagacctgggatttgagataccacaaaccctgctgaacacagtgtctgttcagcaaactaaccagcattccctacagcctagggcagacaatagtatagaagtctggaaaaaaacaaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtagaggctacaggggttagcctggactaaaggcatccttgtcttttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaactttcacttcttcctattgtaatcgtgtgccatggatctgatctgtaccatgaccctacataaggctggatggcacctcaggctgagggccccaatgtatgtgtggctgtgggtgtgggtgggagtgtgtctgctgagtaaggaacacgattttcaagattctaaagctcaattcaagtgacacattaatgataaactcagatctgatcaagagtccggatttctaacagtccttgctttggggggtgtgctgacaacttagctcaggtgccttacatcttttctaatcacagtgttgcatatgagcctgccctcactccctctgcagaatccctttgcacctgagaccctactgaagtggctggtagaaaaaggggcctgagtggaggattatcagtatcacgatttgcaggattcccttctgggcttcattctggaaacttttgttagggctgcttttcttaagtgcccacatttgatggagggtggaaataatttgaatgtatttgatttataagttttttttttttttttgggttaaaagatggttgtagcatttaaaatggaaaattttctccttggtttgctagtatcttgggtgtattctctgtaagtgtagctcaaataggtcatcatgaaaggttaaaaaagcgaggtggccatgttatgctggtggttaaggccagggcctctccaaccactgtgccactgacttgctgtgtgaccctgggcaagtcacttaactataaggtgcctcagttttccttctgttaaaatggggataataatactgacctacctcaaagggcagttttgaggcatgactaatgctttttagaaagcattttgggatccttcagcacaggaattctcaagacctgagtattttttataataggaatgtccaccatgaacttgatacgtccgtgtgtcccagatgctgtcattagtctatatggttctccaagaaactgaatgaatccattggagaagcggtggataactagccagacaaaatttgagaatacataaacaacgcattgccacggaaacatacagaggatgccttttctgtgattgggtgggattttttccctttttatgtgggatatagtagttacttgtgacaagaataattttggaataatttctattaatatcaactctgaagctaattgtactaatctgagattgtgtttgttcataataaaagtgaagtgaatctgattgcaaaaaa

SEQ ID NO: 2 - protein, 359 aa, Homo sapiens
MPAHLLQDDISSSYTTTTTITAPPSRVLQNGGDKLETMPLYLEDDIRPDIKDDIYDPTYKDKEGPSPKVEYVWRNIILMSLLHLGALYGITLIPTCKFYTWLWGVFYYFVSALGITAGAHRLWSHRSYKARLPLRLFLIIANTMAFQNDVYEWARDHRAHHKFSETHADPHNSRRGFFFSHVGWLLVRKHPAVKEKGSTLDLSDLEAEKLVMFQRRYYKPGLLMMCFILPTLVPWYFWGETFQNSVFVATFLRYAVVLNATWLVNSAAHLFGYRPYDKNISPRENILVSLGAVGEGFHNYHHSFPYDYSASEYRWHINFTTFFIDCMAALGLAYDRKKVSKAAILARIKRTGDGNYKSG

SEQ ID NO: 3 - DNA, 21 bp, Homo sapiens
uaucucuagcuccuauacctt

SEQ ID NO: 4 - DNA, 21 bp, Homo sapiens
gguauaggagcuagagauatt

SEQ ID NO: 5 - DNA, 21 bp, Homo sapiens
gaauggaggagauaaguugtt

SEQ ID NO: 6 - DNA, 21 bp, Homo sapiens
caacuuaucuccuccauuctt

SEQ ID NO: 7 - DNA, 21 bp, Homo sapiens
gaaugaugucuaugaauggtt

SEQ ID NO: 8 - DNA, 21 bp, Homo sapiens
ccauucauagacaucauuctt

SEQ ID NO: 9 - DNA, 21 bp, Homo sapiens
acacaugcugauccucauatt

SEQ ID NO: 10 - DNA, 21 bp, Homo sapiens
uaugaggaucagcaugugutt

SEQ ID NO: 11 - DNA, 21 bp, Homo sapiens
ucguccuuaugacaagaactt

SEQ ID NO: 12 - DNA, 21 bp, Homo sapiens
guucuugucauaaggacgatt

SEQ ID NO: 13 - DNA, 21 bp, Homo sapiens
gauaugcuguggugcuuaatt

SEQ ID NO: 14 - DNA, 21 bp, Homo sapiens
uuaagcaccacagcauauctt

SEQ ID NO: 15 - DNA, 21 bp, Homo sapiens
agaaugaugucuaugaaugtt

SEQ ID NO: 16 - DNA, 21 bp, Homo sapiens
cauucauagacaucauucutt

SEQ ID NO: 17 - DNA, 21 bp, Homo sapiens
cgacauucgcccugauauatt

SEQ ID NO: 18 - DNA, 21 bp, Homo sapiens
uauaucagggcgaaugucgtt

SEQ ID NO: 19 - DNA, 21 bp, Homo sapiens
ggaguacgcuagacuuguctt

SEQ ID NO: 20 - DNA, 21 bp, Homo sapiens
gacaagucuagcguacucctt

SEQ ID NO: 21 - DNA, 20 bp, Homo sapiens
agtgggccggcatcttggct

SEQ ID NO: 22 - DNA, 22 bp, Homo sapiens
aagtgggccggcatcttggctc

SEQ ID NO: 23 - DNA, 24 bp, Homo sapiens
caagtgggccggcatcttggctct

SEQ ID NO: 24 - DNA, 20 bp, Homo sapiens
tgggccggcatcttggctct

SEQ ID NO: 25 - DNA, 22 bp, Homo sapiens
gtgggccggcatcttggctctc

SEQ ID NO: 26 - DNA, 24 bp, Homo sapiens
agtgggccggcatcttggctctcg

SEQ ID NO: 27 - DNA, 20 bp, Homo sapiens
ggccggcatcttggctctcg

SEQ ID NO: 28 - DNA, 22 bp, Homo sapiens
gggccggcatcttggctctcgg

SEQ ID NO: 29 - DNA, 24 bp, Homo sapiens
tgggccggcatcttggctctcgga

SEQ ID NO: 30 - DNA, 20 bp, Homo sapiens
ccggcatcttggctctcgga

SEQ ID NO: 31 - DNA, 22 bp, Homo sapiens
gccggcatcttggctctcggat

SEQ ID NO: 32 - DNA, 24 bp, Homo sapiens
ggccggcatcttggctctcggatg